(19) United States
(12) Patent Application Publication
(10) Pub. No.: US 2024/0398948 A1
(43) Pub. Date: Dec. 5, 2024

(54) Targeted Cell Therapies

(71) Applicant: Bastion Therapeutics Limited, London (GB)

(72) Inventors: Timothy Newton, London (GB); Aleksandra Wlodek, London (GB)

(73) Assignee: Bastion Therapeutics Limited, London (GB)

(21) Appl. No.: 18/694,680

(22) PCT Filed: Sep. 23, 2022
(86) PCT No.: PCT/GB2022/052412

§ 371 Date: Mar. 22, 2024

(30) Foreign Application Priority Data
Sep. 24, 2021 (GB) 2113673.4

(51) Int. Cl. A61K 39/00; C07K 14/725; C07K 16/22; C07K 16/28

(52) U.S. Cl. CPC A61K 39/46432; A61K 39/4611; A61K 39/4631; A61K 39/4643; C07K 14/7051; C07K 16/22; C07K 16/2896; C07K 2317/622; C07K 2319/50

(57) ABSTRACT

The invention relates to an artificial T cell receptor, wherein an antigen binding domain of the artificial T cell receptor specifically binds a complement pathway protein, nucleic acids encoding such artificial T cell receptors and cells engineered to express such nucleic acids. The invention also relates to targeting polypeptides comprising an extracellular ligand binding domain and an intracellular domain comprising a transcription factor, and wherein the transcription factor is configured to be released upon binding of the ligand binding domain by a ligand. The invention also relates to cells engineered to express the artificial T cell receptor and the targeting polypeptide, particularly where expression of the artificial T cell receptor is operatively linked to binding of the ligand binding domain. The cells of the invention are useful in medicine, particularly in the treatment of inflammatory conditions.

### FIELD OF THE INVENTION

The present invention is in the field of artificial T cell receptors, such as chimeric antigen receptors, and cell therapies for inflammatory disease, in particular neuroinflammatory disease.

### BACKGROUND

Chimeric antigen receptors (CAR) are modified T-cell receptors (i.e. artificial T cell receptors) that are genetically engineered and typically expressed in T lymphocytes (T cells) to provide so called CAR-T cells. CAR-T cells are either made from a patient's own CD4+ or CD8+ T cells (autologous), or T cells from donors (allogeneic), which are engineered to express the CAR of interest and then (re)introduced into the patient as a therapy, typically for cancer. By targeting these modified T-cells to antigens expressed on the surface of cancer cells (such as CD19) it is possible to coax the immune system to attack and destroy cancer cells that were not previously, or not sufficiently, recognised by the immune system. CAR-T cells bind to and destroy cancer cells through several mechanisms. CAR-T cells mediate MHC-unrestricted cancer cell killing by enabling T cells to bind target cell surface antigens through a single-chain variable fragment (scFv) recognition domain. On binding, the CAR-T cell forms a non-classical immune synapse, required for their effector function. The cells then mediate their anti-tumour effects through the perforin and granzyme axis, the Fas and Fas ligand axis, as well as the release of cytokines to sensitize the tumour stroma. This strong pro-inflammatory mechanism has shown significant efficacy in treating a number of cancers. Typically, a CAR comprises an extracellular antigen recognition domain (usually a scFv fragment), a transmembrane domain (usually derived from CD28), and an intracellular domain that usually comprises several intracellular signalling domains derived from T cell receptors, which activate the T cell upon antigen binding. Due to the significant pro-inflammatory effect of CAR-T cells patients have to be monitored closely during therapy to manage the risk of over-stimulation of the inflammatory mechanisms (such as cytokine storms), a cause of significant patient morbidity and mortality. Anti-cytokine antibodies are usually kept on hand to dampen the immune response in patients at risk of such adverse events.

Inappropriate inflammation and auto-immune reactions where the patient's immune system attacks self-tissues are at the root of a range of different diseases that cause significant morbidity and mortality across the world. Inflammation of nervous tissue, so called neuro-inflammation, has been linked to a number of conditions, such as neurodegenerative diseases including Alzheimer's disease, Parkinson's disease, multiple sclerosis (MS) and motor neurone disease (amyotrophic lateral sclerosis or ALS). Neuro-inflammation can have a number of triggers, such as infection, traumatic brain injury, toxic metabolites, ageing or autoimmune reactions. Inappropriate inflammation in other tissues has been linked to diverse conditions such as Type I diabetes, rheumatoid arthritis and irritable bowel syndrome (IBS). Current treatments for inflammatory conditions typically involve systemic or local delivery of anti-inflammatory drugs, such as non-steroidal anti-inflammatory drugs, steroids and immune-suppressive drugs. While these can be effective to treat acute inflammation, longer term and more severe inflammatory conditions are not effectively treated with such drugs. The existing drugs lack precision and as such can have unwanted side effects, such as limiting a patient's ability to fight against infection. Further, existing drugs are often not sufficiently potent to address more severe inflammatory problems, at least not in doses that are safe for the patient.

Regulatory T cells (T reg cells), also known as suppressor T cells, are T cells that modulate the immune system, maintain tolerance to self-antigens, and can act to prevent autoimmune disease. CD4+T reg cells can supress the activity of effector T cells and they express the biomarkers CD4, FOXP3, and CD25. T reg therapy has shown some promise in providing an alternative to current pharmacological immunosuppressive therapies for treating inflammation-mediated disorders but their effect is short lived and generally insufficient to demonstrate therapeutic utility. Some groups have tried to improve persistence of T reg cells to improve treatment utility, for example, WO 2019/241549 describes T reg cells engineered to express human leukocyte antigen targeting CAR and FoxP3. WO 2019/190879 describes coupling T reg cells to CARs directed to glial cell markers, thereby suppressing CNS associated inflammation, as a potential treatment for neurodegenerative diseases.

Nevertheless, there exists a significant unmet need in the provision of effectively targeted and efficacious therapies for severe inflammatory conditions, particularly neuro-inflammatory and neurodegenerative conditions. In particular, there is a need to target the therapy to inappropriate inflammation whilst leaving health tissue immunocompetent.

The listing or discussion of an apparently prior-published document in this specification should not necessarily be taken as an acknowledgment that the document is part of the state of the art or is common general knowledge.

### SUMMARY OF THE INVENTION

Described herein are biological targeting systems and cell therapies for the treatment of inflammatory disease or disease with inflammatory etiologies or symptoms, in particular, neuro-inflammatory disease or neural disease with inflammatory etiologies or symptoms. The systems and cell therapies described address key problems in the treatment of neuroinflammatory disease specifically with regard to safety and specificity. The systems and cell therapies described introduce a dual-activation checkpoint, or an AND logic gating system to restrict the activity of the cell therapies to sites of inflammation and disease. This the targeting mechanism described herein is able to precisely direct an anti-inflammatory cell therapy to inflamed tissue to treat disease, whilst leaving healthy tissue immunocompetent.

The systems minimally comprise a targeting moiety that identifies a tissue specific target, a gene expression moiety which is activated upon the successful identification of the tissue-specific target and an effector moiety, activated through the conditional gene expression moiety, which identifies a disease-specific target and delivers a therapeutic effect such as modulation of a disease associated pathway.

The cell therapies described minimally comprise a transgenic T regulatory cell (T_{REG}) expressing a targeting polypeptide that targets a tissue and disease specific marker, and a nucleic acid that encodes an effector polypeptide. Expression of the effector polypeptide is controlled by binding of the targeting polypeptide to a tissue specific or disease specific marker. Further, the effector polypeptide is specific for an immune effector molecule that contributes to inflammation or inflammatory disease, and delivers the immune regulatory functions of the T_{REG} cell. Thus, the cell or the system is only activated when it comes into contact with the appropriate tissue, and even then, when the appropriate tissue possesses the hallmarks of disease or inflammation. Also disclosed herein, individually, are specific targeting moieties, which are useful for targeting cells to specific tissue spaces; and effector moieties, which are useful for binding molecules that are associated with disease. Particular targeting moieties are referred to herein as targeting polypeptides and particular effector moieties are referred to herein as artificial T cell receptors (TCR), which can for example be chimeric antigen receptors (CAR) or other engineered T cell receptors. Nevertheless, the present targeting mechanism could be used in multiple cell types, including (but not limited to) regulatory T cells (T regs), mesenchymal stem cells, or cells which could be differentiated into T regs.

Accordingly, in a first aspect the present invention provides an artificial T cell receptor, wherein an antigen binding domain of the artificial T cell receptor specifically binds a complement pathway protein. In a preferred embodiment, the artificial T cell receptor is a chimeric antigen receptor (CAR).

In an embodiment, the antigen binding domain comprises an antibody fragment or derivative thereof. In a further embodiment the antibody fragment comprises a fragment selected from the group comprising Fab, Fab′, F(ab′)₂, Fv, scFv, disulfide-linked Fvs (sdFv), Fd, linear antibodies, and single domain antibodies. In a preferred embodiment the antigen binding domain comprises an scFv antibody fragment.

In an embodiment, the complement pathway protein is selected from the group comprising C1q, C1 r, C1s, C2a, C3, C3a, C3b, C4, C4a, C4b, C5, C5a, C5b, C6, C7, C8, and C9. In a preferred embodiment the complement pathway protein is C1q.

In an embodiment, the artificial T cell receptor comprises an intracellular signalling domain comprising an intracellular signalling domain of CD3zeta, CD28, ICOS, OX-40, or a combination thereof.

In a second aspect, the present invention provides a nucleic acid encoding the artificial T cell receptor according to the invention. In an embodiment the nucleic acid is operatively linked to a transcriptional regulatory sequence, and the transcriptional regulatory sequence is configured to bind a transcription factor. In an embodiment the transcriptional regulatory sequence configured to bind the transcription factor comprises a binding domain for Gal4-VP6, tetR-VP64 (tTA), ZFHD1-VP64, Gal4-KRAB, PIP-VP64, ZF21-16-VP64, ZF43-8-VP64 or FoxP3. In a particularly preferred embodiment the transcriptional regulatory sequence comprises a binding domain for FoxP3.

In a third aspect, the present invention provides a targeting polypeptide, wherein the targeting polypeptide comprises an extracellular domain, a transmembrane domain, and an intracellular domain, wherein the extracellular domain comprises a ligand binding domain and wherein the intracellular domain comprises a transcription factor, and wherein the transcription factor is configured to be released upon binding of the ligand binding domain by a ligand. In a preferred embodiment the extracellular domain and the intracellular domain are heterologous to the transmembrane domain. In a preferred embodiment, the transcription factor is released by proteolytic cleavage.

In an embodiment of the third aspect, the transmembrane domain comprises a notch minimal regulatory region or a notch extended regulatory region.

In a further embodiment of the third aspect, the intracellular domain comprises a cleavage domain configured to be cleaved by a protease. In a preferred embodiment the protease is a type II serine protease. In a particularly preferred embodiment, the intracellular domain comprises a cleavage domain configured to be cleaved by a type II serine protease, a type II serine protease domain comprising a catalytically active region of a serine protease, an inhibitory domain comprising an amino acid sequence that inhibits the catalytically active region of the type II serine protease when the ligand binding domain is not bound by a ligand, and the transcription factor. In a preferred embodiment the catalytically active region of the serine protease comprises an active domain of thrombin, Hepatitis C virus Ns3 serine protease, or a TVMV protease.

In a further embodiment of the third aspect, the ligand binding domain comprises an amino acid sequence that specifically reacts with a benzyl guanine derivative or an O2-benzylcytosine (BC) derivative. In an embodiment the ligand binding domain comprises a SNAP-Tag or a CLIP-Tag.

In a fourth aspect, the present invention provides a targeting polypeptide, wherein the targeting polypeptide comprises a ligand binding domain, a transmembrane domain, and a transcription factor, wherein the transmembrane domain is located between the ligand binding domain and the transcription factor, and wherein the transcription factor is cleavably linked to the transmembrane domain. In a preferred embodiment, the transmembrane domain and the transcription factor are linked by a cleavable peptide linker. In a preferred embodiment, the cleavable linker comprises at least one self-cleaving peptide. The at least one self-cleaving peptide may comprise a 2A self-cleaving peptide. In a preferred embodiment, the 2A self-cleaving peptide comprises a P2A peptide, an E2A peptide, an F2A peptide, and/or a T2A peptide, or tandem or triple arrangements of such peptides.

In a preferred embodiment of any targeting polypeptide according to the invention the ligand binding domain specifically binds a tissue-associated antigen. The tissue-associated antigen may be a tissue specific marker. In an embodiment, the tissue-associated antigen is a neuronal marker present at a neuronal synapse. In a preferred embodiment, the neuronal marker present at a neuronal synapse is a neuronal antigen. In a particularly preferred embodiment the neuronal antigen is neurexin or neuroligin. Thus, in a preferred embodiment, the ligand binding domain comprises a neurexin polypeptide or a neuroligin binding fragment of the neurexin polypeptide. In an embodiment, the ligand binding domain comprises an amino acid sequence at least about 90%, 95%, 97%, 98%, 99%, or 100% identical to the amino acid sequence set forth in SEQ ID NO: 1. In an embodiment, the neurexin polypeptide or a neuroligin binding fragment thereof comprises an amino acid variant that reduces binding to neuroligin compared to a wild-type neurexin polypeptide or neuroligin binding fragment thereof. In an embodiment, the amino acid variant is selected from the group comprising S111A, D162A, I210A, N212A, I210A:D141A, and combinations thereof. In preferred embodiments, the ligand binding domain comprises an amino acid sequence at least about 90%, 95%, 97%, 98%, 99%, or 100% identical to the amino acid sequence set forth in any one of SEQ ID NOs: 2 to 6. It is particularly preferred that the targeting polypeptide comprises an amino acid sequence at least about 90%, 95%, 97%, 98%, 99%, or 100% identical to the amino acid sequence set forth in any one of SEQ ID NOs: 14 to 19. In an alternative embodiment, the ligand binding domain comprises an amino acid sequence that is at least about 90%, 95%, 97%, 98%, 99%, or 100% identical to the amino acid sequence set forth in SEQ ID NO: 7.

In an alternative embodiment of the targeting polypeptides of the invention, the tissue-associated antigen is an antigen associated with: i) inflammatory bowel disease, such as Carcinoembryonic antigens, GLUT2, or GLUT5; ii) Rheumatoid arthritis, such as Type II Collagen or Citrullinated vimentin; or iii) Type 1 diabetes, such as Insulin or pro-Insulin.

In an embodiment of any targeting polypeptide of the invention, the transcription factor is heterologous to the extracellular domain, the transmembrane domain and/or the rest of the intracellular domain. In an embodiment, the transcription factor is selected from the group comprising Gal4-VP6, tetR-VP64 (tTA), ZFHD1-VP64, Gal4-KRAB, PIP-VP64, ZF21-16-VP64, ZF43-8-VP64, LAIR2, METTL7A, RTKN2, FoxP3, BACH2, Cish, ZEB2, EOMES, ZNF683 (Hobit), AML1, ReIA, RORγt, TIP60/HDAC7, STAT3, IRF4, USP7, LEF1, GATA-1, GATA-3, and STAT5. In a preferred embodiment, the transcription factor is FoxP3.

In a fifth aspect, the present invention provides a nucleic acid encoding the targeting polypeptide according to the invention. In an embodiment, the nucleic acid is operatively linked to a transcriptional regulatory sequence, and the transcriptional regulatory sequence is configured to bind a transcription factor. In an embodiment, the transcriptional regulatory sequence configured to bind the transcription factor comprises a binding domain for Gal4-VP6, tetR-VP64 (tTA), ZFHD1-VP64, Gal4-KRAB, PIP-VP64, ZF21-16-VP64, ZF43-8-VP64 or FoxP3. In a preferred embodiment, the transcriptional regulatory sequence comprises a binding domain for FoxP3.

In a sixth aspect, the present invention provides a cell engineered to express a nucleic acid encoding a targeting polypeptide, wherein the targeting polypeptide comprises a ligand binding domain, wherein the cell further comprises a nucleic acid encoding an artificial T cell receptor, and wherein an antigen binding domain of the artificial T cell receptor specifically binds a biomarker. In an embodiment, the nucleic acid encoding the artificial T cell receptor is operatively linked to a transcriptional regulatory sequence, and the transcriptional regulatory sequence is configured to bind a transcription factor. In an embodiment, the ligand binding domain specifically binds a tissue-associated antigen. In an embodiment, the tissue-associated antigen is a tissue specific marker. In a preferred embodiment, the targeting polypeptide is a targeting polypeptide according to the third or fourth aspects of the invention. In an embodiment, the biomarker is a biomarker of inflammation, an inflammatory mediator, and/or a disease-associated biomarker. In an embodiment, the biomarker of inflammation is a complement pathway protein. In an embodiment, the complement pathway protein is selected from the group comprising C1q, C1r, C1s, C2a, C3, C3a, C3b, C4, C4a, C4b, C5, C5a, C5b, C6, C7, C8, and C9. In a preferred embodiment, the complement pathway protein is C1q.

In a seventh aspect, the present invention provides a cell comprising: i) the nucleic acid according to the second aspect; and ii) the nucleic acid according to the fifth aspect. In an embodiment, the nucleic acid encoding the targeting polypeptide comprises a constitutively active promoter or enhancer operatively coupled to the coding region for the targeting polypeptide. In a further embodiment, the transcriptional regulatory sequence that is operatively linked to the nucleic acid encoding the artificial T cell receptor is configured to bind the same transcription factor that is cleavably linked to the ligand binding domain of the targeting polypeptide. In a preferred embodiment, release of the transcription factor from the targeting polypeptide activates expression of the artificial T cell receptor. In an embodiment, the transcription factor is selected from the group comprising Gal4-VP6, tetR-VP64 (tTA), ZFHD1-VP64, Gal4-KRAB, PIP-VP64, ZF21-16-VP64, ZF43-8-VP64 and FoxP3. In a preferred embodiment, the transcription factor is FoxP3.

In an embodiment of the seventh aspect, the cell is an immune cell. In a preferred embodiment, the immune cell is a T lymphocyte. In a most preferred embodiment, the T lymphocyte is a regulatory T lymphocyte (T reg).

In an alternative embodiment of the seventh aspect, the cell is a mesenchymal stem cell. In a preferred embodiment, the mesenchymal stem cell is a type II mesenchymal stem cell or an adipose-derived stem cell.

In a further alternative embodiment of the seventh aspect, the cell is a CD34+ stem cell or an induced pluripotent stem cell.

In an eighth aspect, the present invention provides a pharmaceutical composition comprising a cell according to the sixth or seventh aspects, further comprising a pharmaceutically acceptable carrier, diluent, or excipient. In an embodiment, the pharmaceutical composition is formulated for intravenous injection.

In a ninth aspect, then present invention provides a cell according to the sixth or seventh aspects, or a pharmaceutical composition according to the eighth aspect, for use in medicine.

In an embodiment, the present invention provides the cell or pharmaceutical composition for use according to the ninth aspect, wherein the cell or pharmaceutical composition is for use in treating an inflammatory disorder in a subject. In an embodiment, the cell or pharmaceutical composition is for use in treating an inflammatory disorder of the nervous system. It is envisaged that the inflammatory disorder of the nervous system may be selected from the group comprising multiple sclerosis, chronic inflammatory demyelinating polyneuropathy, an encephalitis, a traumatic brain injury, myasthenia gravis, and amyotrophic lateral sclerosis. In particular, the cell or pharmaceutical composition is for use in treating amyotrophic lateral sclerosis.

In a preferred embodiment of the uses provided herein, the cell is either autologous to the subject, or allogenic to the subject.

In a tenth aspect, the present invention provides a nucleic acid vector comprising the nucleic acid according to the second aspect, and/or the nucleic acid according to the fifth aspect.

In an embodiment, the nucleic acid vector is: i) a viral vector, preferably a retroviral vector, an adenoviral vector or an adeno-associated viral vector; ii) a non-polymeric vector, preferably a liposome or a gold nanoparticle; or iii) a polymeric vector, preferably a dendrimer, a dendrigraft, a polymeric micelle or a poly(β-amino ester) vector. In an embodiment, the nucleic acid vector may be delivered as a transposon, such as a PiggyBack or Sleeping Beauty transposon. In an embodiment, the nucleic acid vector may be a plasmid flanked by regions for homologous recombination for CRISPR/Cas type knock-in, for example Cas9.

In an eleventh aspect, the present invention provides a method of making a cell according to the sixth or seventh aspects comprising contacting a cell with: i) the nucleic acid according to the second aspect; ii) the nucleic acid according to the fifth aspect; and/or iii) the nucleic acid vector according to the tenth aspect.

In a twelfth aspect, the present invention provides a biological targeting system comprising a) a targeting polypeptide comprising a domain that specifically binds to a tissue specific marker; b) an effector polypeptide wherein the effector polypeptide specifically binds a disease specific antigen or an immune effector molecule; and c) a cargo selected from an: extracellular vesicle, a protein-coated vesicle, a liposome, a dendrimer, a micelle, a biodegradable particle comprising P-selectin, endothelial selectin (E-selectin) and ICAM-1, an artificial nanostructure, an engineered viral particle, a bacterial cell, a transposon such as a PiggyBack or Sleeping Beauty transposon, and a plasmid flanked by regions for homologous recombination for CRISPR/Cas type knock-in, for example Cas9.

In an embodiment, the targeting polypeptide of the twelfth aspect is any targeting polypeptide described herein, such as those of the third and fourth aspects. In an embodiment, the effector polypeptide of the twelfth aspect is a artificial T cell receptor as described herein, such as that of the first aspect.

In a thirteenth aspect, the present invention provides a biological targeting system according to the twelfth aspect for use in medicine. In an embodiment, the biological targeting system is for use in treating an inflammatory disorder in a subject. In an embodiment, the inflammatory disorder is an inflammatory disorder of the nervous system. In a preferred embodiment the inflammatory disorder of the nervous system is selected from the group comprising multiple sclerosis, chronic inflammatory demyelinating polyneuropathy, an encephalitis, a traumatic brain injury, myasthenia gravis, and amyotrophic lateral sclerosis. In a particularly preferred embodiment, the inflammatory disorder of the nervous system is amyotrophic lateral sclerosis.

In a fourteenth aspect, the present invention provides a method of treating an inflammatory disorder in an individual in need thereof comprising administering to the individual in need thereof the cells, compositions, and systems described herein. Preferred inflammatory disorders are those described herein. In certain embodiments, the cell is autologous to the individual in need thereof. In certain embodiments, the cell is allogenic to the individual in need thereof. In certain embodiments, the cell is characterized by an elimination half-life of greater than about 45 days. In certain embodiments, the method comprises administering a second dose of the cell according to the invention.

### BRIEF DESCRIPTION OF FIGURES

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A illustrates a non-limiting embodiment of the mechanism of action of a targeting polypeptide.

FIG. 1B illustrates the mechanism of induction of a C1q CAR by a transcription factor liberated from a targeting polypeptide of the disclosure.

FIG. 2 shows a Western blot of expression of WT targeting polypeptide that comprise a neurexin targeting domain. WTA, WTB, WTC and WTD represent transfection optimization conditions. WTE and WTF are non-transfected negative controls

FIG. 3 shows induction of luciferase activity of targeting polypeptides expressed in HEK293 cells that have a luciferase gene under the control of the GAL4 response element.

FIG. 4 shows a comparison of the mechanisms of action of a SynNotch targeting polypeptide mediated system described herein with a SynNotch independent targeting polypeptide mediated system described herein. Thus, in the SynNotch dependent system Neuroligin binding could trigger transcription factor release by the targeting polypeptide, or in the SynNotch independent system transcription factor release could be constitutive.

FIG. 5 shows that C1q-coated tosyl-activated beads stimulate CD69 expression in Tregs by interacting with the constitutively expressed CAR of the invention.

FIG. 6 shows that binding of neuroligin coated beads to SynNotch receptor in T reg cells transfected with FLAG-tagged CAR gene, drives expression of CAR that is detectable by anti-FLAG antibody.

FIG. 7 shows that binding of neuroligin coated beads to SynNotch receptor in T reg cells transfected with C1q CAR gene, drives expression of CAR and the CAR is activated by C1q, detected by determination of CD69 expression.

FIG. 8 shows a comparison of the tissue location of SynNotch Tregs and SynNotch independent (neurexin tether combined with FoxP3 via P2A site) T regs in SOD1 mice (model animal for ALS).

FIG. 9 shows a comparison of the efficacy of SynNotch independent T regs in a murine experimental autoimmune encephalomyelitis (EAE) study.

FIG. 10 shows the open reading frame arrangement of the SynNotch construct according to an embodiment of the invention represented by SEQ ID NO: 14.

FIG. 11 shows a multiple sequence alignment of the neurexin fragments used in the examples represented by SEQ ID NOs: 1-6.

FIG. 12 shows a flow cytometry comparison of phenotypic Treg regulator, FoxP3 and CAR levels in human Tregs under normal conditions and under pro-inflammatory conditions. 1) Tregs have naturally high FoxP3 levels, but a significant fraction can lose FoxP3 expression. 2) Tregs with Reflection technology (RT) according to an embodiment of the present invention (here a tissue tether-P2A-FOXP3 transcript, under the control of a constitutive promoter, which triggers the expression of an α-C1q CAR under the control of FOXP3 response elements) have consistently high FoxP3 levels across the population and express high levels of Chimeric Antigen Receptor (CAR). 3) Tregs lose FoxP3 expression when exposed to pro-inflammatory cytokines, which can cause them to lose their anti-inflammatory properties. 4) Tregs with according to the invention are resistant to pro-inflammatory cytokines and maintain high CAR expression.

### DETAILED DESCRIPTION

The present targeting technology employs two receptors. The first receptor detects a tissue antigen, targeting cells to a tissue of interest. In a preferred embodiment this receptor targets Neuroligin, a receptor found on the post-synaptic membrane, to direct anti-inflammatory cells to neurons and neuromuscular junctions. The second receptor, an artificial T cell receptor such as a Chimeric Antigen Receptor (CAR) which is linked to the first via transcriptional regulation, targets an inflammatory antigen, in a preferred embodiment C1q. The combination of these two receptors targets anti-inflammatory cells to inflamed parts of an organ system, rather than to the whole organ, or to any inflamed area. A key advantage of the presently described system is that expression of the first receptor leads to expression of the second receptor via a transcription factor, allowing the anti-inflammatory activity to be restricted to target tissue. CAR T cells have previously been directed to single antigens (e.g. CD19 in treating cancer) or multiple cell surface antigens to improve specificity but the presently described combination of tissue-specific targeting with one receptor and diagnostic marker targeting with another receptor has not been previously described or contemplated. The targeting of a diagnostic marker of inflammation (e.g. C1q) does not need to be characteristic of a specific disease, but rather a sign that a particular tissue is suffering from inflammation. Targeting a membrane associated antigen (e.g. C1q) as opposed to a fixed receptor, represents a significant departure from previous approaches, as other CAR-T programs target receptors on the cell surface. In general it is known, a torque is required to trigger CARs- and that is normally met by membranous proteins, i.e. proteins that are anchored to the membrane. The C1 complex is only membrane associated when activated. The present inventors have surprisingly found that using a CAR to target a protein that is only associated with a membrane, provides sufficient torque to activate the CAR upon binding.

Further, it is also counter intuitive to target complement, such as C1q, as a ‘diagnostic marker’ of disease with a CAR T cell. As these proteins are widely expressed throughout the body, they are not an obvious target for CAR, but when combined with the targeting aspects of the present invention in an ‘AND’ gate configuration they become a powerful system for precisely directing anti-inflammatory activity to inflamed tissues. Neither Neuroligin/neurexin nor complement are indicative of ALS on their own, but in combination they highlight an inflamed neuron and neuroinflammation, which provides significant promise for treating ALS.

In one aspect, described herein is a mammalian cell comprising: (a) a targeting nucleic acid wherein the targeting nucleic acid comprises a coding region for a targeting polypeptide comprising: (i) an extracellular domain, wherein the extracellular domain specifically binds to a tissue specific marker; (ii) a transmembrane domain; and (iii) an intracellular domain comprising a transcription factor heterologous to the extracellular domain or the transmembrane domain; and (b) an effector nucleic acid, wherein the effector nucleic acid comprises: (i) a transcriptional regulatory sequence configured to be bound by the transcription factor heterologous to the extracellular domain; and (ii) an effector coding region operatively coupled to the transcriptional regulatory sequence configured to be bound by the transcription factor heterologous to the extracellular domain; wherein the effector coding region encodes a polypeptide that specifically binds an immune effector molecule.

In another aspect described herein is a targeting polypeptide, comprising an extracellular domain, a transmembrane domain, and an intracellular domain, wherein the extracellular domain comprises a ligand binding domain and wherein the intracellular domain comprises a transcription factor, wherein the transcription factor is configured to be released upon binding of the ligand binding domain by a ligand.

In another aspect described herein is a targeting polypeptide, comprising a ligand binding domain, a transmembrane domain, and a transcription factor, wherein the transmembrane domain is located between the ligand binding domain and the transcription factor, and wherein the transcription factor is cleavably linked to the transmembrane domain, preferably with at least one self-cleaving peptide.

In another aspect described herein is an artificial T cell receptor, in particular a chimeric antigen receptor, wherein an antigen binding domain of the artificial T cell receptor specifically binds a complement pathway protein. In certain embodiments, the complement pathway protein comprises C1q, C1r, C1s, C2a, C3, C3a, C3b, C4, C4a, C4b, C5, C5a, C5b, C6, C7, C8, or C9. In certain embodiments, the complement pathway protein comprises C1q. In a preferred embodiment, the artificial T cell receptor is a CAR or other engineered T cell receptor.

In another aspect described herein is a biological targeting system comprising a) a targeting polypeptide comprising a domain that specifically binds to a tissue specific marker; b) an effector polypeptide wherein the effector polypeptide specifically binds an immune effector molecule; and c) and a cargo selected from an: extracellular vesicle, a protein-coated vesicle, a liposome, a dendrimer, a micelle, a biodegradable particle comprising P-selectin, endothelial selectin (E-selectin) and ICAM-1, an artificial nanostructure, an engineered viral particle, a plasmid, a transposon, and a bacterial cell.

### Certain Definitions

In the present description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

As used herein a “therapeutic amount” is a dosage amount of a therapeutic intended to produce one or more beneficial effects useful for treating a condition for which the compounds and cells are provided. Some specific therapeutic amounts are discussed in detail herein.

As used herein “treating” or “treatment” refers to the intervention in a disease state intended to produce one or more beneficial effects. For neurodegenerative disease treatment includes methods that are intended to cause, or do cause, stable disease, partial response, complete response, extension of progression-free survival, extension of overall survival, improvement of numbness, improvement of paralysis, memory loss slow-down, delay in memory loss, delay in tremor progression or a prevention or reduction in neurodegeneration, delay in limb strength loss, delay of weakness, delay of atrophy. In certain cases, the therapeutic methods described herein may be used as maintenance after successful treatment or to prevent recurrence or relapse. It is understood that not all individuals will respond to the same degree, or at all, to a given administration of therapeutic cell therapy, however even if no response is detected these individuals are nonetheless considered to have been treated.

The terms “polypeptide” and “protein” are used interchangeably to refer to a polymer of amino acid residues, and are not limited to a minimum length. Polypeptides, including the provided antibodies and antibody chains, receptors and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, the term “receptor” or “receptors” refer to protein molecules inside the target cell or on its surface that receive a chemical signal.

As used herein, the term “tether”, refers to a polypeptide chain expressed upon the cell surface with affinity for a target sufficient to ‘link’ the cell to a structure containing the target. This structure could be another cell, extracellular matrix, bone, cartilage, tissue, or an artificial surface. In certain embodiments, the tether can be expressed on the cell's surface and link the cell for an extended amount of time to a tissue where the protein is present. In certain embodiments, the tether can be exogenously introduced to the surface of a cell, and link the cell to a target protein. In certain embodiments, the tether will be used as an anchor between one entity and another one.

As used herein, the term “heterologous” refers to a nucleotide or amino acid sequence that is from a different source (e.g., gene, polypeptide, or organism) compared to the amino acid or nucleotide sequence to which it refers to as being heterologous. Heterologous includes biological sequences derived from different organisms or to sequences derived from different sources (e.g., genes or proteins) of the same organism. Heterologous sequences include recombinant DNA molecules comprising nucleotide sequences from different sources, fusion proteins comprising amino acid sequences from different sources, and epitope or purification tags of natural or synthetic origin.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.OD. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “artificial T cell receptor” refers to any T cell receptor that has either been modified from an naturally occurring T cell receptor, for example by mutation, or that has been engineered so as to have substantially the same properties of an artificial T cell receptor or CAR. The term “Chimeric Antigen Receptor” or “CAR” is used to refer generally to any engineered T cell receptor and refers in particular to a polypeptide or set of polypeptides which when in an immunomodulatory cell, provides the cell with specificity for a target location or molecule, for example, an inflamed synapse, and with intracellular signal generation. The term “artificial T cell receptor” herein is intended to include and encompass CAR as defined herein as the preferred embodiment, although other forms of artificial T cell receptor are also envisaged and included as embodiments of the invention, as would be understood by a person of skill in the art. In some embodiments, a CAR comprises at least an extracellular antigen binding domain, a transmembrane domain and a cytoplasmic signalling domain (also referred to herein as “an intracellular signalling domain”) comprising a functional signalling domain derived from a stimulatory molecule and/or costimulatory molecule as defined below. An antigen binding domain can suitably be derived from an antibody, an antibody fragment, the VH or VL chain of an antibody or any one or more CDRs associated with any one or more VH or VL chains, and in certain cases all six CDRs derived from an antibody molecule. In certain embodiments, the antigen binding domain of the CAR comprises an scFv derived from an antibody of a known and useful specificity. In some embodiments, the antigen binding domain of the artificial T cell receptor or CAR comprises a scFv derived from an isolated antibody that specifically binds to a C1q protein. For example, the antigen binding domain may be derived from a humanized version of antibody M1, described in WO/2016/073685, the teaching of which is incorporated herein by reference. An example humanized M1 has a light chain variable domain comprising the sequence of SEQ ID NO: 41 and a heavy chain variable domain comprising the sequence of SEQ ID NO: 42. Accordingly, the antibody fragment may comprise a sequence according to either SEQ ID NO: 41 or 42 or a fragment thereof. Preferably, the antigen binding fragment may comprise one or more, preferably all three, of the hyper variable sequences derived from SEQ ID NO: 41 or 42, which are underlined in the sequence table at the end of the examples of the present application and designated as SEQ ID Nos: 60 to 65. Thus, the antigen binding fragment of the artificial T cell receptor of any embodiment of the invention may comprise one, two or all three of SEQ ID NO: 60, SEQ ID NO: 61 and SEQ ID NO: 62; and/or one two or all three of SEQ ID NO: 63, SEQ ID NO: 64 and SEQ ID NO: 65. The hypervariable/complementarity determining regions may be determined according to the method of Chothia, Kabat, or IMGT. In some embodiments that may be combined with any of the preceding embodiments, the artificial T cell receptor comprises a light chain variable domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 41. In some embodiments that may be combined with any of the preceding embodiments, the artificial T cell receptor comprises a heavy chain variable domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 42. In some embodiments, the polypeptide or set of polypeptides are in the same polypeptide chain (e.g., comprise a chimeric fusion protein). In some embodiments, the polypeptide or set of polypeptides are not contiguous with each other, e.g., are in different polypeptide chains. In some embodiments, the polypeptide or set of polypeptides include a dimerization switch that, upon the presence of a dimerization molecule, can couple the polypeptides to one another, e.g., can couple an antigen binding domain to an intracellular signalling domain. In one embodiment, the stimulatory molecule of the CAR is the zeta chain associated with the T cell receptor complex. In one aspect, the cytoplasmic signalling domain comprises a primary signalling domain (e.g., a primary signalling domain of CD3-zeta). In one embodiment, the cytoplasmic signalling domain further comprises one or more functional signalling domains of at least one costimulatory molecule as defined below. In one embodiment, the costimulatory molecule is a costimulatory molecule described herein, e.g., CD27, ICOS, and/or CD28. In one embodiment, the CAR comprises a chimeric fusion protein comprising an extracellular antigen binding domain, a transmembrane domain and an intracellular signalling domain comprising a functional signalling domain of a stimulatory molecule. In one embodiment, the CAR comprises a chimeric fusion protein comprising an extracellular antigen binding domain, a transmembrane domain and an intracellular signalling domain comprising a functional signalling domain of a co-stimulatory molecule and a functional signalling domain of a stimulatory molecule. In one embodiment, the CAR comprises a chimeric fusion protein comprising an extracellular antigen binding domain, a transmembrane domain and an intracellular signalling domain comprising two functional signalling domains of one or more co-stimulatory molecule(s) and a functional signalling domain of a stimulatory molecule. In one embodiment, the CAR comprises a chimeric fusion protein comprising an extracellular antigen binding domain, a transmembrane domain and an intracellular signalling domain comprising at least two functional signalling domains of one or more co-stimulatory molecule(s) and a functional signalling domain of a stimulatory molecule. In one embodiment, the CAR comprises an optional leader sequence at the amino-terminus (N-terminus) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. The artificial T cell receptors/chimeric antigen receptors or targeting polypeptides described herein can be encoded by a nucleic acid for delivery to the cell of an individual to be treated. In an embodiment, the artificial T cell receptor or chimeric antigen receptor of the invention may be assembled in the cell, rather than expressed as a fusion protein in the cell. For example, the chimeric antigen domain expressed in the cell may comprise an extracellular domain, a transmembrane domain and an intracellular domain, where the extracellular domain comprises an amino acid sequence that specifically reacts with a benzyl guanine (BG) derivative or an O2-benzylcytosine (BC) derivative. In an embodiment the extracellular domain may comprise a SNAP-Tag or a CLIP-Tag. The complement binding functionality of the chimeric antigen receptor in this embodiment may be provided by a BG or BC conjugated antibody that specifically reacts with the complement target, and which is attached to the extracellular domain post-translationally. As the skilled person would appreciate, other variations on this system are possible and are intended to be encompassed by the terms “artificial T cell receptor” or “chimeric antigen receptor”. Thus, as indicted above in the context of this application, the term “chimeric antigen receptor” is intended to mean any polypeptide, or set of polypeptides, which when in an immunomodulatory cell, provides the cell with specificity for a target location or molecule.

The term “biomarker of inflammation” as used as used herein, refers to any molecule or cell that is associated with a pro-inflammatory state. This could include pro-inflammatory cytokines (such as interleukin-1 (IL-1), IL-6, IL-12, and IL-18, tumor necrosis factor alpha (TNF-α), interferon gamma (IFNγ), and granulocyte-macrophage colony stimulating factor (GM-CSF)), Neurofilament Light chain (Nf-L), complement proteins as described herein, antibodies, T helper cells, cytotoxic T cells, Natural Killer T cells, gamma/delta T cells, macrophages, dendritic cells, bacterial or viral antigens, particles or cells, allergens and any other molecule or cell that can promote inflammation.

The term “antibody,” as used herein, refers to a protein, or polypeptide sequence which specifically binds a target molecule. The antibody may be derived from an immunoglobulin molecule or otherwise designed. The target molecule may comprise a protein, polypeptide, carbohydrate, or lipid.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulphide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulphide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies). The term “scFv” or “single chain variable fragment” refers to a single-domain antibody-like construct. The ScFv may be a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. The portion of a CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one embodiment, the antigen binding domain of a CAR comprises an antibody fragment. In a further embodiment, the CAR comprises an antibody fragment that comprises an scFv.

A polynucleotide is a type of nucleic acid comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. Vectors capable of directing the expression of genes to which they are operatively linked are referred to herein as “expression vectors.” Suitable vectors comprise plasmids, bacterial artificial chromosomes, yeast artificial chromosomes, viral vectors, transposons and the like. In the expression vectors regulatory elements such as promoters, enhancers, polyadenylation signals for use in controlling transcription can be derived from mammalian, microbial, viral or insect genes. The expression vectors described herein have one or more promoters or enhancers operatively coupled to a polypeptide to be expressed by the expression vector. In certain embodiments, the promoters are selectively inducible either by the administration of an agent to an individual that has been administered the vector, or in response to a biological stimulus, such as the liberation of transcription factor that can bind the promoter. One example of an inducible system is the Tet-On system, which utilizes the rtTA (reverse tetracycline-controlled transactivator) of Gossen et al. See Gossen M et al., “Transcriptional activation by tetracyclines in mammalian cells.” Science. 1995 Jun. 23; 268 (5218):1766-9. The expression vectors described herein can be replicated in a host to produce a sufficient amount of expression vector to administer to an individual or to transduce cells from an individual. The ability to replicate in a host, can be conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed as expression vectors. In certain embodiments, the viral vector is a gamma retrovirus. Viral expression vectors comprising nucleic acids encoding the molecules described herein can be generated in cell culture by the transfection of one or more plasmids that comprise the nucleic acid of interest, a packaging plasmid, and an envelope plasmid. Exemplary systems for lentivirus production are described in Dull T et al, “A Third Generation Lentivirus Vector with a Conditional Packaging System.” J Virol. 1998. 72(11):8463-8471; or Dull T. et al., “Self-Inactivating Lentivirus for Safe and Efficient In Vivo Gene Delivery.” J Virol. 1998. 72(12): 9873-9880. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors that are for genomic integration can target several safe landing sites such as the AAVS1 gene in humans or another animal.

As used herein, the terms “homologous,” “homology,” or “percent homology” when used herein to describe an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

In certain embodiments, described herein, are cells in frozen form after transduction with vectors comprising a targeting and/or an immune effector polypeptide. The cells can be provided in a suitable vial such as a cryovial or other vessel capable of withstanding temperatures down to at least about −80° C. In certain embodiments, the cryoprotectant comprises glycerol, DMSO or a combination thereof. In certain embodiments, the frozen cells are contained in a suitable vial or container able to withstand freezing by liquid nitrogen.

As used herein, the term “T reg phenotype” refer to a general set of functions and behaviours of a cell exerting T reg capabilities. In some embodiments, it is FoxP3 expression. In some embodiments, it is anti-inflammatory cytokine production. In some embodiments, it is T cell suppression. In some embodiments, it is a regenerative function.

### Therapeutic Targeting Systems

Described herein are systems of specific targeting polypeptides and immune effector molecules. Nucleic acids encoding the targeting polypeptides and immune effector molecules are introduced to a cell that is useful for treating a neuroinflammatory or an autoimmune disease, or an inflammatory condition in the body. Such cells are suitably any cell according to the claims, such as T cells, regulatory T cells, mesenchymal stem cells, and type II mesenchymal stem cells.

The cell systems described herein are suitably manufactured from an immune cell population, an immune cell population with regulatory characteristics, a T cell population, T cell precursors, or a stem cell population. In one embodiment, the cell systems described herein are manufactured from regulatory T cells (T regs). T regs are a specialized subpopulation cells that act to suppress an immune response, thereby maintaining homeostasis and self-tolerance. It has been shown that T regs are able to inhibit T cell proliferation and cytokine production and play a critical role in preventing autoimmunity and inflammation. Different subsets with various functions of T reg cells exist. T regs can be identified by flow cytometry among other methods. One marker of T reg cells is positivity for the FoxP3 transcription factor. Selected surface markers such as CD25high and CD127low can also serve as surrogate markers to detect T regs.

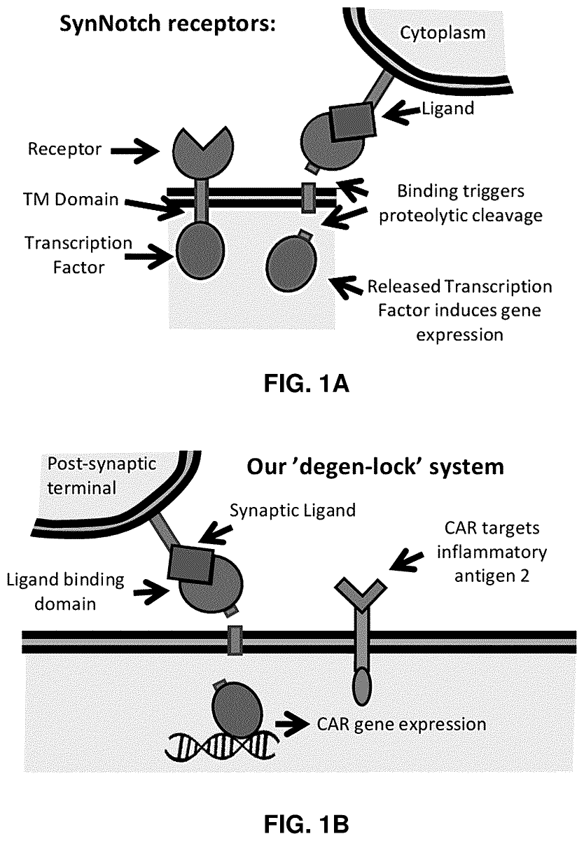

Referring to FIG. 1A, in a non-limiting embodiment a targeting polypeptide comprises an extracellular receptor, a transmembrane domain (TM), and an intracellular domain that comprises a transcription factor. Upon binding of the receptor to an appropriate target cell the transcription factor is released. The released transcription factor may then induce expression of a gene of interest. Referring to FIG. 1B, in “degen-lock” the gene of interest is a chimeric antigen receptor. This chimeric antigen receptor can target and bind an immune effector protein, such as those that participate in pro-inflammatory responses. Upon binding the CAR transduces a signal that activates the immunomodulatory function of the T cell. T regulatory cells for example, exert an immunomodulatory function by the release of the immunosuppressive cytokines IL-10 and TGFβ. The suppressive action of the Treg cell reduces inflammation in the tissue area surrounding the T cell. Such a system may have an improved safety profile. Two events are involved in the activation of the T cell effector function: 1) an appropriate tissue or disease specific marker bound by the targeting polypeptide; and 2) the presence of a disease associated antigen or inflammation (or inflammatory mediators) at the tissue.

Optionally, in order to increase the persistence of the transgenic Treg and/or endogenous Treg, or to increase the immune suppressive function of the transgenic Treg. The Treg may further comprise a nucleic acid encoding a regulatory T cell cytokine, chemokine or transcription factor that promotes regulatory T lymphocyte function. The nucleic acid can be inducible by the same transcription factor that induces the CAR, or may be constitutively active.

In certain embodiments, the cells used herein in the systems described are mammalian cells. In certain embodiments, the cells used herein in the cell systems described are human cells. In certain embodiments, the cells used herein in the systems described are immune cells. In certain embodiments, the immune cells used herein in the systems described are T lymphocytes. In certain embodiments, the immune cells used herein in the cell systems described are CD4+T lymphocytes. In certain embodiments, the T lymphocyte cells used herein in the cell systems described are regulatory T lymphocytes. In certain embodiments the regulatory T lymphocytes express FoxP3. Other such cells include non-T cell immune cells with regulatory function such as regulatory NK cell subsets, and regulatory B cell subsets.

In additional aspects, the cells used in the systems described herein are mesenchymal stem cells (MSC), adipose-derived stromal/stem cells (ADSCs), CD34+ hematopoietic stem or progenitor cells (such as those described in WO 2019/210042), or CD34+ induced pluripotent stem cells. Canonically, MSCs are CD73 CD90, CD105 and CD11b−, CD14−, CD19−, CD79−, CD45− and HLA-DR−. ADSCs can be differentiated from MSCs based on lack of expression of CD105; ADSCs also show high expression of CD49d and low expression of Stro-1. These cells can be isolated from the bone marrow or adipose tissue, and further induced to develop an anti-inflammatory phenotype. Type II mesenchymal stem cells can also be used herein to target autoimmune and inflammatory responses. Type II MSCs express anti-inflammatory cytokines and molecules such as IL-10, IDO, and PGE2. Anti-inflammatory mesenchymal stem cells may be made by inducing primary MSC with TLR3 ligands (e.g., polyinosinic-polycytidylic acid (poly(I:C) or poly(rI):poly(rC)).

The targeting polypeptides and effector molecules herein can also comprise a component of an acellular system. The acellular system can comprise extracellular vesicles, protein-coated vesicles, liposomes, dendrimers, micelles, biodegradable particles based on P-selectin, endothelial selectin (E-selectin) and ICAM-1, artificial nanostructures, engineered viral particles, plasmids, transposons, or bacterial cells.

### Targeting Polypeptides

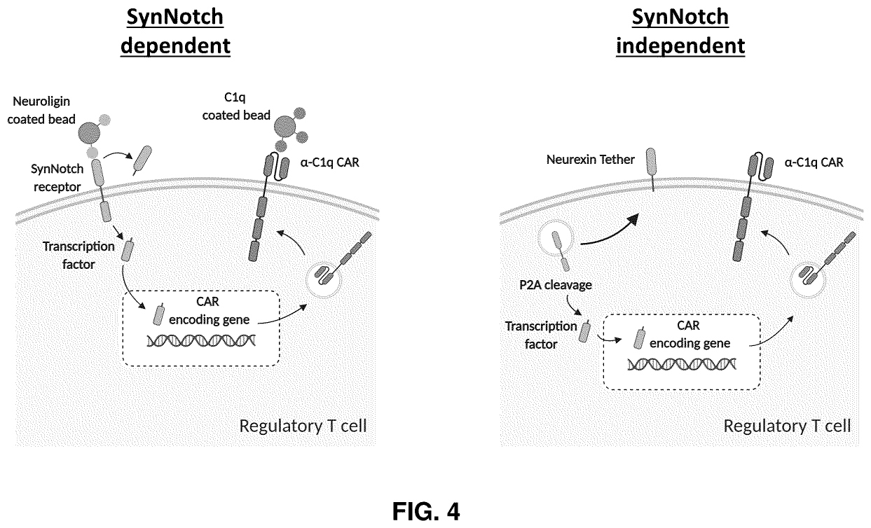

There are broadly speaking two classes of targeting polypeptides described herein and forming part of the invention. The first comprises a chimeric receptor having an extracellular domain comprising a ligand binding domain for directing the cell to the target tissue, a transmembrane domain and an intracellular domain comprising a cleavable transcription factor. Binding of the ligand binding domain triggers release of the transcription factor and subsequent expression of downstream genes, such as the gene encoding the CAR. This type of receptor can comprise a SynNotch polypeptide, which facilitates the cleavage of the transcription factor in response to target binding. This first type of targeting polypeptide is not necessarily expressed as a complete fusion polypeptide but may be assembled post-translationally, such as via a SNAPtag receptor, as described herein. Thus, the key features are that it has an extracellular targeting domain and an intracellular cleavable transcription factor. In a preferred embodiment the transcription factor is FoxP3. The second type of targeting polypeptide may be independent of the SynNotch system and instead use a viral self-cleaving peptide to release the transcription factor. This type of targeting polypeptide may comprise a tether polypeptide alongside the transcription factor. When the peptide is cleaved, the tether inserts into the cell membrane to provide the targeting function while the cleaved transcription factor stimulates downstream gene expression, such as driving expression of the CAR. Thus, in this instance it is not binding of the external target ligand (e.g. polypeptide) that stimulates release of the transcription factor, but the presence of the external tether is associated with CAR expression by virtue of the co-expression of the targeting polypeptide with the transcription factor. Expression of the targeting polypeptide in this instance may be constitutive in the cell. FIG. 4 provides an illustration of these two systems in action.

Thus, some targeting polypeptides described herein comprise an extracellular domain, a transmembrane domain, and an intracellular domain, wherein the extracellular domain comprises a ligand binding domain and wherein the intracellular domain comprises a transcription factor, wherein the transcription factor is configured to be released upon binding of the ligand binding domain by a ligand. The targeting polypeptide can comprise one or more variants that reduce binding to a specific target without completely abrogating such binding. In this way, a cell expressing the targeting polypeptide is allowed some movement around the site of disease or inflammation while being retained at the site. Such variants (mutants) may be used to tune the affinity of the targeting polypeptide to enhance therapeutic efficacy in any given therapeutic. Thus, the affinity of the targeting polypeptide may be tuned to specific therapeutic situations or targets to provide improved therapeutic outcomes. Such therapies could be personalised to the patient.

In certain embodiments, the targeting polypeptide comprises a portion of a neurexin polypeptide or a fragment of a neurexin polypeptide that binds neuroligin.

In certain embodiments, the targeting polypeptide comprises a portion of a neuroligin polypeptide or a fragment of a neuroligin polypeptide that binds neurexin.

The extracellular domain (or tether) of any targeting polypeptide of the invention can comprise one or more domains that target a neuronal marker. For the avoidance of doubt, any embodiment of the targeting portion (extracellular domain of the targeting polypeptide) is intended to relate to both classes of targeting polypeptide according to the invention. In certain embodiments, the neuronal marker is present at a neuronal synapse. In certain embodiments, the extracellular domain comprises a ligand binding domain that is neurexin or neuroligin. In certain embodiments, the extracellular domain comprises an amino acid sequence from neurexin. In order to reduce the affinity of the neurexin for the neuroligin receptor and contribute to degen-lock, the neurexin amino acid sequence may comprise one or more amino acid variants corresponding to S111A, D162A, I210A, N212A, D141A, I210A/D141A, compared to wild-type neurexin.

In certain embodiments, the extracellular domain of the targeting polypeptide comprises a neurexin amino acid sequence. In certain embodiments, the extracellular domain of the targeting polypeptide comprises a wild-type neurexin amino acid sequence. In certain embodiments, the extracellular domain comprises an amino acid sequence at least about 90%, 95%, 97%, 98%, 99%, or 100% identical to the amino acid sequence set forth in SEQ ID NO: 1. In certain embodiments, the neurexin amino acid sequence comprises an amino acid sequence identical to the amino acid sequence set forth in SEQ ID NO: 1. The extracellular domain may comprise an amino acid sequence that is 100% identical to any 10, 15, 20, 25, 30, 35, 40, 45, 50, 55, 60, 65, 70, 75, 80, 85, 90, or 100 contiguous amino acids from SEQ ID NO: 1. The extracellular domain may comprise an amino acid sequence that comprises a deletion of 1, 2, 3, 4, 5, 6, 7, 8, 9, 10, 15, 20, 25, 30, 35, 40, 45, 50, 55, 60, 65, 70, 75, 80, 85, 90, 100 or more amino acids from the N or C terminus of SEQ ID NO: 1. The extracellular domain may suitably comprise a signal sequence and/or a spacer sequence, such as a (G₄S)_{X} spacer, where X is equal to 1, 2, 3, or 4.

In certain embodiments, the extracellular domain of the targeting polypeptide comprises a neurexin amino acid sequence. In certain embodiments, the extracellular domain of the targeting polypeptide comprises a mutant neurexin amino acid sequence. In certain embodiments, the neurexin amino acid sequence comprises a mutation from serine at amino acid 61 of SEQ ID NO: 1 (S111 in wild type neurexin). In certain embodiments, the neurexin amino acid sequence comprises an alanine at amino acid 61 of SEQ ID NO: 1. In certain embodiments, the neurexin amino acid sequence comprises an amino acid sequence at least about 90%, 95%, 97%, 98%, 99%, or 100% identical to the amino acid sequence set forth in SEQ ID NO: 2, wherein the mutation at position 61 of SEQ ID NO: 2 is preserved. In certain embodiments, the neurexin amino acid sequence comprises an amino acid sequence identical to the amino acid sequence set forth in SEQ ID NO: 2. The extracellular domain may comprise an amino acid sequence that is 100% identical to any 10, 15, 20, 25, 30, 35, 40, 45, 50, 55, 60, 65, 70, 75, 80, 85, 90, or 100 contiguous amino acids from SEQ ID NO: 2. The extracellular domain may comprise an amino acid sequence that comprises a deletion of 1, 2, 3, 4, 5, 6, 7, 8, 9, 10, 15, 20, 25, 30, 35, 40, 45, 50, 55, 60, 65, 70, 75, 80, 85, 90, 100 or more amino acids from the N or C terminus of SEQ ID NO: 2.

In certain embodiments, the extracellular domain of the targeting polypeptide comprises a neurexin amino acid sequence. In certain embodiments, the extracellular domain of the targeting polypeptide comprises a mutant neurexin amino acid sequence. In certain embodiments, the neurexin amino acid sequence comprises a mutation from aspartic acid at amino acid 111 of SEQ ID NO: 1 (D162 in wild type neurexin). In certain embodiments, the neurexin amino acid sequence comprises an alanine at amino acid 111 of SEQ ID NO: 1. In certain embodiments, the neurexin amino acid sequence comprises an amino acid sequence at least about 90%, 95%, 97%, 98%, 99%, or 100% identical to the amino acid sequence set forth in SEQ ID NO: 3, wherein the mutation at position 111 of SEQ ID NO: 3 is preserved. In certain embodiments, the neurexin amino acid sequence comprises an amino acid sequence identical to the amino acid sequence set forth in SEQ ID NO: 3. The extracellular domain may comprise an amino acid sequence that is 100% identical to any 10, 15, 20, 25, 30, 35, 40, 45, 50, 55, 60, 65, 70, 75, 80, 85, 90, or 100 contiguous amino acids from SEQ ID NO: 3. The extracellular domain may comprise an amino acid sequence that comprises a deletion of 1, 2, 3, 4, 5, 6, 7, 8, 9, 10, 15, 20, 25, 30, 35, 40, 45, 50, 55, 60, 65, 70, 75, 80, 85, 90, 100 or more amino acids from the N or C terminus of SEQ ID NO: 3.

In certain embodiments, the extracellular domain of the targeting polypeptide comprises a neurexin amino acid sequence. In certain embodiments, the extracellular domain of the targeting polypeptide comprises a mutant neurexin amino acid sequence. In certain embodiments, the neurexin amino acid sequence comprises a mutation from isoleucine at amino acid 160 of SEQ ID NO: 1 (I210 in wild type neurexin). In certain embodiments, the neurexin amino acid sequence comprises an alanine at amino acid 160 of SEQ ID NO: 1. In certain embodiments, the neurexin amino acid sequence comprises an amino acid sequence at least about 90%, 95%, 97%, 98%, 99%, or 100% identical to the amino acid sequence set forth in SEQ ID NO: 4, wherein the mutation at position 160 of SEQ ID NO: 4 is preserved. In certain embodiments, the neurexin amino acid sequence comprises an amino acid sequence identical to the amino acid sequence set forth in SEQ ID NO: 4. The extracellular domain may comprise an amino acid sequence that is 100% identical to any 10, 15, 20, 25, 30, 35, 40, 45, 50, 55, 60, 65, 70, 75, 80, 85, 90, or 100 contiguous amino acids from SEQ ID NO: 4. The extracellular domain may comprise an amino acid sequence that comprises a deletion of 1, 2, 3, 4, 5, 6, 7, 8, 9, 10, 15, 20, 25, 30, 35, 40, 45, 50, 55, 60, 65, 70, 75, 80, 85, 90, 100 or more amino acids from the N or C terminus of SEQ ID NO: 4.

In certain embodiments, the extracellular domain of the targeting polypeptide comprises a neurexin amino acid sequence. In certain embodiments, the extracellular domain of the targeting polypeptide comprises a mutant neurexin amino acid sequence. In certain embodiments, the neurexin amino acid sequence comprises a mutation from asparagine at amino acid 162 of SEQ ID NO: 1 (N212 in wild type neurexin). In certain embodiments, the neurexin amino acid sequence comprises an alanine at amino acid 162 of SEQ ID NO: 1. In certain embodiments, the neurexin amino acid sequence comprises an amino acid sequence at least about 90%, 95%, 97%, 98%, 99%, or 100% identical to the amino acid sequence set forth in SEQ ID NO: 5, wherein the mutation at position 162 of SEQ ID NO: 5 is preserved. In certain embodiments, the neurexin amino acid sequence comprises an amino acid sequence identical to the amino acid sequence set forth in SEQ ID NO: 5. The extracellular domain may comprise an amino acid sequence that is 100% identical to any 10, 15, 20, 25, 30, 35, 40, 45, 50, 55, 60, 65, 70, 75, 80, 85, 90, or 100 contiguous amino acids from SEQ ID NO: 5. The extracellular domain may comprise an amino acid sequence that comprises a deletion of 1, 2, 3, 4, 5, 6, 7, 8, 9, 10, 15, 20, 25, 30, 35, 40, 45, 50, 55, 60, 65, 70, 75, 80, 85, 90, 100 or more amino acids from the N or C terminus of SEQ ID NO: 5.

In certain embodiments, the extracellular domain of the targeting polypeptide comprises a neurexin amino acid sequence. In certain embodiments, the extracellular domain of the targeting polypeptide comprises a mutant neurexin amino acid sequence. In certain embodiments, the neurexin amino acid sequence comprises a mutation from aspartic acid at amino acid 91 and a mutation from isoleucine at amino acid 160 of SEQ ID NO: 1 (D141 and I210, respectively in wild type neurexin). In certain embodiments, the neurexin amino acid sequence comprises an alanine at amino acid 91 and 160 of SEQ ID NO: 1. In certain embodiments, the neurexin amino acid sequence comprises an amino acid sequence at least about 90%, 95%, 97%, 98%, 99%, or 100% identical to the amino acid sequence set forth in SEQ ID NO: 6. In certain embodiments, the neurexin amino acid sequence comprises an amino acid sequence identical to the amino acid sequence set forth in SEQ ID NO: 6. The extracellular domain may comprise an amino acid sequence that is 100% identical to any 10, 15, 20, 25, 30, 35, 40, 45, 50, 55, 60, 65, 70, 75, 80, 85, 90, or 100 contiguous amino acids from SEQ ID NO: 6. The extracellular domain may comprise an amino acid sequence that comprises a deletion of 1, 2, 3, 4, 5, 6, 7, 8, 9, 10, 15, 20, 25, 30, 35, 40, 45, 50, 55, 60, 65, 70, 75, 80, 85, 90, 100 or more amino acids from the N or C terminus of SEQ ID NO: 6.

In certain embodiments, the extracellular domain of the targeting polypeptide comprises an scFv that targets neuroligin. In certain embodiments, the scFv that targets neuroligin comprises an amino acid sequence at least about 90%, 95%, 97%, 98%, 99%, or 100% identical to the sequence set forth in SEQ ID NO: 7. In certain embodiments, the neuroligin scFv comprises the complementarity determining regions from SEQ ID NO: 7, wherein the complementarity determining regions are determined according to the method of Kabat. In certain embodiments, the neuroligin scFv comprises the complementarity determining regions from SEQ ID NO: 7, wherein the complementarity determining regions are determined according to the IMGT method. In certain embodiments, the neuroligin scFv comprises the complementarity determining regions from SEQ ID NO: 7, wherein the complementarity determining regions are determined according to the method of Chothia. In certain embodiments, the neuroligin scFv comprises the complementarity determining regions from SEQ ID NO: 6, wherein the complementarity determining regions are determined according to any combination of the method of Chothia, Kabat, or IMGT.

The extracellular domain of the targeting polypeptide may also comprise one or more fusions with a polypeptide labelling tag. Alternatively, the polypeptide labelling tag may comprise at least about 60%, 70%, 80% or 90% of the amino acid sequence of the extracellular domain. In certain embodiments, the polypeptide labelling tag comprises a SNAP-Tag or a CLIP-Tag. A SNAP-tag, is a 20 kDa mutant of the DNA repair protein 06-alkylguanine-DNA alkyltransferase that reacts specifically and rapidly with benzylguanine (BG) derivatives. See Keppler, A., Gendreizig, S., Gronemeyer, T. et al. (2003) Nat. Biotechnol. 21, 86. The CLIP-Tag is an engineered version of the SNAP-tag permitting it to react specifically with O2-benzylcytosine (BC) derivatives. See Gautier, A., Juillerat, A., Heinis, C. et al. (2008) Chem. Biol. 15, 128. Appropriately a benzylguanine or a derivative or benzylcytosine or a derivative can be used to modify a polypeptide such that introduction of the BC or OC labelled polypeptide into the body of an individual would label the cells of this disclosure with the desired BC or OC modified polypeptide. The polypeptide labelling tag may suitably comprise a leader sequence and/or a spacer domain allowing efficient cell surface expression of the targeting polypeptide comprising the labelling tag.

The targeting polypeptides described herein comprise a transmembrane domain to anchor the targeting polypeptide to the cell. Further, the transmembrane domain may contribute to the functional aspects of the targeting domain by providing catalytic activity that cleaves the intracellular domain releasing a polypeptide comprising an amino acid sequence configured to bind a regulatory sequence. Such regulated cleavable transmembrane systems may comprise a portion of a Notch protein.

Notch is transmembrane receptor protein that is present in vertebrates and invertebrates. Signal transduction through Notch from the extracellular space to the intracellular space requires the transmembrane portion of the Notch protein. The engagement of notch on cells leads to two-step proteolysis of the notch receptor that ultimately causes the release of the intracellular portion of the receptor from the membrane into the cytoplasm. The extracellular domain and the intracellular domains of notch can be replaced with heterologous polypeptides, while preserving this transmembrane based cleavage. Thus, creating a chimeric Notch protein by replacing the extracellular domain of Notch with a suitable antigen or ligand binding domain (e.g., a heterologous receptor or antibody-based polypeptide) allows for the construction of a molecular sensor that allows release of the intracellular portion by a non-delta-based ligand. Further, engineering of the intracellular domain, to replace the Notch intracellular domain with a heterologous transcription factor, allows the chimeric Notch to release the heterologous transcription factor. Such transcription factors and their targets are described herein in more detail, but in brief, can be used to induce activation of a variety of genes under the control of a regulatory sequence that binds the heterologous transcription factor.

In certain embodiments, the transmembrane domain comprises a portion of the mammalian Notch protein. The portion of the mammalian Notch protein may be derived from a human notch, such as that shown in SEQ ID NO: 8. In certain embodiments, the portion of the Notch protein is the Notch regulatory region. In certain embodiments, the notch regulatory region comprises a Lin 12-Notch repeat, an S2 proteolytic cleavage site, and an S3 proteolytic cleavage site The notch transmembrane domain may further comprise N and C terminal spacers to allow for efficient expression and activation of a polypeptide comprising heterologous extracellular and intracellular domains, these spacers may be at least 1, 2, 3, 4, 5, 6, 7, 8, 9, 10, 11, 12, 13, 14, 15, 16, 17, 18, 19, 20 or more amino acids not derived from Notch or the corresponding intracellular or extracellular domains. In certain embodiments, the notch transmembrane domain comprises an amino acid sequence at least about 90%, 95%, 97%, 97%, 99%, or 100% identical to SEQ ID NO 9. In certain embodiments, the notch transmembrane domain comprises an amino acid sequence at least about 90%, 95%, 97%, 97%, 99%, or 100% identical to SEQ ID NO 10. In certain embodiments, the notch transmembrane domain comprises an amino acid sequence at least about 90%, 95%, 97%, 97%, 99%, or 100% identical to SEQ ID NO 11. Systems deploying a chimeric Notch protein are described in U.S. Pat. No. 9,834,608; Morsut L., et al. “Engineering Customized Cell Sensing and Response Behaviors Using Synthetic Notch Receptors,” Cell 2016 Feb. 11; 164(4):780-91; and Roybal K. T., et al. “Engineering T Cells with Customized Therapeutic Response Programs Using Synthetic Notch Receptors” Cell 2016 Oct. 6; 167(2):419-432.

The intracellular domain of the described targeting polypeptides comprises one or more polypeptides that are configured to bind a regulatory sequence. Such polypeptides can comprise all or part of a transcription factor. In certain embodiments, the transcription factor polypeptide comprises a DNA binding domain and an activating domain. The DNA binding domain and the activating domain may be derived from the same transcription factor or different transcription factors. In certain embodiments, the transcription factor may be configured to bind a regulatory sequence associated with an exogenous nucleic acid, a regulatory sequence associated with an endogenous gene, or both.

In certain embodiments, the transcription factor comprises Gal4-VP6, tetR-VP64 (tTA), ZFHD1-VP64, Gal4-KRAB, PIP-VP64, ZF21-16-VP64, ZF43-8-VP64 or FoxP3. In certain embodiments, the transcription factor comprises Gal4. In certain embodiments, the transcription factor comprises an amino acid sequence at least about 90%, 95%, 97%, 97%, 99%, or 100% identical to SEQ ID NO: 12. In certain embodiments, the transcription factor comprises Gal4-VP6. In certain embodiments, the transcription factor comprises FOXP3. In certain embodiments, the transcription factor comprises an amino acid sequence at least about 90%, 95%, 97%, 97%, 99%, or 100% identical to SEQ ID NO: 13.

Alternatively, if a notch-based system is not used for the targeting polypeptide a serine protease-based system can be used. In such a system the transmembrane domain lacks catalytic activity, and can be any suitable transmembrane domain that serves to anchor the extracellular domain to the cell. In a serine protease based system the intracellular domain comprises a cleavage domain configured to be cleaved by a type II serine protease, a type II serine protease domain comprising a catalytically active region of a serine protease, an inhibitory domain comprising an amino acid sequence that inhibits the catalytically active region of the type II serine protease when the ligand binding domain is bound by a ligand, and the transcription factor. Upon binding of ligand or antigen to the ligand binding domain, steric hinderance of the type II serine protease domain by the inhibitory domain is relieved, allowing the type II serine protease domain to cleave the cleavage domain releasing the transcription factor(s). This type of system is compatible with the same transcription factors already described herein including Gal4-VP6, tetR-VP64 (tTA), ZFHD1-VP64, Gal4-KRAB, or FoxP3. In certain embodiments, the catalytically active region of the serine protease comprises an active domain of thrombin, Hepatitis C virus Ns3 serine protease, or a TVMV protease.

In certain embodiments, the targeting polypeptide comprises an amino acid sequence at least about 90%, 95%, 97%, 97%, 99%, or 100% identical to SEQ ID NO 14. In certain embodiments, the targeting polypeptide comprises an amino acid sequence identical to SEQ ID NO 14. In certain embodiments, the targeting polypeptide lack a signal sequence, and/or is expressed on the cell surface of a cell beginning with the amino acids ASSLGA.

In certain embodiments, the targeting polypeptide comprises an amino acid sequence at least about 90%, 95%, 97%, 97%, 99%, or 100% identical to SEQ ID NO 15. In certain embodiments, the targeting polypeptide comprises an amino acid sequence identical to SEQ ID NO 15. In certain embodiments, the targeting polypeptide lacks a signal sequence, and/or is expressed on the cell surface of a cell beginning with the amino acids ASSLGA.

In certain embodiments, the targeting polypeptide comprises an amino acid sequence at least about 90%, 95%, 97%, 97%, 99%, or 100% identical to SEQ ID NO 16. In certain embodiments, the targeting polypeptide comprises an amino acid sequence identical to SEQ ID NO 16. In certain embodiments, the targeting polypeptide lacks a signal sequence, and/or is expressed on the cell surface of a cell beginning with the amino acids ASSLGA.

In certain embodiments, the targeting polypeptide comprises an amino acid sequence at least about 90%, 95%, 97%, 97%, 99%, or 100% identical to SEQ ID NO 17. In certain embodiments, the targeting polypeptide comprises an amino acid sequence identical to SEQ ID NO 17. In certain embodiments, the targeting polypeptide lacks a signal sequence, and/or is expressed on the cell surface of a cell beginning with the amino acids ASSLGA.

In certain embodiments, the targeting polypeptide comprises an amino acid sequence at least about 90%, 95%, 97%, 97%, 99%, or 100% identical to SEQ ID NO 18. In certain embodiments, the targeting polypeptide comprises an amino acid sequence identical to SEQ ID NO 18. In certain embodiments, the targeting polypeptide lack a signal sequence, and/or is expressed on the cell surface of a cell beginning with the amino acids ASSLGA.

In certain embodiments, the targeting polypeptide comprises an amino acid sequence at least about 90%, 95%, 97%, 97%, 99%, or 100% identical to SEQ ID NO 19. In certain embodiments, the targeting polypeptide comprises an amino acid sequence identical to SEQ ID NO 19. In certain embodiments, the targeting polypeptide lack a signal sequence, and/or is expressed on the cell surface of a cell beginning with the amino acids ASSLGA.

The targeting polypeptides are encoded by nucleic acids and operatively coupled to a regulatory sequence that allows for constitutive or inducible activation of the targeting polypeptides. Such regulatory sequences comprise a CMV promoter, a β-actin promoter, an RSV promoter, an EF1α promoter, an SV40 promoter, human ubiquitin C promoter, PGK promoter, doxycycline-inducible promoter, and any combination thereof.

The targeting polypeptides described herein can be encoded by nucleic acids and delivered to cells using the expression vectors described herein. In certain embodiments, the targeting polypeptides are encoded by nucleic acids and delivered to cells using a retroviral vector. In certain embodiments, the targeting polypeptides are encoded by nucleic acids and delivered to cells using an adenoviral vector or adeno associated viral (AAV) vector. In certain embodiments, the targeting polypeptides are encoded by nucleic acids and delivered to cells using a lentiviral vector. In certain embodiments, the targeting polypeptides are encoded by nucleic acids and delivered to cells using electroporation.

### Immune Effector Molecules

In certain embodiments, described herein, are immune effector molecules and nucleic acids encoding the immune effector molecules. In certain embodiments, the immune effector molecule comprises an antigen binding domain, that is a component of an extracellular domain of the immune effector molecule. The immune effector molecules also comprise a transmembrane domain and one or more intracellular signalling domains. The one or more intracellular signalling domains may function in T cell signalling and comprise CD3-zeta, CD27, ICOS, and/or CD28. In certain embodiments, the immune effector molecule comprises a chimeric antigen receptor (CAR). In some embodiments, the immune effector molecule comprises a T cell receptor. In certain embodiments, the antigen binding domain of the chimeric antigen receptor comprises an scFv. In certain embodiments, the antigen binding domain of the chimeric antigen receptor comprises a binding domain that specifically binds to a disease associated antigen or a pro-inflammatory molecule. In certain embodiments, the pro inflammatory molecule is a cytokine, a chemokine, a bacterial derived molecule such as LPS or peptidoglycan, or a molecule derived from an apoptotic cells such as phosphatidyl serine. In certain embodiments, the antigen binding domain of the chimeric antigen receptor comprises a binding domain that specifically binds to a complement pathway protein or polypeptide. In certain embodiments, the complement pathway protein comprises C1q, C1 r, C1s, C2a, C3, C3a, C3b, C4, C4a, C4b, C5, C5a, C5b, C6, C7, C8, or C9. In certain embodiments, the complement pathway protein comprises C1q.

The antigen binding domain of the immune effector molecule may be derived from an antibody or an scFv. In certain embodiments, the antigen binding domain is derived from an anti-C1q monoclonal antibody or scFV. Certain C1q antibodies useable in the current immune effector molecules are described in WO 2008/035527 and U.S. Pat. No. 10,316,081. The complementarity determining regions from a C1q antibody can be used to construct the antigen binding domain of the described immune effector molecules, wherein the complementarity determining regions are determined according to any combination of the methods of Chothia, Kabat, or IMGT.

The immune effector molecules described herein are encoded by nucleic acids and can be operatively coupled to a regulatory sequence. This allows for inducible expression for the immune effector molecule. In certain embodiments, the regulatory sequence is configured to be bound by the transcription factor that is released upon engagement of the targeting polypeptide. In certain embodiments, the regulatory sequence is configured to be bound by Gal4-VP6, tetR-VP64 (tTA), ZFHD1-VP64, Gal4-KRAB, or FoxP3. In certain embodiments, the regulatory sequence is configured to be bound by FoxP3.

The immune effector molecules described herein can be encoded by nucleic acids and delivered to cells using the expression vectors described herein. In certain embodiments, the immune effector molecules are encoded by nucleic acids and delivered to cells using a retroviral vector. In certain embodiments, the immune effector molecules are encoded by nucleic acids and delivered to cells using a lentiviral vector. In certain embodiments, the immune effector molecules are encoded by nucleic acids and delivered to cells using an adenoviral vector or an AAV vector. In certain embodiments, the immune effector molecules are encoded by nucleic acids and delivered to cells using electroporation.

The cells described herein in addition to comprising a targeting polypeptide and/or an immune effector molecule may also comprise a nucleic acid encoding a regulatory T cell cytokine, chemokine or transcription factor that promotes regulatory T lymphocyte function. In certain embodiments, the transcription factor that promotes regulatory T lymphocyte function comprises STAT5b. In certain embodiments, the polypeptide that promotes regulatory T lymphocyte function comprises IL-2, IL-5, IL-4, IL-7, IL-15, or IL-37. In certain embodiments, the polypeptide that promotes regulatory T lymphocyte function comprises IL-2. Nucleic acids encoding a regulatory T cell cytokines, chemokines or transcription factors that promote regulatory T lymphocyte function are operatively coupled to a regulatory sequence. In certain embodiments, the regulatory sequence is inducible. In certain embodiments, the regulatory sequence is constitutive.

Cells administered comprising the targeting polypeptides and the immune effector molecules described herein can deliver highly-localized factors to promote regulatory T lymphocyte function, namely anti-inflammatory effects on surrounding tissue. In certain embodiments, the polypeptide that promotes regulatory T lymphocyte function is under control of a constitutive promoter. In certain embodiments, the polypeptide that promotes regulatory T lymphocyte function is under control of an inducible promoter. The inducible promoter may be under a promoter configured to be bound by a transcription factor associated with the targeting polypeptide and may be the same transcription factor that activates transcription of the immune effector molecule. Thus, activation of the targeting polypeptide by a suitable tissue specific marker or ligand results in activation of expression of both the immune effector molecule and expression of a polypeptide that promotes regulatory T lymphocyte function. In certain embodiments, the polypeptide that promotes regulatory T lymphocyte function comprises IL-2, IL-5, IL-4, IL-7, IL-15, or IL-37.

### Methods of Making

Described herein are methods of making populations of cells comprising targeting polypeptides and immune effector molecules, the method comprising contacting the cell population with a nucleic acid encoding a chimeric antigen receptor and/or an immune effector molecule. In certain embodiments, the cell population is a population comprising CD4+ T cells that are at least about 75%, 80%, 85%, 90%, 95%, 98%, 99% or more CD4+. In certain embodiments, the nucleic acid or nucleic acids used to contact the cell is encoded in an expression vector such as a lentivirus vector, adenovirus, AAV or a retrovirus vector. In certain embodiments, the nucleic acids are introduced to the population of cells by electroporation.

The cell source of the population of cells can be from the individual ultimately treated, and therefore autologous. The cell source of the population of cells can be from an HLA-matched individual, and therefore heterologous. The cell source can be so called universal T cells, which have disruptions in the HLA locus or the TCR α and β chains.

Autologous or heterologous cells are isolated from the peripheral blood mononuclear cells (PBMC) of the individual. The leukocyte population can be isolated by leukapheresis, positive, or negative selection and then subjected to transduction or transfection with nucleic acid encoding targeting polypeptides and/or immune effector molecules of this disclosure.

Nucleic acids cells encoding targeting polypeptides and/or immune effector molecules can be introduced into cells in a variety of ways, including by the viral transduction, electroporation, or lipid assisted transfection of nucleic acids encoding each of the targeting polypeptide and the immune effector molecules.

In one such process, illustrated here by way of example, cells, for example, T cells, are collected from a donor (for example, a patient to be treated with an autologous chimeric antigen receptor T cell product) via apheresis (e.g., leukapheresis). Collected cells may then be optionally purified, for example, by an elutriation step. Paramagnetic particles, for example, anti-CD3/anti-CD28-coated paramagnetic particles, may then be added to the cell population, to expand the T cells. The process may also include a transduction step, wherein nucleic acid encoding one or more desired proteins, for example, a CAR, for example a CAR targeting C1q, is introduced into the cell. The nucleic acid may be introduced in a lentiviral vector. The cells, e.g., the lentiviral transduced cells, may then be expanded for a period of days, for example, 1, 2, 3, 4, 5, 6, 7, 8, 9, 10, or more days. After expansion cells can be collected and washed by centrifugation, the overall transduction efficiency determined, by flow cytometry for example, suspended in an appropriate diluent and administered to a patient. Alternatively, cells may be refrigerated or frozen and shipped to a site where they are administered to the individual in need thereof.

Other cell types such as mesenchymal stem cells and adipose-derived stromal/stem cells can also be used in the method described herein. Isolation of mesenchymal stem cells can be from the bone-marrow or adipose tissue. Method of isolating mesenchymal stem cells are described for example in US 2002/0045260 A1 or US 2011/0076770 A1. Mesenchymal stem cells can further be induced to develop an anti-inflammatory phenotype by treatment with TLR3 ligand either before or after they have been transfected or transduced with nucleic acids encoding the targeting polypeptides. Method of inducing mesenchymal stem cells include those described in US 2014/0017787 A1.

### Methods of Use and Medical Uses

Described herein are methods of delivering a cargo to a specific tissue and activating a desired effect. This effect may be a therapeutic effect, a diagnostic effect, or some other effect useful for research and discovery. The usefulness in such an approach is a result of the presence of a targeting polypeptide and a polypeptide that serves an effector function. The effector function can vary dependent on the exact application. The cargo may be a cell as described herein. Alternatively, the cargo may be extracellular vesicles, protein-coated vesicles, liposomes, dendrimers, micelles, biodegradable particles based on P-selectin, endothelial selectin (E-selectin) and ICAM-1, artificial nanostructures, engineered viral particles, plasmids, transposons, or bacterial cells. The cargo can further comprise therapeutically or diagnostically useful components such as fluorescent, radioactive, or luminescent cargo. Administration of the cargo comprising the targeting polypeptide and a polypeptide that serves an effector function targets the cargo to an appropriate location based upon specificity of the targeting polypeptide for a tissue specific marker.

In certain embodiments, described herein is a molecule with a targeting function and an effector function. In certain embodiments, the targeting function targets the antibody to neurexin or neuroligin and the effector function binds to a complement related protein.

The cells comprising targeting polypeptides and inducible immune effector molecules are for use in methods of treating a neuroinflammatory disease or an autoimmune neurological disease in a mammal. The mammal can be a human individual. In an embodiment, the method of treating the neuroinflammatory disease comprises administering a plurality or population of the cells that express a targeting polypeptide and comprise an inducible CAR construct activatable by the targeting polypeptide's engagement with an appropriate ligand to an individual in need thereof. In certain embodiments, the cells are autologous to the individual being administered the cells. In certain embodiments, the cells are heterologous to the individual being administered the cells, but are HLA matched. In certain embodiments, the cells are not HLA matched but are “universal T cells.” These are made by targeting genomic sequences in the constant regions of the endogenous a or R subunits of the TCR or disrupting the HLA-A locus of MHC gene complex, the expression of TCR or the HLA class I antigens is abrogated, and the resulting T cells are not capable of recognizing allogeneic antigens, thus leading to the elimination of GVHD. In certain embodiments, the plurality of cells or cell population is a population comprising CD4+ T cells that are at least about 75%, 80%, 85%, 90%, 95%, 98%, 99% or more CD4+. In certain embodiments, the plurality of cells or cell population is a population comprising CD4+ T cells that are at least about 75%, 80%, 85%, 90%, 95%, 98%, 99% or more positive for FoxP3 expression.

Also described are methods of treatment using other anti-inflammatory cell types such as regulatory NK cell subsets, regulatory B cell subsets, induced pluripotent stem cells, CD34+ stem cells or the mesenchymal stem cells and adipose-derived stromal/stem cells described herein.

Neuroinflammatory diseases generally display infiltration of cytotoxic T cells, Th1 cells, Th17 cells, or phagocytic cells into one or more neuronal tissues. Neuroinflammatory diseases can also be marked by the presence of autoantibodies, or elevated inflammatory mediators such as chemokines or cytokines at neuronal tissues. These neuronal tissues comprise, for example, the brain, spinal cord, neurons, or neuro-muscular junctions. The neuroinflammatory disease can suitably be a motor neuron disease, demyelinating disease, neurodegenerative disease, brain or spinal cord injury.

Neuroinflammatory diseases treatable by the cells, methods and medical uses described herein comprise acute disseminated encephalomyelitis (ADEM), acute Optic Neuritis (AON), transverse myelitis, neuromyelitis optica, multiple sclerosis, relapsing-remitting multiple sclerosis, secondary-progressive multiple sclerosis, primary-progressive multiple sclerosis (PPMS), progressive-relapsing multiple sclerosis (PRMS), Alzheimer's disease, Parkinson's disease, Huntington's disease, Lou Gehrig's disease (amyotrophic lateral sclerosis), Creutzfeldt Jakob disease, multiple sclerosis, diffuse Lewy body disease, leukoencephalitis, autoimmune encephalitis, meningitis, temporal lobe epilepsy, traumatic brain injury, inflammatory spinal cord injury, myasthenia gravis. In certain embodiments, the neuroinflammatory disease treatable by the cells and methods described herein is a motor neuron disease selected from amyotrophic lateral sclerosis (ALS), progressive bulbar palsy (PBP), progressive muscular atrophy (PMA), primary lateral sclerosis (PLS), and combinations thereof. In a certain embodiment, the neuroinflammatory disease treatable by the cells and methods described herein is amyotrophic lateral sclerosis (ALS).

The cells described herein can also be used to treat autoimmune neurological diseases. Such diseases are defined by the development of adaptive immune responses to self-antigens expressed in neural tissues, such as myelin basic protein or myelin oligodendrocyte glycoprotein. Autoimmune neurological diseases treated by the cells and methods described herein comprise multiple sclerosis, myasthenia gravis, Guillain-Barre, and any combination thereof. The multiple sclerosis treated may be relapsing-remitting multiple sclerosis, secondary-progressive multiple sclerosis, primary-progressive multiple sclerosis, progressive-relapsing multiple sclerosis, or any combination thereof.

The cells comprising targeting polypeptides and inducible immune effector molecules are for use in treating, and can be used in the manufacture of a medicament for treating, a neuroinflammatory or autoimmune neurological disease. The neuroinflammatory diseases comprise acute disseminated encephalomyelitis (ADEM), acute Optic Neuritis (AON), transverse myelitis, neuromyelitis optica, multiple sclerosis, relapsing-remitting multiple sclerosis, secondary-progressive multiple sclerosis, primary-progressive multiple sclerosis (PPMS), progressive-relapsing multiple sclerosis (PRMS), Alzheimer's disease, Parkinson's disease, Huntington's disease, Lou Gehrig's disease (amyotrophic lateral sclerosis), Creutzfeldt Jakob disease, multiple sclerosis, diffuse Lewy body disease, leukoencephalitis, meningitis, temporal lobe epilepsy, traumatic brain injury, inflammatory spinal cord injury. The neuroinflammatory disease may be a motor neuron disease. The motor neuron disease may be selected from amyotrophic lateral sclerosis (ALS), progressive bulbar palsy (PBP), progressive muscular atrophy (PMA), primary lateral sclerosis (PLS), and combinations thereof. In a certain embodiment, the neuroinflammatory disease treatable by the cells and methods described herein is amyotrophic lateral sclerosis (ALS).

The cells comprising targeting polypeptides and inducible immune effector molecules described herein can be administered in a manner consistent with the disease being treated. Cells may be administered intravenously, subcutaneously, intradermally, or intrathecally as required by the neuroinflammatory/autoimmune neurological disease being treated. For example, to easily traffic to sites of inflammation in the brain or spinal cord the cells may be administered intrathecally. Intravenous, subcutaneous, or intradermal administration may be more suitable for administration to individuals with MS or motor neuron disease.

The cells comprising targeting polypeptides and inducible immune effector molecules described herein can be administered in a therapeutically effective amount. The total amount of cells administered to an individual can be administered in a single-dose or split over multiple doses to help minimize infusion reaction side-effects. In certain embodiments, a therapeutically effective dose of cells comprises about 1×10⁵ cells/kg to about 1×10⁷ cells/kg. These numbers refer to doses of positive cells, determined for example, by a parallel transduction of cells. The actual total number of cells given may be higher to account for the transduction efficiency. The number of cells above can refer to the number of CD4+ T cells or the number of FOXP3+CD4+ T cells.

Individuals may be administered a dosage of the cells comprising targeting polypeptides and inducible immune effector molecules based upon a diagnosis with or a suspected diagnosis with any of the neuroinflammatory disease or autoimmune neurological diseases described herein. Individuals can be administered one dose of cells or multiple doses based upon a reemergence of systems or no improvement in symptoms. A subsequent dose can comprise the same, 2×, 3×, 4×, 5×, 6×, 7×, 8×, 9×, or 10× more cells than the first dose. A subsequent dose may be administered after 3, 4, 5, 6, 7, 8, 9, 10, 11, 12 or more weeks.

### Pharmaceutically Acceptable Excipients, Carriers, and Diluents

In certain embodiments, the cells of the current disclosure are included in a pharmaceutical composition comprising one or more pharmaceutically acceptable excipients, carriers, and diluents. In certain embodiments, the cells of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

Also described herein and forming part of the invention are kits comprising one or more of the vectors or nucleic acids described herein in a suitable container and one or more additional components selected from: instructions for use; a diluent, an excipient, a carrier, and a device for administration.

In certain embodiments, described herein is a method of preparing a neuroinflammatory treatment comprising admixing one or more pharmaceutically acceptable excipients, carriers, or diluents and a mammalian cell of the current disclosure. In certain embodiments, the cell expresses a suitable targeting polypeptide and comprises a nucleic acid(s) encoding an inducible CAR.

### EXAMPLES

The following illustrative examples are representative of embodiments of compositions and methods/uses described herein and are not meant to be limiting in any way.

### Example 1—Design and Expression of Neurexin Targeting Polypeptides

An insert encoding a chimeric targeting polypeptide was designed as follows: a 20 amino-acid signal-peptide sequence (MALPVTALLLPLALLLHAARP (SEQ ID NO: 20)) was fused to a myc-tag sequence EQKLISEEDL (SEQ ID NO: 21) for ease of detection, the neurexin 1-b polypeptide, the linker and the transmembrane notch sequence and a transcription factor. The synthetic DNA was manufactured by Genescript Ltd, verified by sequencing, and cloned into the pcDNA3.1(+) backbone between the EcoRI and NotI restriction sites. pREF001 contains the natural sequence of the fragment encoding neurexin 1-b polypeptide (SEQ ID NO: 1). pREF002 encodes a neurexin 1-b variant, with a S111A mutation (SEQ ID NO: 2). pREF003 encodes a neurexin 1-b variant, with a D162A mutation (SEQ ID NO: 3). pREF004 encodes a neurexin 1-b variant, with a I210A mutation (SEQ ID NO: 4). pREF005 encodes a neurexin 1-b variant, with a N212A mutation (SEQ ID NO: 5). pREF006 encodes a neurexin 1-b double variant, with a D141A:I210A double mutation (SEQ ID NO: 6).

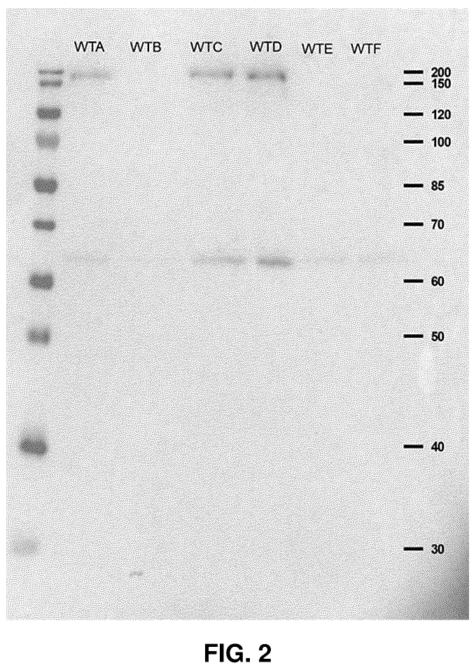

To assess functionality of the receptors, GAL4 Reporter (Luc)-HEK293 cells were transiently transfected with plasmid pREF001 to pREF006 as follows: the cell bank (BPS bioscience) was thawed and maintained in Growth Medium 1B: MEM medium supplemented with 10% FBS, 1% non-essential amino acids, 1 mM Na-pyruvate, 0.5% Penicillin/Streptomycin and 400 μg/ml of geneticin. When cells reached around 90% confluency, they were split at 1:10 ratio. After the 3rd passage, cell stocks of 3×10⁶ cells were frozen in 10% DMSO. Cells were seeded onto a 6-well plate for 24 hours, at a density of 1×10⁶ cells per well in 2 ml of medium without antibiotics. 2 μg of plasmid DNA (Genescript) was diluted into 200 μl of medium without FBS. 6 μl of X-tremeGENE HP DNA Transfection Reagent was added directly into medium containing the diluted DNA, mixed and incubated at 15 min at room temperature to allow complexes to form. Next, DNA complexes were added drop-wise into the medium. Cells were incubated at 37° C. in a 5% CO2 incubator for the next 24 h. Transfection efficiency was determined by detection with c-Myc (9E10) FITC antibody (Santa Cruz Biotechnology) after 24 h and anti-hc-Myc PE Conjugated antibody (R&D Systems) after 48 h. Expression by western blot was assessed and is shown in FIG. 2.

### Example 2—Functional Testing of Neurexin Targeting Polypeptides

For preliminary experiments, pREF020 was prepared from pREF001 as follows: primer pair AGCGAGGAGGATCTGATGTACCAGAGGATGCTGAGGTGC (SEQ ID NO: 25) and GCTGTAGTCCAGGATGGGCACCTCGCCCACCAGC (SEQ ID NO: 26) was used to amplify 828 bp fragment using template pREF001 and HiFi PCR Premix polymerase (Clontech-Takara) as follows: 12.5 μL water was mixed with 12.5 μL polymerase premix, 0.5 μL plasmid DNA template, 1.25 μL 10 uM primer GCCGCCGCGATCGCCatggcattgcccgt (SEQ ID NO: 27) and 1.25 μL 10 uM GCGGCCGGCCGTTTAtcatgatccgagcatgtccag (SEQ ID NO: 28). Fragment was then run on 1% agarose 0.5×TBE gel and was isolated from the gel using MN Cleanup kit. Then, the band was eluted in 15 μL water. Then, 1 μL insert was mixed with 1 uL vector backbone amplified as described above and the InFusion cloning reaction was performed in the presence of 2 μL of InFusion 5× premix (Takara) and 6 μL water at 50° C. for 15 minutes. 1 μL was used to transform chemically competent Escherichia coli HST08 (Takara) by heat shock and cells were selected on LB with 100 μg/mL ampicillin (Melford). Collect clones were identified and confirmed by sequencing.

Assay was performed as follows: Transfections were carried out with 7.5 μL Fugene (Promega) and 2 μg plasmid DNA from midi-prep per well. For pREF020 transfection, DNA concentration was 94.1 ng/uL and 53.1 microlitres of DNA solution were mixed with 18.8 uL Fugene and 3.1 μL OptiMem (Gibco). Mixture was incubated for 20 minutes at room temperature and 30 μL of mixture were added dropwise and plates were mixed gently to rocking from side to side. Plates were incubated for 48 hours at 37° C. 5% CO2.

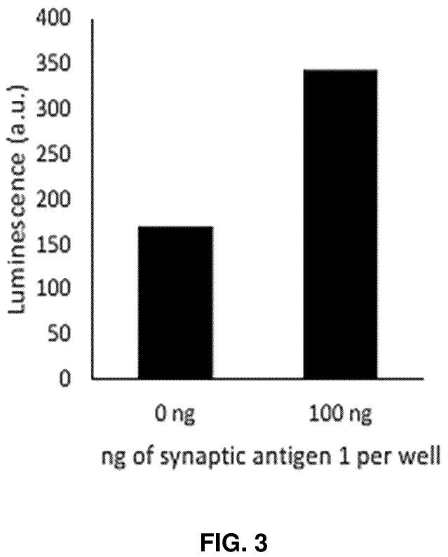

To measure binding of the neurexin mutants to neuroligin, and activation of the GAL4 reporter, cells were seeded onto 96-well plates for a luciferase assay. White bottomed 96 well plate was coated with 100 ng neuroligin (Biotechne) suspended in PBS. After 24 hours PBS was removed. Cells were collected and counted, diluted in fresh DMEM medium and cells were plated at 18′000 cells per well in 100 μL volume. After 24 hours, luciferase assays using Promega kit E1500 were carried out on the wells. Media is removed from the plates and 20 μL lysis buffer was added. After 5 minutes of lysis 100 μL of luciferase assay reagent was mixed into each well and the light signal from each well was recorded with a plate reader. Exemplary induction using pREF001 is shown in FIG. 3.

### Example 3—Transduction of Lymphocytes with Nucleic Acids Encoding Neurexin Targeting Polypeptides

The ORFs of interest from vectors pREF001, pREF002, pREF003, pREF004, pREF005 or pREF006 are subcloned into a pBABE-puro vector using standard molecular biology methods.

2.5×10⁶ Phoenix packaging cells are plated in 9 ml medium/10 cm dish in the afternoon. The cells are transfected with 20 micrograms of DNA ±24 hrs after plating), using the CaPO₄ precipitation method. Briefly, in a 2 ml eppendorf tube 50 microlitres of 2.5M CaCl₂, 20 micrograms of DNA are mixed and distilled sterile water to 500 μL is added. While vortexing the tube, slowly add 500 μL of buffer consisting of 50 mM HEPES pH 7.05, 10 mM KCl, 12 mM D-glucose, 280 mM NaCl, 1.5 mM Na₂HPO₄, dropwise. Then, 1 mL is added to the Phoenix cells and cells are placed back into an incubator.

To produce a viral supernatant, selected virus producing Phoenix packaging cells are cultured overnight in 20-mm Petri dishes at 80% confluence with 5 mL complete Dulbecco's modified Eagle medium (DMEM) (DMEM supplemented with 10% FCS, 4 mmol/L L-glutamine, 1 mmol/L sodium pyruvate, nonessential amino acids, and streptavidin/penicillin). CD4+CD25+ Tregs are isolated from murine splenocytes or human peripheral blood mononuclear cells using a CD4-CD25 Treg isolation kit (Miltenyi Biotech, Bergisch Gladbach, Germany) to achieve a purity of over 90%. The isolated cells are preactivated for 4 to 18 hours with 2 μg/mL Concanavalin A or with plate bounded anti-CD3 and anti-CD28 antibodies (plated at 1 ng/mL and 5 ng/mL, respectively). Activation and following transduction steps are performed either in Biotarget-1serum-free medium (Biological Industries, Beit Haemek, Israel) or in complete medium (RPMI supplemented with 10% FCS); both media are supplemented with 750 U/mL IL-2, 4 mmol/L L-glutamine, 1 mmol/L sodium pyruvate, 0.01 mol/L HEPES, 50 mol/L 2-ME, non-essential amino acids, and streptavidin/penicillin. For retroviral transduction, the viral supernatant is filtered through a 0.45 filter and supplemented with 750 U/mL recombinant mouse IL-2. Next, 750 μL cell-free viral supernatant is administered to each well of 24-well plates pre-coated with 5 g/mL retronectin (TakaraBio Inc, Shiga, Japan). Plates are centrifuged at 1,000G for 30 minutes at room temperature. The activated Tregs, resuspended in 750-L cell-free viral supernatant, are distributed to the same retronectin-coated, virus-containing wells (1.5×10⁶ activated Tregs/well). Plates are then centrifuged again at 1,000G for 1 hour at room temperature and incubated for 5 hours. After incubation, viral supernatant is replaced with either Biotarget-1 or RPMI 1640 complete medium. Plates are incubated in 5% CO₂ for an additional 2 days before further experimentation.

### Example 4—Migration of Tregs Expressing Neurexin Targeting Polypeptides

To assess the ability of Treg cells to migrate to and persist at sites of neural inflammation using neurexin-neuroligin targeting, Tregs are transfected with the two-element “degen-lock.” (Applicant's term for the cells of the invention that are targeted to neuroligin and C1q). Treg cells are cultured with neuroligin-1 and culture supernatants are collected for assessment of IL-10 and TGF-β by ELISA. Cells are collected after the initial culture and placed into 5 μm pore 96 well polycarbonate membrane trans-well coated with or without neuroligin-1, the lower compartment of the trans-well plate is left untreated or treated with CCL4. Cells are cultured for 4 hours and cells in the top and bottom of the plates are counted. Tregs expressing neurexin targeting polypeptides should not migrate from the insert in the presence of neuroligin.

To assess the ability of T regs expressing neurexin targeting polypeptides to migrate to sites of inflammation in vivo, the transduced, expanded murine Tregs were genetically modified and delivered by intravenous infusion to the animals. Synthetic plasmid pREF061 was digested with EcoRI and NotI according to NEB double-digest protocol, and then following fragments were cloned in using InFusion cloning method (Clontech): REF138 TGATTTATGCGTAACGCCATTTTGCAAGGCATGG (SEQ ID NO: 29) and REF139 GATAAGCTTGATATCGAATTTTACTTGTACAGCTCGTCCATGCC (SEQ ID NO: 30) primers were used to amplify 1179 bp fragment from pREF061 containing GFP; REF140 ATGGCGTTACGCATAAATCAATATTGGCTATTGGCCATTGC (SEQ ID NO: 31) and REF141 ATGGCGTTACTTTATAGAGCTCATCCATCCCAAGTGTGA (SEQ ID NO: 32) were used to generate a 2648 bp fragment from pREF044. REF142 GCTCTATAAAGTAACGCCATTTTGCAAGGCA (SEQ ID NO: 33) and REF143 TCGACTCTAGAGTCGCGGCCGAGCATGTCCAG (SEQ ID NO: 34) were used to generate a 2090 bp fragment from pREF123.

pREF171 was prepared from pREF061 digested with EcoRI and NotI and contained fragment 1179 bp amplified with REF138 TGATTTATGCGTAACGCCATTTTGCAAGGCATGG (SEQ ID NO: 35) and REF139 GATAAGCTTGATATCGAATTTTACTTGTACAGCTCGTCCATGCC (SEQ ID NO: 36) from pREF061, a 2648 bp fragment amplified from pREF044 with REF140 ATGGCGTTACGCATAAATCAATATTGGCTATTGGCCATTGC (SEQ ID NO: 37) and REF144 AATCAATGTCTTTATAGAGCTCATCCATCCCAAGTGTGA (SEQ ID NO: 38) and a 3209 bp fragment amplified with REF145 GCTCTATAAAGACATTGATTATTGACTAGTTATTAATAGTAATCAATTACGGG (SEQ ID NO: 39) and REF143 TCGACTCTAGAGTCGCGGCCGAGCATGTCCAG (SEQ ID NO: 40) from pREF031.

Briefly, PCRs were performed using 12.5 uL HiFi PCR premix (CLontech), 12.5 uL water, 1.25 uL 10 μL of each primer, and the fragments were amplified using Thermofisher thermocycler according to the HiFi PCR premix manufacturers instructions. PCR products were then mixed 5:1 with loading dye (Promega) and run on 1% agarose (Melford) gel in 0.5×TBE (Invitrogen) in the presence of 1×SYBRSafe against Promega 1 kb plus molecular weight marker. Fragments of interest were identified using BioRad imager, and the fragments were cut out from the gel, and then isolated from the cut-outs using MN cleanup kit. Afterwards, 1 μL of each fragment and vector were used alongside 2 uL InFusion 5× kit for 15 minutes at 50° C. Then, 2 μL of each reaction was transformed into Escherichia coli HST08 (Stellar cells, Clonetech) and after overnight incubation at 37° C. on LB (Melford) with 50 μg/mL ampicillin (Melford), the correct clones were selected and confirmed by restriction digest and sequencing. MidiPreps (QiaGene) were performed on LB cultures of correct clones.

Spleens from Male C57BL/6J MOUSE—Age Range Cohort—56 to 62 Days were isolated as per standard protocol at BIE Biological Support Unit. Splens were disrupted in PBS using a syringe plunger, and then were a subject of EasySep™ Mouse CD4+CD25+ Regulatory T Cell Isolation Kit II according to manufacturers' protocol without modifications. Briefly, PBS was removed from spleen and spleens were poured into a sterile, plastic petri dish. Spleens were disrupted in Treg sample preparation buffer (PBS with 2% fetal bovine serum (FBS), 1 mM EDTA) and filtered through a 70 μm mesh nylon strainer (BD). Splenocytes centrifuged at 300×g for 10 minutes and resuspend in 2 mL. 100 μL of rat serum were added to the sample. 100 μL/mL of CD4+ T Cell Isolation Cocktail were added to the sample. Sample was mixed by pipetting and incubated at RT for 10 minutes. Streptavidin RapidSpheres, which select for non CD4 lymphocytes, were thoroughly vortexed until no clumps were observed. 150 μL of Streptavidin rapidspheres were added to the sample. Sample was then mixed and incubated at RT for 2.5 minutes. Tube was then placed on the magnet and incubated on the magnet at RT for 2.5 minutes. Then, the supernatant was decanted into a new tube while being kept in contact on the magnet, this removes non-CD4 splenocytes (these were kept on the beads). Supernatant was exposed to magnet and this step repeated to remove any remaining Streptavidin RapidSpheres. The subsequent pre-enriched cells were centrifuged at 200×g for 10 minutes at RT and resuspended in 0.5 mL of Treg sample preparation buffer. 25 μL of FcR blocker were added to the sample, and the tube was incubated at RT for 5 minutes. 25 μL of the CD25 Regulatory T Cell Positive Selection Cocktail were added to the sample. The incubation time was at RT for 10 minutes. 10 μL of the PE Selection Cocktail were added to the sample. Mix was incubated at RT for 5 minutes. Dextran rapidspheres were vortexed for 30 seconds. 30 μL of Dextran rapid spheres were added to the sample. Incubation was performed at RT for 5 minutes. Tube was then placed on the magnet and incubated at RT for 5 minutes—the CD4+CD25+ sample (on beads) was topped up to 2.5 mL in Treg sample preparation buffer, and again incubated for RT for 5 minutes on the magnet. This enrichment step for CD25+ cells was repeated 4×. Cells were suspended in complete RPMI-1640 containing 10% FCS, 2 mM L-glutamine, 1 mM pyruvate, 10 mM HEPES, 1×MEM NEAAs, 0.05 mM 2-ME and activated with 4:1 Mouse T cell activator dynabeads (Invitrogen). 1000 u/mL human recombinant IL-2 (Peprotech) was added from 10 mM acetic acid solution.

Preparation of the ecotropic lentivirus was performed as follows: in each case 7 micrograms of the cargo vector (pREF170, to generate ‘Alternate Tregs’ or pREF171, to generate ‘SynNotch Tregs’, or pREF061 (GFP only, to generate ‘WT Tregs’) from high-quality midiprep (Qiagen, prepared according to manufacturers' instruction) were mixed with 600 uL distilled water, and then added to the Lenti-X Packaging One Shot vial (Takara). The tube was vortexed, and incubated at room temperature for 10 minutes for the nanoparticle complexes to be formed, according to the manufacturer's instruction. Mixture was then applied to 80% confluent HEK293T culture, and after overnight incubation at 37° C. 5% CO2 the medium was replaced with fresh DMEM (Gibco) with 10% FCS (Sigma) and 1 mM pyruvate (Gibco). The lentivirus was collected after 48 hours and 72 hours and was pooled, and concentrated using Lenti-X Concentrator (Takara). Pooled stocks of the same cargo. Centrifuged at 500 g for 10 min to removed any debris. Transferred clarified supernatant to a sterile 50 mL falcons and combine 1 volume of Lenti-X Concentrator (10 mL) with 3 volumes of clarified supernatant (30 mL). Mixed by gentle inversion. Incubated mixture at 4° C. for 60 minutes. Centrifuged the sample at 1500 g for 45 minutes at 4° C. to concentrate the supernatant. After centrifugation, an off-white pellet was be visible. The pellet was resuspended in 500 μL of complete RPMI to be added to the culture.

Protransduzin fibres were prepared as described in as follows: the 10 mg/mL DMSO solution of protransduzin (JPT) was mixed with PBS to make 1 mg/mL solution with 10 min incubation at RT. 50 μL of 1 mg/mL protransduzin solution was added to 500 uL MLV-enveloped virus and incubated for 15 minutes at RT. Finally, 450 μL of activated Tregs stimulated with CD3/CD28 were mixed with 550 uL complex of MLV-virus and protransduzin and were placed in 24 well plates at 37° C. and 5% CO2. Cells were expanded in the medium described above, 7 days after transduction the cells were sorted for eGFP expression, and re-stimulated and further expanded as described above for an additional 7 days prior to freezing in medium containing 90% FCS and 10% DMSO.

B6.Cg-Tg(SOD1*G93A)1Gur/J mice, stock number 004435, at 8 weeks old were purchased from The Jackson Laboratory, USA. Animals were acclimatised for 7 days prior to weekly weighing and health monitoring check. Food and water were provided Ad libitum and animals were kept in temperature and humidity-controlled environment, with 12 h/12 h light/dark cycle. All procedures were carried out under HO project licence PPL P15A1884A at Medicines Discovery Catapult facilities. ⁸⁹Zr was purchased from commercial radiotracer supplier (PETNET). Syringe activity was measured before and after the i.v. injection using the BriTec well counter and the times of measurement noted using a clock synchronised to the PET system. The injected dose was calculated as the difference of the pre and post injection syringe activities after decay, and corrected to the time of injection. Mice were injected with 2×10\hat{ }6 Tregs labelled when mice reached 120 days of age. Cells were administered intravenously through the tail vein (in the total volume of ˜100 ul in PBS). Mice were euthanised and tissues were collected for biodistribution study. Blood, lungs, liver, brain, stomach, kidneys, small intestine, large intestine, right muscle, left muscle, spinal cord, and tail (injection site) were dissected from each animal for gamma counter ex-vivo analysis. Hydraulic extrusion of the spinal cord was performed for both lumbar and thoracic regions.

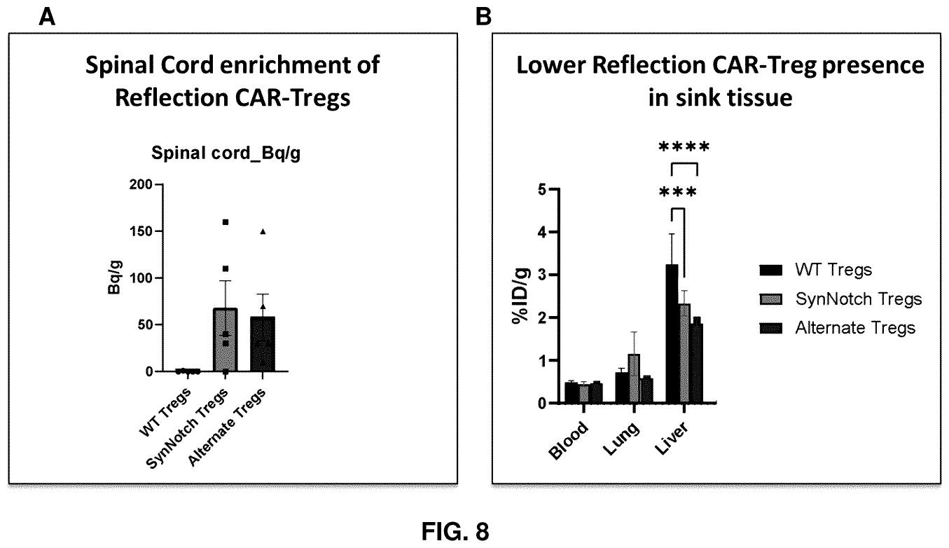

Biodistribution data revealed increased radiation dose in the liver in unmodified Tregs-treated group compared to SynNotch (pREF171-transduced Tregs)- and Alternate (pREF170-transduced Tregs)-treated groups. This result suggests that transduced Tregs might have lower risk to accumulate in the liver compared to a non-modified Tregs. The spinal cord appeared to be more susceptible to infiltration of to SynNotch (pREF171-transduced Tregs)- and Alternate (pREF170-transduced Tregs) Tregs (P=0.1 vs. WT Tregs), whereas the brain to infiltrations of WT Tregs. Results are shown in FIG. 8.

### Example 5—Targeting of Tregs In Vivo to Inflamed Synapse

CD4+ T cells are isolated, then are enriched for CD25+ cells prior to sorting live CD4+CD25hi Tregs using FACS. Sorted cells are stimulated. One day later, cells are transduced with lentivirus carrying the sequence encoding anti-neuroligin receptor and the anti-C1q CAR as well as a fluorescent protein co-expressed using a 2A self-cleaving peptide sequence, a multiplicity of infection of 10 virus particles:1 cell. At day 7, cells are purified with magnetic selection, re-stimulated and expanded for an additional 5 days prior to injection. 5×10⁶ murine CAR Tregs and untargeted T regs are injected to SOD1 G93A animals intravenously into the tail vein on day 50. Saline-injected (PBS) mice serve as a control. GFP fluorescence coming from the modified Tregs is used to document the localisation of the Tregs into inflamed synapses skeletal muscles and spinal cord.

### Example 6—Efficacy of Tregs Expressing Neurexin Targeting Polypeptides Gating a C1q CAR in a Mouse Models of ALS

In vivo efficacy of Tregs expressing neurexin targeting polypeptides gating a C1q CAR can be tested in the SOD1-G93A transgenic mouse. These mice express a G93A mutant form of human SOD1 and are useful in studying neuromuscular disorders such as Amyotrophic Lateral Sclerosis.

A cohort of 75 SOD1 G93A (TG) and 15 wild-type (WT) littermate female mice are divided into four experimental groups, each of 15 animals: WT mice are treated with vehicle, TG mice are treated with vehicle or pREF001 transduced T regs that also express the C1q CAR. Three other groups are treated with the best-performing groups of Tregs from example 5. The mice are analysed for 70 days between the 50^{th} and 120^{th} day. Body weight is monitored once per week between days 50 and 90, and three times per week between days 91 and 120. Wire hanging abilities are measured at three time-points: as a baseline, at week 14 and week 16. An open-field test is performed at two points, at baseline and week 16. Fine motor kinematic gait analysis at baseline and at week 15 is also tested. Terminal sampling is performed to confirm the efficacy by histology, including motor neuron count Iba-1 quantitation.

Tregs were prepared as described in Example 4. EAE kit EK-2110 (MOG35-55/CFA emulsion) and pertussis toxin were purchased from Hooke Laboratories, Inc. MA, USA. 21 μL of stock solution containing 5 μg pertussis toxin in 25 μL glycerol buffer was diluted with 4.2 mL of sterile phosphate buffer saline (PBS calcium and magnesium free) to obtain 100 ng in 100 μL dosing solution. Female C57BL/6J were purchased from Charles River, UK, and were housed at Pharmidex Pharmaceutical Ltd and used at five to eight weeks of age. Female C57BL/6J mice (n=35) were injected subcutaneously with total volume of 200 μL of MOG35-55/CFA (Complete Freund Adjuvant) emulsion, 100 μL each on upper back and on lower back on day 0. After 2 hrs of MOG35-55/CFA emulsion, 100 μL containing 100 ng of pertussis toxin (PTX) was injected intraperitoneally on day 0 and repeated PTX injection again on day 2.

Unmodified Treg cells (1.5×106) and pREF170-transduced Treg Cells (1.5×106) were freeze in a mixture of FCS and DMSO. After thawed individually, they were transferred to 15 mL Falcon with 5 mL of 1×PBS separately and centrifuged at 300 g for 5 minutes. Supernatant was completely removed and suspended the pellet with 1.5 mL of sterile PBS separately to obtain 1×105 cells in 100 μL.

A 100 μL of unmodified Treg cells (100,000 cells) (n=11)/or pREF170-transduced Treg cells (100,000 cells) (n=11)/PBS (n=13) was injected into each mouse on day 11 intraperitoneally.

Daily clinical scores were recorded for each experimental group and the data was displayed as mean group clinical score ±standard error of the mean (SEM) for each day of the clinical score measurements.

During the experiment, total of 6 mice PBS group (n=3), WT Treg cells group (n=2) and AT Treg cells group (n=1) were euthanised for health reasons before the experimental endpoint. These mice clinical scores were included from the data set until euthanised and not scored 5 for those mice for the remaining days. Data was included for 7 mice which showed late onset of EAE symptoms PBS group (n=5) AT Treg cells group (n=2) to avoid between group and intra group variance.

Two way ordinary ANOVA with Dunnett's multiple comparisons test was used to test the hypothesis that treatment was ineffective in improving clinical scores of EAE and considered significant, if p<0.05.

In the untreated group (PBS), 12 mice had mild hind limb paresis in which 9 mice went into complete bilateral hind limb paralysis. Some mice showed mild hind limb paresis on day 18 (n=3) day 19 (n=1) and day 22 (n=1) only. 6 mice partially recovered from complete bilateral hind limb paralysis and 1 mouse fully recovered. 6 mice did not recover even partially from complete bilateral hind limb paralysis in which 3 were humanely killed on day 15 (n=1) and on day 18 (n=2) as the mice conditions did not show improvement.

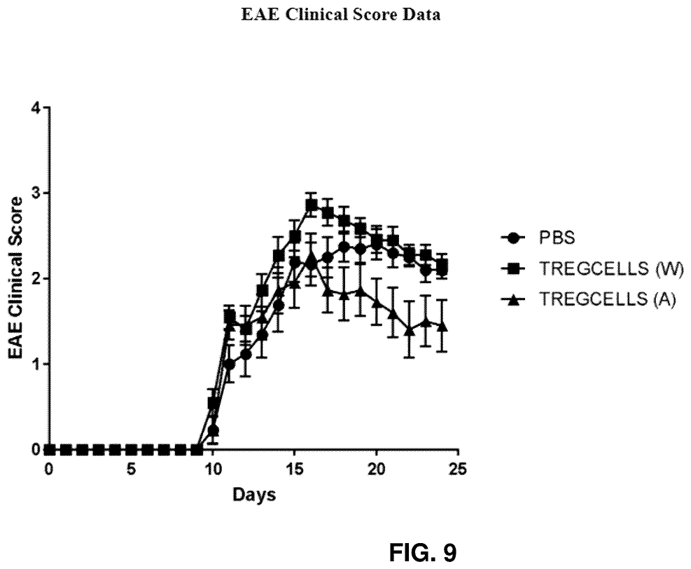

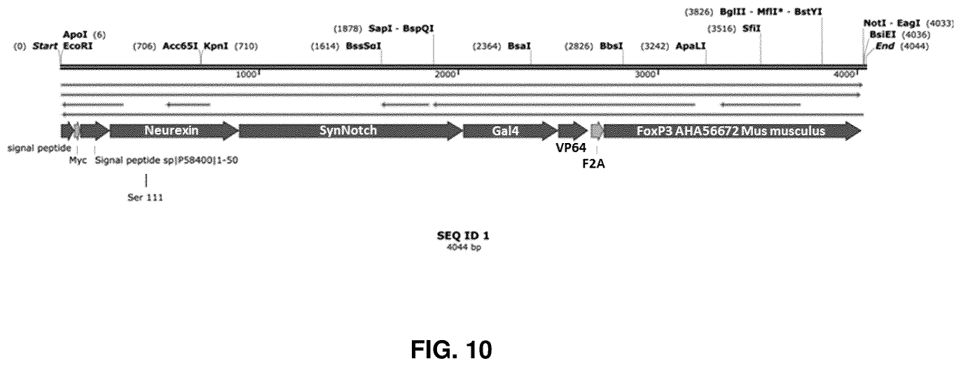

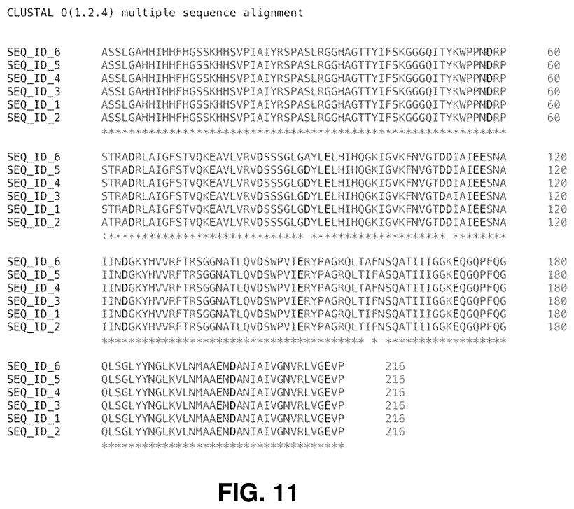

In the pREF170-transduced-Treg treated group, 10 mice had mild hind limb paresis in which all mice went into complete bilateral hind limb paralysis. All mice showed mild hind limb paresis before 16 days. 8 mice partially recovered from complete bilateral hind limb paralysis and 1 mouse fully recovered. 2 mice did not recover even partially from complete bilateral hind limb paralysis and were humanely killed on day 20 (n=1) and on day 21 (n=1) as the mice conditions did not show improvement. 9 mice had mild hind limb paresis in which 2 mice showed mild hind limb paresis on day 18 and day 19 only and 6 mice went into complete bilateral hind limb paralysis. 2 mice showed only mild tail tone loss and never had any other symptoms. 6 mice partially recovered from complete bilateral hind limb paralysis and 4 mice fully recovered. 1 mouse recovered from complete bilateral hind limb paralysis to mild hind limb paresis however it was humanely killed on day 20 as the mouse showed very limited movement. The results are illustrated in the FIG. 9.

### Example 7—C1q Activates Tregs Through CAR Binding

Murine Tregs were transfected with a construct driving constitutive expression of a CAR of the invention with an antigen determining region comprising ScFv directed to C1q (construct pREF043). Beads were coated with C1q in the carbonate buffer. The carbonate buffer 0.05 M pH 9.5 was filter-sterilised through a Sartorius 0.2 um filter; The choice of the buffer was dictated by the isoelectric point of C1q chains (pI of chains A (8.87) and B (9.07). The pI of chain C is close to 7 (7.07)). Tosyl-activated Dynabeads (500 μl) (6×10⁸ to 7×10⁸ beads per ml; M-280, Thermofisher, sold as 30 mg beads per mL) are pelleted by placing the tube in the powerful magnetic field of a magnetic particle concentrator (Stemcell).

Following removal of storage buffer, the beads are washed once with 1 ml of coating buffer (0.05 M carbonate, pH 9.5). After a final concentration, 250 μl of coating buffer was added, the beads were suspended, and 250 μl of human C1q (0.4 mg/ml of coating buffer) was added. Coupling of C1q was performed by gentle rotation for 24 h at 37° C. Wash beads three times with phosphate-buffered saline (PBS; pH 7.2) containing 2% bovine serum albumin (BSA). Following an overnight wash at 4° C. with the same buffer, the beads were suspended in sterile-filtered 0.5 ml of PBS-1% BSA and stored at 4 degC.

Cells were exposed to C1q-coated tosyl-activated beads and activation of T regs was determined by detection of CD69 expression. As a positive control, PHA—0.25 ng/ml; in the 1 ml culture, 0.25 μl added of the original stock (Cat No: 11082132001 from Sigma).

Briefly, after 24 hours, the Tregs were collected by centrifugation at 300 g for 5 minutes. The supernatant was removed and 500 uL PBS were added to the pellet. 1 μL of Zombie violet in DMSO (Biolegend) were added, and the sample was incubated for 15 minutes at RT in the dark. Then, the sample was centrifugated at 300 g for 5 minutes. The sample was suspended in PBS with 10% FCS for staining with the anti-CD69 antibody. Biolegend antibody against CD69 conjugated with PE has been used for detection (the Armenian Hamster IgG H1.2F3 antibody conjugated with PE. Recommended concentration of this antibody is 50.25 μg per sample. Original stock was 0.2 mg/ml, so 1.25 uL of antibody was used for staining condition. Staining was performed for 15 minutes at 4° C. Three washes by 300 g for 5 minutes. Then the samples were taken for flow analysis). Cytometer:LSRFortessa A (LSRFortessa) was used for the analysis. Zombie violet was detected using 405 nm excitation and 450/50 nm bandwidth emission filter. The fluorescence signal from the CD69 detected with the PE-conjugated antibody was measured using 561 nm excitation 586/15 emission bandwidth channel.

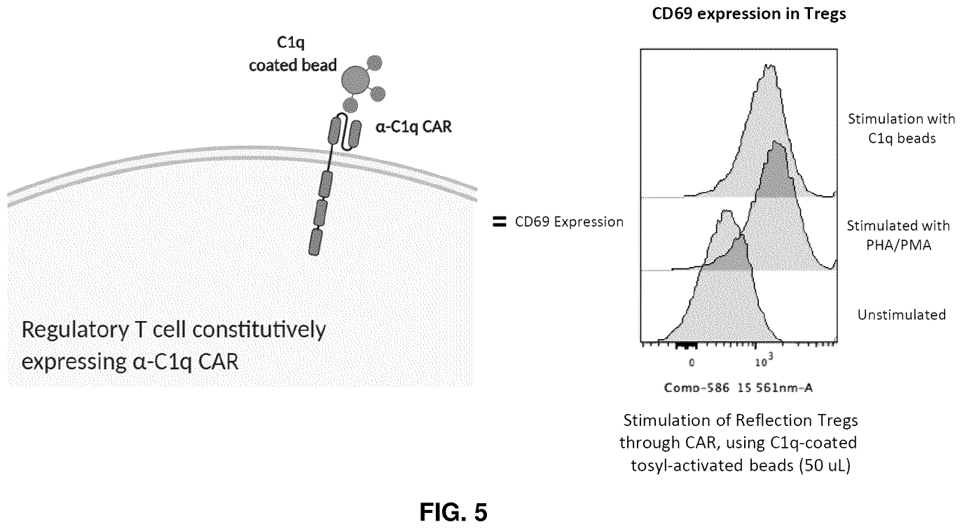

The results are displayed in FIG. 5, which shows that CD69 expression is significantly increased in cells stimulated with C1q beads over unstimulated cells and those stimulated with PHA/PMA

### Example 8—C1q CAR Induced by SynNotch Neurexin Receptor Binding Neuroligin

Mouse tregs transduced with ecotropic lentoivirus carrying pREF060 were placed on the magnet in 15 mL falcon for 2.5 minutes and decanted to a new tube, and spinned down for 5 minutes at 300 g and re-suspended in RPMI-1640 medium supplemented with 10% FCS (Hyclone), 2 mM L-glutamine, 1 mM Na-pyruvate, 10 mM HEPES, 1×PenStrep, 1×NEAAs (MEM) and 0.05 mM 2-mercaptoethanol, and cultivate ON without any added IL-2.

C1q beads were prepared and used as described in example 7.

Neuroligin ligand used here was derived from a mouse myeloma cell line, NS0-derived human Neuroligin 1/NLGN1 protein Gln46-Ser677, Deletion: aa 279-287, with a C-terminal 6-His tag (Biotechne, 6446-NL-050). Dynabeads™ His-Tag Isolation and Pulldown 10103D were coated with neuroligin as follows: beads were thoroughly resuspended in the vial (vortexed for 30 sec). Transferred 50 μL (2 mg) Dynabeads™ magnetic beads to a microcentrifuge tube. Place the tube on a magnet for 2 min. Aspirate and discard the supernatant. Added neuroligin from 100× diluted in PBS, the total volume was 200 uL PBS and the final concentration was 1 μg/mL. Incubated the beads with the ligand on the orbital shaker for 10 min at room temperature. Placed the tube on the magnet for 2 min, then discarded the supernatant. Washed the beads 4 times with 300 μL PBS by placing the tube on a magnet for 2 min and discard the supernatant. Resuspended the beads thoroughly between each washing step. Kept until used in the fridge.

Stimulation of pREF060 Tregs was performed in duplicate. Neuroligin-coated beads were added to 6 well plate wells, the control ‘stimulation’ was performed with uncoated beads. Wells contained 2 mL total volume of complete RPMI (with 2 mM glutamine, 1 mM Na-pyruvate, 1×MEM NEAA, 1×PenStrep, 0.05 mM 2-mercaptoethanol, 10% FCS (Hyclone), 10 mM HEPES buffer) at 1×10⁶ cells/mL (2 mln in total per well). Beads were added in 2:1 ratio of Tregs: beads calculated as follows: 2 mg of beads and these were suspended in 300 μL of final volume; dynabeads are 6.5×10⁷ beads per mg, therefore the entire preparation contained 1.3×10⁸ beads. As a result, 5 μL of beads suspension were added per well. For mock-stimulation, 50 μL of bare beads were suspended in 250 μL of PBS, washed once using 300 g 5 min spin-down and resuspended in 300 uL PBS. Also 5 μL of beads were added when appropriate. Cells were returned to the 37° C. 5% CO2 incubator for 24 hours from stimulation.

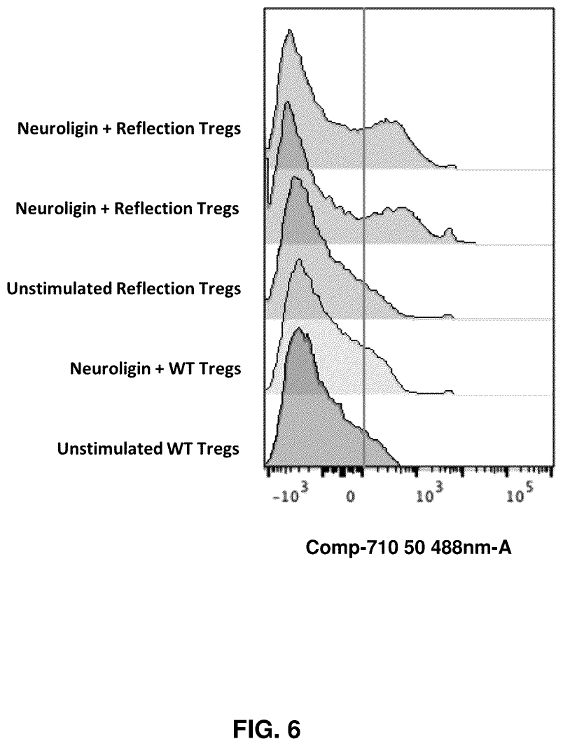

Samples were centrifuged at 300 g for 5 minutes and resuspended in 500 uL PBS with 1 uL zombie violet viability dye in DMSO (Biolegend). Samples were incubated for 30 minutes at RT, and then washed in PBS+10% FCS. Then the samples were incubated with 500 uL PBS+10% FCS in the presence of 1 uL Anti-DDDDK tag (Binds to FLAG® tag sequence) antibody [M2] (PerCP) (ab117514) for 15 minutes in the fridge. Samples were then washed three times, each time with resuspension in 1000 μL of PBS+10% FCS, and were finally resuspended in 1000 μL of PBS+10% FCS. Centrifugation steps were always 300 g for 5 minutes at RT. Samples were kept on ice until measured at Babraham Institute flow cytometry facility. Results of the flow cytometry are shown in FIG. 6. This demonstrates that the AND gate construct works, in that stimulation of the SynNotch receptor leads to release of FoxP3 and expression and display of CAR.

### Example 9—AND Gate Activates Murine T Regs Through C1q CAR

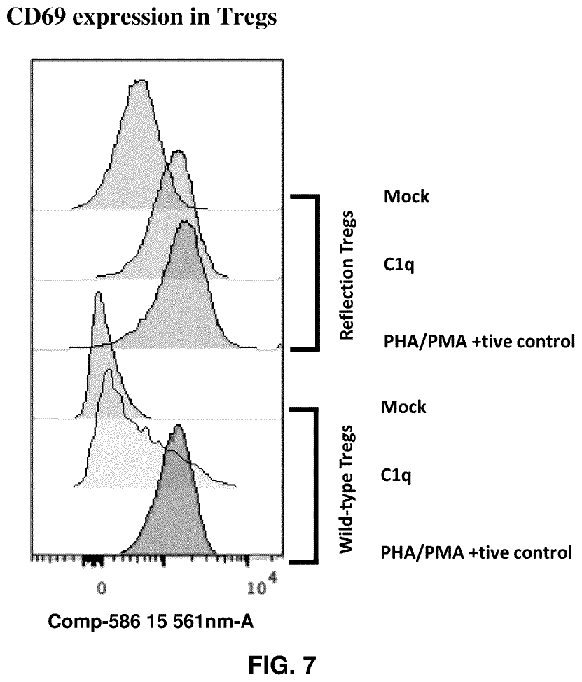

Murine T regs were transfected with the SynNotch neurexin targeting polypeptide of the invention and the anti-C1q CAR of the invention, operatively linked to a FoxP3 binding transcriptional activator. CD69 is an early activation marker of T lymphocytes. Detection of CD69 associated with full functionality of the AND-gate construct pREF060 was performed using PE-conjugated mouse-CD69 specific antibody. Induction was performed after 24 hours from stimulation with C1q-coated tosyl-activated beads upon previous NLGN-1 beads stimulation. The results are displayed in FIG. 7, which shows that the AND gate of the invention drives expression of CD69 and is therefore capable of activating T regs.

After the stimulation the neuroligin beads, as detailed in Example 8, beads were removed from the sample of 500′000 Tregs by exposing through the strong magnetic field of the Stemcell EasySep magnet and incubation for 2.5 minutes at RT. The cells were then placed in 1 mLwells of 24 well plate in complete RPMI (with 2 mM glutamine (Gibco), 1 mM Na-pyruvate (Gibco), 1×MEM NEAA (Gibco), 1×PenStrep (Gibco), 0.05 mM 2-mercaptoethanol (Gibco), 10% FCS (Hyclone), 10 mM HEPES buffer). Note, that no IL-2 was added to the culture to avoid any non-specific stimulation. 50 μL of C1q bead suspension prepared as described above were added when appropriate, when no stimulation was intended 50 μL of beads not conjugated with C1q and washed with PBS were added as ‘Mock’ beads for ‘mock stimulation’. For positive controls of activation and CD69 detection, PMA—200 μg/ml; in the 1 ml culture, 0.75 μl added of the original stock (Ref: P1585-1 MG from Sigma). PHA—0.25 ng/ml; in the 1 ml culture, 0.25 μl added of the original stock (Cat No: 11082132001 from Sigma).

After 24 hours, the Tregs were collected by centrifugation at 300 g for 5 minutes. The supernatant was removed and 500 uL PBS were added to the pellet. 1 μL of Zombie violet in DMSO (Biolegend) were added, and the sample was incubated for 15 minutes at RT in the dark. Then, the sample was centrifugated at 300 g for 5 minutes. The sample was suspended in PBS with 10% FCS for staining with the anti-CD69 antibody. Biolegend antibody against CD69 conjugated with PE has been used for detection (the Armenian Hamster IgG H1.2F3 antibody conjugated with PE. Recommended concentration of this antibody is <0.25 μg per sample. Original stock was 0.2 mg/ml, so 1.25 uL of antibody was used for staining condition. Staining was performed for 15 minutes at 4° C. Three washes by 300 g for 5 minutes. Then the samples were taken for flow analysis). Cytometer:LSRFortessa A (LSRFortessa) was used for the analysis. Zombie violet was detected using 405 nm excitation and 450/50 nm bandwidth emission filter. The fluorescence signal from the CD69 detected with the PE-conjugated antibody was measured using 561 nm excitation 586/15 emission bandwidth channel. The results of the signal are showed in the FIG. 7.

The results here showed the full activation circuit of the pREF060 and-gate of our prototype cell therapeutic. We have shown here that the pREF060 Tregs after stimulation with neuroligin, but only in the presence of C1q, express CD69, an early activation marker promoting Treg immunosuppressive function. This means our cell therapy prototype can be used for targeting to inflamed synapse, as well as the similar system can be used for detection of any disease-specific set of two antigens.

### Example 10—Design of Neurexin Tether for SynNotch Independent Targeting

An insert comprising a chimeric targeting polypeptide to act as a membrane anchored tether was designed as follows: an 18 amino acid signal peptide sequence MSMLFYTLITAFLIGIQA (SEQ ID NO: 22) was fused to a myc-tag sequence EQKLISEEDL (SEQ ID NO: 21) for ease of detection, the neurexin 1-b polypeptide of SEQ ID NO: 1 and a membrane anchor polypeptide sequence GGGGSGGGGSGGGGS (SEQ ID NO: 23). The full tethered neurexin sequence comprising SEQ ID NO: 1 is provided as SEQ ID NO: 24.

### Example 11—SynNotch Independent Targeting System Demonstrates Significant Efficacy In Vivo

A study on the efficacy of synNotch independent (neurexin tether) targeting system (Alternate) of the invention was performed on an experimentally induced mouse model of multiple scleroses known as experimental autoimmune encephalomyelitis (EAE). This model is produced by administering a myelin basic protein peptide (MBP) fragment that induces an autoimmune response directed to the myelin sheath surrounding motor neurons. The EAE score gives a measurement of neurological impairment with 0 being no obvious changed over motor function in normal mice and 5 being the most severe, typically paralysis and recommended euthanasia. In this study, there were 3 treatment groups of mice PBS, Wildtype T regs (W) and Alternate Targeted Tregs (A) of the invention. EAE was induced at day 1 and mice were dosed with 100,000 cells by i.p. at day 15. The results are displayed in FIG. 9, which shows that cognitive impairment in all groups starts at around day 10 and continues up to day 15 when the cells were administered. In the two control groups (PBS and W) the cognitive impairment stabilized and drops slights up to the end of the study. However, in the test group who received the cells of the invention, the cognitive decline appears to reverse and finishes much lower than the control groups at the end of the study. This may suggest that the cells of the invention not only halted the cognitive decline but appear to have reversed it in part. This may be due to a reduction in inflammation at motor neurons.

### Example 12—FoxP3 Expression in Tregs Maintained in Inflammatory Conditions

In this experiment regulatory T cells were transfected with Reflection Therapeutics technology according an embodiment of the present invention (in this case a tissue tether-P2A-FOXP3 transcript, under the control of a constitutive promoter, which triggers the expression of an a-C1q CAR under the control of FOXP3 response elements). These cells were cultured under normal, or ‘pro-inflammatory’ conditions, achieved by the addition of IL-1β, IL-6 and TNFα.

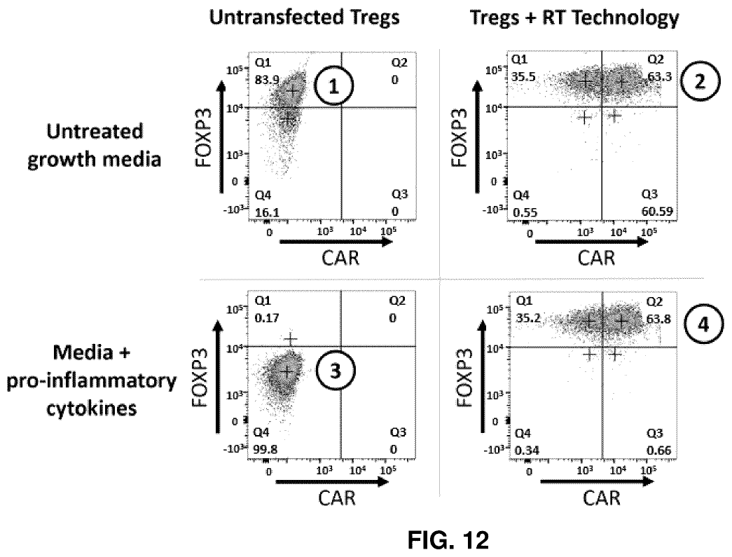

The results of this experiment are provided in FIG. 12. Human regulatory T cells, isolated from PBMCs, were either untransfected, or transfected with a plasmid bearing Reflection Therapeutics ‘degen-lock’ targeting technology according to an embodiment of the invention which includes: a tissue targeting receptor, FOXP3, and a CAR which is under the control of a FOXP3 response element. 24, 48 and 72 hours after transfection cells were treated with IL-2 at 1000 μ/mL, and in addition cells in the ‘pro-inflammatory’ condition were treated with IL-6 (100 ng/ml), IL-1b (100 ng/ml) and TNF-alpha (100 ng/ml). 96 hours after transfection cells were collected and analysed by flow cytometry. Data presented shows levels of FOXP3 and Chimeric Antigen Receptor (FLAG tag).

This experiment demonstrates that the present invention can increase and stabilise FOXP3 levels in regulatory T cells, even in highly inflammatory conditions (4), in contrast to untransfected cells which lose FOXP3 expression (3). Furthermore this stable and high level of FOXP3 expression is linked to Chimeric Antigen Receptor expression, ensuring cells without FOXP3, and thus the anti-inflammatory properties of Tregs, are unlikely to express CARs, preventing the development of a pro-inflammatory CAR-T subpopulation. This suggests that the cells of the present invention will maintain a stable regulatory T cell phenotype when targeted to neuronal tissue, thereby reducing inflammation in the target tissue.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Sequences Forming Part of the Application as Filed:

| SEQ | | | |
|---|---|---|---|
| ID | | | |
| NO: | Sequence | Origin | Description |
| 1 | ASSLGAHHIHHFHGSSKHHSVPIAIYRSPASLRGG | WT | Neurexin-segment |
| | HAGTTYIFSKGGGQITYKWPPNDRPSTRADRLAI | | derived from the WT |
| | GFSTVQKEAVLVRVDSSSGLGDYLELHIHQGKIGV | | sequence, |
| | KFNVGTDDIAIEESNAIINDGKYHVVRFTRSGGNA | | responsible for |
| | TLQVDSWPVIERYPAGRQLTIFNSQATIIIGGKEQG | | docking the CAR- |
| | QPFQGQLSGLYYNGLKVLNMAAENDANIAIVGNV | | Treg into a |
| | RLVGEVP | | neuroligin- |
| | | | expressing locus |
| 2 | ASSLGAHHIHHFHGSSKHHSVPIAIYRSPASLRGG | S111A | Neurexin-segment |
| | HAGTTYIFSKGGGQITYKWPPNDRPATRADRLAI | | derived from the WT |
| | GFSTVQKEAVLVRVDSSSGLGDYLELHIHQGKIGV | | sequence, |
| | KFNVGTDDIAIEESNAIINDGKYHVVRFTRSGGNA | | responsible for |
| | TLQVDSWPVIERYPAGRQLTIFNSQATIIIGGKEQG | | docking the CAR- |
| | QPFQGQLSGLYYNGLKVLNMAAENDANIAIVGNV | | Treg into a |
| | RLVGEVP | | neuroligin- |
| | | | expressing locus - |
| | | | tuned affinity mutant |
| 3 | ASSLGAHHIHHFHGSSKHHSVPIAIYRSPASLRGG | D162A | Neurexin-segment |
| | HAGTTYIFSKGGGQITYKWPPNDRPSTRADRLAI | | derived from the WT |
| | GFSTVQKEAVLVRVDSSSGLGDYLELHIHQGKIGV | | sequence, |
| | KFNVGTDAIAIEESNAIINDGKYHVVRFTRSGGNA | | responsible for |
| | TLQVDSWPVIERYPAGRQLTIFNSQATIIIGGKEQG | | docking the CAR- |
| | QPFQGQLSGLYYNGLKVLNMAAENDANIAIVGNV | | Treg into a |
| | RLVGEVP | | neuroligin- |
| | | | expressing locus - |
| | | | tuned affinity mutant |
| 4 | ASSLGAHHIHHFHGSSKHHSVPIAIYRSPASLRGG | I210A | Neurexin-segment |
| | HAGTTYIFSKGGGQITYKWPPNDRPSTRADRLAI | | derived from the WT |
| | GFSTVQKEAVLVRVDSSSGLGDYLELHIHQGKIGV | | sequence, |
| | KFNVGTDDIAIEESNAIINDGKYHVVRFTRSGGNA | | responsible for |
| | TLQVDSWPVIERYPAGRQLTAFNSQATIIIGGKEQ | | docking the CAR- |
| | GQPFQGQLSGLYYNGLKVLNMAAENDANIAIVGN | | Treg into a |
| | VRLVGEVP | | neuroligin- |
| | | | expressing locus - |
| | | | tuned affinity mutant |
| 5 | ASSLGAHHIHHFHGSSKHHSVPIAIYRSPASLRGG | N212A | Neurexin-segment |
| | HAGTTYIFSKGGGQITYKWPPNDRPSTRADRLAI | | derived from the WT |
| | GFSTVQKEAVLVRVDSSSGLGDYLELHIHQGKIGV | | sequence, |
| | KFNVGTDDIAIEESNAIINDGKYHVVRFTRSGGNA | | responsible for |
| | TLQVDSWPVIERYPAGRQLTIFASQATIIIGGKEQG | | docking the CAR- |
| | QPFQGQLSGLYYNGLKVLNMAAENDANIAIVGNV | | Treg into a |
| | RLVGEVP | | neuroligin- |
| | | | expressing locus - |
| | | | tuned affinity mutant |
| 6 | ASSLGAHHIHHFHGSSKHHSVPIAIYRSPASLRGG | D141A/ | Neurexin-segment |
| | HAGTTYIFSKGGGQITYKWPPNDRPSTRADRLAI | I210A | derived from the WT |
| | GFSTVQKEAVLVRVDSSSGLGAYLELHIHQGKIGV | | sequence, |
| | KFNVGTDDIAIEESNAIINDGKYHVVRFTRSGGNA | | responsible for |
| | TLQVDSWPVIERYPAGRQLTAFNSQATIIIGGKEQ | | docking the CAR- |
| | GQPFQGQLSGLYYNGLKVLNMAAENDANIAIVGN | | Treg into |
| | VRLVGEVP | | a |
| | | | neuroligin- |
| | | | expressing locus - |
| | | | tuned affinity mutant |
| 7 | MALPVTALLLPLALLLHAARPEQKLISEEDLAAADI | scFv | Sequence of |
| | VLTQTPLSLPVSLGDQASISCRSSQNIVHSNGNTY | | neuroligin- |
| | LEWYLQKPGQSPKLLIYKVSNRFSGVPDRFSGSG | | recognising |
| | SGTDFTLKISRVEAEDLGVYFCFQGSLLPWTFGG | | receptor in scFv |
| | GTKLEIKRADAAPTVSGSTSGSGKPGSGEGSEFT | | form, followed by |
| | SPASSSEVKLMESGGGSVKPGGSLKLSCVASGF | | synNotch and using |
| | TFSDYYMYWVRQTPEKRLEWVATISDGGNYPYY | | Gal4/VP64 as a |
| | PDSVKGRFTISRDNARNNLYLQMTSLKSEDTAMY | | transcription factor. |
| | YCARGGAYSRSDGRFYYAMDYWGQGTSVTVSS | | This is the full ORF |
| | ARILDYSFTGGAGRDIPPPQIEEACELPECQVDAG | | of the construct. |
| | NKVCNLQCNNHACGWDGGDCSLNFNDPWKNCT | | |
| | QSLQCWKYFSDGHCDSQCNSAGCLFDGFDCQL | | |
| | TEGQCNPLYDQYCKDHFSDGHCDQGCNSAECE | | |
| | WDGLDCAEHVPERLAAGTLVLVVLLPPDQLRNNS | | |
| | FHFLRELSHVLHTNVVFKRDAQGQQMIFPYYGHE | | |
| | EELRKHPIKRSTVGWATSSLLPGTSGGRQRRELD | | |
| | PMDIRGSIVYLEIDNRQCVQSSSQCFQSATDVAAF | | |
| | LGALASLGSLNIPYKIEAVKSEPVEPPLPSQLHLMY | | |
| | VAAAAFVLLFFVGCGVLLSRKRRRMKLLSSIEQAC | | |
| | DICRLKKLKCSKEKPKCAKCLKNNWECRYSPKTK | | |
| | RSPLTRAHLTEVESRLERLEQLFLLIFPREDLDMIL | | |
| | KMDSLQDIKALLTGLFVQDNVNKDAVTDRLASVE | | |
| | TDMPLTLRQHRISATSSSEESSNKGQRQLTVSAA | | |
| | AGGSGGSGGSDALDDFDLDMLGSDALDDFDLDM | | |
| | LGSDALDDFDLDMLGSDALDDFDLDMLGS | | |
| 8 | PECQFPASSPCLGGNPCYNQGTCEPTSESPFYR | Human | This is a protein |
| | CLCPAKFNGLLCHILDYSFGGGAGRDIPPPLIEEA | notch | sequence of human |
| | CELPECQEDAGNKVCSLQCNNHACGWDGGDCS | | Notch, that can be |
| | LNFNDPWKNCTQSLQCWKYFSDGHCDSQpcvgC | | used instead of |
| | NSAGCLFDGFDCQRAEGQCNPLYDQYCKDHFSD | | mouse Notch in the |
| | GHCDQGCNSAECEWDGLDCAEHVPERLAAGTLV | | synNotch. |
| | VVVLMPPEQLRNSSFHFLRELSRVLHTNVVFKRD | | |
| | AHGQQMIFPYYGREEELRKHPIKRAAEGWAAPDA | | |
| | LLGQVKASLLPGGSEGGRRRRELDPMDVRGSIVY | | |
| | LEIDNRQCVQASSQCFQSATDVAAFLGALASLGS | | |
| | LNIPYKIEAVQSETVEPPPPAQLHFMYVAAAAFVL | | |
| | LFFVGCGVLLSRKR | | |
| 9 | PPQIEEACELPECQVDAGNKVCNLQCNNHACGW | Mouse | This is a protein |
| | DGGDCSLNFNDPWKNCTQSLQCWKYFSDGHCD | Notch | sequence of mouse |
| | SQCNSAGCLFDGFDCQLTEGQCNPLYDQYCKDH | | Notch-1, that can be |
| | FSDGHCDQGCNSAECEWDGLDCAEHVPERLAA | | used in the |
| | GTLVLVVLLPPDQLRNNSFHFLRELSHVLHTNVVF | | synNotch. |
| | KRDAQGQQMIFPYYGHEEELRKHPIKRSTVGWAT | | |
| | SSLLPGTSGGRQRRELDPMDIRGSIVYLEIDNRQC | | |
| | VQSSSQCFQSATDVAAFLGALASLGSLNIPYKIEA | | |
| | VKSEPVEPPLPSQLHLMYVAAAAFVLLFFVGCGV | | |
| | LLS | | |
| 10 | PCVGSNPCYNQGTCEPTSENPFYRCLCPAKFNG | Notch 1 | |
| | LLCHILDYSFTGGAGRDIPPPQIEEACELPECQVD | beta from | |
| | AGNKVCNLQCNNHACGWDGGDCSLNFNDPWKN | mice | |
| | CTQSLQCWKYFSDGHCDSQCNSAGCLFDGFDC | | |
| | QLTEGQCNPLYDQYCKDHFSDGHCDQGCNSAE | | |
| | CEWDGLDCAEHVPERLAAGTLVLVVLLPPDQLRN | | |
| | NSFHFLRELSHVLHTNVVFKRDAQGQQMIFPYYG | | |
| | HEEELRKHPIKRSTVGWATSSLLPGTSGGRQRRE | | |
| | LDPMDIRGSIVYLEIDNRQCVQSSSQCFQSATDVA | | |
| | AFLGALASLGSLNIPYKIEAVKSEPVEPPLPSQLHL | | |
| | MYVAAAAFVLLFFVGCGVLLS | | |
| 11 | PCVGSNPCYNQGTCEPTSENPFYRCLCPAKFNG | Notch | Notch From our |
| | LLCHILDYSFTGGAGRDIPPPQIEEACELPECQVD | From our | construct |
| | AGNKVCNLQCNNHACGWDGGDCSLNFNDPWKN | construct | |
| | CTQSLQCWKYFSDGHCDSQCNSAGCLFDGFDC | | |
| | QLTEGQCNPLYDQYCKDHFSDGHCDQGCNSAE | | |
| | CEWDGLDCAEHVPERLAAGTLVLVVLLPPDQLRN | | |
| | NSFHFLRELSHVLHTNVVFKRDAQGQQMIFPYYG | | |
| | HEEELRKHPIKRSTVGWATSSLLPGTSGGRQRRE | | |
| | LDPMDIRGSIVYLEIDNRQCVQSSSQCFQSATDVA | | |
| | AFLGALASLGSLNIPYKIEAVKSEPVEPPLPSQLHL | | |
| | MYVAAAAFVLLFFVGCGVLLSRKRRR | | |
| 12 | QLCIQKLMKLLSSIEQACDICRLKKLKCSKEKPKCA | Gal4 | Transcription factor |
| | KCLKNNWECRYSPKTKRSPLTRAHLTEVESRLER | | Gal4, can be used |
| | LEQLFLLIFPREDLDMILKMDSLQDIKALLTGLFVQ | | for driving inducible |
| | DNVNKDAVTDRLASVETDMPLTLRQHRISATSSS | | expression |
| | EESSNKGQRQLTVSAAAGGSGGSGGSDALDDFD | | |
| | LDMLGSDALDDFDLDMLGSDALDDFDLDMLGSD | | |
| | ALDDFDLDMLGSGSGVKQTLNFDLLKLAGDVESN | | |
| | PGPVD | | |
| 13 | RPAKPMAPSLALGPSPGVLPSWKTAPKGSELLGT | FoxP3 | Mouse FoxP3 |
| | RGSGGPFQGRDLRSGAHTSSSLNPLPPSQLQLP | | |
| | TVPLVMVAPSGARLGPSPHLQALLQDRPHFMHQ | | |
| | LSTVDAHAQTPVLQVRPLDNPAMISLPPPSAATG | | |
| | VFSLKARPGLPPGINVASLEWVSREPALLCTFPRS | | |
| | GTPRKDSNLLAAPQGSYPLLANGVCKWPGCEKV | | |
| | FEEPEEFLKHCQADHLLDEKGKAQCLLQREVVQS | | |
| | LEQQLELEKEKLGAMQAHLAGKMALAKAPSVAS | | |
| | MDKSSCCIVATSTQGSVLPAWSAPREAPDGGLFA | | |
| | VRRHLWGSHGNSSFPEFFHNMDYFKYHNMRPPF | | |
| | TYATLIRWAILEAPERQRTLNEIYHWFTRMFAYFR | | |
| | NHPATWKNAIRHNLSLHKCFVRVESEKGAVWTV | | |
| | DEFEFRKKRSQRPNKCSNPCP | | |
| 14 | MALPVTALLLPLALLLHAARPEQKLISEEDLMYQR | Entire | Entire construct |
| | MLRCGAELGSPGGGGGGGGGGGAGGRLALLWI | construct | ORF protein |
| | VPLTLSGLLGVAWGASSLGAHHIHHFHGSSKHHS | WT | sequence, |
| | VPIAIYRSPASLRGGHAGTTYIFSKGGGQITYKWP | | containing SEQ ID: |
| | PNDRPSTRADRLAIGFSTVQKEAVLVRVDSSSGL | | 1 WT neurexin |
| | GDYLELHIHQGKIGVKFNVGTDDIAIEESNAIINDG | | domain, followed by |
| | KYHVVRFTRSGGNATLQVDSWPVIERYPAGRQLT | | the Gal4 and FoxP3 |
| | IFNSQATIIIGGKEQGQPFQGQLSGLYYNGLKVLN | | after P2A. |
| | MAAENDANIAIVGNVRLVGEVPGGGGSPCVGSN | | |
| | PCYNQGTCEPTSENPFYRCLCPAKFNGLLCHILD | | |
| | YSFTGGAGRDIPPPQIEEACELPECQVDAGNKVC | | |
| | NLQCNNHACGWDGGDCSLNFNDPWKNCTQSLQ | | |
| | CWKYFSDGHCDSQCNSAGCLFDGFDCQLTEGQ | | |
| | CNPLYDQYCKDHFSDGHCDQGCNSAECEWDGL | | |
| | DCAEHVPERLAAGTLVLVVLLPPDQLRNNSFHFL | | |
| | RELSHVLHTNVVFKRDAQGQQMIFPYYGHEEELR | | |
| | KHPIKRSTVGWATSSLLPGTSGGRQRRELDPMDI | | |
| | RGSIVYLEIDNRQCVQSSSQCFQSATDVAAFLGA | | |
| | LASLGSLNIPYKIEAVKSEPVEPPLPSQLHLMYVAA | | |
| | AAFVLLFFVGCGVLLSRKRRRQLCIQKLMKLLSSI | | |
| | EQACDICRLKKLKCSKEKPKCAKCLKNNWECRYS | | |
| | PKTKRSPLTRAHLTEVESRLERLEQLFLLIFPREDL | | |
| | DMILKMDSLQDIKALLTGLFVQDNVNKDAVTDRLA | | |
| | SVETDMPLTLRQHRISATSSSEESSNKGQRQLTV | | |
| | SAAAGGSGGSGGSDALDDFDLDMLGSDALDDFD | | |
| | LDMLGSDALDDFDLDMLGSDALDDFDLDMLGSG | | |
| | SGVKQTLNFDLLKLAGDVESNPGPVDMPNPRPA | | |
| | KPMAPSLALGPSPGVLPSWKTAPKGSELLGTRGS | | |
| | GGPFQGRDLRSGAHTSSSLNPLPPSQLQLPTVPL | | |
| | VMVAPSGARLGPSPHLQALLQDRPHFMHQLSTV | | |
| | DAHAQTPVLQVRPLDNPAMISLPPPSAATGVFSL | | |
| | KARPGLPPGINVASLEWVSREPALLCTFPRSGTP | | |
| | RKDSNLLAAPQGSYPLLANGVCKWPGCEKVFEE | | |
| | PEEFLKHCQADHLLDEKGKAQCLLQREVVQSLEQ | | |
| | QLELEKEKLGAMQAHLAGKMALAKAPSVASMDK | | |
| | SSCCIVATSTQGSVLPAWSAPREAPDGGLFAVRR | | |
| | HLWGSHGNSSFPEFFHNMDYFKYHNMRPPFTYA | | |
| | TLIRWAILEAPERQRTLNEIYHWFTRMFAYFRNHP | | |
| | ATWKNAIRHNLSLHKCFVRVESEKGAVWTVDEFE | | |
| | FRKKRSQRPNKCSNPCP | | |
| 15 | MALPVTALLLPLALLLHAARPEQKLISEEDLMYQR | | Entire construct |
| | MLRCGAELGSPGGGGGGGGGGGAGGRLALLWI | | ORF protein |
| | VPLTLSGLLGVAWGASSLGAHHIHHFHGSSKHHS | | sequence, |
| | VPIAIYRSPASLRGGHAGTTYIFSKGGGQITYKWP | | containing SEQ ID: |
| | PNDRPATRADRLAIGFSTVQKEAVLVRVDSSSGL | | 2 WT neurexin |
| | GDYLELHIHQGKIGVKFNVGTDDIAIEESNAIINDG | | domain, followed by |
| | KYHVVRFTRSGGNATLQVDSWPVIERYPAGRQLT | | the Gal4 and FoxP3 |
| | IFNSQATIIIGGKEQGQPFQGQLSGLYYNGLKVLN | | after P2A. |
| | MAAENDANIAIVGNVRLVGEVPGGGGSPCVGSN | | |
| | PCYNQGTCEPTSENPFYRCLCPAKFNGLLCHILD | | |
| | YSFTGGAGRDIPPPQIEEACELPECQVDAGNKVC | | |
| | NLQCNNHACGWDGGDCSLNFNDPWKNCTQSLQ | | |
| | CWKYFSDGHCDSQCNSAGCLFDGFDCQLTEGQ | | |
| | CNPLYDQYCKDHFSDGHCDQGCNSAECEWDGL | | |
| | DCAEHVPERLAAGTLVLVVLLPPDQLRNNSFHFL | | |
| | RELSHVLHTNVVFKRDAQGQQMIFPYYGHEEELR | | |
| | KHPIKRSTVGWATSSLLPGTSGGRQRRELDPMDI | | |
| | RGSIVYLEIDNRQCVQSSSQCFQSATDVAAFLGA | | |
| | LASLGSLNIPYKIEAVKSEPVEPPLPSQLHLMYVAA | | |
| | AAFVLLFFVGCGVLLSRKRRRQLCIQKLMKLLSSI | | |
| | EQACDICRLKKLKCSKEKPKCAKCLKNNWECRYS | | |
| | PKTKRSPLTRAHLTEVESRLERLEQLFLLIFPREDL | | |
| | DMILKMDSLQDIKALLTGLFVQDNVNKDAVTDRLA | | |
| | SVETDMPLTLRQHRISATSSSEESSNKGQRQLTV | | |
| | SAAAGGSGGSGGSDALDDFDLDMLGSDALDDFD | | |
| | LDMLGSDALDDFDLDMLGSDALDDFDLDMLGSG | | |
| | SGVKQTLNFDLLKLAGDVESNPGPVDMPNPRPA | | |
| | KPMAPSLALGPSPGVLPSWKTAPKGSELLGTRGS | | |
| | GGPFQGRDLRSGAHTSSSLNPLPPSQLQLPTVPL | | |
| | VMVAPSGARLGPSPHLQALLQDRPHFMHQLSTV | | |
| | DAHAQTPVLQVRPLDNPAMISLPPPSAATGVFSL | | |
| | KARPGLPPGINVASLEWVSREPALLCTFPRSGTP | | |
| | RKDSNLLAAPQGSYPLLANGVCKWPGCEKVFEE | | |
| | PEEFLKHCQADHLLDEKGKAQCLLQREVVQSLEQ | | |
| | QLELEKEKLGAMQAHLAGKMALAKAPSVASMDK | | |
| | SSCCIVATSTQGSVLPAWSAPREAPDGGLFAVRR | | |
| | HLWGSHGNSSFPEFFHNMDYFKYHNMRPPFTYA | | |
| | TLIRWAILEAPERQRTLNEIYHWFTRMFAYFRNHP | | |
| | ATWKNAIRHNLSLHKCFVRVESEKGAVWTVDEFE | | |
| | FRKKRSQRPNKCSNPCP | | |
| 16 | MALPVTALLLPLALLLHAARPEQKLISEEDLMYQR | | Entire construct |
| | MLRCGAELGSPGGGGGGGGGGGAGGRLALLWI | | ORF protein |
| | VPLTLSGLLGVAWGASSLGAHHIHHFHGSSKHHS | | sequence, |
| | VPIAIYRSPASLRGGHAGTTYIFSKGGGQITYKWP | | containing SEQ ID: |
| | PNDRPSTRADRLAIGFSTVQKEAVLVRVDSSSGL | | 3 WT neurexin |
| | GDYLELHIHQGKIGVKFNVGTDAIAIEESNAIINDG | | domain, followed by |
| | KYHVVRFTRSGGNATLQVDSWPVIERYPAGRQLT | | the Gal4 and FoxP3 |
| | IFNSQATIIIGGKEQGQPFQGQLSGLYYNGLKVLN | | after P2A. |
| | MAAENDANIAIVGNVRLVGEVPGGGGSPCVGSN | | |
| | PCYNQGTCEPTSENPFYRCLCPAKFNGLLCHILD | | |
| | YSFTGGAGRDIPPPQIEEACELPECQVDAGNKVC | | |
| | NLQCNNHACGWDGGDCSLNFNDPWKNCTQSLQ | | |
| | CWKYFSDGHCDSQCNSAGCLFDGFDCQLTEGQ | | |
| | CNPLYDQYCKDHFSDGHCDQGCNSAECEWDGL | | |
| | DCAEHVPERLAAGTLVLVVLLPPDQLRNNSFHFL | | |
| | RELSHVLHTNVVFKRDAQGQQMIFPYYGHEEELR | | |
| | KHPIKRSTVGWATSSLLPGTSGGRQRRELDPMDI | | |
| | RGSIVYLEIDNRQCVQSSSQCFQSATDVAAFLGA | | |
| | LASLGSLNIPYKIEAVKSEPVEPPLPSQLHLMYVAA | | |
| | AAFVLLFFVGCGVLLSRKRRRQLCIQKLMKLLSSI | | |
| | EQACDICRLKKLKCSKEKPKCAKCLKNNWECRYS | | |
| | PKTKRSPLTRAHLTEVESRLERLEQLFLLIFPREDL | | |
| | DMILKMDSLQDIKALLTGLFVQDNVNKDAVTDRLA | | |
| | SVETDMPLTLRQHRISATSSSEESSNKGQRQLTV | | |
| | SAAAGGSGGSGGSDALDDFDLDMLGSDALDDFD | | |
| | LDMLGSDALDDFDLDMLGSDALDDFDLDMLGSG | | |
| | SGVKQTLNFDLLKLAGDVESNPGPVDMPNPRPA | | |
| | KPMAPSLALGPSPGVLPSWKTAPKGSELLGTRGS | | |
| | GGPFQGRDLRSGAHTSSSLNPLPPSQLQLPTVPL | | |
| | VMVAPSGARLGPSPHLQALLQDRPHFMHQLSTV | | |
| | DAHAQTPVLQVRPLDNPAMISLPPPSAATGVFSL | | |
| | KARPGLPPGINVASLEWVSREPALLCTFPRSGTP | | |
| | RKDSNLLAAPQGSYPLLANGVCKWPGCEKVFEE | | |
| | PEEFLKHCQADHLLDEKGKAQCLLQREVVQSLEQ | | |
| | QLELEKEKLGAMQAHLAGKMALAKAPSVASMDK | | |
| | SSCCIVATSTQGSVLPAWSAPREAPDGGLFAVRR | | |
| | HLWGSHGNSSFPEFFHNMDYFKYHNMRPPFTYA | | |
| | TLIRWAILEAPERQRTLNEIYHWFTRMFAYFRNHP | | |
| | ATWKNAIRHNLSLHKCFVRVESEKGAVWTVDEFE | | |
| | FRKKRSQRPNKCSNPCP | | |
| 17 | MALPVTALLLPLALLLHAARPEQKLISEEDLMYQR | | Entire construct |
| | MLRCGAELGSPGGGGGGGGGGGAGGRLALLWI | | ORF protein |
| | VPLTLSGLLGVAWGASSLGAHHIHHFHGSSKHHS | | sequence, |
| | VPIAIYRSPASLRGGHAGTTYIFSKGGGQITYKWP | | containing SEQ ID: |
| | PNDRPSTRADRLAIGFSTVQKEAVLVRVDSSSGL | | 4 WT neurexin |
| | GDYLELHIHQGKIGVKFNVGTDDIAIEESNAIINDG | | domain, followed by |
| | KYHVVRFTRSGGNATLQVDSWPVIERYPAGRQLT | | the Gal4 and FoxP3 |
| | AFNSQATIIIGGKEQGQPFQGQLSGLYYNGLKVLN | | after P2A. |
| | MAAENDANIAIVGNVRLVGEVPGGGGSPCVGSN | | |
| | PCYNQGTCEPTSENPFYRCLCPAKFNGLLCHILD | | |
| | YSFTGGAGRDIPPPQIEEACELPECQVDAGNKVC | | |
| | NLQCNNHACGWDGGDCSLNFNDPWKNCTQSLQ | | |
| | CWKYFSDGHCDSQCNSAGCLFDGFDCQLTEGQ | | |
| | CNPLYDQYCKDHFSDGHCDQGCNSAECEWDGL | | |
| | DCAEHVPERLAAGTLVLVVLLPPDQLRNNSFHFL | | |
| | RELSHVLHTNVVFKRDAQGQQMIFPYYGHEEELR | | |
| | KHPIKRSTVGWATSSLLPGTSGGRQRRELDPMDI | | |
| | RGSIVYLEIDNRQCVQSSSQCFQSATDVAAFLGA | | |
| | LASLGSLNIPYKIEAVKSEPVEPPLPSQLHLMYVAA | | |
| | AAFVLLFFVGCGVLLSRKRRRQLCIQKLMKLLSSI | | |
| | EQACDICRLKKLKCSKEKPKCAKCLKNNWECRYS | | |
| | PKTKRSPLTRAHLTEVESRLERLEQLFLLIFPREDL | | |
| | DMILKMDSLQDIKALLTGLFVQDNVNKDAVTDRLA | | |
| | SVETDMPLTLRQHRISATSSSEESSNKGQRQLTV | | |
| | SAAAGGSGGSGGSDALDDFDLDMLGSDALDDFD | | |
| | LDMLGSDALDDFDLDMLGSDALDDFDLDMLGSG | | |
| | SGVKQTLNFDLLKLAGDVESNPGPVDMPNPRPA | | |
| | KPMAPSLALGPSPGVLPSWKTAPKGSELLGTRGS | | |
| | GGPFQGRDLRSGAHTSSSLNPLPPSQLQLPTVPL | | |
| | VMVAPSGARLGPSPHLQALLQDRPHFMHQLSTV | | |
| | DAHAQTPVLQVRPLDNPAMISLPPPSAATGVFSL | | |
| | KARPGLPPGINVASLEWVSREPALLCTFPRSGTP | | |
| | RKDSNLLAAPQGSYPLLANGVCKWPGCEKVFEE | | |
| | PEEFLKHCQADHLLDEKGKAQCLLQREVVQSLEQ | | |
| | QLELEKEKLGAMQAHLAGKMALAKAPSVASMDK | | |
| | SSCCIVATSTQGSVLPAWSAPREAPDGGLFAVRR | | |
| | HLWGSHGNSSFPEFFHNMDYFKYHNMRPPFTYA | | |
| | TLIRWAILEAPERQRTLNEIYHWFTRMFAYFRNHP | | |
| | ATWKNAIRHNLSLHKCFVRVESEKGAVWTVDEFE | | |
| | FRKKRSQRPNKCSNPCP | | |
| 18 | MALPVTALLLPLALLLHAARPEQKLISEEDLMYQR | | Entire construct |
| | MLRCGAELGSPGGGGGGGGGGGGGRLALLWI | | ORF protein |
| | VPLTLSGLLGVAWGASSLGAHHIHHFHGSSKHHS | | sequence, |
| | VPIAIYRSPASLRGGHAGTTYIFSKGGGQITYKWP | | containing SEQ ID: |
| | PNDRPSTRADRLAIGFSTVQKEAVLVRVDSSSGL | | 5 WT neurexin |
| | GDYLELHIHQGKIGVKFNVGTDDIAIEESNAIINDG | | domain, followed by |
| | KYHVVRFTRSGGNATLQVDSWPVIERYPAGRQLT | | the Gal4 and FoxP3 |
| | IFASQATIIIGGKEQGQPFQGQLSGLYYNGLKVLN | | after P2A. |
| | MAAENDANIAIVGNVRLVGEVPGGGGSPCVGSN | | |
| | PCYNQGTCEPTSENPFYRCLCPAKFNGLLCHILD | | |
| | YSFTGGAGRDIPPPQIEEACELPECQVDAGNKVC | | |
| | NLQCNNHACGWDGGDCSLNFNDPWKNCTQSLQ | | |
| | CWKYFSDGHCDSQCNSAGCLFDGFDCQLTEGQ | | |
| | CNPLYDQYCKDHFSDGHCDQGCNSAECEWDGL | | |
| | DCAEHVPERLAAGTLVLVVLLPPDQLRNNSFHFL | | |
| | RELSHVLHTNVVFKRDAQGQQMIFPYYGHEEELR | | |
| | KHPIKRSTVGWATSSLLPGTSGGRQRRELDPMDI | | |
| | RGSIVYLEIDNRQCVQSSSQCFQSATDVAAFLGA | | |
| | LASLGSLNIPYKIEAVKSEPVEPPLPSQLHLMYVAA | | |
| | AAFVLLFFVGCGVLLSRKRRRQLCIQKLMKLLSSI | | |
| | EQACDICRLKKLKCSKEKPKCAKCLKNNWECRYS | | |
| | PKTKRSPLTRAHLTEVESRLERLEQLFLLIFPREDL | | |
| | DMILKMDSLQDIKALLTGLFVQDNVNKDAVTDRLA | | |
| | SVETDMPLTLRQHRISATSSSEESSNKGQRQLTV | | |
| | SAAAGGSGGSGGSDALDDFDLDMLGSDALDDFD | | |
| | LDMLGSDALDDFDLDMLGSDALDDFDLDMLGSG | | |
| | SGVKQTLNFDLLKLAGDVESNPGPVDMPNPRPA | | |
| | KPMAPSLALGPSPGVLPSWKTAPKGSELLGTRGS | | |
| | GGPFQGRDLRSGAHTSSSLNPLPPSQLQLPTVPL | | |
| | VMVAPSGARLGPSPHLQALLQDRPHFMHQLSTV | | |
| | DAHAQTPVLQVRPLDNPAMISLPPPSAATGVFSL | | |
| | KARPGLPPGINVASLEWVSREPALLCTFPRSGTP | | |
| | RKDSNLLAAPQGSYPLLANGVCKWPGCEKVFEE | | |
| | PEEFLKHCQADHLLDEKGKAQCLLQREVVQSLEQ | | |
| | QLELEKEKLGAMQAHLAGKMALAKAPSVASMDK | | |
| | SSCCIVATSTQGSVLPAWSAPREAPDGGLFAVRR | | |
| | HLWGSHGNSSFPEFFHNMDYFKYHNMRPPFTYA | | |
| | TLIRWAILEAPERQRTLNEIYHWFTRMFAYFRNHP | | |
| | ATWKNAIRHNLSLHKCFVRVESEKGAVWTVDEFE | | |
| | FRKKRSQRPNKCSNPCP | | |
| 19 | MALPVTALLLPLALLLHAARPEQKLISEEDLMYQR | | Entire construct |
| | MLRCGAELGSPGGGGGGGGGGGAGGRLALLWI | | ORF protein |
| | VPLTLSGLLGVAWGASSLGAHHIHHFHGSSKHHS | | sequence, |
| | VPIAIYRSPASLRGGHAGTTYIFSKGGGQITYKWP | | containing SEQ ID: |
| | PNDRPSTRADRLAIGFSTVQKEAVLVRVDSSSGL | | 6 WT neurexin |
| | GAYLELHIHQGKIGVKFNVGTDDIAIEESNAIINDG | | domain, followed by |
| | KYHVVRFTRSGGNATLQVDSWPVIERYPAGRQLT | | the Gal4 and FoxP3 |
| | AFNSQATIIIGGKEQGQPFQGQLSGLYYNGLKVLN | | after P2A. |
| | MAAENDANIAIVGNVRLVGEVPGGGGSPCVGSN | | |
| | PCYNQGTCEPTSENPFYRCLCPAKFNGLLCHILD | | |
| | YSFTGGAGRDIPPPQIEEACELPECQVDAGNKVC | | |
| | NLQCNNHACGWDGGDCSLNFNDPWKNCTQSLQ | | |
| | CWKYFSDGHCDSQCNSAGCLFDGFDCQLTEGQ | | |
| | CNPLYDQYCKDHFSDGHCDQGCNSAECEWDGL | | |
| | DCAEHVPERLAAGTLVLVVLLPPDQLRNNSFHFL | | |
| | RELSHVLHTNVVFKRDAQGQQMIFPYYGHEEELR | | |
| | KHPIKRSTVGWATSSLLPGTSGGRQRRELDPMDI | | |
| | RGSIVYLEIDNRQCVQSSSQCFQSATDVAAFLGA | | |
| | LASLGSLNIPYKIEAVKSEPVEPPLPSQLHLMYVAA | | |
| | AAFVLLFFVGCGVLLSRKRRRQLCIQKLMKLLSSI | | |
| | EQACDICRLKKLKCSKEKPKCAKCLKNNWECRYS | | |
| | PKTKRSPLTRAHLTEVESRLERLEQLFLLIFPREDL | | |
| | DMILKMDSLQDIKALLTGLFVQDNVNKDAVTDRLA | | |
| | SVETDMPLTLRQHRISATSSSEESSNKGQRQLTV | | |
| | SAAAGGSGGSGGSDALDDFDLDMLGSDALDDFD | | |
| | LDMLGSDALDDFDLDMLGSDALDDFDLDMLGSG | | |
| | SGVKQTLNFDLLKLAGDVESNPGPVDMPNPRPA | | |
| | KPMAPSLALGPSPGVLPSWKTAPKGSELLGTRGS | | |
| | GGPFQGRDLRSGAHTSSSLNPLPPSQLQLPTVPL | | |
| | VMVAPSGARLGPSPHLQALLQDRPHFMHQLSTV | | |
| | DAHAQTPVLQVRPLDNPAMISLPPPSAATGVFSL | | |
| | KARPGLPPGINVASLEWVSREPALLCTFPRSGTP | | |
| | RKDSNLLAAPQGSYPLLANGVCKWPGCEKVFEE | | |
| | PEEFLKHCQADHLLDEKGKAQCLLQREVVQSLEQ | | |
| | QLELEKEKLGAMQAHLAGKMALAKAPSVASMDK | | |
| | SSCCIVATSTQGSVLPAWSAPREAPDGGLFAVRR | | |
| | HLWGSHGNSSFPEFFHNMDYFKYHNMRPPFTYA | | |
| | TLIRWAILEAPERQRTLNEIYHWFTRMFAYFRNHP | | |
| | ATWKNAIRHNLSLHKCFVRVESEKGAVWTVDEFE | | |
| | FRKKRSQRPNKCSNPCP | | |
| 20 | MALPVTALLLPLALLLHAARP | | signal-peptide on |
| | | | SynNotch construct |
| 21 | EQKLISEEDL | | myc-tag |
| 22 | MSMLFYTLITAFLIGIQA | | signal peptide from |
| | | | tether |
| 23 | GGGGSGGGGSGGGGS | | Membrane anchor |
| | | | on tether |
| 24 | MSMLFYTLITAFLIGIQAEQKLISEEDLASSLGAHHI | | Tethered neurexin |
| | HHFHGSSKHHSVPIAIYRSPASLRGGHAGTTYIFS | | full sequence |
| | KGGGQITYKWPPNDRPSTRADRLAIGFSTVQKEA | | |
| | VLVRVDSSSGLGDYLELHIHQGKIGVKFNVGTDDI | | |
| | AIEESNAIINDGKYHVVRFTRSGGNATLQVDSWPV | | |
| | IERYPAGNNDNERLAIARQRIPYRLGRVVDEWLLD | | |
| | KGRQLTIFNSQATIIIGGKEQGQPFQGQLSGLYYN | | |
| | GLKVLNMAAENDANIAIVGNVRLVGEVPGGGGSG | | |
| | GGGSGGGGSTLVLFGAGFGAVITVVVIVVIIKCFC | | |
| | K | | |
| 25 | AGCGAGGAGGATCTGATGTACCAGAGGATGCT | | |
| | GAGGTGC | | |
| 26 | GCTGTAGTCCAGGATGGGCACCTCGCCCACCA | | |
| | GC | | |
| 27 | GCCGCCGCGATCGCCatggcattgcccgt | | |
| 28 | GCGGCCGGCCGTTTAtcatgatccgagcatgtccag | | |
| 29 | TGATTTATGCGTAACGCCATTTTGCAAGGCATG | | |
| | G | | |
| 30 | GATAAGCTTGATATCGAATTTTACTTGTACAGCT | | |
| | CGTCCATGCC | | |
| 31 | ATGGCGTTACGCATAAATCAATATTGGCTATTG | | |
| | GCCATTGC | | |
| 32 | ATGGCGTTACTTTATAGAGCTCATCCATCCCAA | | |
| | GTGTGA | | |
| 33 | GCTCTATAAAGTAACGCCATTTTGCAAGGCA | | |
| 34 | TCGACTCTAGAGTCGCGGCCGAGCATGTCCAG | | |
| 35 | TGATTTATGCGTAACGCCATTTTGCAAGGCATG | | |
| | G | | |
| 36 | GATAAGCTTGATATCGAATTTTACTTGTACAGCT | | |
| | CGTCCATGCC | | |
| 37 | ATGGCGTTACGCATAAATCAATATTGGCTATTG | | |
| | GCCATTGC | | |
| 38 | AATCAATGTCTTTATAGAGCTCATCCATCCCAAG | | |
| | TGTGA | | |
| 39 | GCTCTATAAAGACATTGATTATTGACTAGTTATT | | |
| | AATAGTAATCAATTACGGG | | |
| 40 | TCGACTCTAGAGTCGCGGCCGAGCATGTCCAG | | |
| 41 | DVQITQSPSSLSASLGERATINCRASKSINKYLAW | | Light chain variable |
| | YQQKPGKAPKLLIYSGSTLQSGIPARFSGSGSGT | | region of |
| | DFTLTISSLEPEDFAMYYCQQHNEYPLTFGQGTK | | humanized M1 |
| | LEIK | | |
| 42 | QVQLVQSGAELKKPGASVKVSCKSSGYHFTSYW | | Heavy chain |
| | MHWVKQAPGQGLEWIGVIHPNSGSINYNEKFESR | | variable region of |
| | VTITVDKSTSTAYMELSSLRSEDTAVYYCAGERDS | | humanized M1 |
| | TEVLPMDYWGQGTTVTVSS | | |

### Additional Sequences Forming Part of the Application as Filed:

SEQ ID NO: 43 - Full (DNA) sequence of pREF060 (synNotch producing Gal4 and Gal4-
inducible CAR):
tggaagggctaattcactcccaaagaagacaagatatccttgatctgtggatctaccacacacaaggctacttccctgattagcag
aactacacaccagggccaggggtcagatatccactgacctttggatggtgctacaagctagtaccagttgagccagataaggta
gaagaggccaataaaggagagaacaccagcttgttacaccctgtgagcctgcatgggatggatgacccggagagagaagtgt
tagagtggaggtttgacagccgcctagcatttcatcacgtggcccgagagctgcatccggagtacttcaagaactgctgatatcga
gcttgctacaagggactttccgctggggactttccagggaggcgtggcctggggggactggggagtggcgagccctcagatcc
tgcatataagcagctgctttttgcctgtactgggtctctctggttagaccagatctgagcctgggagctctctggctaactagggaacc
cactgcttaagcctcaataaagcttgccttgagtgcttcaagtagtgtgtgcccgtctgttgtgtgactctggtaactagagatccctca
gacccttttagtcagtgtggaaaatctctagcagtggcgcccgaacagggacttgaaagcgaaagggaaaccagaggagctct
ctcgacgcaggactcggcttgctgaagcgcgcacggcaagaggcgaggggcggcgactggtgagtacgccaaaaattttgac
tagcggaggctagaaggagagagatgggtgcgagagcgtcagtattaagcgggggagaattagatcgcgatgggaaaaaat
tcggttaaggccagggggaaagaaaaaatataaattaaaacatatagtatgggcaagcagggagctagaacgattcgcagtt
aatcctggcctgttagaaacatcagaaggctgtagacaaatactgggacagctacaaccatcccttcagacaggatcagaaga
acttagatcattatataatacagtagcaaccctctattgtgtgcatcaaaggatagagataaaagacaccaaggaagctttagac
aagatagaggaagagcaaaacaaaagtaagaccaccgcacagcaagcggccggccgctgatcttcagacctggaggagg
agatatgagggacaattggagaagtgaattatataaatataaagtagtaaaaattgaaccattaggagtagcacccaccaagg
caaagagaagagtggtgcagagagaaaaaagagcagtgggaataggagctttgttccttgggttcttgggagcagcaggaag
cactatgggcgcagcgtcaatgacgctgacggtacaggccagacaattattgtctggtatagtgcagcagcagaacaatttgctg
agggctattgaggcgcaacagcatctgttgcaactcacagtctggggcatcaagcagctccaggcaagaatcctggctgtggaa
agatacctaaaggatcaacagctcctggggatttggggttgctctggaaaactcatttgcaccactgctgtgccttggaatgctagtt
ggagtaataaatctctggaacagatttggaatcacacgacctggatggagtgggacagagaaattaacaattacacaagcttaa
tacactccttaattgaagaatcgcaaaaccagcaagaaaagaatgaacaagaattattggaattagataaatgggcaagtttgtg
gaattggtttaacataacaaattggctgtggtatataaaattattcataatgatagtaggaggcttggtaggtttaagaatagtttttgct
gtactttctatagtgaatagagttaggcagggatattcaccattatcgtttcagacccacctcccaaccccgaggggacccgacag
gcccgaaggaatagaagaagaaggtggagagagagacagagacagatccattcgattagtgaacggatctcgacggtatcg
ccaaatggcagtattcatccacaattttaaaagaaaaggggggattggggggtacagtgcaggggaaagaatagtagacata
atagcaacagacatacaaactaaagaattacaaaaacaaattacaaaaattcaaaattttcgggtttattacagggacagcaga
gatccagtttggatcgataagcttgatatcgaattGCATAAATCAATATTGGCTATTGGCCATTGCATACGT
TGTATCTATATCATAAcggagcactgtcctccgaacgtcggagcactgtcctccgaacgtcggagcactgtcctccga
acgtcggagcactgtcctccgaacggagcatgtcctccgaacgtcggagcactgtcctccgaacgactagttaggcgtgtacggt
gggaggcctatataagcagagctcgtttagtgaaccgtcagatcgcctggagacgccatccacgctgttttgacctccatagaag
acaccgggaccgatccagcctctcgacattcgttggGCCACCATGGGAACTAGTCTGCTGTGTTGGATG
GCCCTCTGCTTGTTGGGCGCAGACCACGCTGACGGCCAAGTTCAATTGGTCCAGTCTG
GTGCCGAACTGAAGAAGCCAGGAGCTAGCGTCAAGGTGAGTTGTAAGTCTTCCGGATA
CCATTTCACGAGCTACTGGATGCATTGGGTAAAACAAGCCCCTGGACAAGGTCTCGAGT
GGATAGGGGTAATACATCCTAACTCTGGGTCCATAAATTACAATGAAAAGTTCGAATCCA
GGGTTACCATCACAGTCGATAAGTCCACTTCCACGGCCTATATGGAATTGAGCTCCCTC
AGGAGCGAAGATACAGCTGTATACTACTGCGCTGGTGAACGAGACTCCACTGAGGTGT
TGCCCATGGATTACTGGGGACAAGGCACCACTGTAACGGTCTCTAGCGGTGGGAGTAG
TAGAAGCTCATCATCTGGCGGCGGCGGTTCAGGCGGTGGCGGCGACGTGCAGATCAC
GCAAAGCCCGAGCTCACTCTCCGCCAGTCTGGGTGAACGTGCGACTATAAATTGTAGA
GCCAGCAAGAGCATTAACAAGTATCTCGCCTGGTACCAGCAAAAGCCGGGCAAGGCTC
CTAAACTCCTCATCTATTCCGGATCTACTCTCCAAAGCGGCATACCAGCGAGGTTCTCA
GGAAGCGGATCAGGAACGGATTTTACGCTTACGATATCTAGCCTCGAGCCAGAGGATTT
TGCCATGTACTATTGCCAACAACATAACGAATATCCCTTGACCTTCGGACAAGGGACGA
AACTCGAGATCAAAGATTACAAAGACGATGACGACAAAACCACAACCCCAGCTCCTAGG
CCCCCAACACCTGCACCTACCATCGCTAGCCAGCCTCTGAGCCTGCGTCCCGAGGCAT
GTCGTCCTGCTGCAGGTGGGGCAGTCCACACAAGAGGTCTGGACTTTGCATGTGACTT
TTGGGTCCTCGTAGTCGTCGGTGGCGTCCTGGCCTGTTATTCCCTTCTGGTTACGGTC
GCCTTCATTATATTTTGGGTCAGGAGTAAACGGTCTAGGGGGGGGCACAGCGACTACA
TGAATATGACGCCACGCCGCCCCGGGCCCACAAGAAAACACTACCAACCTTACGCTCC
ACCCAGGGATTTTGCCGCGTACCGGAGCAGGGTAAAGTTCAGCCGCAGTGOGGATGC
ACCAGCGTATCAACAAGGCCAGAATCAACTGTATAATGAGCTGAACTTGGGACGACGA
GAGGAATATGATGTTCTGGATAAACGGCGCGGGAGAGATCCAGAGATGGGCGGAAAG
CCTAGACGTAAGAACCCCCAAGAAGGCCTTTATAATGAGTTGCAGAAAGATAAGATGGC
CGAAGCCTATTCTGAAATAGGCATGAAGGGAGAGCGGCGGCGTGGCAAGGGTCATGA
CGGTCTTTACCAGGGCCTCTCTACCGCAACTAAAGATACCTATGATGCACTTCACATGC
AAGCCCTGCCCCCTAGAGAGTTCGAAGGGTCAGCAGCAGCCGAGGGACGTGGCTCAT
TGCTTACTTGCGGTGACGTTGAAGAAAACCCTGGCCCCAGCGGGATGGTAAGCAAGGG
AGAAGAACTTTTCACGGGAGTAGTTCCTATTCTGGTGGAACTGGACGGGGATGTAAACG
GGCATAAATTCTCTGTCAGCGGGGAAGGGGAAGGCGATGCAACATACGGGAAGCTTAC
CCTCAAATTCATCTGTACGACAGGGAAACTTCCAGTGCCCTGGCCTACACTTGTGACAA
CCCTGACTTATGGGGTCCAATGTTTCTCCAGGTACCCTGATCACATGAAACAGCATGAC
TTCTTCAAGAGCGCAATGCCTGAAGGGTATGTCCAGGAACGCACGATTTTCTTCAAGGA
CGATGGCAATTATAAAACTAGAGCCGAAGTAAAATTTGAGGGAGATACATTGGTAAACA
GGATCGAGCTCAAAGGGATCGATTTCAAAGAGGATGGGAACATCCTGGGGCATAAGTT
GGAGTATAACTATAACTCTCACAATGTCTATATTATGGCCGACAAGCAAAAGAACGGGA
TAAAAGTCAACTTTAAAATAAGGCACAATATAGAGGACGGCAGTGTGCAATTGGCGGAC
CACTATCAACAAAATACCCCGATCGGCGACGGTCCAGTGTTGCTCCCTGATAATCACTA
CCTTTCAACACAAAGTGCACTTAGTAAAGATCCCAATGAGAAGCGGGATCACATGGTCC
TGCTCGAATTTGTCACAGCAGCTGGCATCACACTTGGGATGGATGAGCTCTATAAATAA
TAAACGGCCGGCCGCGGTCATAGCTGTTTCCTGAACAGATCCCGGGTGGCATCCCTGT
GACCCCTCCCCAGTGCCTCTCCTGGCCCTGGAAGTTGCCACTCCAGTGCCCACCAGCC
TTGTCCTAATAAAATTAAGTTGCATCATTTTGTCTGACTAGGTGTCCTTCTATAATATTAT
GGGGTGGAGGGGGGTGGTATGGAGCAAGGGGCAAGTTGGGAAGACAACCTGTAGGG
CCTGCGGGGTCTATTGGGAACCAAGCTGGAGTGCAGTGGCACAATCTTGGCTCACTGC
AATCTCCGCCTCCTGGGTTCAAGCGATTCTCCTGCCTCAGCCTCCCGAGTTGTTGGGAT
TCCAGGCATGCATGACCAGGCTCAGCTAATTTTTGTTTTTTTGGTAGAGACGGGGTTTC
ACCATATTGGCCAGGCTGGTCTCCAACTCCTAATCTCAGGTGATCTACCCACCTTGGCC
TCCCAAATTGCTGGGATTACAGGCGTGAACCACTGCTCCCTTCCCTGTCCTTCTGATTT
TAAAATAACTATACCAGCAGGAGGACGTCCAGACACAGCATAGGCTACCTGGCCATGC
CCAACCGGTGGGACATTTGAGTTGCTTGCTTGGCACTGTCCTCTCATGCGTTGGGTCCA
CTCAGTAGATGCCTGTTGAATTGACATTGATTATTGACTAGTTATTAATAGTAATCAATTA
CGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATG
TTCCCATAGTAACGCCAATAGGGACTTTCCATTGACGTCAATGGGTGGAGTATTTACGG
TAAACTGCCCACTTGGCAGTACATCAAGTGTATCATATGCCAAGTCCGCCCCCTATTGA
CGTCAATGACGGTAAATGGCCCGCCTGGCATTATGCCCAGTACATGACCTTACGGGAC
TTTCCTACTTGGCAGTACATCTACGTATTAGTCATCGCTATTACCATGGTGATGCGGTTT
TGGCAGTACACCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAA
TGTCGTAATAACCCCGCCCCGTTGACGCAAATGGGCGGTAGGCGTGTACGGTGGGAG
GTCTATATAAGCAGAGCTCGTTTAGTGAACCGTCAGAATTTTGTAATACGACTCACTATA
GGGCGGCCGGGAATTCGTCGACTGGATCCGGTACCGAGGAGATCTGCCGCCGCGATC
GCCatggcattgcccgtgaccgccctgctgctgccactggccttgttgctccacgccgcgcggccagaacagaagCTGAT
CAGCGAGGAGGATCTGATGTACCAGAGGATGCTGAGGTGCGGCGCCGAGCTGGGCAG
CCCCGGCGGCGGCGGCGGCGGCGGCGGCGGCGGCGGCGCCGGCGGCAGGCTGGC
CCTGCTGTGGATCGTGCCCCTGACCCTGAGCGGCCTGCTGGGCGTGGCCTGGGGCGC
CAGCAGCCTGGGCGCCCACCACATCCACCACTTCCACGGCAGCAGCAAGCACCACAG
CGTGCCCATCGCCATCTACAGGAGCCCCGCCAGCCTGAGGGGCGGCCACGCCGGCAC
CACCTACATCTTCAGCAAGGGCGGCGGCCAGATCACCTACAAGTGGCCCCCCAACGAC
AGGCCCAGCACCAGGGCCGACAGGCTGGCCATCGGCTTCAGCACCGTGCAGAAGGAG
GCCGTGCTGGTGAGGGTGGACAGCAGCAGCGGCCTGGGCGACTACCTGGAGCTGCAC
ATCCACCAGGGCAAGATCGGCGTGAAGTTCAACGTGGGCACCGACGACATCGCCATCG
AGGAGAGCAACGCCATCATCAACGACGGCAAGTACCACGTGGTGAGGTTCACCAGGAG
CGGCGGCAACGCCACCCTGCAGGTGGACAGCTGGCCCGTGATCGAGAGGTACCCCGC
CGGCAGGCAGCTGACCATCTTCAACAGCCAGGCCACCATCATCATCGGCGGCAAGGA
GCAGGGCCAGCCCTTCCAGGGCCAGCTGAGCGGCCTGTACTACAACGGCCTGAAGGT
GCTGAACATGGCCGCCGAGAACGACGCCAACATCGCCATCGTGGGCAACGTGAGGCT
GGTGGGCGAGGTGCCCATCCTGGACTACAGCTTCACAGGtggcgctgggcgcgacattcccccacc
gcagattgaggaggcctgtgagctgcctgagtgccaggtggatgcaggcaataaggtctgcaacctgcagtgtaataatcacgc
atgtggctgggatggtggcgactgctccctcaacttcaatgacccctggaagaactgcacgcagtctctacagtgctggaagtattt
tagcgacggccactgtgacagccagtgcaactcggccggctgcctctttgatggcttcgactgccagctcaccgagggacagtg
caaccccctgtatgaccagtactgcaaggaccacttcagtgatggccactgcgaccagggctgtaacagtgccgaatgtgagtg
ggatggcctagactgtgctgagcatgtacccgagcggctggcagccggcaccctggtgctggtggtgctgcttccacccgacca
gctacggaacaactccttccactttctgcgggagctcagccacgtgctgcacaccaacgtggtcttcaagcgtgatgcgcaaggc
cagcagatgatcttcccgtactatggccacgaggaagagctgcgcaagcacccaatcaagcgctctacagtgggttgggccac
ctcttcactgcttcctggtaccagtggtgggcgccagcgcagggagctggaccccatggacatccgtggctccattgtctacctgg
agatcgacaaccggcaatgtgtgcagtcatcctcgcagtgcttccagagtgccaccgatgtggctgccttcctaggtgctcttgcgt
cacttggcagcctcaatattccttacaagattgaggccgtgaagagtgagccggtggagcctccgctgccctcgcagctgcacct
catgtacgtggcagcggccgccttcgtgctcctgttctttgtgggctgtggggtgctgctgtcccgcaagcgccggcggatgaagct
gctgagcagcatcgagcaggcctgtgacatctgccggctgaagaaactgaagtgcagcaaagaaaagcccaagtgcgccaa
gtgcctgaagaacaactgggagtgccggtacagccccaagaccaagagaagccccctgaccagagcccacctgaccgagg
tggaaagccggctggaaagactggaacagctgtttctgctgatcttcccacgcgaggacctggacatgatcctgaagatggaca
gcctgcaggacatcaaggccctgctgaccggcctgttcgtgcaggacaacgtgaacaaggacgccgtgaccgacagactggc
cagcgtggaaaccgacatgcccctgaccctgcggcagcacagaatcagcgccaccagcagcagcgaggaaagcagcaac
aagggccagcggcagctgacagtgtctgctgctgcaggcggaagcggaggctctggcggatctgatgccctggacgacttcga
cctggatatgctgggcagcgacgccctggatgattttgatctggacatgctgggatctgacgctctggacgatttcgatctcgacatg
ttgggatcagatgcactggatgactttgacctggacatgctcggatcatgaTAAACGGCCGGCCGCGGTCATAGC
TGTTTCCTGGGCCGCGACTCTAGAGTCGACCTGCAGGCATGCAAGCTTGATATCAAGC
TTATCGATAATCAACCTCTGGATTACAAAATTTGtgAAAGATTGACTGGTATTCTTAACTAT
GTTGCTCCTTTTACGCTATGTGGATACGCTGCTTTAATGCCTTTGTATCATGCTATTGCT
TCCCGTATGGCTTTCATTTTCTCCTCCTTGTATAAATCCTGGTTGCTGTCTCTTTATGAG
GAGTTGTGGCCCGTTGTCAGGCAACGTGGCGTGGTGTGCACTGTGTTTGCTGACGCAA
CCCCCACTGGTTGGGGCATTGCCACCACCTGTCAGCTCCTTTCCGGGACTTTCGCTTTC
CCCCTCCCTATTGCCACGGCGGAACTCATCGCCGCCTGCCTTGCCCGCTGCTGGACAG
GGGCTCGGCTGTTGGGCACTGACAATTCCGTGGTGTTGTCGGGGAAATCATCGTCCTT
TCCTTGGCTGCTCGCCTGTGTTGCCACCTGGATTCTGCGCGGGACGTCCTTCTGCTAC
GTCCCTTCGGCCCTCAATCCAGCGGACCTTCCTTCCCAATTCTACCGGGTAGGGGAGG
CGCTTTTCCCAAGGCAGTCTGGAGCATGCGCTTTAGCAGCCCCGCTGGGCACTTGGCG
CTACACAAGTGGCCTCTGGCCTCGCACACATTCCACATCCACCGGTAGGCGCCAACCG
GCTCCGTTCTTTGGTGGCCCCTTCGCGCCACCTTCTACTCCTCCCCTAGTCAGGAAGTT
CCCCCCCGCCCCGCAGCTCGCGTCGTGCAGGACGTGACAAATGGAAGTAGCACGTCT
CACTAGTCTCGTGCAGATGGACAGCACCGCTGAGCAATGGAAGCGGGTAGGCCTTTGG
GGCAGCGGCCAATAGCAGCTTTGCTCCTTCGCTTTCTGGGCTCAGAGGCTGGGAAGGG
GTGGGTCCGGGGGGGGCTCAGGGGGGGGCTCAGGGGGGGGGGGGCGCCCGAAG
GTCCTCCGGAGGCCCGGCATTCTGCACGCTTCAAAAGCGCACGTCTGCCGCGCTGTTC
TCCTCTTCCTCATCTCCGGGCCTTTCGACCTGCAGCCCAAGCTTACCATGACCGAGTAC
AAGCCCACGGTGCGCCTCGCCACCCGCGACGACGTCCCCAGGGCCGTACGCACCCTC
GCCGCCGCGTTCGCCGACTACCCCGCCACGCGCCACACCGTCGATCCGGACCGCCAC
ATCGAGCGGGTCACCGAGCTGCAAGAACTCTTCCTCACGCGCGTCGGGCTCGACATCG
GCAAGGTGTGGGTCGCGGACGACGGCGCCGCGGTGGCGGTCTGGACCACGCCGGAG
AGCGTCGAAGCGGGGGCGGTGTTCGCCGAGATCGGCCCGCGCATGGCCGAGTTGAG
CGGTTCCCGGCTGGCCGCGCAGCAACAGATGGAAGGCCTCCTGGCGCCGCACCGGC
CCAAGGAGCCCGCGTGGTTCCTGGCCACCGTCGGCGTCTCGCCCGACCACCAGGGCA
AGGGTCTGGGCAGCGCCGTCGTGCTCCCCGGAGTGGAGGCGGCCGAGCGCGCCGGG
GTGCCCGCCTTCCTGGAGACCTCCGCGCCCCGCAACCTCCCCTTCTACGAGCGGCTC
GGCTTCACCGTCACCGCCGACGTCGAGGTGCCCGAAGGACCGCGCACCTGGTGCATG
ACCCGCAAGCCCGGTGCCTGACTCGAGGGAATTAATTCGAGCTCGGTACCTTTAAGAC
CAATGACTTACAAGGCAGCTGTAGATCTTAGCCACTTTTTAAAAGAAAAGGGGGGACTG
GAAGGGCTAATTCACTCCCAACGAAGACAAGATCTGCTTTTTGCTTGTACTGGGTCTCT
CTGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGTGA
CTCTGGTAACTAGAGATCCCTCAGACCCTTTTAGTCAGTGTGGAAAATCTCTAGCAgcatct
agaattaattccgtgtattctatagtgtcacctaaatcgtatgtgtatgatacataaggttatgtattaattgtagccgcgttctaacgac
aatatgtacaagcctaattgtgtagcatctggcttactgaagcagaccctatcatctctctcgtaaactgccgtcagagtcggtttggtt
ggacgaaccttctgagtttctggtaacgccgtcccgcacccggaaatggtcagcgaaccaatcagcagggtcatcgctagccag
atcctctacgccggacgcatcgtggccggcatcaccggcgccacaggtgcggttgctggcgcctatatcgccgacatcaccgat
ggggaagatcgggctcgccacttcgggctcatgagcgcttgtttcggcgtgggtatggtggcaggccccgtggccgggggactgt
tgggcgccatctccttgcatgcaccattccttgcggcggcggtgctcaacggcctcaacctactactgggctgcttcctaatgcagg
agtcgcataagggagagcgtcgaatggtgcactctcagtacaatctgctctgatgccgcatagttaagccagccccgacacccg
ccaacacccgctgacgcgccctgacgggcttgtctgctcccggcatccgcttacagacaagctgtgaccgtctccgggagctgc
atgtgtcagaggttttcaccgtcatcaccgaaacgcgcgagacgaaagggcctcgtgatacgcctatttttataggttaatgtcatg
ataataatggtttcttagacgtcaggtggcacttttcggggaaatgtgcgcggaacccctatttgtttatttttctaaatacattcaaatat
gtatccgctcatgagacaataaccctgataaatgcttcaataatattgaaaaaggaagagtatgagtattcaacatttccgtgtcgc
ccttattcccttttttgcggcattttgccttcctgtttttgctcacccagaaacgctggtgaaagtaaaagatgctgaagatcagttgggt
gcacgagtgggttacatcgaactggatctcaacagcggtaagatccttgagagttttcgccccgaagaacgttttccaatgatgag
cacttttaaagttctgctatgtggcgcggtattatcccgtattgacgccgggcaagagcaactcggtcgccgcatacactattctcag
aatgacttggttgagtactcaccagtcacagaaaagcatcttacggatggcatgacagtaagagaattatgcagtgctgccataa
ccatgagtgataacactgcggccaacttacttctgacaacgatcggaggaccgaaggagctaaccgcttttttgcacaacatggg
ggatcatgtaactcgccttgatcgttgggaaccggagctgaatgaagccataccaaacgacgagcgtgacaccacgatgcctgt
agcaatggcaacaacgttgcgcaaactattaactggcgaactacttactctagcttcccggcaacaattaatagactggatggag
gcggataaagttgcaggaccacttctgcgctcggcccttccggctggctggtttattgctgataaatctggagccggtgagcgtggg
tctcgcggtatcattgcagcactggggccagatggtaagccctcccgtatcgtagttatctacacgacggggagtcaggcaactat
ggatgaacgaaatagacagatcgctgagataggtgcctcactgattaagcattggtaactgtcagaccaagtttactcatatatact
ttagattgatttaaaacttcatttttaatttaaaaggatctaggtgaagatcctttttgataatctcatgaccaaaatcccttaacgtgagtt
ttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagag
cgcagataccaaatactgttcttctagtgtagccgtagttaggccaccacttcaagaactctgtagcaccgcctacatacctcgctct
gctaatcctgttaccagtggctgctgccagtggcgataagtcgtgtcttaccgggttggactcaagacgatagttaccggataagg
cgcagcggtcgggctgaacggggggttcgtgcacacagcccagcttggagcgaacgacctacaccgaactgagatacctac
agcgtgagctatgagaaagcgccacgcttcccgaagggagaaaggcggacaggtatccggtaagcggcagggtcggaaca
ggagagcgcacgagggagcttccagggggaaacgcctggtatctttatagtcctgtcgggtttcgccacctctgacttgagcgtcg
atttttgtgatgctcgtcaggggggggagcctatggaaaaacgccagcaacgcggcctttttacggttcctggccttttgctggcctt
ttgctcacatgttctttcctgcgttatcccctgattctgtggataaccgtattaccgcctttgagtgagctgataccgctcgccgcagccg
aacgaccgagcgcagcgagtcagtgagcgaggaagcggaagagcgcccaatacgcaaaccgcctctccccgcgcgttggc
cgattcattaatgcagctgtggaatgtgtgtcagttagggtgtggaaagtccccaggctccccagcaggcagaagtatgcaaagc
atgcatctcaattagtcagcaaccaggtgtggaaagtccccaggctccccagcaggcagaagtatgcaaagcatgcatctcaat
tagtcagcaaccatagtcccgcccctaactccgcccatcccgcccctaactccgcccagttccgcccattctccgccccatggctg
actaattttttttatttatgcagaggccgaggccgcctcggcctctgagctattccagaagtagtgaggaggcttttttggaggcctag
gcttttgcaaaaagcttggacacaagacaggcttgcgagatatgtttgagaataccactttatcccgcgtcagggagaggcagtg
cgtaaaaagacgcggactcatgtgaaatactggtttttagtgcgccagatctctataatctcgcgcaacctattttcccctcgaacac
tttttaagccgtagataaacaggctgggacacttcacatgagcgaaaaatacatcgtcacctgggacatgttgcagatccatgca
cgtaaactcgcaagccgactgatgccttctgaacaatggaaaggcattattgccgtaagccgtggcggtctgtaccgggtgcgtta
ctggcgcgtgaactgggtattcgtcatgtcgataccgtttgtatttccagctacgatcacgacaaccagcgcgagcttaaagtgctg
aaacgcgcagaaggcgatggcgaaggcttcatcgttattgatgacctggtggataccggtggtactgcggttgcgattcgtgaaat
gtatccaaaagcgcactttgtcaccatcttcgcaaaaccggctggtcgtccgctggttgatgactatgttgttgatatcccgcaagat
acctggattgaacagccgtgggatatgggcgtcgtattcgtcccgccaatctccggtcgctaatcttttcaacgcctggcactgccg
ggcgttgttctttttaacttcaggcgggttacaatagtttccagtaagtattctggaggctgcatccatgacacaggcaaacctgagc
gaaaccctgttcaaaccccgctttaaacatcctgaaacctcgacgctagtccgccgctttaatcacggcgcacaaccgcctgtgc
agtcggcccttgatggtaaaaccatccctcactggtatcgcatgattaaccgtctgatgtggatctggcgcggcattgacccacgc
gaaatcctcgacgtccaggcacgtattgtgatgagcgatgccgaacgtaccgacgatgatttatacgatacggtgattggctacc
gtggcggcaactggatttatgagtgggccccggatctttgtgaaggaaccttacttctgtggtgtgacataattggacaaactaccta
cagagatttaaagctctaaggtaaatataaaatttttaagtgtataatgtgttaaactactgattctaattgtttgtgtattttagattccaa
cctatggaactgatgaatgggagcagtggtggaatgcctttaatgaggaaaacctgttttgctcagaagaaatgccatctagtgat
gatgaggctactgctgactctcaacattctactcctccaaaaaagaagagaaaggtagaagaccccaaggactttccttcagaat
tgctaagttttttgagtcatgctgtgtttagtaatagaactcttgcttgctttgctatttacaccacaaaggaaaaagctgcactgctatac
aagaaaattatggaaaaatattctgtaacctttataagtaggcataacagttataatcataacatactgttttttcttactccacacagg
catagagtgtctgctattaataactatgctcaaaaattgtgtacctttagctttttaatttgtaaaggggttaataaggaatatttgatgtat
agtgccttgactagagatcataatcagccataccacatttgtagaggttttacttgctttaaaaaacctcccacacctccccctgaac
ctgaaacataaaatgaatgcaattgttgttgttaacttgtttattgcagcttataatggttacaaataaagcaatagcatcacaaatttc
acaaataaagcatttttttcactgcattctagttgtggtttgtccaaactcatcaatgtatcttatcatgtctggatcaactggataactca
agctaaccaaaatcatcccaaacttcccaccccataccctattaccactgccaattacctgtggtttcatttactctaaacctgtgattc
ctctgaattattttcattttaaagaaattgtatttgttaaatatgtactacaaacttagtagt
SEQ ID NO: 44 - Protein sequence of synNotch (receptor 1) of pREF060:
MALPVTALLLPLALLLHAARPEQKLISEEDLMYQRMLRCGAELGSPGGGGGGGGGGGG
GRLALLWIVPLTLSGLLGVAWGASSLGAHHIHHFHGSSKHHSVPIAIYRSPASLRGGHAGTT
YIFSKGGGQITYKWPPNDRPSTRADRLAIGFSTVQKEAVLVRVDSSSGLGDYLELHIHQGKI
GVKFNVGTDDIAIEESNAIINDGKYHVVRFTRSGGNATLQVDSWPVIERYPAGRQLTIFNSQ
ATIIIGGKEQGQPFQGQLSGLYYNGLKVLNMAAENDANIAIVGNVRLVGEVPILDYSFTGGA
GRDIPPPQIEEACELPECQVDAGNKVCNLQCNNHACGWDGGDCSLNFNDPWKNCTQSLQ
CWKYFSDGHCDSQCNSAGCLFDGFDCQLTEGQCNPLYDQYCKDHFSDGHCDQGCNSAE
CEWDGLDCAEHVPERLAAGTLVLVVLLPPDQLRNNSFHFLRELSHVLHTNVVFKRDAQGQ
QMIFPYYGHEEELRKHPIKRSTVGWATSSLLPGTSGGRQRRELDPMDIRGSIVYLEIDNRQ
CVQSSSQCFQSATDVAAFLGALASLGSLNIPYKIEAVKSEPVEPPLPSQLHLMYVAAAAFVL
LFFVGCGVLLSRKRRRMKLLSSIEQACDICRLKKLKCSKEKPKCAKCLKNNWECRYSPKTK
RSPLTRAHLTEVESRLERLEQLFLLIFPREDLDMILKMDSLQDIKALLTGLFVQDNVNKDAVT
DRLASVETDMPLTLRQHRISATSSSEESSNKGQRQLTVSAAAGGSGGSGGSDALDDFDLD
MLGSDALDDFDLDMLGSDALDDFDLDMLGSDALDDFDLDMLGS
SEQ ID NO: 45 - Protein sequence of CAR (receptor 2) of pREF060:
MGTSLLCWMALCLLGADHADGQVQLVQSGAELKKPGASVKVSCKSSGYHFTSYWMHWV
KQAPGQGLEWIGVIHPNSGSINYNEKFESRVTITVDKSTSTAYMELSSLRSEDTAVYYCAG
ERDSTEVLPMDYWGQGTTVTVSSGGSSRSSSSGGGGSGGGGDVQITQSPSSLSASLGE
RATINCRASKSINKYLAWYQQKPGKAPKLLIYSGSTLQSGIPARFSGSGSGTDFTLTISSLEP
EDFAMYYCQQHNEYPLTFGQGTKLEIKDYKDDDDKTTTPAPRPPTPAPTIASQPLSLRPEA
CRPAAGGAVHTRGLDFACDFWVLVVVGGVLACYSLLVTVAFIIFWVRSKRSRGGHSDYMN
MTPRRPGPTRKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRRGKGHDGLY
QGLSTATKDTYDALHMQALPPREFEGSAAAEGRGSLLTCGDVEENPGPSGMVSKGEELF
TGVVPILVELDGDVNGHKFSVSGEGEGDATYGKLTLKFICTTGKLPVPWPTLVTTLTYGVQ
CFSRYPDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYK
SEQ ID NO: 46 - CAR sequence used in examples:
MGTSLLCWMALCLLGADHADGQVQLVQSGAELKKPGASVKVSCKSSGYHFTSYWMHWV
KQAPGQGLEWIGVIHPNSGSINYNEKFESRVTITVDKSTSTAYMELSSLRSEDTAVYYCAG
ERDSTEVLPMDYWGQGTTVTVSSGGSSRSSSSGGGGSGGGGDVQITQSPSSLSASLGE
RATINCRASKSINKYLAWYQQKPGKAPKLLIYSGSTLQSGIPARFSGSGSGTDFTLTISSLEP
EDFAMYYCQQHNEYPLTFGQGTKLEIKDYKDDDDKTTTPAPRPPTPAPTIASQPLSLRPEA
CRPAAGGAVHTRGLDFACDFWVLVVVGGVLACYSLLVTVAFIIFWVRSKRSRGGHSDYMN
MTPRRPGPTRKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRRGKGHDGLY
QGLSTATKDTYDALHMQALPPR
SEQ ID NO: 47 - Human Neurexin sequence >NP_620072.1 neurexin-1 isoform beta1
[Homo sapiens]
MYQRMLRCGAELGSPGGGGGGGGGGGAGGRLALLWIVPLTLSGLLGVAWGASSLGAHH
IHHFHGSSKHHSVPIAIYRSPASLRGGHAGTTYIFSKGGGQITYKWPPNDRPSTRADRLAIG
FSTVQKEAVLVRVDSSSGLGDYLELHIHQGKIGVKFNVGTDDIAIEESNAIINDGKYHVVRFT
RSGGNATLQVDSWPVIERYPAGRQLTIFNSQATIIIGGKEQGQPFQGQLSGLYYNGLKVLN
MAAENDANIAIVGNVRLVGEVPSSMTTESTATAMQSEMSTSIMETTTTLATSTARRGKPPT
KEPISQTTDDILVASAECPSDDEDIDPCEPSSGGLANPTRAGGREPYPGSAEVIRESSSTTG
MVVGIVAAAALCILILLYAMYKYRNRDEGSYHVDESRNYISNSAQSNGAVVKEKQPSSAKS
SNKNKKNKDKEYYV
SEQ ID NO: 48 - Tether → Cleavable peptide → FoxP3 - (Neurexin tether and FoxP3
linked by the P2A):
MSMLFYTLITAFLIGIQAEQKLISEEDLASSLGAHHIHHFHGSSKHHSVPIAIYRSPASLRGGH
AGTTYIFSKGGGQITYKWPPNDRPSTRADRLAIGFSTVQKEAVLVRVDSSSGLGDYLELHIH
QGKIGVKFNVGTDDIAIEESNAIINDGKYHVVRFTRSGGNATLQVDSWPVIERYPAGNNDNE
RLAIARQRIPYRLGRVVDEWLLDKGRQLTIFNSQATIIIGGKEQGQPFQGQLSGLYYNGLKV
LNMAAENDANIAIVGNVRLVGEVPGGGGSGGGGSGGGGSTLVLFGAGFGAVITVVVIVVIIK
CFCKGSGVKQTLNFDLLKLAGDVESNPGPVDMPNPRPAKPMAPSLALGPSPGVLPSWKT
APKGSELLGTRGSGGPFQGRDLRSGAHTSSSLNPLPPSQLQLPTVPLVMVAPSGARLGPS
PHLQALLQDRPHFMHQLSTVDAHAQTPVLQVRPLDNPAMISLPPPSAATGVFSLKARPGLP
PGINVASLEWVSREPALLCTFPRSGTPRKDSNLLAAPQGSYPLLANGVCKWPGCEKVFEE
PEEFLKHCQADHLLDEKGKAQCLLQREVVQSLEQQLELEKEKLGAMQAHLAGKMALAKAP
SVASMDKSSCCIVATSTQGSVLPAWSAPREAPDGGLFAVRRHLWGSHGNSSFPEFFHNM
DYFKYHNMRPPFTYATLIRWAILEAPERQRTLNEIYHWFTRMFAYFRNHPATWKNAIRHNL
SLHKCFVRVESEKGAVWTVDEFEFRKKRSQRPNKCSNPCP
SEQ ID NO: 49 - pREF020 sequence - lentiviral vector with synNotch with WT neurexin
sequence
AACAAAATATTAACGCTTACAATTTCCATTCGCCATTCAGGCTGCGCAACTGTTGGGAA
GGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACGA
CGGCCAGTGCCAAGCTGATCTATACATTGAATCAATATTGGCAATTAGCCATATTAGTC
ATTGGTTATATAGCATAAATCAATATTGGCTATTGGCCATTGCATACGTTGTATCTATAT
CATAATATGTACATTTATATTGGCTCATGTCCAATATGACCGCCATGTTGACATTGATTA
TTGACTAGTTATTAATAGTAATCAATTACGGGGTCATTAGTTCATAGCCCATATATGGAG
TTCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTA
TCATATGCCAAGTCCGCCCCCTATTGACGTCAATGACGGTAAATGGCCCGCCTGGCAT
TATGCCCAGTACATGACCTTACGGGACTTTCCTACTTGGCAGTACATCTACGTATTAGT
CATCGCTATTACCATGGTGATGCGGTTTTGGCAGTACACCAATGGGCGTGGATAGCGG
TTTGACTCACGGGGATTTCCAAGTCTCCACCCCATTGACGTCAATGGGAGTTTGTTTTG
GCACCAAAATCAACGGGACTTTCCAAAATGTCGTAATAACCCCGCCCCGTTGACGCAA
ATGGGCGGTAGGCGTGTACGGTGGGAGGTCTATATAAGCAGAGCTCGTTTAGTGAACC
GTCAGAATTTTGTAATACGACTCACTATAGGGCGGCCGGGAATTCGTCGACTGGATCC
GGTACCGAGGAGATCTGCCGCCGCGATCGCCatggcattgcccgtgaccgccctgctgctgccactggc
cttgttgctccacgccgcgcggccagaacagaagCTGATCAGCGAGGAGGATCTGATGTACCAGAGG
ATGCTGAGGTGCGGCGCCGAGCTGGGCAGCCCCGGCGGCGGCGGCGGCGGCGGCG
GCGGCGGCGGCGCCGGCGGCAGGCTGGCCCTGCTGTGGATCGTGCCCCTGACCCTG
AGCGGCCTGCTGGGCGTGGCCTGGGGCGCCAGCAGCCTGGGCGCCCACCACATCCA
CCACTTCCACGGCAGCAGCAAGCACCACAGCGTGCCCATCGCCATCTACAGGAGCCC
CGCCAGCCTGAGGGGCGGCCACGCCGGCACCACCTACATCTTCAGCAAGGGCGGCG
GCCAGATCACCTACAAGTGGCCCCCCAACGACAGGCCCAGCACCAGGGCCGACAGGC
TGGCCATCGGCTTCAGCACCGTGCAGAAGGAGGCCGTGCTGGTGAGGGTGGACAGCA
GCAGCGGCCTGGGCGACTACCTGGAGCTGCACATCCACCAGGGCAAGATCGGCGTGA
AGTTCAACGTGGGCACCGACGACATCGCCATCGAGGAGAGCAACGCCATCATCAACG
ACGGCAAGTACCACGTGGTGAGGTTCACCAGGAGCGGCGGCAACGCCACCCTGCAG
GTGGACAGCTGGCCCGTGATCGAGAGGTACCCCGCCGGCAGGCAGCTGACCATCTTC
AACAGCCAGGCCACCATCATCATCGGCGGCAAGGAGCAGGGCCAGCCCTTCCAGGGC
CAGCTGAGCGGCCTGTACTACAACGGCCTGAAGGTGCTGAACATGGCCGCCGAGAAC
GACGCCAACATCGCCATCGTGGGCAACGTGAGGCTGGTGGGCGAGGTGCCCATCCTG
GACTACAGCTTCACAGGtggcgctgggcgcgacattcccccaccgcagattgaggaggcctgtgagctgcctgagt
gccaggtggatgcaggcaataaggtctgcaacctgcagtgtaataatcacgcatgtggctgggatggtggcgactgctccctca
acttcaatgacccctggaagaactgcacgcagtctctacagtgctggaagtattttagcgacggccactgtgacagccagtgca
actcggccggctgcctctttgatggcttcgactgccagctcaccgagggacagtgcaaccccctgtatgaccagtactgcaagg
accacttcagtgatggccactgcgaccagggctgtaacagtgccgaatgtgagtgggatggcctagactgtgctgagcatgtac
ccgagcggctggcagccggcaccctggtgctggtggtgctgcttccacccgaccagctacggaacaactccttccactttctgcg
ggagctcagccacgtgctgcacaccaacgtggtcttcaagcgtgatgcgcaaggccagcagatgatcttcccgtactatggcca
cgaggaagagctgcgcaagcacccaatcaagcgctctacagtgggttgggccacctcttcactgcttcctggtaccagtggtgg
gcgccagcgcagggagctggaccccatggacatccgtggctccattgtctacctggagatcgacaaccggcaatgtgtgcagt
catcctcgcagtgcttccagagtgccaccgatgtggctgccttcctaggtgctcttgcgtcacttggcagcctcaatattccttacaa
gattgaggccgtgaagagtgagccggtggagcctccgctgccctcgcagctgcacctcatgtacgtggcagcggccgccttcgt
gctcctgttctttgtgggctgtggggtgctgctgtcccgcaagcgccggcggatgaagctgctgagcagcatcgagcaggcctgt
gacatctgccggctgaagaaactgaagtgcagcaaagaaaagcccaagtgcgccaagtgcctgaagaacaactgggagtg
ccggtacagccccaagaccaagagaagccccctgaccagagcccacctgaccgaggtggaaagccggctggaaagactg
gaacagctgtttctgctgatcttcccacgcgaggacctggacatgatcctgaagatggacagcctgcaggacatcaaggccctg
ctgaccggcctgttcgtgcaggacaacgtgaacaaggacgccgtgaccgacagactggccagcgtggaaaccgacatgccc
ctgaccctgcggcagcacagaatcagcgccaccagcagcagcgaggaaagcagcaacaagggccagcggcagctgaca
gtgtctgctgctgcaggcggaagcggaggctctggcggatctgatgccctggacgacttcgacctggatatgctgggcagcgac
gccctggatgattttgatctggacatgctgggatctgacgctctggacgatttcgatctcgacatgttgggatcagatgcactggatg
actttgacctggacatgctcggatcatgaTAAACGGCCGGCCGCGGTCATAGCTGTTTCCTGAACAG
ATCCCGGGTGGCATCCCTGTGACCCCTCCCCAGTGCCTCTCCTGGCCCTGGAAGTTG
CCACTCCAGTGCCCACCAGCCTTGTCCTAATAAAATTAAGTTGCATCATTTTGTCTGACT
AGGTGTCCTTCTATAATATTATGGGGTGGAGGGGGGTGGTATGGAGCAAGGGGCAAG
TTGGGAAGACAACCTGTAGGGCCTGCGGGGTCTATTGGGAACCAAGCTGGAGTGCAG
TGGCACAATCTTGGCTCACTGCAATCTCCGCCTCCTGGGTTCAAGCGATTCTCCTGCC
TCAGCCTCCCGAGTTGTTGGGATTCCAGGCATGCATGACCAGGCTCAGCTAATTTTTGT
TTTTTTGGTAGAGACGGGGTTTCACCATATTGGCCAGGCTGGTCTCCAACTCCTAATCT
CAGGTGATCTACCCACCTTGGCCTCCCAAATTGCTGGGATTACAGGCGTGAACCACTG
CTCCCTTCCCTGTCCTTCTGATTTTAAAATAACTATACCAGCAGGAGGACGTCCAGACA
CAGCATAGGCTACCTGGCCATGCCCAACCGGTGGGACATTTGAGTTGCTTGCTTGGCA
CTGTCCTCTCATGCGTTGGGTCCACTCAGTAGATGCCTGTTGAATTGGGTACGCGGCC
AGCTTGGCTGTGGAATGTGTGTCAGTTAGGGTGTGGAAAGTCCCCAGGCTCCCCAGCA
GGCAGAAGTATGCAAAGCATGCATCTCAATTAGTCAGCAACCAGGTGTGGAAAGTCCC
CAGGCTCCCCAGCAGGCAGAAGTATGCAAAGCATGCATCTCAATTAGTCAGCAACCAT
AGTCCCGCCCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGCCTCGGCCT
CTGAGCTATTCCAGAAGTAGTGAGGAGGCTTTTTTGGAGGCCTAGGCTTTTGCAAAAA
GCTCCCGGGAGCTTGTATATCCATTTTCGGATCTGATCAAGAGACAGGATGAGGATCG
TTTCGCATGATTGAACAAGATGGATTGCACGCAGGTTCTCCGGCCGCTTGGGTGGAGA
GGCTATTCGGCTATGACTGGGCACAACAGACAATCGGCTGCTCTGATGCCGCCGTGTT
CCGGCTGTCAGCGCAGGGGCGCCCGGTTCTTTTTGTCAAGACCGACCTGTCCGGTGC
CCTGAATGAACTGCAGGACGAGGCAGCGCGGCTATCGTGGCTGGCCACGACGGGCG
TTCCTTGCGCAGCTGTGCTCGACGTTGTCACTGAAGCGGGAAGGGACTGGCTGCTATT
GGGCGAAGTGCCGGGGCAGGATCTCCTGTCATCTCACCTTGCTCCTGCCGAGAAAGT
ATCCATCATGGCTGATGCAATGCGGCGGCTGCATACGCTTGATCCGGCTACCTGCCCA
TTCGACCACCAAGCGAAACATCGCATCGAGCGAGCACGTACTCGGATGGAAGCCGGT
CTTGTCGATCAGGATGATCTGGACGAAGAGCATCAGGGGCTCGCGCCAGCCGAACTG
TTCGCCAGGCTCAAGGCGCGCATGCCCGACGGCGAGGATCTCGTCGTGACCCATGGC
GATGCCTGCTTGCCGAATATCATGGTGGAAAATGGCCGCTTTTCTGGATTCATCGACTG
TGGCCGGCTGGGTGTGGCCGACCGCTATCAGGACATAGCGTTGGCTACCCGTGATAT
TGCTGAAGAGCTTGGCGGCGAATGGGCTGACCGCTTCCTCGTGCTTTACGGTATCGCC
GCTCCCGATTCGCAGCGCATCGCCTTCTATCGCCTTCTTGACGAGTTCTTCTGAGCGG
GACTCTGGGGTTCGAAATGACCGACCAAGCGACGCCCAACCTGCCATCACGAGATTTC
GATTCCACCGCCGCCTTCTATGAAAGGTTGGGCTTCGGAATCGTTTTCCGGGACGCCG
GCTGGATGATCCTCCAGCGCGGGGATCTCATGCTGGAGTTCTTCGCCCACCCCAACTT
GTTTATTGCAGCTTATAATGGTTACAAATAAAGCAATAGCATCACAAATTTCACAAATAA
AGCATTTTTTTCACTGCATTCTAGTTGTGGTTTGTCCAAACTCATCAATGTATOTTATCAT
GTCTGTATACCGTCGACCTCTAGCTAGAGCTTGGCGTAATCATGGTCATAGCTGTTTCC
TGTGTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCACT
GCCCGCTTTCCAGTCGGGAAACCTGTCGTGCCAGCTGCATTAATGAATCGGCCAACGC
GCGGGGAGAGGCGGTTTGCGTATTGGGCGCTCTTCCGCTTCCTCGCTCACTGACTCG
CTGCGCTCGGTCGTTCGGCTGCGGCGAGCGGTATCAGCTCACTCAAAGGCGGTAATA
CGGTTATCCACAGAATCAGGGGATAACGCAGGAAAGAACATGTGAGCAAAAGGCCAGC
AAAAGGCCAGGAACCGTAAAAAGGCCGCGTTGCTGGCGTTTTTCCATAGGCTCCGCCC
CCCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGT
CTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAA
CTACGGCTACACTAGAAGAACAGTATTTGGTATCTGCGCTCTGCTGAAGCCAGTTACCT
TCGGAAAAAGAGTTGGTAGCTCTTGATCCGGCAAACAAACCACCGCTGGTAGCGGTTT
TTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTC
ATGAGATTATCAAAAAGGATCTTCACCTAGATCCTTTTAAATTAAAAATGAAGTTTTAAAT
CAATCTAAAGTATATATGAGTAAACTTGGTCTGACAGTTACCAATGCTTAATCAGTGAGG
CACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGC
GAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGC
CGGGAAGCTAGAGTAAGTAGTTCGCCAGTTAATAGTTTGCGCAACGTTGTTGCCATTG
CTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCATC
ATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAAGGGCGA
CACGGAAATGTTGAATACTCATACTCTTCCTTTTTCAATATTATTGAAGCATTTATCAGG
GTTATTGTCTCATGAGCGGATACATATTTGAATGTATTTAGAAAAATAAACAAATAGGGG
TTCCGCGCACATTTCCCCGAAAAGTGCCACCTGACGCGCCCTGTAGCGGCGCATTAAG
CGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACG
GTTTTTCGCCCTTTGACGTTGGAGTCCACGTTCTTTAATAGTGGACTCTTGTTCCAAACT
GGAACAACACTCAACCCTATCTCGGTCTATTCTTTTGATTTATAAGGGATTTTGCCGATT
TCGGCCTATTGGTTAAAAAATGAGCTGATTTAACAAAAATTTAACGCGAATTTT
SEQ ID NO: 50 - Linearised by PCR backbone sequence:
ATCCTGGACTACAGCTTCACAGGtggcgctgggcgcgacattcccccaccgcagattgaggaggcctgtgagc
tgcctgagtgccaggtggatgcaggcaataaggtctgcaacctgcagtgtaataatcacgcatgtggctgggatggtggcgact
gctccctcaacttcaatgacccctggaagaactgcacgcagtctctacagtgctggaagtattttagcgacggccactgtgacag
ccagtgcaactcggccggctgcctctttgatggcttcgactgccagctcaccgagggacagtgcaaccccctgtatgaccagta
ctgcaaggaccacttcagtgatggccactgcgaccagggctgtaacagtgccgaatgtgagtgggatggcctagactgtgctg
agcatgtacccgagcggctggcagccggcaccctggtgctggtggtgctgcttccacccgaccagctacggaacaactccttcc
actttctgcgggagctcagccacgtgctgcacaccaacgtggtcttcaagcgtgatgcgcaaggccagcagatgatcttcccgta
ctatggccacgaggaagagctgcgcaagcacccaatcaagcgctctacagtgggttgggccacctcttcactgcttcctggtac
cagtggtgggcgccagcgcagggagctggaccccatggacatccgtggctccattgtctacctggagatcgacaaccggcaa
tgtgtgcagtcatcctcgcagtgcttccagagtgccaccgatgtggctgccttcctaggtgctcttgcgtcacttggcagcctcaatat
tccttacaagattgaggccgtgaagagtgagccggtggagcctccgctgccctcgcagctgcacctcatgtacgtggcagcggc
cgccttcgtgctcctgttctttgtgggctgtggggtgctgctgtcccgcaagcgccggoggatgaagctgctgagcagcatcgagc
aggcctgtgacatctgccggctgaagaaactgaagtgcagcaaagaaaagcccaagtgcgccaagtgcctgaagaacaac
tgggagtgccggtacagccccaagaccaagagaagccccctgaccagagcccacctgaccgaggtggaaagccggctgg
aaagactggaacagctgtttctgctgatcttcccacgcgaggacctggacatgatcctgaagatggacagcctgcaggacatca
aggccctgctgaccggcctgttcgtgcaggacaacgtgaacaaggacgccgtgaccgacagactggccagcgtggaaacc
gacatgcccctgaccctgcggcagcacagaatcagcgccaccagcagcagcgaggaaagcagcaacaagggccagcgg
cagctgacagtgtctgctgctgcaggcggaagcggaggctctggcggatctgatgccctggacgacttcgacctggatatgctg
ggcagcgacgccctggatgattttgatctggacatgctgggatctgacgctctggacgatttcgatctcgacatgttgggatcagat
gcactggatgactttgacctggacatgctcggatcatgagatccttgacttgcggccgcaactcccacctgcaacatgcgtgactg
actgaggccgcgactctagagtcgacctgcaggcatgcaagcttgatatcaagcttatcgataatcaacctctggattacaaaatt
tgtgaaagattgactggtattcttaactatgttgctccttttacgctatgtggatacgctgctttaatgcctttgtatcatgctattgcttccc
gtatggctttcattttctcctccttgtataaatcctggttgctgtctctttatgaggagttgtggcccgttgtcaggcaacgtggcgtggtgt
gcactgtgtttgctgacgcaacccccactggttggggcattgccaccacctgtcagctcctttccgggactttcgctttccccctccct
attgccacggcggaactcatcgccgcctgccttgcccgctgctggacaggggctcggctgttgggcactgacaattccgtggtgtt
gtcggggaaatcatcgtcctttccttggctgctcgcctgtgttgccacctggattctgcgcgggacgtccttctgctacgtcccttcgg
ccctcaatccagcggaccttccttcccgcggcctgctgccggctctgcggcctcttccgcgtcttcgccttcgccctcagacgagtc
ggatctccctttgggccgcctccccgcatcgataccgtcgacctcgagggaattaattcgagctcggtacctttaagaccaatgac
ttacaaggcagctgtagatcttagccactttttaaaagaaaaggggggactggaagggctaattcactcccaacgaagacaag
atctgctttttgcttgtactgggtctctctggttagaccagatctgagcctgggagctctctggctaactagggaacccactgcttaag
cctcaataaagcttgccttgagtgcttcaagtagtgtgtgcccgtctgttgtgtgactctggtaactagagatccctcagacccttttag
tcagtgtggaaaatctctagcagcatctagaattaattccgtgtattctatagtgtcacctaaatcgtatgtgtatgatacataaggtta
tgtattaattgtagccgcgttctaacgacaatatgtacaagcctaattgtgtagcatctggcttactgaagcagaccctatcatctctct
cgtaaactgccgtcagagtcggtttggttggacgaaccttctgagtttctggtaacgccgtcccgcacccggaaatggtcagcga
accaatcagcagggtcatcgctagccagatcctctacgccggacgcatcgtggccggcatcaccggcgccacaggtgcggtt
gctggcgcctatatcgccgacatcaccgatggggaagatcgggctcgccacttcgggctcatgagcgcttgtttcggcgtgggta
tggtggcaggccccgtggccgggggactgttgggcgccatctccttgcatgcaccattccttgcggcggcggtgctcaacggcct
caacctactactgggctgcttcctaatgcaggagtcgcataagggagagcgtcgaatggtgcactctcagtacaatctgctctgat
gccgcatagttaagccagccccgacacccgccaacacccgctgacgcgccctgacgggcttgtctgctcccggcatccgctta
cagacaagctgtgaccgtctccgggagctgcatgtgtcagaggttttcaccgtcatcaccgaaacgcgcgagacgaaagggc
ctcgtgatacgcctatttttataggttaatgtcatgataataatggtttcttagacgtcaggtggcacttttcggggaaatgtgcgcgga
acccctatttgtttatttttctaaatacattcaaatatgtatccgctcatgagacaataaccctgataaatgcttcaataatattgaaaaa
ggaagagtatgagtattcaacatttccgtgtcgcccttattcccttttttgcggcattttgccttcctgtttttgctcacccagaaacgctg
gtgaaagtaaaagatgctgaagatcagttgggtgcacgagtgggttacatcgaactggatctcaacagcggtaagatccttgag
agttttcgccccgaagaacgttttccaatgatgagcacttttaaagttctgctatgtggcgcggtattatcccgtattgacgccgggca
agagcaactcggtcgccgcatacactattctcagaatgacttggttgagtactcaccagtcacagaaaagcatcttacggatggc
atgacagtaagagaattatgcagtgctgccataaccatgagtgataacactgcggccaacttacttctgacaacgatcggagga
ccgaaggagctaaccgcttttttgcacaacatgggggatcatgtaactcgccttgatcgttgggaaccggagctgaatgaagcc
ataccaaacgacgagcgtgacaccacgatgcctgtagcaatggcaacaacgttgcgcaaactattaactggcgaactacttac
tctagcttcccggcaacaattaatagactggatggaggcggataaagttgcaggaccacttctgcgctcggcccttccggctggc
tggtttattgctgataaatctggagccggtgagcgtgggtctcgcggtatcattgcagcactggggccagatggtaagccctcccg
tatcgtagttatctacacgacggggagtcaggcaactatggatgaacgaaatagacagatcgctgagataggtgcctcactgatt
aagcattggtaactgtcagaccaagtttactcatatatactttagattgatttaaaacttcatttttaatttaaaaggatctaggtgaaga
tcctttttgataatctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaag
agctaccaactctttttccgaaggtaactggcttcagcagagcgcagataccaaatactgttcttctagtgtagccgtagttaggcc
accacttcaagaactctgtagcaccgcctacatacctcgctctgctaatcctgttaccagtggctgctgccagtggcgataagtcgt
gtcttaccgggttggactcaagacgatagttaccggataaggcgcagcggtcgggctgaacggggggttcgtgcacacagccc
agcttggagcgaacgacctacaccgaactgagatacctacagcgtgagctatgagaaagcgccacgcttcccgaagggaga
aaggcggacaggtatccggtaagcggcagggtcggaacaggagagcgcacgagggagcttccagggggaaacgcctggt
atctttatagtcctgtcgggtttcgccacctctgacttgagcgtcgatttttgtgatgctcgtcaggggggcggagcctatggaaaaa
cgccagcaacgcggcctttttacggttcctggccttttgctggccttttgctcacatgttctttcctgcgttatcccctgattctgtggataa
ccgtattaccgcctttgagtgagctgataccgctcgccgcagccgaacgaccgagcgcagcgagtcagtgagcgaggaagc
ggaagagcgcccaatacgcaaaccgcctctccccgcgcgttggccgattcattaatgcagctgtggaatgtgtgtcagttagggt
gtggaaagtccccaggctccccagcaggcagaagtatgcaaagcatgcatctcaattagtcagcaaccaggtgtggaaagtc
cccaggctccccagcaggcagaagtatgcaaagcatgcatctcaattagtcagcaaccatagtcccgcccctaactccgccca
tcccgcccctaactccgcccagttccgcccattctccgccccatggctgactaattttttttatttatgcagaggccgaggccgcctc
ggcctctgagctattccagaagtagtgaggaggcttttttggaggcctaggcttttgcaaaaagcttggacacaagacaggcttgc
gagatatgtttgagaataccactttatcccgcgtcagggagaggcagtgcgtaaaaagacgcggactcatgtgaaatactggttt
ttagtgcgccagatctctataatctcgcgcaacctattttcccctcgaacactttttaagccgtagataaacaggctgggacacttca
catgagcgaaaaatacatcgtcacctgggacatgttgcagatccatgcacgtaaactcgcaagccgactgatgccttctgaaca
atggaaaggcattattgccgtaagccgtggcggtctgtaccgggtgcgttactggcgcgtgaactgggtattcgtcatgtcgatac
cgtttgtatttccagctacgatcacgacaaccagcgcgagcttaaagtgctgaaacgcgcagaaggcgatggcgaaggcttcat
cgttattgatgacctggtggataccggtggtactgcggttgcgattcgtgaaatgtatccaaaagcgcactttgtcaccatcttcgca
aaaccggctggtcgtccgctggttgatgactatgttgttgatatcccgcaagatacctggattgaacagccgtgggatatgggcgt
cgtattcgtcccgccaatctccggtcgctaatcttttcaacgcctggcactgccgggcgttgttctttttaacttcaggcgggttacaat
agtttccagtaagtattctggaggctgcatccatgacacaggcaaacctgagcgaaaccctgttcaaaccccgctttaaacatcc
tgaaacctcgacgctagtccgccgctttaatcacggcgcacaaccgcctgtgcagtcggcccttgatggtaaaaccatccctcac
tggtatcgcatgattaaccgtctgatgtggatctggcgcggcattgacccacgcgaaatcctcgacgtccaggcacgtattgtgat
gagcgatgccgaacgtaccgacgatgatttatacgatacggtgattggctaccgtggcggcaactggatttatgagtgggcccc
ggatctttgtgaaggaaccttacttctgtggtgtgacataattggacaaactacctacagagatttaaagctctaaggtaaatataa
aatttttaagtgtataatgtgttaaactactgattctaattgtttgtgtattttagattccaacctatggaactgatgaatgggagcagtgg
tggaatgcctttaatgaggaaaacctgttttgctcagaagaaatgccatctagtgatgatgaggctactgctgactctcaacattcta
ctcctccaaaaaagaagagaaaggtagaagaccccaaggactttccttcagaattgctaagttttttgagtcatgctgtgtttagta
atagaactcttgcttgctttgctatttacaccacaaaggaaaaagctgcactgctatacaagaaaattatggaaaaatattctgtaa
cctttataagtaggcataacagttataatcataacatactgttttttcttactccacacaggcatagagtgtctgctattaataactatgc
tcaaaaattgtgtacctttagctttttaatttgtaaaggggttaataaggaatatttgatgtatagtgccttgactagagatcataatcag
ccataccacatttgtagaggttttacttgctttaaaaaacctcccacacctccccctgaacctgaaacataaaatgaatgcaattgtt
gttgttaacttgtttattgcagcttataatggttacaaataaagcaatagcatcacaaatttcacaaataaagcatttttttcactgcatt
ctagttgtggtttgtccaaactcatcaatgtatcttatcatgtctggatcaactggataactcaagctaaccaaaatcatcccaaactt
cccaccccataccctattaccactgccaattacctgtggtttcatttactctaaacctgtgattcctctgaattattttcattttaaagaaat
tgtatttgttaaatatgtactacaaacttagtagttggaagggctaattcactcccaaagaagacaagatatccttgatctgtggatct
accacacacaaggctacttccctgattagcagaactacacaccagggccaggggtcagatatccactgacctttggatggtgct
acaagctagtaccagttgagccagataaggtagaagaggccaataaaggagagaacaccagcttgttacaccctgtgagcct
gcatgggatggatgacccggagagagaagtgttagagtggaggtttgacagccgcctagcatttcatcacgtggcccgagagc
tgcatccggagtacttcaagaactgctgatatcgagcttgctacaagggactttccgctggggactttccagggaggcgtggcctg
ggcgggactggggagtggcgagccctcagatcctgcatataagcagctgctttttgcctgtactgggtctctctggttagaccagat
ctgagcctgggagctctctggctaactagggaacccactgcttaagcctcaataaagcttgccttgagtgcttcaagtagtgtgtgc
ccgtctgttgtgtgactctggtaactagagatccctcagacccttttagtcagtgtggaaaatctctagcagtggcgcccgaacagg
gacttgaaagcgaaagggaaaccagaggagctctctcgacgcaggactcggcttgctgaagcgcgcacggcaagaggcg
aggggcggcgactggtgagtacgccaaaaattttgactagcggaggctagaaggagagagatgggtgcgagagcgtcagta
ttaagcgggggagaattagatcgcgatgggaaaaaattcggttaaggccagggggaaagaaaaaatataaattaaaacatat
agtatgggcaagcagggagctagaacgattcgcagttaatcctggcctgttagaaacatcagaaggctgtagacaaatactgg
gacagctacaaccatcccttcagacaggatcagaagaacttagatcattatataatacagtagcaaccctctattgtgtgcatcaa
aggatagagataaaagacaccaaggaagctttagacaagatagaggaagagcaaaacaaaagtaagaccaccgcacag
caagcggccggccgctgatcttcagacctggaggaggagatatgagggacaattggagaagtgaattatataaatataaagta
gtaaaaattgaaccattaggagtagcacccaccaaggcaaagagaagagtggtgcagagagaaaaaagagcagtgggaa
taggagctttgttccttgggttcttgggagcagcaggaagcactatgggcgcagcgtcaatgacgctgacggtacaggccagac
aattattgtctggtatagtgcagcagcagaacaatttgctgagggctattgaggcgcaacagcatctgttgcaactcacagtctgg
ggcatcaagcagctccaggcaagaatcctggctgtggaaagatacctaaaggatcaacagctcctggggatttggggttgctct
ggaaaactcatttgcaccactgctgtgccttggaatgctagttggagtaataaatctctggaacagatttggaatcacacgacctg
gatggagtgggacagagaaattaacaattacacaagcttaatacactccttaattgaagaatcgcaaaaccagcaagaaaag
aatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccat
tatcgtttcagacccacctcccaaccccgaggggacccgacaggcccgaaggaatagaagaagaaggtggagagagaga
cagagacagatccattcgattagtgaacggatctcgacggtatcgccaaatggcagtattcatccacaattttaaaagaaaagg
ggggattggggggtacagtgcaggggaaagaatagtagacataatagcaacagacatacaaactaaagaattacaaaaac
aaattacaaaaattcaaaattttcgggtttattacagggacagcagagatccagtttggatcgataagcttgatatcgaattgggta
ggggaggcgcttttcccaaggcagtctggagcatgcgctttagcagccccgctgggcacttggcgctacacaagtggcctctgg
cctcgcacacattccacatccaccggtaggcgccaaccggctccgttctttggtggccccttcgcgccaccttctactcctccccta
gtcaggaagttcccccccgccccgcagctcgcgtcgtgcaggacgtgacaaatggaagtagcacgtctcactagtctcgtgca
gatggacagcaccgctgagcaatggaagcgggtaggcctttggggcagcggccaatagcagctttgctccttcgctttctgggct
cagaggctgggaaggggtgggtccggggggggctcaggggcgggctcaggggggggcgggcgcccgaaggtcctcc
ggaggcccggcattctgcacgcttcaaaagcgcacgtctgccgcgctgttctcctcttcctcatctccgggcctttcgaattctcacg
cgtcaagtggagcaaggcaggtggacagtggatcgccgccaccatggcattgcccgtgaccgccctgctgctgccactggcct
tgttgctccacgccgcgcggccagaacagaagCTGATCAGCGAGGAGGATCTG
SEQ ID NO: 51 - Plasmid pREF061 sequence - handling vector, expresses GFP,
lentiviral packing vector
tggaagggctaattcactcccaaagaagacaagatatccttgatctgtggatctaccacacacaaggctacttccctgattagca
gaactacacaccagggccaggggtcagatatccactgacctttggatggtgctacaagctagtaccagttgagccagataagg
tagaagaggccaataaaggagagaacaccagcttgttacaccctgtgagcctgcatgggatggatgacccggagagagaag
tgttagagtggaggtttgacagccgcctagcatttcatcacgtggcccgagagctgcatccggagtacttcaagaactgctgatat
cgagcttgctacaagggactttccgctggggactttccagggaggcgtggcctggggggactggggagtggcgagccctcag
atcctgcatataagcagctgctttttgcctgtactgggtctctctggttagaccagatctgagcctgggagctctctggctaactaggg
aacccactgcttaagcctcaataaagcttgccttgagtgcttcaagtagtgtgtgcccgtctgttgtgtgactctggtaactagagatc
cctcagacccttttagtcagtgtggaaaatctctagcagtggcgcccgaacagggacttgaaagcgaaagggaaaccagagg
agctctctcgacgcaggactcggcttgctgaagcgcgcacggcaagaggcgaggggcggcgactggtgagtacgccaaaa
attttgactagcggaggctagaaggagagagatgggtgcgagagcgtcagtattaagcgggggagaattagatcgcgatggg
aaaaaattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcagacaggat
cagaagaacttagatcattatataatacagtagcaaccctctattgtgtgcatcaaaggatagagataaaagacaccaaggaa
gctttagacaagatagaggaagagcaaaacaaaagtaagaccaccgcacagcaagcggccggccgctgatcttcagacct
ggaggaggagatatgagggacaattggagaagtgaattatataaatataaagtagtaaaaattgaaccattaggagtagcacc
caccaaggcaaagagaagagtggtgcagagagaaaaaagagcagtgggaataggagctttgttccttgggttcttgggagca
gcaggaagcactatgggcgcagcgtcaatgacgctgacggtacaggccagacaattattgtctggtatagtgcagcagcaga
acaatttgctgagggctattgaggcgcaacagcatctgttgcaactcacagtctggggcatcaagcagctccaggcaagaatcc
tggctgtggaaagatacctaaaggatcaacagctcctggggatttggggttgctctggaaaactcatttgcaccactgctgtgcctt
ggaatgctagttggagtaataaatctctggaacagatttggaatcacacgacctggatggagtgggacagagaaattaacaatt
acacaagcttaatacactccttaattgaagaatcgcaaaaccagcaagaaaagaatgaacaagaattattggaattagataaa
tgggcaagtttgtggaattggtttaacataacaaattggctgtggtatataaaattattcataatgatagtaggaggcttggtaggttta
agaatagtttttgctgtactttctatagtgaatagagttaggcagggatattcaccattatcgtttcagacccacctcccaaccccgag
gggacccgacaggcccgaaggaatagaagaagaaggtggagagagagacagagacagatccattcgattagtgaacgg
atctcgacggtatcgccaaatggcagtattcatccacaattttaaaagaaaaggggggattggggggtacagtgcaggggaaa
gaatagtagacataatagcaacagacatacaaactaaagaattacaaaaacaaattacaaaaattcaaaattttcgggtttatta
cagggacagcagagatccagtttggatcgataagcttgatatcgaattcctgcagccccgataaaataaaagattttatttagtctc
cagaaaaaggggggaatgaaagaccccacctgtaggtttggcaagctagctgcagtaacgccattttgcaaggcatggaaaa
ataccaaaccaagaatagagaagttcagatcaagggcgggtacatgaaaatagctaacgttgggccaaacaggatatctgc
ggtgagcagtttcggccccggcccggggccaagaacagatggtcaccgcagtttcggccccggcccgaggccaagaacag
atggtccccagatatggcccaaccctcagcagtttcttaagacccatcagatgtttccaggctcccccaaggacctgaaatgacc
ctgcgccttatttgaattaaccaatcagcctgcttctcgcttctgttcgcgcgcttctgcttcccgagctctataaaagagctcacaac
ccctcactcggcgcgccagtcctccgacagactgagtcgcccgggggggatccaccggtcgccaccatggtgagcaagggc
gaggagctgttcaccggggtggtgcccatcctggtcgagctggacggcgacgtaaacggccacaagttcagcgtgtccggcg
agggcgagggcgatgccacctacggcaagctgaccctgaagttcatctgcaccaccggcaagctgcccgtgccctggcccac
cctcgtgaccaccctgacctacggcgtgcagtgcttcagccgctaccccgaccacatgaagcagcacgacttcttcaagtccgc
catgcccgaaggctacgtccaggagcgcaccatcttcttcaaggacgacggcaactacaagacccgcgccgaggtgaagttc
gagggcgacaccctggtgaaccgcatcgagctgaagggcatcgacttcaaggaggacggcaacatcctggggcacaagct
ggagtacaactacaacagccacaacgtctatatcatggccgacaagcagaagaacggcatcaaggtgaacttcaagatccg
ccacaacatcgaggacggcagcgtgcagctcgccgaccactaccagcagaacacccccatcggcgacggccccgtgctgc
tgcccgacaaccactacctgagcacccagtccgccctgagcaaagaccccaacgagaagcgcgatcacatggtcctgctgg
agttcgtgaccgccgccgggatcactctcggcatggacgagctgtacaagtaaagcggccgcgactctagagtcgacctgca
ggcatgcaagcttgatatcaagcttatcgataatcaacctctggattacaaaatttgtgaaagattgactggtattcttaactatgttg
ctccttttacgctatgtggatacgctgctttaatgcctttgtatcatgctattgcttccccgtatggctttcattttctcctccttgtataaatcct
ggttgctgtctctttatgaggagttgtggcccgttgtcaggcaacgtggcgtggtgtgcactgtgtttgctgacgcaacccccactggt
tggggcattgccaccacctgtcagctcctttccgggactttcgctttccccctccctattgccacggcggaactcategccgcctgcc
ttgcccgctgctggacaggggctcggctgttgggcactgacaattccgtggtgttgtcggggaaatcatcgtcctttccttggctgct
cgcctgtgttgccacctggattctgcgcgggacgtccttctgctacgtcccttcggccctcaatccagcggaccttccttcccaattct
accgggtaggggaggcgcttttcccaaggcagtctggagcatgcgctttagcagccccgctgggcacttggcgctacacaagt
ggcctctggcctcgcacacattccacatccaccggtaggcgccaaccggctccgttctttggtggccccttcgcgccaccttctact
cctcccctagtcaggaagttcccccccgccccgcagctcgcgtcgtgcaggacgtgacaaatggaagtagcacgtctcactagt
ctcgtgcagatggacagcaccgctgagcaatggaagcgggtaggcctttggggcagcggccaatagcagctttgctccttcgct
ttctgggctcagaggctgggaaggggtgggtccggggggggctcaggggcgggctcaggggggggggggcccgaa
ggtcctccggaggcccggcattctgcacgcttcaaaagcgcacgtctgccgcgctgttctcctcttcctcatctccgggcctttcga
cctgcagcccaagcttaccatgaccgagtacaagcccacggtgcgcctcgccacccgcgacgacgtccccagggccgtacg
caccctcgccgccgcgttcgccgactaccccgccacgcgccacaccgtcgatccggaccgccacatcgagcgggtcaccga
gctgcaagaactcttcctcacgcgcgtcgggctcgacatcggcaaggtgtgggtcgcggacgacggcgccgcggtggcggtct
ggaccacgccggagagcgtcgaagcgggggcggtgttcgccgagatcggcccgcgcatggccgagttgagcggttcccgg
ctggccgcgcagcaacagatggaaggcctcctggcgccgcaccggcccaaggagcccgcgtggttcctggccaccgtcggc
gtctcgcccgaccaccagggcaagggtctgggcagcgccgtcgtgctccccggagtggaggcggccgagcgcgccggggt
gcccgccttcctggagacctccgcgccccgcaacctccccttctacgagcggctcggcttcaccgtcaccgccgacgtcgaggt
gcccgaaggaccgcgcacctggtgcatgacccgcaagcccggtgcctgactcgagggaattaattcgagctcggtacctttaa
gaccaatgacttacaaggcagctgtagatcttagccactttttaaaagaaaaggggggactggaagggctaattcactcccaac
gaagacaagatctgctttttgcttgtactgggtctctctggttagaccagatctgagcctgggagctctctggctaactagggaaccc
actgcttaagcctcaataaagcttgccttgagtgcttcaagtagtgtgtgcccgtctgttgtgtgactctggtaactagagatccctca
gacccttttagtcagtgtggaaaatctctagcagcatctagaattaattccgtgtattctatagtgtcacctaaatcgtatgtgtatgata
cataaggttatgtattaattgtagccgcgttctaacgacaatatgtacaagcctaattgtgtagcatctggcttactgaagcagaccc
tatcatctctctcgtaaactgccgtcagagtcggtttggttggacgaaccttctgagtttctggtaacgccgtcccgcacccggaaat
ggtcagcgaaccaatcagcagggtcatcgctagccagatcctctacgccggacgcatcgtggccggcatcaccggcgccac
aggtgcggttgctggcgcctatatcgccgacatcaccgatggggaagatcgggctcgccacttcgggctcatgagcgcttgtttc
ggcgtgggtatggtggcaggccccgtggccgggggactgttgggcgccatctccttgcatgcaccattccttgcggcggcggtg
ctcaacggcctcaacctactactgggctgcttcctaatgcaggagtcgcataagggagagcgtcgaatggtgcactctcagtac
aatctgctctgatgccgcatagttaagccagccccgacacccgccaacacccgctgacgcgccctgacgggcttgtctgctccc
ggcatccgcttacagacaagctgtgaccgtctccgggagctgcatgtgtcagaggttttcaccgtcatcaccgaaacgcgcgag
acgaaagggcctcgtgatacgcctatttttataggttaatgtcatgataataatggtttcttagacgtcaggtggcacttttcggggaa
atgtgcgcggaacccctatttgtttatttttctaaatacattcaaatatgtatccgctcatgagacaataaccctgataaatgcttcaat
aatattgaaaaaggaagagtatgagtattcaacatttccgtgtcgcccttattcccttttttgcggcattttgccttcctgtttttgctcacc
cagaaacgctggtgaaagtaaaagatgctgaagatcagttgggtgcacgagtgggttacatcgaactggatctcaacagcggt
aagatccttgagagttttcgccccgaagaacgttttccaatgatgagcacttttaaagttctgctatgtggcgcggtattatcccgtatt
gacgccgggcaagagcaactcggtcgccgcatacactattctcagaatgacttggttgagtactcaccagtcacagaaaagca
tcttacggatggcatgacagtaagagaattatgcagtgctgccataaccatgagtgataacactgcggccaacttacttctgaca
acgatcggaggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaactattaactg
gcgaactacttactctagcttcccggcaacaattaatagactggatggaggcggataaagttgcaggaccacttctgcgctcggc
ccttccggctggctggtttattgctgataaatctggagccggtgagcgtgggtctcgcggtatcattgcagcactggggccagatg
gtaagccctcccgtatcgtagttatctacacgacggggagtcaggcaactatggatgaacgaaatagacagatcgctgagata
ggtgcctcactgattaagcattggtaactgtcagaccaagtttactcatatatactttagattgatttaaaacttcatttttaatttaaaag
gatctaggtgaagatcctttttgataatctcatgaccaaaatcccttaacgtgagttttcgttccactgagcgtcagaccccgtagaa
aagatcaaaggatcttcttgagatcctttttttctgcgcgtaatctgctgcttgcaaacaaaaaaaccaccgctaccagcggtggttt
gtttgccggatcaagagctaccaactctttttccgaaggtaactggcttcagcagagcgcagataccaaatactgttcttctagtgta
gccgtagttaggccaccacttcaagaactctgtagcaccgcctacatacctcgctctgctaatcctgttaccagtggctgctgcca
gtggcgataagtcgtgtcttaccgggttggactcaagacgatagttaccggataaggcgcagcggtcgggctgaacggggggtt
cgtgcacacagcccagcttggagcgaacgacctacaccgaactgagatacctacagcgtgagctatgagaaagcgccacgc
ttcccgaagggagaaaggcggacaggtatccggtaagcggcagggtcggaacaggagagcgcacgagggagcttccagg
gggaaacgcctggtatctttatagtcctgtcgggtttcgccacctctgacttgagcgtcgatttttgtgatgctcgtcaggggggcgg
agcctatggaaaaacgccagcaacgcggcctttttacggttcctggccttttgctggccttttgctcacatgttctttcctgcgttatccc
ctgattctgtggataaccgtattaccgcctttgagtgagctgataccgctcgccgcagccgaacgaccgagcgcagcgagtcag
tgagcgaggaagcggaagagcgcccaatacgcaaaccgcctctccccgcgcgttggccgattcattaatgcagctgtggaat
gtgtgtcagttagggtgtggaaagtccccaggctccccagcaggcagaagtatgcaaagcatgcatctcaattagtcagcaacc
aggtgtggaaagtccccaggctccccagcaggcagaagtatgcaaagcatgcatctcaattagtcagcaaccatagtcccgc
ccctaactccgcccatcccgcccctaactccgcccagttccgcccattctccgccccatggctgactaattttttttatttatgcagag
gccgaggccgcctcggcctctgagctattccagaagtagtgaggaggcttttttggaggcctaggcttttgcaaaaagcttggac
acaagacaggcttgcgagatatgtttgagaataccactttatcccgcgtcagggagaggcagtgcgtaaaaagacgcggactc
atgtgaaatactggtttttagtgcgccagatctctataatctcgcgcaacctattttcccctcgaacactttttaagccgtagataaaca
ggctgggacacttcacatgagcgaaaaatacatcgtcacctgggacatgttgcagatccatgcacgtaaactcgcaagccgac
tgatgccttctgaacaatggaaaggcattattgccgtaagccgtggcggtctgtaccgggtgcgttactggcgcgtgaactgggta
ttcgtcatgtcgataccgtttgtatttccagctacgatcacgacaaccagcgcgagcttaaagtgctgaaacgcgcagaaggcga
tggcgaaggcttcatcgttattgatgacctggtggataccggtggtactgcggttgcgattcgtgaaatgtatccaaaagcgcacttt
gtcaccatcttcgcaaaaccggctggtcgtccgctggttgatgactatgttgttgatatcccgcaagatacctggattgaacagccg
tgggatatgggcgtcgtattcgtcccgccaatctccggtcgctaatcttttcaacgcctggcactgccgggcgttgttctttttaacttca
ggcgggttacaatagtttccagtaagtattctggaggctgcatccatgacacaggcaaacctgagcgaaaccctgttcaaaccc
cgctttaaacatcctgaaacctcgacgctagtccgccgctttaatcacggcgcacaaccgcctgtgcagtcggcccttgatggta
aaaccatccctcactggtatcgcatgattaaccgtctgatgtggatctggcgcggcattgacccacgcgaaatcctcgacgtcca
ggcacgtattgtgatgagcgatgccgaacgtaccgacgatgatttatacgatacggtgattggctaccgtggcggcaactggattt
atgagtgggccccggatctttgtgaaggaaccttacttctgtggtgtgacataattggacaaactacctacagagatttaaagctct
aaggtaaatataaaatttttaagtgtataatgtgttaaactactgattctaattgtttgtgtattttagattccaacctatggaactgatga
atgggagcagtggtggaatgcctttaatgaggaaaacctgttttgctcagaagaaatgccatctagtgatgatgaggctactgctg
actctcaacattctactcctccaaaaaagaagagaaaggtagaagaccccaaggactttccttcagaattgctaagttttttgagt
catgctgtgtttagtaatagaactcttgcttgctttgctatttacaccacaaaggaaaaagctgcactgctatacaagaaaattatgg
aaaaatattctgtaacctttataagtaggcataacagttataatcataacatactgttttttcttactccacacaggcatagagtgtctg
ctattaataactatgctcaaaaattgtgtacctttagctttttaatttgtaaaggggttaataaggaatatttgatgtatagtgccttgact
agagatcataatcagccataccacatttgtagaggttttacttgctttaaaaaacctcccacacctccccctgaacctgaaacata
aaatgaatgcaattgttgttgttaacttgtttattgcagcttataatggttacaaataaagcaatagcatcacaaatttcacaaataaa
gcatttttttcactgcattctagttgtggtttgtccaaactcatcaatgtatcttatcatgtctggatcaactggataactcaagctaacca
aaatcatcccaaacttcccaccccataccctattaccactgccaattacctgtggtttcatttactctaaacctgtgattcctctgaatt
attttcattttaaagaaattgtatttgttaaatatgtactacaaacttagtagt
SEQ ID NO: 52 - pREF0123 sequence - Tether with Gal4 under auto-cleavable peptide
tggaagggctaattcactcccaaagaagacaagatatccttgatctgtggatctaccacacacaaggctacttccctgattagca
gaactacacaccagggccaggggtcagatatccactgacctttggatggtgctacaagctagtaccagttgagccagataagg
tagaagaggccaataaaggagagaacaccagcttgttacaccctgtgagcctgcatgggatggatgacccggagagagaag
tgttagagtggaggtttgacagccgcctagcatttcatcacgtggcccgagagctgcatccggagtacttcaagaactgctgatat
cgagcttgctacaagggactttccgctggggactttccagggaggcgtggcctggggggactggggagtggcgagccctcag
atcctgcatataagcagctgctttttgcctgtactgggtctctctggttagaccagatctgagcctgggagctctctggctaactaggg
aacccactgcttaagcctcaataaagcttgccttgagtgcttcaagtagtgtgtgcccgtctgttgtgtgactctggtaactagagatc
cctcagacccttttagtcagtgtggaaaatctctagcagtggcgcccgaacagggacttgaaagcgaaagggaaaccagagg
agctctctcgacgcaggactcggcttgctgaagcgcgcacggcaagaggcgaggggcggcgactggtgagtacgccaaaa
attttgactagcggaggctagaaggagagagatgggtgcgagagcgtcagtattaagcgggggagaattagatcgcgatggg
aaaaaattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcagacaggat
cagaagaacttagatcattatataatacagtagcaaccctctattgtgtgcatcaaaggatagagataaaagacaccaaggaa
gctttagacaagatagaggaagagcaaaacaaaagtaagaccaccgcacagcaagcggccggccgctgatcttcagacct
ggaggaggagatatgagggacaattggagaagtgaattatataaatataaagtagtaaaaattgaaccattaggagtagcacc
caccaaggcaaagagaagagtggtgcagagagaaaaaagagcagtgggaataggagctttgttccttgggttcttgggagca
gcaggaagcactatgggcgcagcgtcaatgacgctgacggtacaggccagacaattattgtctggtatagtgcagcagcaga
acaatttgctgagggctattgaggcgcaacagcatctgttgcaactcacagtctggggcatcaagcagctccaggcaagaatcc
tggctgtggaaagatacctaaaggatcaacagctcctggggatttggggttgctctggaaaactcatttgcaccactgctgtgcctt
ggaatgctagttggagtaataaatctctggaacagatttggaatcacacgacctggatggagtgggacagagaaattaacaatt
acacaagcttaatacactccttaattgaagaatcgcaaaaccagcaagaaaagaatgaacaagaattattggaattagataaa
tgggcaagtttgtggaattggtttaacataacaaattggctgtggtatataaaattattcataatgatagtaggaggcttggtaggttta
agaatagtttttgctgtactttctatagtgaatagagttaggcagggatattcaccattatcgtttcagacccacctcccaaccccgag
gggacccgacaggcccgaaggaatagaagaagaaggtggagagagagacagagacagatccattcgattagtgaacgg
atctcgacggtatcgccaaatggcagtattcatccacaattttaaaagaaaaggggggattggggggtacagtgcaggggaaa
gaatagtagacataatagcaacagacatacaaactaaagaattacaaaaacaaattacaaaaattcaaaattttcgggtttatta
cagggacagcagagatccagtttggatcgataagcttgatatcgaattgtaacgccattttgcaaggcatggaaaaataccaaa
ccaagaatagagaagttcagatcaagggcgggtacatgaaaatagctaacgttgggccaaacaggatatctgcggtgagca
gtttcggccccggcccggggccaagaacagatggtcaccgcagtttcggccccggcccgaggccaagaacagatggtcccc
agatatggcccaaccctcagcagtttcttaagacccatcagatgtttccaggctcccccaaggacctgaaatgaccctgcgcctt
atttgaattaaccaatcagcctgcttctcgcttctgttcgcgcgcttctgcttcccgagctctataaaagagctcacaacccctcactc
ggcgcgccagtcctccgacagactgagtcgcccggggggGATCCGCCACCATGAGCATGCTGTTCTATA
CCCTGATCACCGCCTTTCTGATCGGCATCCAGGCCGAGCAGAAGCTGATCTCCGAGGA
AGATCTGGCCTCTAGCCTGGGCGCTCACCACATCCACCACTTTCACGGCAGCAGCAAG
CACCACTCTGTGCCTATCGCCATCTACAGAAGCCCCGCTTCTCTGAGAGGCGGCCATG
CTGGAACCACCTACATCTTCTCTAAAGGCGGCGGACAGATCACCTACAAGTGGCCTCC
AAACGACAGACCCAGCACCAGAGCCGATAGACTGGCCATCGGCTTCAGCACCGTGCA
GAAAGAAGCCGTGCTCGTCAGAGTGGACAGCAGCTCTGGACTGGGCGACTACCTGGA
ACTGCACATCCATCAGGGCAAGATCGGCGTGAAGTTCAACGTGGGCACCGACGACATT
GCCATCGAGGAAAGCAACGCCATCATCAACGACGGCAAGTACCACGTCGTGCGGTTC
ACAAGAAGCGGCGGCAACGCTACACTGCAGGTCGACTCTTGGCCCGTGATCGAGAGA
TACCCTGCCGGCAACAACGACAACGAGAGACTGGCTATCGCCAGACAGAGAATCCCCT
ACAGACTGGGAAGAGTGGTGGACGAGTGGCTGCTGGATAAGGGCAGACAGCTGACCA
TCTTCAATAGCCAGGCCACCATCATCATCGGCGGAAAAGAGCAGGGCCAGCCTTTCCA
GGGACAGCTGAGCGGACTGTACTACAACGGCCTGAAGGTGCTGAACATGGCCGCTGA
GAACGACGCCAATATCGCTATCGTGGGCAACGTGCGGCTCGTGGGAGAAGTTCCTGG
CGGCGGAGGATCTGGCGGAGGTGGAAGTGGCGGAGGCGGATCTACACTGGTGCTGT
TTGGCGCTGGCTTCGGCGCCGTGATTACAGTGGTGGTCATCGTCGTGATCATCAAGTG
TTTCTGCAAAGAAGGACGCGGCTCCCTGCTTACGTGTGGCGATGTTGAAGAAAATCCT
GGGCCTTCCGGCGCCATGAAGCTGCTGAGCAGCATCGAGCAGGCCTGCGACATCTGC
AGGCTGAAGAAGCTGAAGTGCAGCAAGGAGAAGCCCAAGTGCGCCAAGTGCCTGAAG
AACAACTGGGAGTGCAGGTACAGCCCCAAGACCAAGAGGAGCCCCCTGACCAGGGCC
CACCTGACCGAGGTGGAGAGCAGGCTGGAGAGGCTGGAGCAGCTGTTCCTGCTGATC
TTCCCCAGGGAGGACCTGGACATGATCCTGAAGATGGACAGCCTGCAGGACATCAAG
GCCCTGCTGACCGGCCTGTTCGTGCAGGACAACGTGAACAAGGACGCCGTGACCGAC
AGGCTGGCCAGCGTGGAGACCGACATGCCCCTGACCCTGAGGcagcacagaatcagcgccac
cagcagcagcgaggaaagcagcaacaagggccagcggcagctgacagtgtctgctgctgcaggcggaagcggaggctct
ggcggatctgatgccctggacgacttcgacctggatatgctgggcagcgacgccctggatgattttgatctggacatgctgggatc
tgacgctctggacgatttcgatctcgacatgttgggatcagatgcactggatgactttgacctggacatgctcggatcatgaggcc
gcgactctagagtcgacctgcaggcatgcaagcttgatatcaagcttatcgataatcaacctctggattacaaaatttgtgaaaga
ttgactggtattcttaactatgttgctccttttacgctatgtggatacgctgctttaatgcctttgtatcatgctattgcttccccgtatggcttt
cattttctcctccttgtataaatcctggttgctgtctctttatgaggagttgtggcccgttgtcaggcaacgtggcgtggtgtgcactgtgt
ttgctgacgcaacccccactggttggggcattgccaccacctgtcagctcctttccgggactttcgctttccccctccctattgccacg
gcggaactcatcgccgcctgccttgcccgctgctggacaggggctcggctgttgggcactgacaattccgtggtgttgtcgggga
aatcatcgtcctttccttggctgctcgcctgtgttgccacctggattctgcgcgggacgtccttctgctacgtcccttcggccctcaatc
cagcggaccttccttcccaattctaccgggtaggggaggcgcttttcccaaggcagtctggagcatgcgctttagcagccccgct
gggcacttggcgctacacaagtggcctctggcctcgcacacattccacatccaccggtaggcgccaaccggctccgttctttggt
ggccccttcgcgccaccttctactcctcccctagtcaggaagttcccccccgccccgcagctcgcgtcgtgcaggacgtgacaa
atggaagtagcacgtctcactagtctcgtgcagatggacagcaccgctgagcaatggaagcgggtaggcctttggggcagcg
gccaatagcagctttgctccttcgctttctgggctcagaggctgggaaggggtgggtccggggggggctcaggggcgggctca
ggggggggcgggcgcccgaaggtcctccggaggcccggcattctgcacgcttcaaaagcgcacgtctgccgcgctgttctc
ctcttcctcatctccgggcctttcgacctgcagcccaagcttaccatgaccgagtacaagcccacggtgcgcctcgccacccgcg
acgacgtccccagggccgtacgcaccctcgccgccgcgttcgccgactaccccgccacgcgccacacegtcgatccggacc
gccacatcgagcgggtcaccgagctgcaagaactcttcctcacgcgcgtcgggctcgacatcggcaaggtgtgggtcgcgga
cgacggcgccgcggtggcggtctggaccacgccggagagcgtcgaagcgggggcggtgttcgccgagatcggcccgcgca
tggccgagttgagcggttcccggctggccgcgcagcaacagatggaaggcctcctggcgccgcaccggcccaaggagccc
gcgtggttcctggccaccgtcggcgtctcgcccgaccaccagggcaagggtctgggcagcgccgtcgtgctccccggagtgg
aggcggccgagcgcgccggggtgcccgccttcctggagacctccgcgccccgcaacctccccttctacgagcggctcggcttc
accgtcaccgccgacgtcgaggtgcccgaaggaccgcgcacctggtgcatgacccgcaagcccggtgcctgactcgaggg
aattaattcgagctcggtacctttaagaccaatgacttacaaggcagctgtagatcttagccactttttaaaagaaaaggggggac
tggaagggctaattcactcccaacgaagacaagatctgctttttgcttgtactgggtctctctggttagaccagatctgagcctggg
agctctctggctaactagggaacccactgcttaagcctcaataaagcttgccttgagtgcttcaagtagtgtgtgcccgtctgttgtgt
gactctggtaactagagatccctcagacccttttagtcagtgtggaaaatctctagcagcatctagaattaattccgtgtattctatag
tgtcacctaaatcgtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaaccttctgagtttctgg
taacgccgtcccgcacccggaaatggtcagcgaaccaatcagcagggtcatcgctagccagatcctctacgccggacgcatc
gtggccggcatcaccggcgccacaggtgcggttgctggcgcctatatcgccgacatcaccgatggggaagatcgggctcgcc
acttcgggctcatgagcgcttgtttcggcgtgggtatggtggcaggccccgtggccgggggactgttgggcgccatctccttgcat
gcaccattccttgcggcggcggtgctcaacggcctcaacctactactgggctgcttcctaatgcaggagtcgcataagggagag
cgtcgaatggtgcactctcagtacaatctgctctgatgccgcatagttaagccagccccgacacccgccaacacccgctgacgc
gccctgacgggcttgtctgctcccggcatccgcttacagacaagctgtgaccgtctccgggagctgcatgtgtcagaggttttcac
cgtcatcaccgaaacgcgcgagacgaaagggcctcgtgatacgcctatttttataggttaatgtcatgataataatggtttcttaga
cgtcaggtggcacttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttattcccttttttgcgg
cattttgccttcctgtttttgctcacccagaaacgctggtgaaagtaaaagatgctgaagatcagttgggtgcacgagtgggttacat
cgaactggatctcaacagcggtaagatccttgagagttttcgccccgaagaacgttttccaatgatgagcacttttaaagttctgct
atgtggcgcggtattatcccgtattgacgccgggcaagagcaactcggtcgccgcatacactattctcagaatgacttggttgagt
actcaccagtcacagaaaagcatcttacggatggcatgacagtaagagaattatgcagtgctgccataaccatgagtgataac
actgcggccaacttacttctgacaacgatcggaggaccgaaggagctaaccgcttttttgcacaacatgggggatcatgtaactc
gccttgatcgttgggaaccggagctgaatgaagccataccaaacgacgagcgtgacaccacgatgcctgtagcaatggcaac
aacgttgcgcaaactattaactggcgaactacttactctagcttcccggcaacaattaatagactggatggaggcggataaagtt
gcaggaccacttctgcgctcggcccttccggctggctggtttattgctgataaatctggagccggtgagcgtgggtctcgcggtatc
attgcagcactggggccagatggtaagccccccgtatcgtagttatctacacgacggggagtcaggcaactatggatgaacg
aaatagacagatcgctgagataggtgcctcactgattaagcattggtaactgtcagaccaagtttactcatatatactttagattgat
ttaaaacttcatttttaatttaaaaggatctaggtgaagatcctttttgataatctcatgaccaaaatcccttaacgtgagttttcgttcca
ctgagcgtcagaccccgtagaaaagatcaaaggatcttcttgagatcctttttttctgcgcgtaatctgctgcttgcaaacaaaaaa
accaccgctaccagcggtggtttgtttgccggatcaagagctaccaactctttttccgaaggtaactggcttcagcagagcgcag
ataccaaatactgttcttctagtgtagccgtagttaggccaccacttcaagaactctgtagcaccgcctacatacctcgctctgctaa
tcctgttaccagtggctgctgccagtggcgataagtcgtgtcttaccgggttggactcaagacgatagttaccggataaggcgca
gcggtcgggctgaacggggggttcgtgcacacagcccagcttggagcgaacgacctacaccgaactgagatacctacagcg
tgagctatgagaaagcgccacgcttcccgaagggagaaaggcggacaggtatccggtaagcggcagggtcggaacagga
gagcgcacgagggagcttccagggggaaacgcctggtatctttatagtcctgtcgggtttcgccacctctgacttgagcgtcgattt
ttgtgatgctcgtcaggggggcggagcctatggaaaaacgccagcaacgcggcctttttacggttcctggccttttgctggccttttg
ctcacatgttctttcctgcgttatcccctgattctgtggataaccgtattaccgcctttgagtgagctgataccgctcgccgcagccga
acgaccgagcgcagcgagtcagtgagcgaggaagcggaagagcgcccaatacgcaaaccgcctctccccgcgcgttggc
cgattcattaatgcagctgtggaatgtgtgtcagttagggtgtggaaagtccccaggctccccagcaggcagaagtatgcaaag
catgcatctcaattagtcagcaaccaggtgtggaaagtccccaggctccccagcaggcagaagtatgcaaagcatgcatctca
attagtcagcaaccatagtcccgcccctaactccgcccatcccgcccctaactccgcccagttccgcccattctccgccccatgg
ctgactaattttttttatttatgcagaggccgaggccgcctcggcctctgagctattccagaagtagtgaggaggcttttttggaggcc
taggcttttgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctattttcccctcgaa
cactttttaagccgtagataaacaggctgggacacttcacatgagcgaaaaatacatcgtcacctgggacatgttgcagatccat
gcacgtaaactcgcaagccgactgatgccttctgaacaatggaaaggcattattgccgtaagccgtggcggtctgtaccgggtg
cgttactggcgcgtgaactgggtattcgtcatgtcgataccgtttgtatttccagctacgatcacgacaaccagcgcgagcttaaag
tgctgaaacgcgcagaaggcgatggcgaaggcttcatcgttattgatgacctggtggataccggtggtactgcggttgcgattcgt
gaaatgtatccaaaagcgcactttgtcaccatcttcgcaaaaccggctggtcgtccgctggttgatgactatgttgttgatatcccgc
aagatacctggattgaacagccgtgggatatgggcgtcgtattcgtcccgccaatctccggtcgctaatcttttcaacgcctggca
ctgccgggcgttgttctttttaacttcaggcgggttacaatagtttccagtaagtattctggaggctgcatccatgacacaggcaaac
ctgagcgaaaccctgttcaaaccccgctttaaacatcctgaaacctcgacgctagtccgccgctttaatcacggcgcacaaccg
cctgtgcagtcggcccttgatggtaaaaccatccctcactggtatcgcatgattaaccgtctgatgtggatctggcgcggcattgac
ccacgcgaaatcctcgacgtccaggcacgtattgtgatgagcgatgccgaacgtaccgacgatgatttatacgatacggtgattg
gctaccgtggcggcaactggatttatgagtgggccccggatctttgtgaaggaaccttacttctgtggtgtgacataattggacaaa
ctacctacagagatttaaagctctaaggtaaatataaaatttttaagtgtataatgtgttaaactactgattctaattgtttgtgtattttag
attccaacctatggaactgatgaatgggagcagtggtggaatgcctttaatgaggaaaacctgttttgctcagaagaaatgccat
ctagtgatgatgaggctactgctgactctcaacattctactcctccaaaaaagaagagaaaggtagaagaccccaaggactttc
cttcagaattgctaagttttttgagtcatgctgtgtttagtaatagaactcttgcttgctttgctatttacaccacaaaggaaaaagctgc
actgctatacaagaaaattatggaaaaatattctgtaacctttataagtaggcataacagttataatcataacatactgttttttcttact
ccacacaggcatagagtgtctgctattaataactatgctcaaaaattgtgtacctttagctttttaatttgtaaaggggttaataagga
atatttgatgtatagtgccttgactagagatcataatcagccataccacatttgtagaggttttacttgctttaaaaaacctcccacac
ctccccctgaacctgaaacataaaatgaatgcaattgttgttgttaacttgtttattgcagcttataatggttacaaataaagcaatag
catcacaaatttcacaaataaagcatttttttcactgcattctagttgtggtttgtccaaactcatcaatgtatcttatcatgtctggatca
actggataactcaagctaaccaaaatcatcccaaacttcccaccccataccctattaccactgccaattacctgtggtttcatttact
ctaaacctgtgattcctctgaattattttcattttaaagaaattgtatttgttaaatatgtactacaaacttagtagt
SEQ IN NO: 53 - pREF044 sequence - Gal4-inducible CAR
AACAAAATATTAACGCTTACAATTTCCATTCGCCATTCAGGCTGCGCAACTGTTGGGAA
GGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACGA
CGGCCAGTGCCAAGCTGATCTATACATTGAATCAATATTGGCAATTAGCCATATTAGTC
ATTGGTTATATAGCATAAATCAATATTGGCTATTGGCCATTGCATACGTTGTATCTATAT
CATAAcggagcactgtcctccgaacgtcggagcactgtcctccgaacgtcggagcactgtcctccgaacgtcggagcactgt
cctccgaacggagcatgtcctccgaacgtcggagcactgtcctccgaacgactagttaggcgtgtacggtgggaggcctatata
agcagagctcgtttagtgaaccgtcagatcgcctggagacgccatccacgctgttttgacctccatagaagacaccgggaccg
atccagcctctcgacattcgttggGCCACCATGGGAACTAGTCTGCTGTGTTGGATGGCCCTCTGC
TTGTTGGGCGCAGACCACGCTGACGGCCAAGTTCAATTGGTCCAGTCTGGTGCCGAAC
TGAAGAAGCCAGGAGCTAGCGTCAAGGTGAGTTGTAAGTCTTCCGGATACCATTTCAC
GAGCTACTGGATGCATTGGGTAAAACAAGCCCCTGGACAAGGTCTCGAGTGGATAGG
GGTAATACATCCTAACTCTGGGTCCATAAATTACAATGAAAAGTTCGAATCCAGGGTTA
CCATCACAGTCGATAAGTCCACTTCCACGGCCTATATGGAATTGAGCTCCCTCAGGAG
CGAAGATACAGCTGTATACTACTGCGCTGGTGAACGAGACTCCACTGAGGTGTTGCCC
ATGGATTACTGGGGACAAGGCACCACTGTAACGGTCTCTAGCGGTGGGAGTAGTAGAA
GCTCATCATCTGGCGGCGGCGGTTCAGGCGGTGGCGGCGACGTGCAGATCACGCAAA
GCCCGAGCTCACTCTCCGCCAGTCTGGGTGAACGTGCGACTATAAATTGTAGAGCCAG
CAAGAGCATTAACAAGTATCTCGCCTGGTACCAGCAAAAGCCGGGCAAGGCTCCTAAA
CTCCTCATCTATTCCGGATCTACTCTCCAAAGCGGCATACCAGCGAGGTTCTCAGGAA
GCGGATCAGGAACGGATTTTACGCTTACGATATCTAGCCTCGAGCCAGAGGATTTTGC
CATGTACTATTGCCAACAACATAACGAATATCCCTTGACCTTCGGACAAGGGACGAAAC
TCGAGATCAAAGATTACAAAGACGATGACGACAAAACCACAACCCCAGCTCCTAGGCC
CCCAACACCTGCACCTACCATCGCTAGCCAGCCTCTGAGCCTGCGTCCCGAGGCATGT
CGTCCTGCTGCAGGTGGGGCAGTCCACACAAGAGGTCTGGACTTTGCATGTGACTTTT
GGGTCCTCGTAGTCGTCGGTGGCGTCCTGGCCTGTTATTCCCTTCTGGTTACGGTCGC
CTTCATTATATTTTGGGTCAGGAGTAAACGGTCTAGGGGGGGGCACAGCGACTACATG
AATATGACGCCACGCCGCCCCGGGCCCACAAGAAAACACTACCAACCTTACGCTCCAC
CCAGGGATTTTGCCGCGTACCGGAGCAGGGTAAAGTTCAGCCGCAGTGCGGATGCAC
CAGCGTATCAACAAGGCCAGAATCAACTGTATAATGAGCTGAACTTGGGACGACGAGA
GGAATATGATGTTCTGGATAAACGGCGCGGGAGAGATCCAGAGATGGGCGGAAAGCC
TAGACGTAAGAACCCCCAAGAAGGCCTTTATAATGAGTTGCAGAAAGATAAGATGGCC
GAAGCCTATTCTGAAATAGGCATGAAGGGAGAGCGGCGGCGTGGCAAGGGTCATGAC
GGTCTTTACCAGGGCCTCTCTACCGCAACTAAAGATACCTATGATGCACTTCACATGCA
AGCCCTGCCCCCTAGAGAGTTCGAAGGGTCAGCAGCAGCCGAGGGACGTGGCTCATT
GCTTACTTGCGGTGACGTTGAAGAAAACCCTGGCCCCAGCGGGATGGTAAGCAAGGG
AGAAGAACTTTTCACGGGAGTAGTTCCTATTCTGGTGGAACTGGACGGGGATGTAAAC
GGGCATAAATTCTCTGTCAGCGGGGAAGGGGAAGGCGATGCAACATACGGGAAGCTT
ACCCTCAAATTCATCTGTACGACAGGGAAACTTCCAGTGCCCTGGCCTACACTTGTGAC
AACCCTGACTTATGGGGTCCAATGTTTCTCCAGGTACCCTGATCACATGAAACAGCATG
ACTTCTTCAAGAGCGCAATGCCTGAAGGGTATGTCCAGGAACGCACGATTTTCTTCAAG
GACGATGGCAATTATAAAACTAGAGCCGAAGTAAAATTTGAGGGAGATACATTGGTAAA
CAGGATCGAGCTCAAAGGGATCGATTTCAAAGAGGATGGGAACATCCTGGGGCATAAG
TTGGAGTATAACTATAACTCTCACAATGTCTATATTATGGCCGACAAGCAAAAGAACGG
GATAAAAGTCAACTTTAAAATAAGGCACAATATAGAGGACGGCAGTGTGCAATTGGCG
GACCACTATCAACAAAATACCCCGATCGGCGACGGTCCAGTGTTGCTCCCTGATAATC
ACTACCTTTCAACACAAAGTGCACTTAGTAAAGATCCCAATGAGAAGCGGGATCACATG
GTCCTGCTCGAATTTGTCACAGCAGCTGGCATCACACTTGGGATGGATGAGCTCTATA
AATAATAAACGGCCGGCCGCGGTCATAGCTGTTTCCTGAACAGATCCCGGGTGGCATC
CCTGTGACCCCTCCCCAGTGCCTCTCCTGGCCCTGGAAGTTGCCACTCCAGTGCCCAC
CAGCCTTGTCCTAATAAAATTAAGTTGCATCATTTTGTCTGACTAGGTGTCCTTCTATAA
TATTATGGGGTGGAGGGGGGTGGTATGGAGCAAGGGGCAAGTTGGGAAGACAACCTG
TAGGGCCTGCGGGGTCTATTGGGAACCAAGCTGGAGTGCAGTGGCACAATCTTGGCT
CACTGCAATCTCCGCCTCCTGGGTTCAAGCGATTCTCCTGCCTCAGCCTCCCGAGTTG
TTGGGATTCCAGGCATGCATGACCAGGCTCAGCTAATTTTTGTTTTTTTGGTAGAGACG
GGGTTTCACCATATTGGCCAGGCTGGTCTCCAACTCCTAATCTCAGGTGATCTACCCAC
CTTGGCCTCCCAAATTGCTGGGATTACAGGCGTGAACCACTGCTCCCTTCCCTGTCCT
TCTGATTTTAAAATAACTATACCAGCAGGAGGACGTCCAGACACAGCATAGGCTACCTG
GCCATGCCCAACCGGTGGGACATTTGAGTTGCTTGCTTGGCACTGTCCTCTCATGCGT
TGGGTCCACTCAGTAGATGCCTGTTGAATTGGGTACGCGGCCAGCTTGGCTGTGGAAT
GTGTGTCAGTTAGGGTGTGGAAAGTCCCCAGGCTCCCCAGCAGGCAGAAGTATGCAA
AGCATGCATCTCAATTAGTCAGCAACCAGGTGTGGAAAGTCCCCAGGCTCCCCAGCAG
GCAGAAGTATGCAAAGCATGCATCTCAATTAGTCAGCAACCATAGTCCCGCCCCTAACT
CCGCCCATCCCGCCCCTAACTCCGCCCAGTTCCGCCCATTCTCCGCCCCATGGCTGA
CTAATTTTTTTTATTTATGCAGAGGCCGAGGCCGCCTCGGCCTCTGAGCTATTCCAGAA
GTAGTGAGGAGGCTTTTTTGGAGGCCTAGGCTTTTGCAAAAAGCTCCCGGGAGCTTGT
ATATCCATTTTCGGATCTGATCAAGAGACAGGATGAGGATCGTTTCGCATGATTGAACA
AGATGGATTGCACGCAGGTTCTCCGGCCGCTTGGGTGGAGAGGCTATTCGGCTATGA
CTGGGCACAACAGACAATCGGCTGCTCTGATGCCGCCGTGTTCCGGCTGTCAGCGCA
GGGGCGCCCGGTTCTTTTTGTCAAGACCGACCTGTCCGGTGCCCTGAATGAACTGCAG
GACGAGGCAGCGCGGCTATCGTGGCTGGCCACGACGGGCGTTCCTTGCGCAGCTGT
GCTCGACGTTGTCACTGAAGCGGGAAGGGACTGGCTGCTATTGGGCGAAGTGCCGGG
GCAGGATCTCCTGTCATCTCACCTTGCTCCTGCCGAGAAAGTATCCATCATGGCTGAT
GCAATGCGGCGGCTGCATACGCTTGATCCGGCTACCTGCCCATTCGACCACCAAGCG
AAACATCGCATCGAGCGAGCACGTACTCGGATGGAAGCCGGTCTTGTCGATCAGGATG
ATCTGGACGAAGAGCATCAGGGGCTCGCGCCAGCCGAACTGTTCGCCAGGCTCAAGG
CGCGCATGCCCGACGGCGAGGATCTCGTCGTGACCCATGGCGATGCCTGCTTGCCGA
ATATCATGGTGGAAAATGGCCGCTTTTCTGGATTCATCGACTGTGGCCGGCTGGGTGT
GGCCGACCGCTATCAGGACATAGCGTTGGCTACCCGTGATATTGCTGAAGAGCTTGGC
GGCGAATGGGCTGACCGCTTCCTCGTGCTTTACGGTATCGCCGCTCCCGATTCGCAG
CGCATCGCCTTCTATCGCCTTCTTGACGAGTTCTTCTGAGCGGGACTCTGGGGTTCGA
AATGACCGACCAAGCGACGCCCAACCTGCCATCACGAGATTTCGATTCCACCGCCGCC
TTCTATGAAAGGTTGGGCTTCGGAATCGTTTTCCGGGACGCCGGCTGGATGATCCTCC
AGCGCGGGGATCTCATGCTGGAGTTCTTCGCCCACCCCAACTTGTTTATTGCAGCTTAT
AATGGTTACAAATAAAGCAATAGCATCACAAATTTCACAAATAAAGCATTTTTTTCACTG
CATTCTAGTTGTGGTTTGTCCAAACTCATCAATGTATCTTATCATGTCTGTATACCGTCG
ACCTCTAGCTAGAGCTTGGCGTAATCATGGTCATAGCTGTTTCCTGTGTGAAATTGTTA
TCCGCTCACAATTCCACACAACATACGAGCCGGAAGCATAAAGTGTAAAGCCTGGGGT
GCCTAATGAGTGAGCTAACTCACATTAATTGCGTTGCGCTCACTGCCCGCTTTCCAGTC
GGGAAACCTGTCGTGCCAGCTGCATTAATGAATCGGCCAACGCGCGGGGAGAGGCGG
TTTGCGTATTGGGCGCTCTTCCGCTTCCTCGCTCACTGACTCGCTGCGCTCGGTCGTT
CGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGA
AGAACAGTATTTGGTATCTGCGCTCTGCTGAAGCCAGTTACCTTCGGAAAAAGAGTTGG
TAGCTCTTGATCCGGCAAACAAACCACCGCTGGTAGCGGTTTTTTTGTTTGCAAGCAGC
AGATTACGCGCAGAAAAAAAGGATCTCAAGAAGATCCTTTGATCTTTTCTACGGGGTCT
GACGCTCAGTGGAACGAAAACTCACGTTAAGGGATTTTGGTCATGAGATTATCAAAAAG
GATCTTCACCTAGATCCTTTTAAATTAAAAATGAAGTTTTAAATCAATCTAAAGTATATAT
GAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTGGT
GTCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAG
TCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCG
GGGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCG
GATACATATTTGAATGTATTTAGAAAAATAAACAAATAGGGGTTCCGCGCACATTTCCCC
GAAAAGTGCCACCTGACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCGCTT
TCTTCCCTTCCTTTCTCGCCACGTTCGCCGGCTTTCCCCGTCAAGCTCTAAATCGGGG
GCTCCCTTTAGGGTTCCGATTTAGTGCTTTACGGCACCTCGACCCCAAAAAACTTGATT
AGGGTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCAACC
CTATCTCGGTCTATTCTTTTGATTTATAAGGGATTTTGCCGATTTCGGCCTATTGGTTAA
AAAATGAGCTGATTTAACAAAAATTTAACGCGAATTTT
SEQ ID NO: 54 - pREF031 - transient expression vector for the first receptor (synNotch
WT neurexin sequence)
AACAAAATATTAACGCTTACAATTTCCATTCGCCATTCAGGCTGCGCAACTGTTGGGAA
GGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACGA
CGGCCAGTGCCAAGCTGATCTATACATTGAATCAATATTGGCAATTAGCCATATTAGTC
ATTGGTTATATAGCATAAATCAATATTGGCTATTGGCCATTGCATACGTTGTATCTATAT
CATAATATGTACATTTATATTGGCTCATGTCCAATATGACCGCCATGTTGACATTGATTA
TTGACTAGTTATTAATAGTAATCAATTACGGGGTCATTAGTTCATAGCCCATATATGGAG
TTCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTA
TCATATGCCAAGTCCGCCCCCTATTGACGTCAATGACGGTAAATGGCCCGCCTGGCAT
TATGCCCAGTACATGACCTTACGGGACTTTCCTACTTGGCAGTACATCTACGTATTAGT
CATCGCTATTACCATGGTGATGCGGTTTTGGCAGTACACCAATGGGCGTGGATAGCGG
TTTGACTCACGGGGATTTCCAAGTCTCCACCCCATTGACGTCAATGGGAGTTTGTTTTG
GCACCAAAATCAACGGGACTTTCCAAAATGTCGTAATAACCCCGCCCCGTTGACGCAA
ATGGGCGGTAGGCGTGTACGGTGGGAGGTCTATATAAGCAGAGCTCGTTTAGTGAACC
GTCAGAATTTTGTAATACGACTCACTATAGGGCGGCCGGGAATTCGTCGACTGGATCC
GGTACCGAGGAGATCTGCCGCCGCGATCGCCatggcattgcccgtgaccgccctgctgctgccactggc
cttgttgctccacgccgcgcggccagaacagaagCTGATCAGCGAGGAGGATCTGATGTACCAGAGG
ATGCTGAGGTGCGGCGCCGAGCTGGGCAGCCCCGGGGGGGGGGGGCGGCGGCG
GCGGCGGCGCCGGCGGCAGGCTGGCCCTGCTGTGGATCGTGCCCCTGACCCTGAGC
GGCCTGCTGGGCGTGGCCTGGGGCGCCAGCAGCCTGGGCGCCCACCACATCCACCA
CTTCCACGGCAGCAGCAAGCACCACAGCGTGCCCATCGCCATCTACAGGAGCCCCGC
CAGCCTGAGGGGCGGCCACGCCGGCACCACCTACATCTTCAGCAAGGGCGGCGGCC
AGATCACCTACAAGTGGCCCCCCAACGACAGGCCCAGCACCAGGGCCGACAGGCTGG
CCATCGGCTTCAGCACCGTGCAGAAGGAGGCCGTGCTGGTGAGGGTGGACAGCAGCA
GCGGCCTGGGCGACTACCTGGAGCTGCACATCCACCAGGGCAAGATCGGCGTGAAGT
TCAACGTGGGCACCGACGACATCGCCATCGAGGAGAGCAACGCCATCATCAACGACG
GCAAGTACCACGTGGTGAGGTTCACCAGGAGCGGCGGCAACGCCACCCTGCAGGTG
GACAGCTGGCCCGTGATCGAGAGGTACCCCGCCGGCAGGCAGCTGACCATCTTCAAC
AGCCAGGCCACCATCATCATCGGCGGCAAGGAGCAGGGCCAGCCCTTCCAGGGCCA
GCTGAGCGGCCTGTACTACAACGGCCTGAAGGTGCTGAACATGGCCGCCGAGAACGA
CGCCAACATCGCCATCGTGGGCAACGTGAGGCTGGTGGGCGAGGTGCCCATCCTGGA
CTACAGCTTCACAGGtggcgctgggcgcgacattcccccaccgcagattgaggaggcctgtgagctgcctgagtgcc
aggtggatgcaggcaataaggtctgcaacctgcagtgtaataatcacgcatgtggctgggatggtggcgactgctccctcaactt
caatgacccctggaagaactgcacgcagtctctacagtgctggaagtattttagcgacggccactgtgacagccagtgcaactc
ggccggctgcctctttgatggcttcgactgccagctcaccgagggacagtgcaaccccctgtatgaccagtactgcaaggacca
cttcagtgatggccactgcgaccagggctgtaacagtgccgaatgtgagtgggatggcctagactgtgctgagcatgtacccga
gcggctggcagccggcaccctggtgctggtggtgctgcttccacccgaccagctacggaacaactccttccactttctgcggga
gctcagccacgtgctgcacaccaacgtggtcttcaagcgtgatgcgcaaggccagcagatgatcttcccgtactatggccacga
ggaagagctgcgcaagcacccaatcaagcgctctacagtgggttgggccacctcttcactgcttcctggtaccagtggtgggcg
ccagcgcagggagctggaccccatggacatccgtggctccattgtctacctggagatcgacaaccggcaatgtgtgcagtcatc
ctcgcagtgcttccagagtgccaccgatgtggctgccttcctaggtgctcttgcgtcacttggcagcctcaatattccttacaagattg
aggccgtgaagagtgagccggtggagcctccgctgccctcgcagctgcacctcatgtacgtggcagcggccgccttcgtgctc
ctgttctttgtgggctgtggggtgctgctgtcccgcaagcgccggcggatgaagctgctgagcagcatcgagcaggcctgtgaca
tctgccggctgaagaaactgaagtgcagcaaagaaaagcccaagtgcgccaagtgcctgaagaacaactgggagtgccgg
tacagccccaagaccaagagaagccccctgaccagagcccacctgaccgaggtggaaagccggctggaaagactggaac
agctgtttctgctgatcttcccacgcgaggacctggacatgatcctgaagatggacagcctgcaggacatcaaggccctgctga
ccggcctgttcgtgcaggacaacgtgaacaaggacgccgtgaccgacagactggccagcgtggaaaccgacatgcccctga
ccctgcggcagcacagaatcagcgccaccagcagcagcgaggaaagcagcaacaagggccagcggcagctgacagtgt
ctgctgctgcaggcggaagcggaggctctggcggatctgatgccctggacgacttcgacctggatatgctgggcagcgacgcc
ctggatgattttgatctggacatgctgggatctgacgctctggacgatttcgatctcgacatgttgggatcagatgcactggatgactt
tgacctggacatgctcggatcatgaTAAACGGCCGGCCGCGGTCATAGCTGTTTCCTGAACAGATC
CCGGGTGGCATCCCTGTGACCCCTCCCCAGTGCCTCTCCTGGCCCTGGAAGTTGCCA
CTCCAGTGCCCACCAGCCTTGTCCTAATAAAATTAAGTTGCATCATTTTGTCTGACTAG
GTGTCCTTCTATAATATTATGGGGTGGAGGGGGGTGGTATGGAGCAAGGGGCAAGTTG
GGAAGACAACCTGTAGGGCCTGCGGGGTCTATTGGGAACCAAGCTGGAGTGCAGTGG
CACAATCTTGGCTCACTGCAATCTCCGCCTCCTGGGTTCAAGCGATTCTCCTGCCTCA
GCCTCCCGAGTTGTTGGGATTCCAGGCATGCATGACCAGGCTCAGCTAATTTTTGTTTT
TTTGGTAGAGACGGGGTTTCACCATATTGGCCAGGCTGGTCTCCAACTCCTAATCTCA
GGTGATCTACCCACCTTGGCCTCCCAAATTGCTGGGATTACAGGCGTGAACCACTGCT
CCCTTCCCTGTCCTTCTGATTTTAAAATAACTATACCAGCAGGAGGACGTCCAGACACA
GCATAGGCTACCTGGCCATGCCCAACCGGTGGGACATTTGAGTTGCTTGCTTGGCACT
GTCCTCTCATGCGTTGGGTCCACTCAGTAGATGCCTGTTGAATTGGGTACGCGGCCAG
CTTGGCTGTGGAATGTGTGTCAGTTAGGGTGTGGAAAGTCCCCAGGCTCCCCAGCAG
GCAGAAGTATGCAAAGCATGCATCTCAATTAGTCAGCAACCAGGTGTGGAAAGTCCCC
AGGCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCTC
CGCCCCATGGCTGACTAATTTTTTTTATTTATGCAGAGGCCGAGGCCGCCTCGGCCTC
TGAGCTATTCCAGAAGTAGTGAGGAGGCTTTTTTGGAGGCCTAGGCTTTTGCAAAAAG
CTCCCGGGAGCTTGTATATCCATTTTCGGATCTGATCAAGAGACAGGATGAGGATCGTT
TCGCATGATTGAACAAGATGGATTGCACGCAGGTTCTCCGGCCGCTTGGGTGGAGAG
GCTATTCGGCTATGACTGGGCACAACAGACAATCGGCTGCTCTGATGCCGCCGTGTTC
CGGCTGTCAGCGCAGGGGCGCCCGGTTCTTTTTGTCAAGACCGACCTGTCCGGTGCC
CTGAATGAACTGCAGGACGAGGCAGCGCGGCTATCGTGGCTGGCCACGACGGGCGTT
CCTTGCGCAGCTGTGCTCGACGTTGTCACTGAAGCGGGAAGGGACTGGCTGCTATTG
GGCGAAGTGCCGGGGCAGGATCTCCTGTCATCTCACCTTGCTCCTGCCGAGAAAGTAT
CCATCATGGCTGATGCAATGCGGCGGCTGCATACGCTTGATCCGGCTACCTGCCCATT
CGACCACCAAGCGAAACATCGCATCGAGCGAGCACGTACTCGGATGGAAGCCGGTCT
TGTCGATCAGGATGATCTGGACGAAGAGCATCAGGGGCTCGCGCCAGCCGAACTGTT
CGCCAGGCTCAAGGCGCGCATGCCCGACGGCGAGGATCTCGTCGTGACCCATGGCG
ATGCCTGCTTGCCGAATATCATGGTGGAAAATGGCCGCTTTTCTGGATTCATCGACTGT
GGCCGGCTGGGTGTGGCCGACCGCTATCAGGACATAGCGTTGGCTACCCGTGATATT
GCTGAAGAGCTTGGCGGCGAATGGGCTGACCGCTTCCTCGTGCTTTACGGTATCGCC
GCTCCCGATTCGCAGCGCATCGCCTTCTATCGCCTTCTTGACGAGTTCTTCTGAGCGG
GACTCTGGGGTTCGAAATGACCGACCAAGCGACGCCCAACCTGCCATCACGAGATTTC
GATTCCACCGCCGCCTTCTATGAAAGGTTGGGCTTCGGAATCGTTTTCCGGGACGCCG
GCTGGATGATCCTCCAGCGCGGGGATCTCATGCTGGAGTTCTTCGCCCACCCCAACTT
GTTTATTGCAGCTTATAATGGTTACAAATAAAGCAATAGCATCACAAATTTCACAAATAA
AGCATTTTTTTCACTGCATTCTAGTTGTGGTTTGTCCAAACTCATCAATGTATCTTATCAT
GTCTGTATACCGTCGACCTCTAGCTAGAGCTTGGCGTAATCATGGTCATAGCTGTTTCC
TGTGTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCACT
GCCCGCTTTCCAGTCGGGAAACCTGTCGTGCCAGCTGCATTAATGAATCGGCCAACGC
GCGGGGAGAGGCGGTTTGCGTATTGGGCGCTCTTCCGCTTCCTCGCTCACTGACTCG
CTGCGCTCGGTCGTTCGGCTGCGGCGAGCGGTATCAGCTCACTCAAAGGCGGTAATA
CGGTTATCCACAGAATCAGGGGATAACGCAGGAAAGAACATGTGAGCAAAAGGCCAGC
AAAAGGCCAGGAACCGTAAAAAGGCCGCGTTGCTGGCGTTTTTCCATAGGCTCCGCCC
CCCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGT
CTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAA
CTACGGCTACACTAGAAGAACAGTATTTGGTATCTGCGCTCTGCTGAAGCCAGTTACCT
TCGGAAAAAGAGTTGGTAGCTCTTGATCCGGCAAACAAACCACCGCTGGTAGCGGTTT
TTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTC
ATGAGATTATCAAAAAGGATCTTCACCTAGATCCTTTTAAATTAAAAATGAAGTTTTAAAT
CAATCTAAAGTATATATGAGTAAACTTGGTCTGACAGTTACCAATGCTTAATCAGTGAGG
CACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGC
GAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGC
CGGGAAGCTAGAGTAAGTAGTTCGCCAGTTAATAGTTTGCGCAACGTTGTTGCCATTG
CTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCATC
ATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAAGGGCGA
CACGGAAATGTTGAATACTCATACTCTTCCTTTTTCAATATTATTGAAGCATTTATCAGG
GTTATTGTCTCATGAGCGGATACATATTTGAATGTATTTAGAAAAATAAACAAATAGGGG
TTCCGCGCACATTTCCCCGAAAAGTGCCACCTGACGCGCCCTGTAGCGGCGCATTAAG
CGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACG
GTTTTTCGCCCTTTGACGTTGGAGTCCACGTTCTTTAATAGTGGACTCTTGTTCCAAACT
GGAACAACACTCAACCCTATCTCGGTCTATTCTTTTGATTTATAAGGGATTTTGCCGATT
TCGGCCTATTGGTTAAAAAATGAGCTGATTTAACAAAAATTTAACGCGAATTTT
SEQ ID NO: 55 - Construct pREF043 sequence - constitutively expressed Gal4 and CAR
under control of Gal4-inducible element
tggaagggctaattcactcccaaagaagacaagatatccttgatctgtggatctaccacacacaaggctacttccctgattagca
gaactacacaccagggccaggggtcagatatccactgacctttggatggtgctacaagctagtaccagttgagccagataagg
tagaagaggccaataaaggagagaacaccagcttgttacaccctgtgagcctgcatgggatggatgacccggagagagaag
tgttagagtggaggtttgacagccgcctagcatttcatcacgtggcccgagagctgcatccggagtacttcaagaactgctgatat
cgagcttgctacaagggactttccgctggggactttccagggaggcgtggcctggggggactggggagtggcgagccctcag
atcctgcatataagcagctgctttttgcctgtactgggtctctctggttagaccagatctgagcctgggagctctctggctaactaggg
aacccactgcttaagcctcaataaagcttgccttgagtgcttcaagtagtgtgtgcccgtctgttgtgtgactctggtaactagagatc
cctcagacccttttagtcagtgtggaaaatctctagcagtggcgcccgaacagggacttgaaagcgaaagggaaaccagagg
agctctctcgacgcaggactcggcttgctgaagcgcgcacggcaagaggcgaggggcggcgactggtgagtacgccaaaa
attttgactagcggaggctagaaggagagagatgggtgcgagagcgtcagtattaagcgggggagaattagatcgcgatggg
aaaaaattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcagacaggat
cagaagaacttagatcattatataatacagtagcaaccctctattgtgtgcatcaaaggatagagataaaagacaccaaggaa
gctttagacaagatagaggaagagcaaaacaaaagtaagaccaccgcacagcaagcggccggccgctgatcttcagacct
ggaggaggagatatgagggacaattggagaagtgaattatataaatataaagtagtaaaaattgaaccattaggagtagcacc
caccaaggcaaagagaagagtggtgcagagagaaaaaagagcagtgggaataggagctttgttccttgggttcttgggagca
gcaggaagcactatgggcgcagcgtcaatgacgctgacggtacaggccagacaattattgtctggtatagtgcagcagcaga
acaatttgctgagggctattgaggcgcaacagcatctgttgcaactcacagtctggggcatcaagcagctccaggcaagaatcc
tggctgtggaaagatacctaaaggatcaacagctcctggggatttggggttgctctggaaaactcatttgcaccactgctgtgcctt
ggaatgctagttggagtaataaatctctggaacagatttggaatcacacgacctggatggagtgggacagagaaattaacaatt
acacaagcttaatacactccttaattgaagaatcgcaaaaccagcaagaaaagaatgaacaagaattattggaattagataaa
tgggcaagtttgtggaattggtttaacataacaaattggctgtggtatataaaattattcataatgatagtaggaggcttggtaggttta
agaatagtttttgctgtactttctatagtgaatagagttaggcagggatattcaccattatcgtttcagacccacctcccaaccccgag
gggacccgacaggcccgaaggaatagaagaagaaggtggagagagagacagagacagatccattcgattagtgaacgg
atctcgacggtatcgccaaatggcagtattcatccacaattttaaaagaaaaggggggattggggggtacagtgcaggggaaa
gaatagtagacataatagcaacagacatacaaactaaagaattacaaaaacaaattacaaaaattcaaaattttcgggtttatta
cagggacagcagagatccagtttggatcgataagcttgatatcgaattGCATAAATCAATATTGGCTATTGGCC
ATTGCATACGTTGTATCTATATCATAAcggagcactgtcctccgaacgtcggagcactgtcctccgaacgtcg
gagcactgtcctccgaacgtcggagcactgtcctccgaacggagcatgtcctccgaacgtcggagcactgtcctccgaacgac
tagttaggcgtgtacggtgggaggcctatataagcagagctcgtttagtgaaccgtcagatcgcctggagacgccatccacgct
gttttgacctccatagaagacaccgggaccgatccagcctctcgacattcgttggGCCACCATGGGAACTAGTCTG
CTGTGTTGGATGGCCCTCTGCTTGTTGGGCGCAGACCACGCTGACGGCCAAGTTCAAT
TGGTCCAGTCTGGTGCCGAACTGAAGAAGCCAGGAGCTAGCGTCAAGGTGAGTTGTAA
GTCTTCCGGATACCATTTCACGAGCTACTGGATGCATTGGGTAAAACAAGCCCCTGGA
CAAGGTCTCGAGTGGATAGGGGTAATACATCCTAACTCTGGGTCCATAAATTACAATGA
AAAGTTCGAATCCAGGGTTACCATCACAGTCGATAAGTCCACTTCCACGGCCTATATGG
AATTGAGCTCCCTCAGGAGCGAAGATACAGCTGTATACTACTGCGCTGGTGAACGAGA
CTCCACTGAGGTGTTGCCCATGGATTACTGGGGACAAGGCACCACTGTAACGGTCTCT
AGCGGTGGGAGTAGTAGAAGCTCATCATCTGGCGGCGGCGGTTCAGGCGGTGGCGG
CGACGTGCAGATCACGCAAAGCCCGAGCTCACTCTCCGCCAGTCTGGGTGAACGTGC
GACTATAAATTGTAGAGCCAGCAAGAGCATTAACAAGTATCTCGCCTGGTACCAGCAAA
AGCCGGGCAAGGCTCCTAAACTCCTCATCTATTCCGGATCTACTCTCCAAAGCGGCAT
ACCAGCGAGGTTCTCAGGAAGCGGATCAGGAACGGATTTTACGCTTACGATATCTAGC
CTCGAGCCAGAGGATTTTGCCATGTACTATTGCCAACAACATAACGAATATCCCTTGAC
CTTCGGACAAGGGACGAAACTCGAGATCAAAGATTACAAAGACGATGACGACAAAACC
ACAACCCCAGCTCCTAGGCCCCCAACACCTGCACCTACCATCGCTAGCCAGCCTCTGA
GCCTGCGTCCCGAGGCATGTCGTCCTGCTGCAGGTGGGGCAGTCCACACAAGAGGTC
TGGACTTTGCATGTGACTTTTGGGTCCTCGTAGTCGTCGGTGGCGTCCTGGCCTGTTA
TTCCCTTCTGGTTACGGTCGCCTTCATTATATTTTGGGTCAGGAGTAAACGGTCTAGGG
GCGGGCACAGCGACTACATGAATATGACGCCACGCCGCCCCGGGCCCACAAGAAAAC
ACTACCAACCTTACGCTCCACCCAGGGATTTTGCCGCGTACCGGAGCAGGGTAAAGTT
CAGCCGCAGTGCGGATGCACCAGCGTATCAACAAGGCCAGAATCAACTGTATAATGAG
CTGAACTTGGGACGACGAGAGGAATATGATGTTCTGGATAAACGGCGCGGGAGAGAT
CCAGAGATGGGCGGAAAGCCTAGACGTAAGAACCCCCAAGAAGGCCTTTATAATGAGT
TGCAGAAAGATAAGATGGCCGAAGCCTATTCTGAAATAGGCATGAAGGGAGAGCGGC
GGCGTGGCAAGGGTCATGACGGTCTTTACCAGGGCCTCTCTACCGCAACTAAAGATAC
CTATGATGCACTTCACATGCAAGCCCTGCCCCCTAGAGAGTTCGAAGGGTCAGCAGCA
GCCGAGGGACGTGGCTCATTGCTTACTTGCGGTGACGTTGAAGAAAACCCTGGCCCC
AGCGGGATGGTAAGCAAGGGAGAAGAACTTTTCACGGGAGTAGTTCCTATTCTGGTGG
AACTGGACGGGGATGTAAACGGGCATAAATTCTCTGTCAGCGGGGAAGGGGAAGGCG
ATGCAACATACGGGAAGCTTACCCTCAAATTCATCTGTACGACAGGGAAACTTCCAGTG
CCCTGGCCTACACTTGTGACAACCCTGACTTATGGGGTCCAATGTTTCTCCAGGTACC
CTGATCACATGAAACAGCATGACTTCTTCAAGAGCGCAATGCCTGAAGGGTATGTCCA
GGAACGCACGATTTTCTTCAAGGACGATGGCAATTATAAAACTAGAGCCGAAGTAAAAT
TTGAGGGAGATACATTGGTAAACAGGATCGAGCTCAAAGGGATCGATTTCAAAGAGGA
TGGGAACATCCTGGGGCATAAGTTGGAGTATAACTATAACTCTCACAATGTCTATATTAT
GGCCGACAAGCAAAAGAACGGGATAAAAGTCAACTTTAAAATAAGGCACAATATAGAG
GACGGCAGTGTGCAATTGGCGGACCACTATCAACAAAATACCCCGATCGGCGACGGT
CCAGTGTTGCTCCCTGATAATCACTACCTTTCAACACAAAGTGCACTTAGTAAAGATCC
CAATGAGAAGCGGGATCACATGGTCCTGCTCGAATTTGTCACAGCAGCTGGCATCACA
CTTGGGATGGATGAGCTCTATAAATAATAAACGGCCGGCCGCGGTCATAGCTGTTTCC
TGAACAGATCCCGGGTGGCATCCCTGTGACCCCTCCCCAGTGCCTCTCCTGGCCCTG
GAAGTTGCCACTCCAGTGCCCACCAGCCTTGTCCTAATAAAATTAAGTTGCATCATTTT
GTCTGACTAGGTGTCCTTCTATAATATTATGGGGTGGAGGGGGGTGGTATGGAGCAAG
GGGCAAGTTGGGAAGACAACCTGTAGGGCCTGCGGGGTCTATTGGGAACCAAGCTGG
AGTGCAGTGGCACAATCTTGGCTCACTGCAATCTCCGCCTCCTGGGTTCAAGCGATTC
TCCTGCCTCAGCCTCCCGAGTTGTTGGGATTCCAGGCATGCATGACCAGGCTCAGCTA
ATTTTTGTTTTTTTGGTAGAGACGGGGTTTCACCATATTGGCCAGGCTGGTCTCCAACT
CCTAATCTCAGGTGATCTACCCACCTTGGCCTCCCAAATTGCTGGGATTACAGGCGTG
AACCACTGCTCCCTTCCCTGTCCTTCTGATTTTAAAATAACTATACCAGCAGGAGGACG
TCCAGACACAGCATAGGCTACCTGGCCATGCCCAACCGGTGGGACATTTGAGTTGCTT
GCTTGGCACTGTCCTCTCATGCGTTGGGTCCACTCAGTAGATGCCTGTTGAATTGACAT
TGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTACATCA
AGTGTATCATATGCCAAGTCCGCCCCCTATTGACGTCAATGACGGTAAATGGCCCGCC
TGGCATTATGCCCAGTACATGACCTTACGGGACTTTCCTACTTGGCAGTACATCTACGT
ATTAGTCATCGCTATTACCATGGTGATGCGGTTTTGGCAGTACACCAATGGGCGTGGAT
AGCGGTTTGACTCACGGGGATTTCCAAGTCTCCACCCCATTGACGTCAATGGGAGTTT
GTTTTGGCACCAAAATCAACGGGACTTTCCAAAATGTCGTAATAACCCCGCCCCGTTGA
CGCAAATGGGGGGTAGGCGTGTACGGTGGGAGGTCTATATAAGCAGAGCTCGTTTAGT
GAACCGTCAGAATTTTGTAATACGACTCACTATAGGGCGGCCGGGAATTCGTCGACTG
GATCCGGTACCGAGGAGATCTGCCGCCGCGATCGCCatgaagctgctgagcagcatcgagcaggc
ctgtgacatctgccggctgaagaaactgaagtgcagcaaagaaaagcccaagtgcgccaagtgcctgaagaacaactggg
agtgccggtacagccccaagaccaagagaagccccctgaccagagcccacctgaccgaggtggaaagccggctggaaag
actggaacagctgtttctgctgatcttcccacgcgaggacctggacatgatcctgaagatggacagcctgcaggacatcaaggc
cctgctgaccggcctgttcgtgcaggacaacgtgaacaaggacgccgtgaccgacagactggccagcgtggaaaccgacat
gcccctgaccctgcggcagcacagaatcagcgccaccagcagcagcgaggaaagcagcaacaagggccagcggcagct
gacagtgtctgctgctgcaggcggaagcggaggctctggcggatctgatgccctggacgacttcgacctggatatgctgggcag
cgacgccctggatgattttgatctggacatgctgggatctgacgctctggacgatttcgatctcgacatgttgggatcagatgcact
ggatgactttgacctggacatgctcggatcatgaTAAACGGCCGGCCGCGGTCATAGCTGTTTCCTGGG
CCGCGACTCTAGAgtcgacctgcaggcatgcaagcttgatatcaagcttatcgataatcaacctctggattacaaaattt
gtgaaagattgactggtattcttaactatgttgctccttttacgctatgtggatacgctgctttaatgcctttgtatcatgctattgcttcccc
gtatggctttcattttctcctccttgtataaatcctggttgctgtctctttatgaggagttgtggcccgttgtcaggcaacgtggcgtggtgt
gcactgtgtttgctgacgcaacccccactggttggggcattgccaccacctgtcagctcctttccgggactttcgctttccccctccct
attgccacggcggaactcatcgccgcctgccttgcccgctgctggacaggggctcggctgttgggcactgacaattccgtggtgtt
gtcggggaaatcatcgtcctttccttggctgctcgcctgtgttgccacctggattctgcgcgggacgtccttctgctacgtcccttcgg
ccctcaatccagcggaccttccttcccaattctaccgggtaggggaggcgcttttcccaaggcagtctggagcatgcgctttagca
gccccgctgggcacttggcgctacacaagtggcctctggcctcgcacacattccacatccaccggtaggcgccaaccggctcc
gttctttggtggccccttcgcgccaccttctactcctcccctagtcaggaagttcccccccgccccgcagctcgcgtcgtgcaggac
gtgacaaatggaagtagcacgtctcactagtctcgtgcagatggacagcaccgctgagcaatggaagcgggtaggcctttggg
gcagcggccaatagcagctttgctccttcgctttctgggctcagaggctgggaaggggtgggtccggggggggctcaggggc
gggctcaggggggggcgggcgcccgaaggtcctccggaggcccggcattctgcacgcttcaaaagcgcacgtctgccgcg
ctgttctcctcttcctcatctccgggcctttcgacctgcagcccaagcttaccatgaccgagtacaagcccacggtgcgcctcgcca
cccgcgacgacgtccccagggccgtacgcaccctcgccgccgcgttcgccgactaccccgccacgcgccacaccgtcgatc
cggaccgccacatcgagcgggtcaccgagctgcaagaactcttcctcacgcgcgtcgggctcgacatcggcaaggtgtgggt
cgcggacgacggcgccgcggtggcggtctggaccacgccggagagcgtcgaagcgggggcggtgttcgccgagatcggc
ccgcgcatggccgagttgagcggttcccggctggccgcgcagcaacagatggaaggcctcctggcgccgcaccggcccaa
ggagcccgcgtggttcctggccaccgtcggcgtctcgcccgaccaccagggcaagggtctgggcagegccgtcgtgctcccc
ggagtggaggcggccgagcgcgccggggtgcccgccttcctggagacctccgcgccccgcaacctccccttctacgagcgg
ctcggcttcaccgtcaccgccgacgtcgaggtgcccgaaggaccgcgcacctggtgcatgacccgcaagcccggtgcctgac
tcgagggaattaattcgagctcggtacctttaagaccaatgacttacaaggcagctgtagatcttagccactttttaaaagaaaag
gggggactggaagggctaattcactcccaacgaagacaagatctgctttttgcttgtactgggtctctctggttagaccagatctga
gcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagcatctagaattaattccgtgt
attctatagtgtcacctaaatcgtatgtgtatgatacataaggttatgtattaattgtagccgcgttctaacgacaatatgtacaagcct
aattgtgtagcatctggcttactgaagcagaccctatcatctctctcgtaaactgccgtcagagtcggtttggttggacgaaccttctg
agtttctggtaacgccgtcccgcacccggaaatggtcagcgaaccaatcagcagggtcatcgctagccagatcctctacgccg
gacgcatcgtggccggcatcaccggcgccacaggtgcggttgctggcgcctatatcgccgacatcaccgatggggaagatcg
ggctcgccacttcgggctcatgagcgcttgtttcggcgtgggtatggtggcaggccccgtggccgggggactgttgggcgccatc
tccttgcatgcaccattccttgcggcggcggtgctcaacggcctcaacctactactgggctgcttcctaatgcaggagtcgcataa
gggagagcgtcgaatggtgcactctcagtacaatctgctctgatgccgcatagttaagccagccccgacacccgccaacaccc
gctgacgcgccctgacgggcttgtctgctcccggcatccgcttacagacaagctgtgaccgtctccgggagctgcatgtgtcaga
ggttttcaccgtcatcaccgaaacgcgcgagacgaaagggcctcgtgatacgcctatttttataggttaatgtcatgataataatgg
tttcttagacgtcaggtggcacttttcggggaaatgtgcgcggaacccctatttgtttatttttctaaatacattcaaatatgtatccgctc
atgagacaataaccctgataaatgcttcaataatattgaaaaaggaagagtatgagtattcaacatttccgtgtcgcccttattccct
tttttgcggcattttgccttcctgtttttgctcacccagaaacgctggtgaaagtaaaagatgctgaagatcagttgggtgcacgagtg
ggttacatcgaactggatctcaacagcggtaagatccttgagagttttcgccccgaagaacgttttccaatgatgagcacttttaaa
gttctgctatgtggcgcggtattatcccgtattgacgccgggcaagagcaactcggtcgccgcatacactattctcagaatgacttg
gttgagtactcaccagtcacagaaaagcatcttacggatggcatgacagtaagagaattatgcagtgctgccataaccatgagt
gataacactgcggccaacttacttctgacaacgatcggaggaccgaaggagctaaccgcttttttgcacaacatgggggatcat
gtaactcgccttgatcgttgggaaccggagctgaatgaagccataccaaacgacgagcgtgacaccacgatgcctgtagcaat
ggcaacaacgttgcgcaaactattaactggcgaactacttactctagcttcccggcaacaattaatagactggatggaggcgga
taaagttgcaggaccacttctgcgctcggcccttccggctggctggtttattgctgataaatctggagccggtgagcgtgggtctcg
cggtatcattgcagcactggggccagatggtaagccctcccgtatcgtagttatctacacgacggggagtcaggcaactatggat
gaacgaaatagacagatcgctgagataggtgcctcactgattaagcattggtaactgtcagaccaagtttactcatatatactttag
attgatttaaaacttcatttttaatttaaaaggatctaggtgaagatcctttttgataatctcatgaccaaaatcccttaacgtgagttttc
gttccactgagcgtcagaccccgtagaaaagatcaaaggatcttcttgagatcctttttttctgcgcgtaatctgctgcttgcaaaca
aaaaaaccaccgctaccagcggtggtttgtttgccggatcaagagctaccaactctttttccgaaggtaactggcttcagcagag
cgcagataccaaatactgttcttctagtgtagccgtagttaggccaccacttcaagaactctgtagcaccgcctacatacctcgctc
tgctaatcctgttaccagtggctgctgccagtggcgataagtcgtgtcttaccgggttggactcaagacgatagttaccggataag
gcgcagcggtcgggctgaacggggggttcgtgcacacagcccagcttggagcgaacgacctacaccgaactgagataccta
cagcgtgagctatgagaaagcgccacgcttcccgaagggagaaaggcggacaggtatccggtaagcggcagggtcggaa
caggagagcgcacgagggagcttccagggggaaacgcctggtatctttatagtcctgtcgggtttcgccacctctgacttgagcg
tcgatttttgtgatgctcgtcaggggggcggagcctatggaaaaacgccagcaacgcggcctttttacggttcctggccttttgctgg
ccttttgctcacatgttctttcctgcgttatcccctgattctgtggataaccgtattaccgcctttgagtgagctgataccgctcgccgca
gccgaacgaccgagcgcagcgagtcagtgagcgaggaagcggaagagcgcccaatacgcaaaccgcctctccccgcgc
gttggccgattcattaatgcagctgtggaatgtgtgtcagttagggtgtggaaagtccccaggctccccagcaggcagaagtatg
caaagcatgcatctcaattagtcagcaaccaggtgtggaaagtccccaggctccccagcaggcagaagtatgcaaagcatgc
atctcaattagtcagcaaccatagtcccgcccctaactccgcccatcccgcccctaactccgcccagttccgcccattctccgccc
catggctgactaattttttttatttatgcagaggccgaggccgcctcggcctctgagctattccagaagtagtgaggaggcttttttgg
aggcctaggcttttgcaaaaagcttggacacaagacaggcttgcgagatatgtttgagaataccactttatcccgcgtcagggag
aggcagtgcgtaaaaagacgcggactcatgtgaaatactggtttttagtgcgccagatctctataatctcgcgcaacctattttccc
ctcgaacactttttaagccgtagataaacaggctgggacacttcacatgagcgaaaaatacatcgtcacctgggacatgttgca
gatccatgcacgtaaactcgcaagccgactgatgccttctgaacaatggaaaggcattattgccgtaagccgtggcggtctgtac
cgggtgcgttactggcgcgtgaactgggtattcgtcatgtcgataccgtttgtatttccagctacgatcacgacaaccagcgcgag
cttaaagtgctgaaacgcgcagaaggcgatggcgaaggcttcatcgttattgatgacctggtggataccggtggtactgcggttg
cgattcgtgaaatgtatccaaaagcgcactttgtcaccatcttcgcaaaaccggctggtcgtccgctggttgatgactatgttgttgat
atcccgcaagatacctggattgaacagccgtgggatatgggcgtcgtattcgtcccgccaatctccggtcgctaatcttttcaacgc
ctggcactgccgggcgttgttctttttaacttcaggcgggttacaatagtttccagtaagtattctggaggctgcatccatgacacag
gcaaacctgagcgaaaccctgttcaaaccccgctttaaacatcctgaaacctcgacgctagtccgccgctttaatcacggcgca
caaccgcctgtgcagtcggcccttgatggtaaaaccatccctcactggtatcgcatgattaaccgtctgatgtggatctggcgcgg
cattgacccacgcgaaatcctcgacgtccaggcacgtattgtgatgagcgatgccgaacgtaccgacgatgatttatacgatac
ggtgattggctaccgtggcggcaactggatttatgagtgggccccggatctttgtgaaggaaccttacttctgtggtgtgacataatt
ggacaaactacctacagagatttaaagctctaaggtaaatataaaatttttaagtgtataatgtgttaaactactgattctaattgtttg
tgtattttagattccaacctatggaactgatgaatgggagcagtggtggaatgcctttaatgaggaaaacctgttttgctcagaaga
aatgccatctagtgatgatgaggctactgctgactctcaacattctactcctccaaaaaagaagagaaaggtagaagacccca
aggactttccttcagaattgctaagttttttgagtcatgctgtgtttagtaatagaactcttgcttgctttgctatttacaccacaaaggaa
aaagctgcactgctatacaagaaaattatggaaaaatattctgtaacctttataagtaggcataacagttataatcataacatactg
ttttttcttactccacacaggcatagagtgtctgctattaataactatgctcaaaaattgtgtacctttagctttttaatttgtaaaggggtt
aataaggaatatttgatgtatagtgccttgactagagatcataatcagccataccacatttgtagaggttttacttgctttaaaaaacc
tcccacacctccccctgaacctgaaacataaaatgaatgcaattgttgttgttaacttgtttattgcagcttataatggttacaaataa
agcaatagcatcacaaatttcacaaataaagcatttttttcactgcattctagttgtggtttgtccaaactcatcaatgtatcttatcatgt
ctggatcaactggataactcaagctaaccaaaatcatcccaaacttcccaccccataccctattaccactgccaattacctgtggt
ttcatttactctaaacctgtgattcctctgaattattttcattttaaagaaattgtatttgttaaatatgtactacaaacttagtagt
SEQ ID NO: 56 - pREF097 - FoxP3-inducible CAR
tggaagggctaattcactcccaaagaagacaagatatccttgatctgtggatctaccacacacaaggctacttccctgattagca
gaactacacaccagggccaggggtcagatatccactgacctttggatggtgctacaagctagtaccagttgagccagataagg
tagaagaggccaataaaggagagaacaccagcttgttacaccctgtgagcctgcatgggatggatgacccggagagagaag
tgttagagtggaggtttgacagccgcctagcatttcatcacgtggcccgagagctgcatccggagtacttcaagaactgctgatat
cgagcttgctacaagggactttccgctggggactttccagggaggcgtggcctggggggactggggagtggcgagccctcag
atcctgcatataagcagctgctttttgcctgtactgggtctctctggttagaccagatctgagcctgggagctctctggctaactaggg
aacccactgcttaagcctcaataaagcttgccttgagtgcttcaagtagtgtgtgcccgtctgttgtgtgactctggtaactagagatc
cctcagacccttttagtcagtgtggaaaatctctagcagtggcgcccgaacagggacttgaaagcgaaagggaaaccagagg
agctctctcgacgcaggactcggcttgctgaagcgcgcacggcaagaggcgaggggcggcgactggtgagtacgccaaaa
attttgactagcggaggctagaaggagagagatgggtgcgagagcgtcagtattaagcgggggagaattagatcgcgatggg
aaaaaattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcagacaggat
cagaagaacttagatcattatataatacagtagcaaccctctattgtgtgcatcaaaggatagagataaaagacaccaaggaa
gctttagacaagatagaggaagagcaaaacaaaagtaagaccaccgcacagcaagcggccggccgctgatcttcagacct
ggaggaggagatatgagggacaattggagaagtgaattatataaatataaagtagtaaaaattgaaccattaggagtagcacc
caccaaggcaaagagaagagtggtgcagagagaaaaaagagcagtgggaataggagctttgttccttgggttcttgggagca
gcaggaagcactatgggcgcagcgtcaatgacgctgacggtacaggccagacaattattgtctggtatagtgcagcagcaga
acaatttgctgagggctattgaggcgcaacagcatctgttgcaactcacagtctggggcatcaagcagctccaggcaagaatcc
tggctgtggaaagatacctaaaggatcaacagctcctggggatttggggttgctctggaaaactcatttgcaccactgctgtgcctt
ggaatgctagttggagtaataaatctctggaacagatttggaatcacacgacctggatggagtgggacagagaaattaacaatt
acacaagcttaatacactccttaattgaagaatcgcaaaaccagcaagaaaagaatgaacaagaattattggaattagataaa
tgggcaagtttgtggaattggtttaacataacaaattggctgtggtatataaaattattcataatgatagtaggaggcttggtaggttta
agaatagtttttgctgtactttctatagtgaatagagttaggcagggatattcaccattatcgtttcagacccacctcccaaccccgag
gggacccgacaggcccgaaggaatagaagaagaaggtggagagagagacagagacagatccattcgattagtgaacgg
atctcgacggtatcgccaaatggcagtattcatccacaattttaaaagaaaaggggggattggggggtacagtgcaggggaaa
gaatagtagacataatagcaacagacatacaaactaaagaattacaaaaacaaattacaaaaattcaaaattttcgggtttatta
cagggacagcagagatccagtttggatcgataagcttgatatcgaattcgtagctgaagaaaggattcataaatgaagttcaatc
cttctcatcaaccccagcccacacctccagcaattgaacttgaaaaaaaaaacctggtttgaaaaattaccgcaaactatattgt
catcaaaaaaaaaaaaaaaaaaaaacacttcctatatttgagatgagagaagagagtgctaggcagtttcctggctgaacac
gccagcccaatacttaaagagagcaactcctgactccgatagagactggatggacccacaagggtgacagcccaggcgga
ccgatcttcccatcccacatcctccggcgcgatgccaaaaagaggctgacggcaactgggccttctgcagagaaagacctcc
gcttcactgccccggctggtcccaagggtcaggaagGCCACCATGGGAACTAGTCTGCTGTGTTGGATG
GCCCTCTGCTTGTTGGGCGCAGACCACGCTGACGGCCAAGTTCAATTGGTCCAGTCTG
GTGCCGAACTGAAGAAGCCAGGAGCTAGCGTCAAGGTGAGTTGTAAGTCTTCCGGATA
CCATTTCACGAGCTACTGGATGCATTGGGTAAAACAAGCCCCTGGACAAGGTCTCGAG
TGGATAGGGGTAATACATCCTAACTCTGGGTCCATAAATTACAATGAAAAGTTCGAATC
CAGGGTTACCATCACAGTCGATAAGTCCACTTCCACGGCCTATATGGAATTGAGCTCC
CTCAGGAGCGAAGATACAGCTGTATACTACTGCGCTGGTGAACGAGACTCCACTGAGG
TGTTGCCCATGGATTACTGGGGACAAGGCACCACTGTAACGGTCTCTAGCGGTGGGA
GTAGTAGAAGCTCATCATCTGGCGGCGGCGGTTCAGGCGGTGGCGGCGACGTGCAGA
TCACGCAAAGCCCGAGCTCACTCTCCGCCAGTCTGGGTGAACGTGCGACTATAAATTG
TAGAGCCAGCAAGAGCATTAACAAGTATCTCGCCTGGTACCAGCAAAAGCCGGGCAAG
GCTCCTAAACTCCTCATCTATTCCGGATCTACTCTCCAAAGCGGCATACCAGCGAGGTT
CTCAGGAAGCGGATCAGGAACGGATTTTACGCTTACGATATCTAGCCTCGAGCCAGAG
GATTTTGCCATGTACTATTGCCAACAACATAACGAATATCCCTTGACCTTCGGACAAGG
GACGAAACTCGAGATCAAAGATTACAAAGACGATGACGACAAAACCACAACCCCAGCT
CCTAGGCCCCCAACACCTGCACCTACCATCGCTAGCCAGCCTCTGAGCCTGCGTCCC
GAGGCATGTCGTCCTGCTGCAGGTGGGGCAGTCCACACAAGAGGTCTGGACTTTGCA
TGTGACTTTTGGGTCCTCGTAGTCGTCGGTGGCGTCCTGGCCTGTTATTCCCTTCTGG
TTACGGTCGCCTTCATTATATTTTGGGTCAGGAGTAAACGGTCTAGGGGGGGGCACAG
CGACTACATGAATATGACGCCACGCCGCCCCGGGCCCACAAGAAAACACTACCAACCT
TACGCTCCACCCAGGGATTTTGCCGCGTACCGGAGCAGGGTAAAGTTCAGCCGCAGT
GCGGATGCACCAGCGTATCAACAAGGCCAGAATCAACTGTATAATGAGCTGAACTTGG
GACGACGAGAGGAATATGATGTTCTGGATAAACGGCGCGGGAGAGATCCAGAGATGG
GCGGAAAGCCTAGACGTAAGAACCCCCAAGAAGGCCTTTATAATGAGTTGCAGAAAGA
TAAGATGGCCGAAGCCTATTCTGAAATAGGCATGAAGGGAGAGCGGCGGCGTGGCAA
GGGTCATGACGGTCTTTACCAGGGCCTCTCTACCGCAACTAAAGATACCTATGATGCA
CTTCACATGCAAGCCCTGCCCCCTAGAGAGTTCGAAGGGTCAGCAGCAGCCTGAggccg
cgactctagagtcgacctgcaggcatgcaagcttgatatcaagcttatcgataatcaacctctggattacaaaatttgtgaaagatt
gactggtattcttaactatgttgctccttttacgctatgtggatacgctgctttaatgcctttgtatcatgctattgcttccccgtatggctttc
attttctcctccttgtataaatcctggttgctgtctctttatgaggagttgtggcccgttgtcaggcaacgtggcgtggtgtgcactgtgttt
gctgacgcaacccccactggttggggcattgccaccacctgtcagctcctttccgggactttcgctttccccctccctattgccacgg
cggaactcatcgccgcctgccttgcccgctgctggacaggggctcggctgttgggcactgacaattccgtggtgttgtcggggaa
atcatcgtcctttccttggctgctcgcctgtgttgccacctggattctgcgcgggacgtccttctgctacgtcccttcggccctcaatcc
agcggaccttccttcccaattctaccgggtaggggaggcgcttttcccaaggcagtctggagcatgcgctttagcagccccgctg
ggcacttggcgctacacaagtggcctctggcctcgcacacattccacatccaccggtaggcgccaaccggctccgttctttggtg
gccccttcgcgccaccttctactcctcccctagtcaggaagttcccccccgccccgcagctcgcgtcgtgcaggacgtgacaaat
ggaagtagcacgtctcactagtctcgtgcagatggacagcaccgctgagcaatggaagcgggtaggcctttggggcagcggc
caatagcagctttgctccttcgctttctgggctcagaggctgggaaggggtgggtccggggggggctcaggggcgggctcag
gggcggggcgggcgcccgaaggtcctccggaggcccggcattctgcacgcttcaaaagcgcacgtctgccgcgctgttctcct
cttcctcatctccgggcctttcgacctgcagcccaagcttaccatgaccgagtacaagcccacggtgcgcctcgccacccgcga
cgacgtccccagggccgtacgcaccctcgccgccgcgttcgccgactaccccgccacgcgccacaccgtcgatccggaccg
ccacatcgagcgggtcaccgagctgcaagaactcttcctcacgcgcgtcgggctcgacatcggcaaggtgtgggtcgcggac
gacggcgccgcggtggcggtctggaccacgccggagagcgtcgaagcgggggcggtgttcgccgagatcggcccgcgcat
ggccgagttgagcggttcccggctggccgcgcagcaacagatggaaggcctcctggcgccgcaccggcccaaggagcccg
cgtggttcctggccaccgtcggcgtctcgcccgaccaccagggcaagggtctgggcagcgccgtcgtgctccccggagtgga
ggcggccgagcgcgccggggtgcccgccttcctggagacctccgcgccccgcaacctccccttctacgagcggctcggcttca
ccgtcaccgccgacgtcgaggtgcccgaaggaccgcgcacctggtgcatgacccgcaagcccggtgcctgactcgaggga
attaattcgagctcggtacctttaagaccaatgacttacaaggcagctgtagatcttagccactttttaaaagaaaaggggggact
ggaagggctaattcactcccaacgaagacaagatctgctttttgcttgtactgggtctctctggttagaccagatctgagcctggga
gctctctggctaactagggaacccactgcttaagcctcaataaagcttgccttgagtgcttcaagtagtgtgtgcccgtctgttgtgtg
actctggtaactagagatccctcagacccttttagtcagtgtggaaaatctctagcagcatctagaattaattccgtgtattctatagt
gtcacctaaatcgtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaaccttctgagtttctgg
taacgccgtcccgcacccggaaatggtcagcgaaccaatcagcagggtcatcgctagccagatcctctacgccggacgcatc
gtggccggcatcaccggcgccacaggtgcggttgctggcgcctatatcgccgacatcaccgatggggaagategggctcgcc
acttcgggctcatgagcgcttgtttcggcgtgggtatggtggcaggccccgtggccgggggactgttgggcgccatctccttgcat
gcaccattccttgcggcggcggtgctcaacggcctcaacctactactgggctgcttcctaatgcaggagtcgcataagggagag
cgtcgaatggtgcactctcagtacaatctgctctgatgccgcatagttaagccagccccgacacccgccaacacccgctgacgc
gccctgacgggcttgtctgctcccggcatccgcttacagacaagctgtgaccgtctccgggagctgcatgtgtcagaggttttcac
cgtcatcaccgaaacgcgcgagacgaaagggcctcgtgatacgcctatttttataggttaatgtcatgataataatggtttcttaga
cgtcaggtggcacttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttattcccttttttgcgg
cattttgccttcctgtttttgctcacccagaaacgctggtgaaagtaaaagatgctgaagatcagttgggtgcacgagtgggttacat
cgaactggatctcaacagcggtaagatccttgagagttttcgccccgaagaacgttttccaatgatgagcacttttaaagttctgct
atgtggcgcggtattatcccgtattgacgccgggcaagagcaactcggtcgccgcatacactattctcagaatgacttggttgagt
actcaccagtcacagaaaagcatcttacggatggcatgacagtaagagaattatgcagtgctgccataaccatgagtgataac
actgcggccaacttacttctgacaacgatcggaggaccgaaggagctaaccgcttttttgcacaacatgggggatcatgtaactc
gccttgatcgttgggaaccggagctgaatgaagccataccaaacgacgagcgtgacaccacgatgcctgtagcaatggcaac
aacgttgcgcaaactattaactggcgaactacttactctagcttcccggcaacaattaatagactggatggaggcggataaagtt
gcaggaccacttctgcgctcggcccttccggctggctggtttattgctgataaatctggagccggtgagcgtgggtctcgcggtatc
attgcagcactggggccagatggtaagccctcccgtatcgtagttatctacacgacggggagtcaggcaactatggatgaacg
aaatagacagatcgctgagataggtgcctcactgattaagcattggtaactgtcagaccaagtttactcatatatactttagattgat
ttaaaacttcatttttaatttaaaaggatctaggtgaagatcctttttgataatctcatgaccaaaatcccttaacgtgagttttcgttcca
ctgagcgtcagaccccgtagaaaagatcaaaggatcttcttgagatcctttttttctgcgcgtaatctgctgcttgcaaacaaaaaa
accaccgctaccagcggtggtttgtttgccggatcaagagctaccaactctttttccgaaggtaactggcttcagcagagcgcag
ataccaaatactgttcttctagtgtagccgtagttaggccaccacttcaagaactctgtagcaccgcctacatacctcgctctgctaa
tcctgttaccagtggctgctgccagtggcgataagtcgtgtcttaccgggttggactcaagacgatagttaccggataaggcgca
gcggtcgggctgaacggggggttcgtgcacacagcccagcttggagcgaacgacctacaccgaactgagatacctacagcg
tgagctatgagaaagcgccacgcttcccgaagggagaaaggcggacaggtatccggtaagcggcagggtcggaacagga
gagcgcacgagggagcttccagggggaaacgcctggtatctttatagtcctgtcgggtttcgccacctctgacttgagcgtcgattt
ttgtgatgctcgtcaggggggcggagcctatggaaaaacgccagcaacgcggcctttttacggttcctggccttttgctggccttttg
ctcacatgttctttcctgcgttatcccctgattctgtggataaccgtattaccgcctttgagtgagctgataccgctcgccgcagccga
acgaccgagcgcagcgagtcagtgagcgaggaagcggaagagcgcccaatacgcaaaccgcctctccccgcgcgttggc
cgattcattaatgcagctgtggaatgtgtgtcagttagggtgtggaaagtccccaggctccccagcaggcagaagtatgcaaag
catgcatctcaattagtcagcaaccaggtgtggaaagtccccaggctccccagcaggcagaagtatgcaaagcatgcatctca
attagtcagcaaccatagtcccgcccctaactccgcccatcccgcccctaactccgcccagttccgcccattctccgccccatgg
ctgactaattttttttatttatgcagaggccgaggccgcctcggcctctgagctattccagaagtagtgaggaggcttttttggaggcc
taggcttttgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctattttcccctcgaa
cactttttaagccgtagataaacaggctgggacacttcacatgagcgaaaaatacatcgtcacctgggacatgttgcagatccat
gcacgtaaactcgcaagccgactgatgccttctgaacaatggaaaggcattattgccgtaagccgtggcggtctgtaccgggtg
cgttactggcgcgtgaactgggtattcgtcatgtcgataccgtttgtatttccagctacgatcacgacaaccagcgcgagcttaaag
tgctgaaacgcgcagaaggcgatggcgaaggcttcatcgttattgatgacctggtggataccggtggtactgcggttgcgattcgt
gaaatgtatccaaaagcgcactttgtcaccatcttcgcaaaaccggctggtcgtccgctggttgatgactatgttgttgatatcccgc
aagatacctggattgaacagccgtgggatatgggcgtcgtattcgtcccgccaatctccggtcgctaatcttttcaacgcctggca
ctgccgggcgttgttctttttaacttcaggcgggttacaatagtttccagtaagtattctggaggctgcatccatgacacaggcaaac
ctgagcgaaaccctgttcaaaccccgctttaaacatcctgaaacctcgacgctagtccgccgctttaatcacggcgcacaaccg
cctgtgcagtcggcccttgatggtaaaaccatccctcactggtatcgcatgattaaccgtctgatgtggatctggcgcggcattgac
ccacgcgaaatcctcgacgtccaggcacgtattgtgatgagcgatgccgaacgtaccgacgatgatttatacgatacggtgattg
gctaccgtggcggcaactggatttatgagtgggccccggatctttgtgaaggaaccttacttctgtggtgtgacataattggacaaa
ctacctacagagatttaaagctctaaggtaaatataaaatttttaagtgtataatgtgttaaactactgattctaattgtttgtgtattttag
attccaacctatggaactgatgaatgggagcagtggtggaatgcctttaatgaggaaaacctgttttgctcagaagaaatgccat
ctagtgatgatgaggctactgctgactctcaacattctactcctccaaaaaagaagagaaaggtagaagaccccaaggactttc
cttcagaattgctaagttttttgagtcatgctgtgtttagtaatagaactcttgcttgctttgctatttacaccacaaaggaaaaagctgc
actgctatacaagaaaattatggaaaaatattctgtaacctttataagtaggcataacagttataatcataacatactgttttttcttact
ccacacaggcatagagtgtctgctattaataactatgctcaaaaattgtgtacctttagctttttaatttgtaaaggggttaataagga
atatttgatgtatagtgccttgactagagatcataatcagccataccacatttgtagaggttttacttgctttaaaaaacctcccacac
ctccccctgaacctgaaacataaaatgaatgcaattgttgttgttaacttgtttattgcagcttataatggttacaaataaagcaatag
catcacaaatttcacaaataaagcatttttttcactgcattctagttgtggtttgtccaaactcatcaatgtatcttatcatgtctggatca
actggataactcaagctaaccaaaatcatcccaaacttcccaccccataccctattaccactgccaattacctgtggtttcatttact
ctaaacctgtgattcctctgaattattttcattttaaagaaattgtatttgttaaatatgtactacaaacttagtagt
SEQ ID NO: 57 - pREF014 - CAR-encoding plasmid, can express constitutively CAR
under SFFV promoter
CTCGAGCTTATTCCAGATGCGTGCGGATGGAATTCGAGCTCGGTACCATGCCAAAAGC
AAAGCGCTATCGCGCCTTACGTTACTGGCCGAAGCCGCTTGGAATAAGGCCGGTGTG
CGTTTGTCTATATGTTATTTTCCACCATATTGCCGTCTTTTGGCAATGTGAGGGCCCGG
AAACCTGGCCCTGTCTTCTTGACGAGCATTCCTAGGGGTCTTTCCCCTCTCGCCAAAG
GAATGCAAGGTCTGTTGAATGTCGTGAAGGAAGCAGTTCCTCTGGAAGCTTCTTGAAG
ACAAACAACGTCTGTAGCGACCCTTTGCAGGCAGCGGAACCCCCCACCTGGCGACAG
GTGCCTCTGCGGCCAAAAGCCACGTGTATAAGATACACCTGCAAAGGCGGCACAACCC
CAGTGCCACGTTGTGAGTTGGATAGTTGTGGAAAGAGTCAAATGGCTCCCCTCAAGCG
TATTCAACAAGGGGCTGAAGGATGCCCAGAAGGTACCCCATTGTATGGGATCTGATCT
GGGGCCTCGGTGCACATGCTTTTCATGTGTTTAGTCGAGGTTAAAAAACGTCTAGGCC
CCCCGAACCACGGGGACGTGGTTTTCCTTTGAAAAACACGATGATAATAACATGACCG
AGTACAAGCCCACGGTGCGCCTCGCCACCCGCGACGACGTCCCCAGGGCCGTACGC
ACCCTCGCCGCCGCGTTCGCCGACTACCCCGCCACGCGCCACACCGTCGATCCGGAC
CGCCACATCGAGCGGGTCACCGAGCTGCAAGAACTCTTCCTCACGCGCGTCGGGCTC
GACATCGGCAAGGTGTGGGTCGCGGACGACGGCGCCGCGGTGGCGGTCTGGACCAC
GCCGGAGAGCGTCGAAGCGGGGGCGGTGTTCGCCGAGATCGGCCCGCGCATGGCCG
AGTTGAGCGGTTCCCGGCTGGCCGCGCAGCAACAGATGGAGGGCCTCCTGGCGCCG
CACCGGCCCAAGGAGCCCGCGTGGTTCCTGGCCACCGTCGGCGTCTCGCCCGACCA
CCAGGGCAAGGGTCTGGGCAGCGCCGTCGTGCTCCCCGGAGTGGAGGCCGCCGAGC
GCGCCGGGGTGCCCGCCTTCCTGGAGACCTCCGCGCCCCGCAACCTCCCCTTCTACG
AGCGGCTCGGCTTCACCGTCACCGCCGACGTCGAGGTGCCCGAAGGACCGCGCACC
TGGTGCATGACCCGCAAGCCCGGTGCCTAGGCTAGCTTGACTGACTGAGTCGACAATC
AACCTTTTGGATTACAAAATTTGTGAAAGATTGACTGGTATTCTTAACTATGTTGCTCCT
TTTACGCTATGTGGATACGCTGCTTTAATGCCTTTGTATCATGCTATTGCTTCCCGTATG
GCTTTCATTTTCTCCTCCTTGTATAAATCCTGGTTGCTGTCTCTTTATGAGGAGTTGTGG
CCCGTTGTCAGGCAACGTGGCGTGGTGTGCACTGTGTTTGCTGACGCAACCCCCACT
GGTTGGGGCATTGCCACCACCTGTCAGCTCCTTTCCGGGACTTTCGCTTTCCCCCTCC
CTATTGCCACGGCGGAACTCATCGCCGCCTGCCTTGCCCGCTGCTGGACAGGGGCTC
GGCTGTTGGGCACTGACAATTCCGTGGTGTTGTCGGGGAAGCTGACGTCCTTTCCATG
GCTGCTCGCCTGTGTTGCCACCTGGATTCTGCGCGGGACGTCCTTCTGCTACGTCCCT
TCGGCCCTCAATCCAGCGGACCTTCCTTCCCGCGGCCTGCTGCCGGCTCTGCGGCCT
CTTCCGCGTCTTCGCCTTCGCCCTCAGACGAGTCGGATCTCCCTTTGGGCCGCCTCCC
CGCCTGGAATTCGAGCTCGGTACCTTTAAGACCAATGACTTACAAGGCAGCTGTAGAT
CTTAGCCACTTTTTAAAAGAAAAGGGGGGACTGGAAGGGCTAATTCACTCCCAACGAA
GACAAGATCTGCTTTTTGCTTGTACTGGGTCTCTCTGGTTAGACCAGATCTGAGCCTGG
GAGCTCTCTGGCTAACTAGGGAACCCACTGCTTAAGCCTCAATAAAGCTTGCCTTGAGT
GCTTCAAGTAGTGTGTGCCCGTCTGTTGTGTGACTCTGGTAACTAGAGATCCCTCAGA
CCCTTTTAGTCAGTGTGGAAAATCTCTAGCAGTCCTGGCCAACGTGAGCACCGTGCTG
ACCTCCAAATATCGTTAAGCTGGAGCCTGGGAGCCGGCCTGGCCCTCCGCCCCCCCC
ACCCCCGCAGCCCACCCCTGGTCTTTGAATAAAGTCTGAGTGAGTGGCCGACAGTGCC
CGTGGAGTTCTCGTGACCTGAGGTGCAGGGCCGGCGCTAGGGACACGTCCGTGCAC
GTGCCGAGGCCCCCTGTGCAGCTGCAAGGGACAGGCCTAGCCCTGCAGGCCTAACTC
CGCCCATCCCGCCCCTAACTCCGCCCAGTTCCGCCCATTCTCCGCCTCATGGCTGACT
AATTTTTTTTATTTATGCAGAGGCCGAGGCCGCCTCGGCCTCTGAGCTATTCCAGAAGT
AGTGAGGACGCTTTTTTGGAGGCCGAGGCTTTTGCAAAGATCGAACAAGAGACAGGAC
CTGCAGGTTAATTAAATTTAAATCATGTGAGCAAAAGGCCAGCAAAAGGCCAGGAACC
GTAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGA
AGAACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGG
TCTGACGCTCAGTGGAACGAAAACTCACGTTAAGGGATTTTGGTCATGAGATTATCAAA
AAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGC
GATCTGTCTATTTCGTTCATCCATAGTTGCATTTAAATGGCCGGCCTGGCGCGCCGTTT
AAACCTAGATATTGATAGTCTGATCGGTCAACGTATAATCGAGTCCTAGCTTTTGCAAA
CATCTATCAAGAGACAGGATCAGCAGGAGGCTTTCGCATGAGTATTCAACATTTCCGTG
TCGCCCTTATTCCCTTTTTTGCGGCATTTTGCCTTCCTGTTTTTGCTCACCCAGAAACGC
TGGTGAAAGTAAAAGATGCTGAAGATCAGTTGGGTGCGCGAGTGGGTTACATCGAACT
GGATCTCAACAGCGGTAAGATCCTTGAGAGTTTTCGCCCCGAAGAACGCTTTCCAATG
ATGAGCACTTTTAAAGTTCTGCTATGTGGCGCGGTATTATCCCGTATTGACGCCGGGCA
AGAGCAACTCGGTCGCCGCATACACTATTCTCAGAATGACTTGGTTGAGTATTCACCAG
TCACAGAAAAGCATCTTACGGATGGCATGACAGTAAGAGAATTATGCAGTGCTGCCATA
ACCATGAGTGATAACACTGCGGCCAACTTACTTCTGACAACGATTGGAGGACCGAAGG
AGCTAACCGCTTTTTTGCACAACATGGGGGATCATGTAACTCGCCTTGATCGTTGGGAA
CCGGAGCTGAATGAAGCCATACCAAACGACGAGCGTGACACCACGATGCCTGTAGCA
ATGGCAACAACCTTGCGTAAACTATTAACTGGCGAACTACTTACTCTAGCTTCCCGGCA
ACAGTTGATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTCGCG
GTATCATTGCAGCACTGGGGCCAGATGGTAAGCCCTCCCGTATCGTAGTTATCTACAC
GACGGGGAGTCAGGCAACTATGGATGAACGAAATAGACAGATCGCTGAGATAGGTGC
CTCACTGATTAAGCATTGGTAACCGATTCTAGGTGCATTGGCGCAGAAAAAAATGCCTG
ATGCGACGCTGCGCGTCTTATACTCCCACATATGCCAGATTCAGCAACGGATACGGCT
TCCCCAACTTGCCCACTTCCATACGTGTCCTCCTTACCAGAAATTTATCCTTAAGATCCC
GAATCGTTTAAACGCGATCGCAGTAATCAATTACGGGGTCATTAGTTCATAGCCCATAT
ATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTACATCA
AGTGTATCATATGCCAAGTACGCCCCCTATTGACGTCAATGACGGTAAATGGCCCGCC
TGGCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGGAT
AGCGGTTTGACTCACGGGGATTTCCAAGTCTCCACCCCATTGACGTCAATGGGAGTTT
GTTTTGGCACCAAAATCAACGGGACTTTCCAAAATGTCGTAACAACTCCGCCCCATTGA
CGCAAATGGGGGGTAGGCGTGTACGGTGGGAGGTCTATATAAGCAGAGCTCGTTTAGT
GAACCGGGGTCTCTCTGGTTAGACCAGATCTGAGCCTGGGAGCTCTCTGGCTAACTAG
GGAACCCACTGCTTAAGCCTCAATAAAGCTTGCCTTGAGTGCTTCAAGTAGTGTGTGCC
CGTCTGTTGTGTGACTCTGGTAACTAGAGATCCCTCAGACCCTTTTAGTCAGTGTGGAA
AATCTCTAGCAGTGGCGCCCGAACAGGGACCTGAAAGCGAAAGGGAAACCAGAGCTC
TCTCGACGCAGGACTCGGCTTGCTGAAGCGCGCACGGCAAGAGGCGAGGGGCGGCG
ACTGGTGAGTACGCCAAAAATTTTGACTAGCGGAGGCTAGAAGGAGAGAGATGGGTGC
GAGAGCGTCAGTATTAAGCGGGGGAGAATTAGATCGCGATGGGAAAAAATTCGGTTAA
GGCCAGGGGGAAAGAAAAAATATAAATTAAAACATATAGTATGGGCAAGCAGGGAGCT
AGAACGATTCGCAGTTAATCCTGGCCTGTTAGAAACATCAGAAGGCTGTAGACAAATAC
TGGGACAGCTACAACCATCCCTTCAGACAGGATCAGAAGAACTTAGATCATTATATAAT
ACAGTAGCAACCCTCTATTGTGTGCATCAAAGGATAGAGATAAAAGACACCAAGGAAG
CTTTAGACAAGATAGAGGAAGAGCAAAACAAAAGTAAGACCACCGCACAGCAAGCGGC
CGCTGATCTTCAGACCTGGAGGAGGAGATATGAGGGACAATTGGAGAAGTGAATTATA
TAAATATAAAGTAGTAAAAATTGAACCATTAGGAGTAGCACCCACCAAGGCAAAGAGAA
GAGTGGTGCAGAGAGAAAAAAGAGCAGTGGGAATAGGAGCTTTGTTCCTTGGGTTCTT
GGGAGCAGCAGGAAGCACTATGGGCGCAGCCTCAATGACGCTGACGGTACAGGCCAG
ACAATTATTGTCTGGTATAGTGCAGCAGCAGAACAATTTGCTGAGGGCTATTGAGGCG
CAACAGCATCTGTTGCAACTCACAGTCTGGGGCATCAAGCAGCTCCAGGCAAGAATCC
TGGCTGTGGAAAGATACCTAAAGGATCAACAGCTCCTGGGGATTTGGGGTTGCTCTGG
AAAACTCATTTGCACCACTGCTGTGCCTTGGAATGCTAGTTGGAGTAATAAATCTCTGG
AACAGATTTGGAATCACACGACCTGGATGGAGTGGGACAGAGAAATTAACAATTACACA
AGCTTAATACACTCCTTAATTGAAGAATCGCAAAACCAGCAAGAAAAGAATGAACAAGA
ATTATTGGAATTAGATAAATGGGCAAGTTTGTGGAATTGGTTTAACATAACAAATTGGCT
GTGGTATATAAAATTATTCATAATGATAGTAGGAGGCTTGGTAGGTTTAAGAATAGTTTT
TGCTGTACTTTCTATAGTGAATAGAGTTAGGCAGGGATATTCACCATTATCGTTTCAGAC
CCACCTCCCAACCCCGAGGGGACCCGACAGGCCCGAAGGAATAGAAGAAGAAGGTGG
AGAGAGAGACAGAGACAGATCCATTCGATTAGTGAACGGATCTCGACGGTATCGGTTA
ACTTTTAAAAGAAAAGGGGGGATTGGGGGGTACAGTGCAGGGGAAAGAATAGTAGACA
TAATAGCAACAGACATACAAACTAAAGAATTACAAAAACAAATTACAAAAATTCAAAATTT
TGGCTCCCGATCGTTGCGTTACACACACAATTACTGCTGATCGAGTGTAGCCTTCGAAT
GAAAGACCCCACCTGTAGGTTTGGCAAGATAGCTGCAGTAACGCCATTTTGCAAGGCA
TGGAAAAATACCAAACCAAGAATAGAGAAGTTCAGATCAAGGGCGGGTACATGAAAAT
AGCTAACGTTGGGCCAAACAGGATATCTGCGGTGAGCAGTTTCGGCCCCGGCCCGGG
GCCAAGAACAGATGGTCACCGCAGTTTCGGCCCCGGCCCGAGGCCAAGAACAGATGG
TCCCCAGATATGGCCCAACCCTCAGCAGTTTCTTAAGACCCATCAGATGTTTCCAGGCT
CCCCCAAGGACCTGAAATGACCCTGCGCCTTATTTGAATTAACCAATCAGCCTGCTTCT
CGCTTCTGTTCGCGCGCTTCTGCTTCCCGAGCTCTATAAAAGAGCTCACAACCCCTCA
CTCGGCGCGCCAGTCCTCCGATTGACTGAGTCGCCCTGATCATTGTCGATCCTACCAT
CCACTCGACACACCCGCCAGGGCCCGCATCCACCATCGCAGACTTATCATGGATCCG
CCACCATGGGAACTAGTCTGCTGTGTTGGATGGCCCTCTGCTTGTTGGGCGCAGACCA
CGCTGACGGCCAAGTTCAATTGGTCCAGTCTGGTGCCGAACTGAAGAAGCCAGGAGC
TAGCGTCAAGGTGAGTTGTAAGTCTTCCGGATACCATTTCACGAGCTACTGGATGCATT
GGGTAAAACAAGCCCCTGGACAAGGTCTCGAGTGGATAGGGGTAATACATCCTAACTC
TGGGTCCATAAATTACAATGAAAAGTTCGAATCCAGGGTTACCATCACAGTCGATAAGT
CCACTTCCACGGCCTATATGGAATTGAGCTCCCTCAGGAGCGAAGATACAGCTGTATA
CTACTGCGCTGGTGAACGAGACTCCACTGAGGTGTTGCCCATGGATTACTGGGGACAA
GGCACCACTGTAACGGTCTCTAGCGGTGGGAGTAGTAGAAGCTCATCATCTGGCGGC
GGCGGTTCAGGCGGTGGCGGCGACGTGCAGATCACGCAAAGCCCGAGCTCACTCTCC
GCCAGTCTGGGTGAACGTGCGACTATAAATTGTAGAGCCAGCAAGAGCATTAACAAGT
ATCTCGCCTGGTACCAGCAAAAGCCGGGCAAGGCTCCTAAACTCCTCATCTATTCCGG
ATCTACTCTCCAAAGCGGCATACCAGCGAGGTTCTCAGGAAGCGGATCAGGAACGGAT
TTTACGCTTACGATATCTAGCCTCGAGCCAGAGGATTTTGCCATGTACTATTGCCAACA
ACATAACGAATATCCCTTGACCTTCGGACAAGGGACGAAACTCGAGATCAAAACCACAA
CCCCAGCTCCTAGGCCCCCAACACCTGCACCTACCATCGCTAGCCAGCCTCTGAGCCT
GCGTCCCGAGGCATGTCGTCCTGCTGCAGGTGGGGCAGTCCACACAAGAGGTCTGGA
CTTTGCATGTGACTTTTGGGTCCTCGTAGTCGTCGGTGGCGTCCTGGCCTGTTATTCC
CTTCTGGTTACGGTCGCCTTCATTATATTTTGGGTCAGGAGTAAACGGTCTAGGGGCG
GGCACAGCGACTACATGAATATGACGCCACGCCGCCCCGGGCCCACAAGAAAACACT
ACCAACCTTACGCTCCACCCAGGGATTTTGCCGCGTACCGGAGCAGGGTAAAGTTCAG
CCGCAGTGCGGATGCACCAGCGTATCAACAAGGCCAGAATCAACTGTATAATGAGCTG
AACTTGGGACGACGAGAGGAATATGATGTTCTGGATAAACGGCGCGGGAGAGATCCA
GAGATGGGCGGAAAGCCTAGACGTAAGAACCCCCAAGAAGGCCTTTATAATGAGTTGC
AGAAAGATAAGATGGCCGAAGCCTATTCTGAAATAGGCATGAAGGGAGAGCGGCGGC
GTGGCAAGGGTCATGACGGTCTTTACCAGGGCCTCTCTACCGCAACTAAAGATACCTA
TGATGCACTTCACATGCAAGCCCTGCCCCCTAGAGAGTTCGAAGGGTCAGCAGCAGCC
GAGGGACGTGGCTCATTGCTTACTTGCGGTGACGTTGAAGAAAACCCTGGCCCCAGC
GGGATGGTAAGCAAGGGAGAAGAACTTTTCACGGGAGTAGTTCCTATTCTGGTGGAAC
TGGACGGGGATGTAAACGGGCATAAATTCTCTGTCAGCGGGGAAGGGGAAGGCGATG
CAACATACGGGAAGCTTACCCTCAAATTCATCTGTACGACAGGGAAACTTCCAGTGCC
CTGGCCTACACTTGTGACAACCCTGACTTATGGGGTCCAATGTTTCTCCAGGTACCCTG
ATCACATGAAACAGCATGACTTCTTCAAGAGCGCAATGCCTGAAGGGTATGTCCAGGA
ACGCACGATTTTCTTCAAGGACGATGGCAATTATAAAACTAGAGCCGAAGTAAAATTTG
AGGGAGATACATTGGTAAACAGGATCGAGCTCAAAGGGATCGATTTCAAAGAGGATGG
GAACATCCTGGGGCATAAGTTGGAGTATAACTATAACTCTCACAATGTCTATATTATGG
CCGACAAGCAAAAGAACGGGATAAAAGTCAACTTTAAAATAAGGCACAATATAGAGGAC
GGCAGTGTGCAATTGGCGGACCACTATCAACAAAATACCCCGATCGGCGACGGTCCA
GTGTTGCTCCCTGATAATCACTACCTTTCAACACAAAGTGCACTTAGTAAAGATCCCAAT
GAGAAGCGGGATCACATGGTCCTGCTCGAATTTGTCACAGCAGCTGGCATCACACTTG
GGATGGATGAGCTCTATAAATAA
SEQ ID NO: 58 - pREF091 DNA sequence of Tether-containing plasmid
tggaagggctaattcactcccaaagaagacaagatatccttgatctgtggatctaccacacacaaggctacttccctgattagca
gaactacacaccagggccaggggtcagatatccactgacctttggatggtgctacaagctagtaccagttgagccagataagg
tagaagaggccaataaaggagagaacaccagcttgttacaccctgtgagcctgcatgggatggatgacccggagagagaag
tgttagagtggaggtttgacagccgcctagcatttcatcacgtggcccgagagctgcatccggagtacttcaagaactgctgatat
cgagcttgctacaagggactttccgctggggactttccagggaggcgtggcctggggggactggggagtggcgagccctcag
atcctgcatataagcagctgctttttgcctgtactgggtctctctggttagaccagatctgagcctgggagctctctggctaactaggg
aacccactgcttaagcctcaataaagcttgccttgagtgcttcaagtagtgtgtgcccgtctgttgtgtgactctggtaactagagatc
cctcagacccttttagtcagtgtggaaaatctctagcagtggcgcccgaacagggacttgaaagcgaaagggaaaccagagg
agctctctcgacgcaggactcggcttgctgaagcgcgcacggcaagaggcgaggggcggcgactggtgagtacgccaaaa
attttgactagcggaggctagaaggagagagatgggtgcgagagcgtcagtattaagcgggggagaattagatcgcgatggg
aaaaaattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcagacaggat
cagaagaacttagatcattatataatacagtagcaaccctctattgtgtgcatcaaaggatagagataaaagacaccaaggaa
gctttagacaagatagaggaagagcaaaacaaaagtaagaccaccgcacagcaagcggccggccgctgatcttcagacct
ggaggaggagatatgagggacaattggagaagtgaattatataaatataaagtagtaaaaattgaaccattaggagtagcacc
caccaaggcaaagagaagagtggtgcagagagaaaaaagagcagtgggaataggagctttgttccttgggttcttgggagca
gcaggaagcactatgggcgcagcgtcaatgacgctgacggtacaggccagacaattattgtctggtatagtgcagcagcaga
acaatttgctgagggctattgaggcgcaacagcatctgttgcaactcacagtctggggcatcaagcagctccaggcaagaatcc
tggctgtggaaagatacctaaaggatcaacagctcctggggatttggggttgctctggaaaactcatttgcaccactgctgtgcctt
ggaatgctagttggagtaataaatctctggaacagatttggaatcacacgacctggatggagtgggacagagaaattaacaatt
acacaagcttaatacactccttaattgaagaatcgcaaaaccagcaagaaaagaatgaacaagaattattggaattagataaa
tgggcaagtttgtggaattggtttaacataacaaattggctgtggtatataaaattattcataatgatagtaggaggcttggtaggttta
agaatagtttttgctgtactttctatagtgaatagagttaggcagggatattcaccattatcgtttcagacccacctcccaaccccgag
gggacccgacaggcccgaaggaatagaagaagaaggtggagagagagacagagacagatccattcgattagtgaacgg
atctcgacggtatcgccaaatggcagtattcatccacaattttaaaagaaaaggggggattggggggtacagtgcaggggaaa
gaatagtagacataatagcaacagacatacaaactaaagaattacaaaaacaaattacaaaaattcaaaattttcgggtttatta
cagggacagcagagatccagtttggatcgataagcttgatatcgaattcctgcagccccgataaaataaaagattttatttagtctc
cagaaaaaggggggaatgaaagaccccacctgtaggtttggcaagctagctgcagtaacgccattttgcaaggcatggaaaa
ataccaaaccaagaatagagaagttcagatcaagggcgggtacatgaaaatagctaacgttgggccaaacaggatatctgc
ggtgagcagtttcggccccggcccggggccaagaacagatggtcaccgcagtttcggccccggcccgaggccaagaacag
atggtccccagatatggcccaaccctcagcagtttcttaagacccatcagatgtttccaggctcccccaaggacctgaaatgacc
ctgcgccttatttgaattaaccaatcagcctgcttctcgcttctgttcgcgcgcttctgcttcccgagctctataaaagagctcacaac
ccctcactcggcgcgccagtcctccgacagactgagtcgcccggggggGATCCGCCACCATGAGCATGCTGT
TCTATACCCTGATCACCGCCTTTCTGATCGGCATCCAGGCCGAGCAGAAGCTGATCTC
CGAGGAAGATCTGGCCTCTAGCCTGGGCGCTCACCACATCCACCACTTTCACGGCAG
CAGCAAGCACCACTCTGTGCCTATCGCCATCTACAGAAGCCCCGCTTCTCTGAGAGGC
GGCCATGCTGGAACCACCTACATCTTCTCTAAAGGGGGGGGACAGATCACCTACAAGT
GGCCTCCAAACGACAGACCCAGCACCAGAGCCGATAGACTGGCCATCGGCTTCAGCA
CCGTGCAGAAAGAAGCCGTGCTCGTCAGAGTGGACAGCAGCTCTGGACTGGGCGACT
ACCTGGAACTGCACATCCATCAGGGCAAGATCGGCGTGAAGTTCAACGTGGGCACCG
ACGACATTGCCATCGAGGAAAGCAACGCCATCATCAACGACGGCAAGTACCACGTCGT
GCGGTTCACAAGAAGCGGCGGCAACGCTACACTGCAGGTCGACTCTTGGCCCGTGAT
CGAGAGATACCCTGCCGGCAACAACGACAACGAGAGACTGGCTATCGCCAGACAGAG
AATCCCCTACAGACTGGGAAGAGTGGTGGACGAGTGGCTGCTGGATAAGGGCAGACA
GCTGACCATCTTCAATAGCCAGGCCACCATCATCATCGGCGGAAAAGAGCAGGGCCA
GCCTTTCCAGGGACAGCTGAGCGGACTGTACTACAACGGCCTGAAGGTGCTGAACAT
GGCCGCTGAGAACGACGCCAATATCGCTATCGTGGGCAACGTGCGGCTCGTGGGAGA
AGTTCCTGGCGGCGGAGGATCTGGCGGAGGTGGAAGTGGCGGAGGCGGATCTACAC
TGGTGCTGTTTGGCGCTGGCTTCGGCGCCGTGATTACAGTGGTGGTCATCGTCGTGAT
CATCAAGTGTTTCTGCAAATGAGCggccgcgactctagagtcgacctgcaggcatgcaagcttgatatcaagct
tatcgataatcaacctctggattacaaaatttgtgaaagattgactggtattcttaactatgttgctccttttacgctatgtggatacgctg
ctttaatgcctttgtatcatgctattgcttccccgtatggctttcattttctcctccttgtataaatcctggttgctgtctctttatgaggagttgt
ggcccgttgtcaggcaacgtggcgtggtgtgcactgtgtttgctgacgcaacccccactggttggggcattgccaccacctgtcag
ctcctttccgggactttcgctttccccctccctattgccacggcggaactcatcgccgcctgccttgcccgctgctggacaggggctc
ggctgttgggcactgacaattccgtggtgttgtcggggaaatcatcgtcctttccttggctgctcgcctgtgttgccacctggattctgc
gcgggacgtccttctgctacgtcccttcggccctcaatccagcggaccttccttcccaattctaccgggtaggggaggcgcttttcc
caaggcagtctggagcatgcgctttagcagccccgctgggcacttggcgctacacaagtggcctctggcctcgcacacattcca
catccaccggtaggcgccaaccggctccgttctttggtggccccttcgcgccaccttctactcctcccctagtcaggaagttccccc
ccgccccgcagctcgcgtcgtgcaggacgtgacaaatggaagtagcacgtctcactagtctcgtgcagatggacagcaccgct
gagcaatggaagcgggtaggcctttggggcagcggccaatagcagctttgctccttcgctttctgggctcagaggctgggaagg
ggtgggtccggggggggctcaggggcgggctcaggggggggcgggcgcccgaaggtcctccggaggcccggcattctg
cacgcttcaaaagcgcacgtctgccgcgctgttctcctcttcctcatctccgggcctttcgacctgcagcccaagcttaccatgacc
gagtacaagcccacggtgcgcctcgccacccgcgacgacgtccccagggccgtacgcaccctcgccgccgegttcgccgac
taccccgccacgcgccacaccgtcgatccggaccgccacatcgagcgggtcaccgagctgcaagaactcttcctcacgcgcg
tcgggctcgacatcggcaaggtgtgggtcgcggacgacggcgccgcggtggcggtctggaccacgccggagagcgtcgaa
gcgggggcggtgttcgccgagatcggcccgcgcatggccgagttgagcggttcccggctggccgcgcagcaacagatggaa
ggcctcctggcgccgcaccggcccaaggagcccgcgtggttcctggccaccgtcggcgtctcgcccgaccaccagggcaag
ggtctgggcagcgccgtcgtgctccccggagtggaggcggccgagcgcgccggggtgcccgccttcctggagacctccgcgc
cccgcaacctccccttctacgagcggctcggcttcaccgtcaccgccgacgtcgaggtgcccgaaggaccgcgcacctggtgc
atgacccgcaagcccggtgcctgactcgagggaattaattcgagctcggtacctttaagaccaatgacttacaaggcagctgta
gatcttagccactttttaaaagaaaaggggggactggaagggctaattcactcccaacgaagacaagatctgctttttgcttgtact
gggtctctctggttagaccagatctgagcctgggagctctctggctaactagggaacccactgcttaagcctcaataaagcttgcc
ttgagtgcttcaagtagtgtgtgcccgtctgttgtgtgactctggtaactagagatccctcagacccttttagtcagtgtggaaaatctc
tagcagcatctagaattaattccgtgtattctatagtgtcacctaaatcgtatgtgtatgatacataaggttatgtattaattgtagccgc
gttctaacgacaatatgtacaagcctaattgtgtagcatctggcttactgaagcagaccctatcatctctctcgtaaactgccgtcag
agtcggtttggttggacgaaccttctgagtttctggtaacgccgtcccgcacccggaaatggtcagcgaaccaatcagcagggtc
atcgctagccagatcctctacgccggacgcatcgtggccggcatcaccggcgccacaggtgcggttgctggcgcctatatcgcc
gacatcaccgatggggaagatcgggctcgccacttcgggctcatgagcgcttgtttcggcgtgggtatggtggcaggccccgtg
gccgggggactgttgggcgccatctccttgcatgcaccattccttgcggcggcggtgctcaacggcctcaacctactactgggct
gcttcctaatgcaggagtcgcataagggagagcgtcgaatggtgcactctcagtacaatctgctctgatgccgcatagttaagcc
agccccgacacccgccaacacccgctgacgcgccctgacgggcttgtctgctcccggcatccgcttacagacaagctgtgacc
gtctccgggagctgcatgtgtcagaggttttcaccgtcatcaccgaaacgcgcgagacgaaagggcctcgtgatacgcctattttt
ataggttaatgtcatgataataatggtttcttagacgtcaggtggcacttttcggggaaatgtgcgcggaacccctatttgtttatttttct
aaatacattcaaatatgtatccgctcatgagacaataaccctgataaatgcttcaataatattgaaaaaggaagagtatgagtatt
caacatttccgtgtcgcccttattcccttttttgcggcattttgccttcctgtttttgctcacccagaaacgctggtgaaagtaaaagatg
ctgaagatcagttgggtgcacgagtgggttacatcgaactggatctcaacagcggtaagatccttgagagttttcgccccgaaga
acgttttccaatgatgagcacttttaaagttctgctatgtggcgcggtattatcccgtattgacgccgggcaagagcaactcggtcgc
cgcatacactattctcagaatgacttggttgagtactcaccagtcacagaaaagcatcttacggatggcatgacagtaagagaat
tatgcagtgctgccataaccatgagtgataacactgcggccaacttacttctgacaacgatcggaggaccgaaggagctaacc
gcttttttgcacaacatgggggatcatgtaactcgccttgatcgttgggaaccggagctgaatgaagccataccaaacgacgag
cgtgacaccacgatgcctgtagcaatggcaacaacgttgcgcaaactattaactggcgaactacttactctagcttcccggcaac
aattaatagactggatggaggcggataaagttgcaggaccacttctgcgctcggcccttccggctggctggtttattgctgataaat
ctggagccggtgagcgtgggtctcgcggtatcattgcagcactggggccagatggtaagccctcccgtatcgtagttatctacac
gacggggagtcaggcaactatggatgaacgaaatagacagatcgctgagataggtgcctcactgattaagcattggtaactgt
cagaccaagtttactcatatatactttagattgatttaaaacttcatttttaatttaaaaggatctaggtgaagatcctttttgataatctca
tgaccaaaatcccttaacgtgagttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatcaagagctaccaactcttttt
ccgaaggtaactggcttcagcagagcgcagataccaaatactgttcttctagtgtagccgtagttaggccaccacttcaagaact
ctgtagcaccgcctacatacctcgctctgctaatcctgttaccagtggctgctgccagtggcgataagtcgtgtcttaccgggttgga
ctcaagacgatagttaccggataaggcgcagcggtcgggctgaacggggggttcgtgcacacagcccagcttggagcgaac
gacctacaccgaactgagatacctacagcgtgagctatgagaaagcgccacgcttcccgaagggagaaaggcggacaggt
atccggtaagcggcagggtcggaacaggagagcgcacgagggagcttccagggggaaacgcctggtatctttatagtcctgt
cgggtttcgccacctctgacttgagcgtcgatttttgtgatgctcgtcaggggggcggagcctatggaaaaacgccagcaacgcg
gcctttttacggttcctggccttttgctggccttttgctcacatgttctttcctgcgttatcccctgattctgtggataaccgtattaccgccttt
gagtgagctgataccgctcgccgcagccgaacgaccgagcgcagcgagtcagtgagcgaggaagcggaagagcgccca
atacgcaaaccgcctctccccgcgcgttggccgattcattaatgcagctgtggaatgtgtgtcagttagggtgtggaaagtcccca
ggctccccagcaggcagaagtatgcaaagcatgcatctcaattagtcagcaaccaggtgtggaaagtccccaggctccccag
caggcagaagtatgcaaagcatgcatctcaattagtcagcaaccatagtcccgcccctaactccgcccatcccgcccctaactc
cgcccagttccgcccattctccgccccatggctgactaattttttttatttatgcagaggccgaggccgcctcggcctctgagctattc
cagaagtagtgaggaggcttttttggaggcctaggcttttgcaaaaagcttggacacaagacaggcttgcgagatatgtttgaga
ataccactttatcccgcgtcagggagaggcagtgcgtaaaaagacgcggactcatgtgaaatactggtttttagtgcgccagatc
tctataatctcgcgcaacctattttcccctcgaacactttttaagccgtagataaacaggctgggacacttcacatgagcgaaaaat
acatcgtcacctgggacatgttgcagatccatgcacgtaaactcgcaagccgactgatgccttctgaacaatggaaaggcattat
tgccgtaagccgtggcggtctgtaccgggtgcgttactggcgcgtgaactgggtattcgtcatgtcgataccgtttgtatttccagcta
cgatcacgacaaccagcgcgagcttaaagtgctgaaacgcgcagaaggcgatggcgaaggcttcatcgttattgatgacctg
gtggataccggtggtactgcggttgcgattcgtgaaatgtatccaaaagcgcactttgtcaccatcttcgcaaaaccggctggtcgt
ccgctggttgatgactatgttgttgatatcccgcaagatacctggattgaacagccgtgggatatgggcgtcgtattcgtcccgcca
atctccggtcgctaatcttttcaacgcctggcactgccgggcgttgttctttttaacttcaggcgggttacaatagtttccagtaagtatt
ctggaggctgcatccatgacacaggcaaacctgagcgaaaccctgttcaaaccccgctttaaacatcctgaaacctcgacgct
agtccgccgctttaatcacggcgcacaaccgcctgtgcagtcggcccttgatggtaaaaccatccctcactggtatcgcatgatta
accgtctgatgtggatctggcgcggcattgacccacgcgaaatcctcgacgtccaggcacgtattgtgatgagcgatgccgaac
gtaccgacgatgatttatacgatacggtgattggctaccgtggcggcaactggatttatgagtgggccccggatctttgtgaagga
accttacttctgtggtgtgacataattggacaaactacctacagagatttaaagctctaaggtaaatataaaatttttaagtgtataat
gtgttaaactactgattctaattgtttgtgtattttagattccaacctatggaactgatgaatgggagcagtggtggaatgcctttaatga
ggaaaacctgttttgctcagaagaaatgccatctagtgatgatgaggctactgctgactctcaacattctactcctccaaaaaaga
agagaaaggtagaagaccccaaggactttccttcagaattgctaagttttttgagtcatgctgtgtttagtaatagaactcttgcttgc
tttgctatttacaccacaaaggaaaaagctgcactgctatacaagaaaattatggaaaaatattctgtaacctttataagtaggcat
aacagttataatcataacatactgttttttcttactccacacaggcatagagtgtctgctattaataactatgctcaaaaattgtgtacct
ttagctttttaatttgtaaaggggttaataaggaatatttgatgtatagtgccttgactagagatcataatcagccataccacatttgtag
aggttttacttgctttaaaaaacctcccacacctccccctgaacctgaaacataaaatgaatgcaattgttgttgttaacttgtttattgc
agcttataatggttacaaataaagcaatagcatcacaaatttcacaaataaagcatttttttcactgcattctagttgtggtttgtccaa
actcatcaatgtatcttatcatgtctggatcaactggataactcaagctaaccaaaatcatcccaaacttcccaccccataccctatt
accactgccaattacctgtggtttcatttactctaaacctgtgattcctctgaattattttcattttaaagaaattgtatttgttaaatatgtac
tacaaacttagtagt
SEQ ID NO: 59 - pREF096 sequence (GFP under FoxP3-inducible promoter)
tggaagggctaattcactcccaaagaagacaagatatccttgatctgtggatctaccacacacaaggctacttccctgattagca
gaactacacaccagggccaggggtcagatatccactgacctttggatggtgctacaagctagtaccagttgagccagataagg
tagaagaggccaataaaggagagaacaccagcttgttacaccctgtgagcctgcatgggatggatgacccggagagagaag
tgttagagtggaggtttgacagccgcctagcatttcatcacgtggcccgagagctgcatccggagtacttcaagaactgctgatat
cgagcttgctacaagggactttccgctggggactttccagggaggcgtggcctggggggactggggagtggcgagccctcag
atcctgcatataagcagctgctttttgcctgtactgggtctctctggttagaccagatctgagcctgggagctctctggctaactaggg
aacccactgcttaagcctcaataaagcttgccttgagtgcttcaagtagtgtgtgcccgtctgttgtgtgactctggtaactagagatc
cctcagacccttttagtcagtgtggaaaatctctagcagtggcgcccgaacagggacttgaaagcgaaagggaaaccagagg
agctctctcgacgcaggactcggcttgctgaagcgcgcacggcaagaggcgaggggcggcgactggtgagtacgccaaaa
attttgactagcggaggctagaaggagagagatgggtgcgagagcgtcagtattaagcgggggagaattagatcgcgatggg
aaaaaattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcagacaggat
cagaagaacttagatcattatataatacagtagcaaccctctattgtgtgcatcaaaggatagagataaaagacaccaaggaa
gctttagacaagatagaggaagagcaaaacaaaagtaagaccaccgcacagcaagcggccggccgctgatcttcagacct
ggaggaggagatatgagggacaattggagaagtgaattatataaatataaagtagtaaaaattgaaccattaggagtagcacc
caccaaggcaaagagaagagtggtgcagagagaaaaaagagcagtgggaataggagctttgttccttgggttcttgggagca
gcaggaagcactatgggcgcagcgtcaatgacgctgacggtacaggccagacaattattgtctggtatagtgcagcagcaga
acaatttgctgagggctattgaggcgcaacagcatctgttgcaactcacagtctggggcatcaagcagctccaggcaagaatcc
tggctgtggaaagatacctaaaggatcaacagctcctggggatttggggttgctctggaaaactcatttgcaccactgctgtgcctt
ggaatgctagttggagtaataaatctctggaacagatttggaatcacacgacctggatggagtgggacagagaaattaacaatt
acacaagcttaatacactccttaattgaagaatcgcaaaaccagcaagaaaagaatgaacaagaattattggaattagataaa
tgggcaagtttgtggaattggtttaacataacaaattggctgtggtatataaaattattcataatgatagtaggaggcttggtaggttta
agaatagtttttgctgtactttctatagtgaatagagttaggcagggatattcaccattatcgtttcagacccacctcccaaccccgag
gggacccgacaggcccgaaggaatagaagaagaaggtggagagagagacagagacagatccattcgattagtgaacgg
atctcgacggtatcgccaaatggcagtattcatccacaattttaaaagaaaaggggggattggggggtacagtgcaggggaaa
gaatagtagacataatagcaacagacatacaaactaaagaattacaaaaacaaattacaaaaattcaaaattttcgggtttatta
cagggacagcagagatccagtttggatcgataagcttgatatcgaattcgtagctgaagaaaggattcataaatgaagttcaatc
cttctcatcaaccccagcccacacctccagcaattgaacttgaaaaaaaaaacctggtttgaaaaattaccgcaaactatattgt
catcaaaaaaaaaaaaaaaaaaaaacacttcctatatttgagatgagagaagagagtgctaggcagtttcctggctgaacac
gccagcccaatacttaaagagagcaactcctgactccgatagagactggatggacccacaagggtgacagcccaggcgga
ccgatcttcccatcccacatcctccggcgcgatgccaaaaagaggctgacggcaactgggccttctgcagagaaagacctcc
gcttcactgccccggctggtcccaagggtcaggaagATGGTAAGCAAGGGAGAAGAACTTTTCACGGGA
GTAGTTCCTATTCTGGTGGAACTGGACGGGGATGTAAACGGGCATAAATTCTCTGTCAG
CGGGGAAGGGGAAGGCGATGCAACATACGGGAAGCTTACCCTCAAATTCATCTGTACG
ACAGGGAAACTTCCAGTGCCCTGGCCTACACTTGTGACAACCCTGACTTATGGGGTCC
AATGTTTCTCCAGGTACCCTGATCACATGAAACAGCATGACTTCTTCAAGAGCGCAATGC
CTGAAGGGTATGTCCAGGAACGCACGATTTTCTTCAAGGACGATGGCAATTATAAAACTA
GAGCCGAAGTAAAATTTGAGGGAGATACATTGGTAAACAGGATCGAGCTCAAAGGGATC
GATTTCAAAGAGGATGGGAACATCCTGGGGCATAAGTTGGAGTATAACTATAACTCTCAC
AATGTCTATATTATGGCCGACAAGCAAAAGAACGGGATAAAAGTCAACTTTAAAATAAGG
CACAATATAGAGGACGGCAGTGTGCAATTGGCGGACCACTATCAACAAAATACCCCGAT
CGGCGACGGTCCAGTGTTGCTCCCTGATAATCACTACCTTTCAACACAAAGTGCACTTA
GTAAAGATCCCAATGAGAAGCGGGATCACATGGTCCTGCTCGAATTTGTCACAGCAGCT
GGCATCACACTTGGGATGGATGAGCTCTATAAATAAggccgcgactctagagtcgacctgcaggcatgc
aagcttgatatcaagcttatcgataatcaacctctggattacaaaatttgtgaaagattgactggtattcttaactatgttgctccttttac
gctatgtggatacgctgctttaatgcctttgtatcatgctattgcttccccgtatggctttcattttctcctccttgtataaatcctggttgctgt
ctctttatgaggagttgtggcccgttgtcaggcaacgtggcgtggtgtgcactgtgtttgctgacgcaacccccactggttggggcat
tgccaccacctgtcagctcctttccgggactttcgctttccccctccctattgccacggeggaactcatcgccgcctgccttgcccgc
tgctggacaggggctcggctgttgggcactgacaattccgtggtgttgtcggggaaatcatcgtcctttccttggctgctcgcctgtgt
tgccacctggattctgcgcgggacgtccttctgctacgtcccttcggccctcaatccagcggaccttccttcccaattctaccgggta
ggggaggcgcttttcccaaggcagtctggagcatgcgctttagcagccccgctgggcacttggcgctacacaagtggcctctgg
cctcgcacacattccacatccaccggtaggcgccaaccggctccgttctttggtggccccttcgcgccaccttctactcctccccta
gtcaggaagttcccccccgccccgcagctcgcgtcgtgcaggacgtgacaaatggaagtagcacgtctcactagtctcgtgca
gatggacagcaccgctgagcaatggaagcgggtaggcctttggggcagcggccaatagcagctttgctccttcgctttctgggct
cagaggctgggaaggggtgggtccggggggggctcaggggcgggctcaggggggggggggcccgaaggtcctcc
ggaggcccggcattctgcacgcttcaaaagcgcacgtctgccgcgctgttctcctcttcctcatctccgggcctttcgacctgcagc
ccaagcttaccatgaccgagtacaagcccacggtgcgcctcgccacccgcgacgacgtccccagggccgtacgcaccctcg
ccgccgcgttcgccgactaccccgccacgcgccacaccgtcgatccggaccgccacatcgagcgggtcaccgagctgcaag
aactcttcctcacgcgcgtcgggctcgacatcggcaaggtgtgggtcgcggacgacggcgccgcggtggcggtctggaccac
gccggagagcgtcgaagcgggggcggtgttcgccgagatcggcccgcgcatggccgagttgagcggttcccggctggccgc
gcagcaacagatggaaggcctcctggcgccgcaccggcccaaggagcccgcgtggttcctggccaccgtcggcgtctcgcc
cgaccaccagggcaagggtctgggcagcgccgtcgtgctccccggagtggaggcggccgagcgcgccggggtgcccgcct
tcctggagacctccgcgccccgcaacctccccttctacgagcggctcggcttcaccgtcaccgccgacgtcgaggtgcccgaa
ggaccgcgcacctggtgcatgacccgcaagcccggtgcctgactcgagggaattaattcgagctcggtacctttaagaccaat
gacttacaaggcagctgtagatcttagccactttttaaaagaaaaggggggactggaagggctaattcactcccaacgaagac
aagatctgctttttgcttgtactgggtctctctggttagaccagatctgagcctgggagctctctggctaactagggaacccactgctt
aagcctcaataaagcttgccttgagtgcttcaagtagtgtgtgcccgtctgttgtgtgactctggtaactagagatccctcagaccctt
ttagtcagtgtggaaaatctctagcagcatctagaattaattccgtgtattctatagtgtcacctaaatcgtatgtgtatgatacataag
gttatgtattaattgtagccgcgttctaacgacaatatgtacaagcctaattgtgtagcatctggcttactgaagcagaccctatcatc
tctctcgtaaactgccgtcagagtcggtttggttggacgaaccttctgagtttctggtaacgccgtcccgcacccggaaatggtcag
cgaaccaatcagcagggtcatcgctagccagatcctctacgccggacgcatcgtggccggcatcaccggcgccacaggtgc
ggttgctggcgcctatatcgccgacatcaccgatggggaagatcgggctcgccacttcgggctcatgagcgcttgtttcggcgtg
ggtatggtggcaggccccgtggccgggggactgttgggcgccatctccttgcatgcaccattccttgcggcggcggtgctcaacg
gcctcaacctactactgggctgcttcctaatgcaggagtcgcataagggagagcgtcgaatggtgcactctcagtacaatctgct
ctgatgccgcatagttaagccagccccgacacccgccaacacccgctgacgcgccctgacgggcttgtctgctcccggcatcc
gcttacagacaagctgtgaccgtctccgggagctgcatgtgtcagaggttttcaccgtcatcaccgaaacgcgcgagacgaaa
gggcctcgtgatacgcctatttttataggttaatgtcatgataataatggtttcttagacgtcaggtggcacttttcggggaaatgtgcg
cggaacccctatttgtttatttttctaaatacattcaaatatgtatccgctcatgagacaataaccctgataaatgcttcaataatattga
aaaaggaagagtatgagtattcaacatttccgtgtcgcccttattcccttttttgcggcattttgccttcctgtttttgctcacccagaaac
gctggtgaaagtaaaagatgctgaagatcagttgggtgcacgagtgggttacatcgaactggatctcaacagcggtaagatcct
tgagagttttcgccccgaagaacgttttccaatgatgagcacttttaaagttctgctatgtggcgcggtattatcccgtattgacgccg
ggcaagagcaactcggtcgccgcatacactattctcagaatgacttggttgagtactcaccagtcacagaaaagcatcttacgg
atggcatgacagtaagagaattatgcagtgctgccataaccatgagtgataacactgcggccaacttacttctgacaacgatcg
gaggaccgaaggagctaaccgcttttttgcacaacatgggggatcatgtaactcgccttgatcgttgggaaccggagctgaatg
aagccataccaaacgacgagcgtgacaccacgatgcctgtagcaatggcaacaacgttgcgcaaactattaactggcgaact
acttactctagcttcccggcaacaattaatagactggatggaggcggataaagttgcaggaccacttctgcgcteggcccttccg
gctggctggtttattgctgataaatctggagccggtgagcgtgggtctcgcggtatcattgcagcactggggccagatggtaagcc
ctcccgtatcgtagttatctacacgacggggagtcaggcaactatggatgaacgaaatagacagatcgctgagataggtgcctc
actgattaagcattggtaactgtcagaccaagtttactcatatatactttagattgatttaaaacttcatttttaatttaaaaggatctagg
tgaagatcctttttgataatctcatgaccaaaatcccttaacgtgagttttcgttccactgagcgtcagaccccgtagaaaagatcaa
aggatcttcttgagatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgttcttctagtgtagccgtagtt
aggccaccacttcaagaactctgtagcaccgcctacatacctcgctctgctaatcctgttaccagtggctgctgccagtggcgata
agtcgtgtcttaccgggttggactcaagacgatagttaccggataaggcgcagcggtcgggctgaacggggggttcgtgcaca
cagcccagcttggagcgaacgacctacaccgaactgagatacctacagcgtgagctatgagaaagcgccacgcttcccgaa
gggagaaaggcggacaggtatccggtaagcggcagggtcggaacaggagagcgcacgagggagcttccagggggaaa
cgcctggtatctttatagtcctgtcgggtttcgccacctctgacttgagcgtcgatttttgtgatgctcgtcaggggggcggagcctatg
gaaaaacgccagcaacgcggcctttttacggttcctggccttttgctggccttttgctcacatgttctttcctgcgttatcccctgattctg
tggataaccgtattaccgcctttgagtgagctgataccgctcgccgcagccgaacgaccgagcgcagcgagtcagtgagcga
ggaagcggaagagcgcccaatacgcaaaccgcctctccccgcgcgttggccgattcattaatgcagctgtggaatgtgtgtca
gttagggtgtggaaagtccccaggctccccagcaggcagaagtatgcaaagcatgcatctcaattagtcagcaaccaggtgtg
gaaagtccccaggctccccagcaggcagaagtatgcaaagcatgcatctcaattagtcagcaaccatagtcccgcccctaact
ccgcccatcccgcccctaactccgcccagttccgcccattctccgccccatggctgactaattttttttatttatgcagaggccgagg
ccgcctcggcctctgagctattccagaagtagtgaggaggcttttttggaggcctaggcttttgcaaaaagcttggacacaagac
aggcttgcgagatatgtttgagaataccactttatcccgcgtcagggagaggcagtgcgtaaaaagacgcggactcatgtgaaa
tactggtttttagtgcgccagatctctataatctcgcgcaacctattttcccctcgaacactttttaagccgtagataaacaggctggg
acacttcacatgagcgaaaaatacatcgtcacctgggacatgttgcagatccatgcacgtaaactcgcaagccgactgatgcct
tctgaacaatggaaaggcattattgccgtaagccgtggcggtctgtaccgggtgcgttactggcgcgtgaactgggtattcgtcat
gtcgataccgtttgtatttccagctacgatcacgacaaccagcgcgagcttaaagtgctgaaacgcgcagaaggcgatggcga
aggcttcatcgttattgatgacctggtggataccggtggtactgcggttgcgattcgtgaaatgtatccaaaagcgcactttgtcacc
atcttcgcaaaaccggctggtcgtccgctggttgatgactatgttgttgatatcccgcaagatacctggattgaacagccgtgggat
atgggcgtcgtattcgtcccgccaatctccggtcgctaatcttttcaacgcctggcactgccgggcgttgttctttttaacttcaggcgg
gttacaatagtttccagtaagtattctggaggctgcatccatgacacaggcaaacctgagcgaaaccctgttcaaaccccgcttta
aacatcctgaaacctcgacgctagtccgccgctttaatcacggcgcacaaccgcctgtgcagtcggcccttgatggtaaaacca
tccctcactggtatcgcatgattaaccgtctgatgtggatctggcgcggcattgacccacgcgaaatcctcgacgtccaggcacgt
attgtgatgagcgatgccgaacgtaccgacgatgatttatacgatacggtgattggctaccgtggcggcaactggatttatgagtg
ggccccggatctttgtgaaggaaccttacttctgtggtgtgacataattggacaaactacctacagagatttaaagctctaaggtaa
atataaaatttttaagtgtataatgtgttaaactactgattctaattgtttgtgtattttagattccaacctatggaactgatgaatgggag
cagtggtggaatgcctttaatgaggaaaacctgttttgctcagaagaaatgccatctagtgatgatgaggctactgctgactctcaa
cattctactcctccaaaaaagaagagaaaggtagaagaccccaaggactttccttcagaattgctaagttttttgagtcatgctgtg
tttagtaatagaactcttgcttgctttgctatttacaccacaaaggaaaaagctgcactgctatacaagaaaattatggaaaaatatt
ctgtaacctttataagtaggcataacagttataatcataacatactgttttttcttactccacacaggcatagagtgtctgctattaataa
ctatgctcaaaaattgtgtacctttagctttttaatttgtaaaggggttaataaggaatatttgatgtatagtgccttgactagagatcat
aatcagccataccacatttgtagaggttttacttgctttaaaaaacctcccacacctccccctgaacctgaaacataaaatgaatg
caattgttgttgttaacttgtttattgcagcttataatggttacaaataaagcaatagcatcacaaatttcacaaataaagcatttttttca
ctgcattctagttgtggtttgtccaaactcatcaatgtatcttatcatgtctggatcaactggataactcaagctaaccaaaatcatccc
aaacttcccaccccataccctattaccactgccaattacctgtggtttcatttactctaaacctgtgattcctctgaattattttcattttaa
agaaattgtatttgttaaatatgtactacaaacttagtagt
SEQ ID NO: 60 - hypervariable region of light chain variable region of humanized M1 -
HM1 HVR-L1
RASKSINKYLA
SEQ ID NO: 61 - hypervariable region of light chain variable region of humanized M1 -
HM1 HVR-L2
SGSTLQS
SEQ ID NO: 62 - hypervariable region of light chain variable region of humanized M1 -
HM1 HVR-L3
QQHNEYPLT
SEQ ID NO: 63 - hypervariable region of heavy chain variable region of humanized M1 -
HM1 HVR-H1
GYHFTSYWMH
SEQ ID NO: 64 - hypervariable region of heavy chain variable region of humanized M1 -
HM1 HVR-H2
VIHPNSGSINYNEKFES
SEQ ID NO: 65 - hypervariable region of heavy chain variable region of humanized M1 -
HM1 HVR-H3
ERDSTEVLPMDY

1. An artificial T cell receptor, wherein an antigen binding domain of the artificial T cell receptor specifically binds a complement pathway protein, preferably, wherein the artificial T cell receptor is a chimeric antigen receptor.

2. The artificial T cell receptor of claim 1, wherein the antigen binding domain comprises an antibody fragment or derivative thereof; preferably wherein the antibody fragment comprises a fragment selected from the group comprising Fab, Fab′, F(ab′)₂, Fv, scFv, disulfide-linked Fvs (sdFv), Fd, linear antibodies, and single domain antibodies; most preferably wherein the antigen binding domain comprises an scFv antibody fragment.

3. The artificial T cell receptor of claim 1 or 2, wherein the complement pathway protein is selected from the group comprising C1q, C1r, C1s, C2a, C3, C3a, C3b, C4, C4a, C4b, C5, C5a, C5b, C6, C7, C8, and C9; preferably wherein the complement pathway protein is C1q.

4. The artificial T cell receptor of any of claims 1 to 3, wherein the chimeric antigen receptor comprises an intracellular signalling domain comprising an intracellular signalling domain of CD3zeta, CD28, ICOS, OX-40, or a combination thereof.

5. A nucleic acid encoding the artificial T cell receptor of any of claims 1 to 4.

6. The nucleic acid of claim 5, wherein the nucleic acid is operatively linked to a transcriptional regulatory sequence, and wherein the transcriptional regulatory sequence is configured to bind a transcription factor; preferably wherein the transcriptional regulatory sequence configured to bind the transcription factor comprises a binding domain for Gal4-VP6, tetR-VP64 (tTA), ZFHD1-VP64, Gal4-KRAB, PIP-VP64, ZF21-16-VP64, ZF43-8-VP64 or FoxP3; most preferably wherein the transcriptional regulatory sequence comprises a binding domain for FoxP3.

7. A targeting polypeptide, wherein the targeting polypeptide comprises an extracellular domain, a transmembrane domain, and an intracellular domain, wherein the extracellular domain comprises a ligand binding domain and wherein the intracellular domain comprises a transcription factor, and wherein the transcription factor is configured to be released upon binding of the ligand binding domain by a ligand; and preferably, wherein the extracellular domain and the intracellular domain are heterologous to the transmembrane domain.

8. The targeting polypeptide of claim 7, wherein the transcription factor is released by proteolytic cleavage.

9. The targeting polypeptide of claim 7 or 8, wherein the transmembrane domain comprises a notch minimal regulatory region or a notch extended regulatory region.

10. The targeting polypeptide of claim 7 or 8, wherein the intracellular domain comprises a cleavage domain configured to be cleaved by a protease; preferably a type II serine protease.

11. The targeting polypeptide of claim 10, wherein the intracellular domain comprises a cleavage domain configured to be cleaved by a type II serine protease, a type II serine protease domain comprising a catalytically active region of a serine protease, an inhibitory domain comprising an amino acid sequence that inhibits the catalytically active region of the type II serine protease when the ligand binding domain is not bound by a ligand, and the transcription factor; preferably
wherein the catalytically active region of the serine protease comprises an active domain of thrombin, Hepatitis C virus Ns3 serine protease, or a TVMV protease.

12. The targeting polypeptide of claim 7 or 8, wherein the ligand binding domain comprises an amino acid sequence that specifically reacts with a benzyl guanine derivative or an O2-benzylcytosine (BC) derivative; preferably wherein the ligand binding domain comprises a SNAP-Tag or a CLIP-Tag.

13. A targeting polypeptide, wherein the targeting polypeptide comprises a ligand binding domain, a transmembrane domain, and a transcription factor, wherein the transmembrane domain is located between the ligand binding domain and the transcription factor, and wherein the transcription factor is cleavably linked to the transmembrane domain; preferably wherein the transmembrane domain and the transcription factor are linked by a cleavable peptide linker.

14. The targeting polypeptide of claim 13, wherein the cleavable linker comprises at least one self-cleaving peptide; preferably wherein the at least one self-cleaving peptide comprises a 2A self-cleaving peptide; more preferably wherein the 2A self-cleaving peptide comprises a P2A peptide, an E2A peptide, an F2A peptide, and/or a T2A peptide, or tandem or triple arrangements of such peptides.

15. The targeting polypeptide of any of claims 7 to 14, wherein the ligand binding domain specifically binds a tissue-associated antigen; preferably wherein the tissue-associated antigen is a tissue specific marker.

16. The targeting polypeptide of claim 15, wherein the tissue-associated antigen is a neuronal marker present at a neuronal synapse; preferably wherein the neuronal marker present at a neuronal synapse is a neuronal antigen; most preferably wherein the neuronal antigen is neurexin or neuroligin.

17. The targeting polypeptide of claim 16, wherein the ligand binding domain comprises a neurexin polypeptide or a neuroligin binding fragment of the neurexin polypeptide.

18. The targeting polypeptide of claim 17, wherein the ligand binding domain comprises an amino acid sequence at least about 90%, 95%, 97%, 98%, 99%, or 100% identical to the amino acid sequence set forth in SEQ ID NO: 1.

19. The targeting polypeptide of claim 17 or 18, wherein the neurexin polypeptide or a neuroligin binding fragment thereof comprises an amino acid variant that reduces binding to neuroligin compared to a wild-type neurexin polypeptide or neuroligin binding fragment thereof.

20. The targeting polypeptide of claim 19, wherein the amino acid variant is selected from the group comprising S111A, D162A, I210A, N212A, I210A:D141A, and combinations thereof; preferably wherein the ligand binding domain comprises an amino acid sequence at least about 90%, 95%, 97%, 98%, 99%, or 100% identical to the amino acid sequence set forth in any one of SEQ ID NOs: 2 to 6.

21. The targeting polypeptide of any of claims 16 to 20, wherein the targeting polypeptide comprises an amino acid sequence at least about 90%, 95%, 97%, 98%, 99%, or 100% identical to the amino acid sequence set forth in any one of SEQ ID NOs: 14 to 19.

22. The targeting polypeptide of claim 16, wherein the ligand binding domain comprises an amino acid sequence that is at least about 90%, 95%, 97%, 98%, 99%, or 100% identical to the amino acid sequence set forth in SEQ ID NO: 7.

23. The targeting polypeptide of claim 15, wherein the tissue-associated antigen is an antigen associated with:
i) inflammatory bowel disease, such as Carcinoembryonic antigens, GLUT2, or GLUT5;
ii) Rheumatoid arthritis, such as Type II Collagen or Citrullinated vimentin; or
iii) Type 1 diabetes, such as Insulin or pro-Insulin.

24. The targeting polypeptide of any of claims 7 to 23, wherein the transcription factor is heterologous to the extracellular domain, the transmembrane domain and/or the rest of the intracellular domain.

25. The targeting polypeptide of any of claims 7 to 24, wherein the transcription factor is selected from the group comprising Gal4-VP6, tetR-VP64 (tTA), ZFHD1-VP64, Gal4-KRAB, PIP-VP64, ZF21-16-VP64, ZF43-8-VP64, LAIR2, METTL7A, RTKN2, FoxP3, BACH2, Cish, ZEB2, EOMES, ZNF683 (Hobit), AML1, ReIA, RORγt, TIP60/HDAC7, STAT3, IRF4, USP7, LEF1, GATA-1, GATA-3, and STAT5; preferably wherein the transcription factor is FoxP3.

26. A nucleic acid encoding the targeting polypeptide according to any of claims 7 to 25.

27. The nucleic acid according to claim 26, wherein the nucleic acid is operatively linked to a transcriptional regulatory sequence, and wherein the transcriptional regulatory sequence is configured to bind a transcription factor.

28. The nucleic acid of claim 27, wherein the transcriptional regulatory sequence configured to bind the transcription factor comprises a binding domain for Gal4-VP6, tetR-VP64 (tTA), ZFHD1-VP64, Gal4-KRAB, PIP-VP64, ZF21-16-VP64, ZF43-8-VP64 or FoxP3; preferably wherein the transcriptional regulatory sequence comprises a binding domain for FoxP3.

29. A cell engineered to express a nucleic acid encoding a targeting polypeptide, wherein the targeting polypeptide comprises a ligand binding domain, wherein the cell further comprises a nucleic acid encoding an artificial T cell receptor, and wherein an antigen binding domain of the artificial T cell receptor specifically binds a biomarker; optionally,
wherein the nucleic acid encoding the artificial T cell receptor is operatively linked to a transcriptional regulatory sequence, and wherein the transcriptional regulatory sequence is configured to bind a transcription factor.

30. The cell according to claim 29, wherein the ligand binding domain specifically binds a tissue-associated antigen; preferably wherein the tissue-associated antigen is a tissue specific marker; and most preferably wherein the targeting polypeptide is a targeting polypeptide according to any of claims 7 to 25.

31. The cell according to claim 29 or 30, wherein the biomarker is a biomarker of inflammation, an inflammatory mediator, and/or a disease-associated biomarker.

32. The cell according to claim 31, wherein the biomarker of inflammation is a complement pathway protein; preferably wherein the complement pathway protein is selected from the group comprising C1q, C1r, C1s, C2a, C3, C3a, C3b, C4, C4a, C4b, C5, C5a, C5b, C6, C7, C8, and C9; more preferably wherein the complement pathway protein is C1q.

33. A cell comprising:
i) the nucleic acid according to any of claims 5 or 6; and
ii) the nucleic acid according to any of claims 26, 27 or 28.

34. The cell according to any of claims 29 to 33, wherein the nucleic acid encoding the targeting polypeptide comprises a constitutively active promoter or enhancer operatively coupled to the coding region for the targeting polypeptide.

35. The cell according to any of claims 29 to 34, wherein the transcriptional regulatory sequence that is operatively linked to the nucleic acid encoding the artificial T cell receptor is configured to bind the same transcription factor that is cleavably linked to the ligand binding domain of the targeting polypeptide; preferably wherein release of the transcription factor from the targeting polypeptide activates expression of the artificial T cell receptor.

36. The cell according to claim 35, wherein the transcription factor is selected from the group comprising Gal4-VP6, tetR-VP64 (tTA), ZFHD1-VP64, Gal4-KRAB, PIP-VP64, ZF21-16-VP64, ZF43-8-VP64 and FoxP3; preferably wherein the transcription factor is FoxP3.

37. The cell according to any of claims 29 to 36, wherein the cell is an immune cell; preferably wherein immune cell is a T lymphocyte; most preferably wherein the T lymphocyte is a regulatory T lymphocyte.

38. The cell according to any of claims 29 to 36, wherein the cell is a mesenchymal stem cell; optionally wherein the mesenchymal stem cell is a type II mesenchymal stem cell or an adipose-derived stem cell.

39. The cell according to any of claims 29 to 36, wherein the cell is a CD34+ hematopoietic stem cell, CD34+ progenitor cell or a CD34+ induced pluripotent stem cell.

40. A pharmaceutical composition comprising the cell according to any of claims 29 to 39 further comprising a pharmaceutically acceptable carrier, diluent, or excipient; preferably wherein the pharmaceutical composition is formulated for intravenous injection.

41. The cell according to any of claims 29 to 39, or the pharmaceutical composition according to claim 40, for use in medicine.

42. The cell or pharmaceutical composition for use according to claim 41, wherein the cell or pharmaceutical composition is for use in treating an inflammatory disorder in a subject.

43. The cell or pharmaceutical composition for use according to claim 42, wherein the inflammatory disorder is an inflammatory disorder of the nervous system; preferably
wherein the inflammatory disorder of the nervous system is selected from the group comprising multiple sclerosis, chronic inflammatory demyelinating polyneuropathy, an encephalitis, a traumatic brain injury, myasthenia gravis, and amyotrophic lateral sclerosis; most preferably
wherein the inflammatory disorder of the nervous system is amyotrophic lateral sclerosis.

44. The cell or pharmaceutical composition for use according to claim 42 or 43, wherein the cell is autologous to the subject, or wherein the cell is allogenic to the subject.

45. A nucleic acid vector comprising the nucleic acid according to claim 5 or 6, and/or the nucleic acid according to any of claims 26, 27 or 28.

46. The nucleic acid vector according to claim 45, wherein the nucleic acid vector is:
i) a viral vector, preferably a retroviral vector, an adenoviral vector or an adeno-associated viral vector;
ii) a non-polymeric vector, preferably a liposome or a gold nanoparticle;
iii) a polymeric vector, preferably a dendrimer, a dendrigraft, a polymeric micelle or a poly(β-amino ester) vector;
iv) a transposon, such as a PiggyBack or Sleeping Beauty transposon; or
v) a plasmid flanked by regions for homologous recombination for CRISPR/Cas knock-in.

47. A method of making a cell according to any of claims 29 to 39 comprising contacting a cell with:
i) the nucleic acid according to claim 5 or 6;
ii) the nucleic acid according to any of claims 26, 27 or 28; and/or
iii) the nucleic acid vector of claim 45 or 46.

48. A biological targeting system comprising a) a targeting polypeptide comprising a domain that specifically binds to a tissue specific marker; b) an effector polypeptide wherein the effector polypeptide specifically binds a disease specific antigen or an immune effector molecule; and c) a cargo selected from an: extracellular vesicle, a protein-coated vesicle, a liposome, a dendrimer, a micelle, a biodegradable particle comprising P-selectin, endothelial selectin (E-selectin) and ICAM-1, an artificial nanostructure, an engineered viral particle, plasmid, a transposon, and a bacterial cell.

49. The biological targeting system according to claim 48 for use in medicine.

50. The biological targeting system for use according to claim 49, wherein the biological targeting system is for use in treating an inflammatory disorder in a subject; preferably wherein the inflammatory disorder is an inflammatory disorder of the nervous system; more preferably wherein the inflammatory disorder of the nervous system is selected from the group comprising multiple sclerosis, chronic inflammatory demyelinating polyneuropathy, an encephalitis, a traumatic brain injury, myasthenia gravis, and amyotrophic lateral sclerosis; most preferably wherein the inflammatory disorder of the nervous system is amyotrophic lateral sclerosis.


SEQUENCE LISTING (65 sequences)

SEQ ID NO: 1 - protein, 216 aa, Homo sapiens
ASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVP

SEQ ID NO: 2 - protein, 216 aa, synthetic construct
  REGION 1..216: note = Neurexin S111A
ASSLGAHHIHHFHGSSKHHSVPIAIYRSPASLRGGHAGTTYIFSKGGGQITYKWPPNDRPATRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVP

SEQ ID NO: 3 - protein, 216 aa, synthetic construct
  REGION 1..216: note = Neurexin D162A
ASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDAIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVP

SEQ ID NO: 4 - protein, 216 aa, synthetic construct
  REGION 1..216: note = Neurexin I210A
ASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTAFNSQATIIIGGKEQGQPFQGQLSGLYYNGLKVLNMAAENDANIAIVGNVRLVGEVP

SEQ ID NO: 5 - protein, 216 aa, synthetic construct
  REGION 1..216: note = Neurexin N212A
ASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFASQATIIIGGKEQGQPFQGQLSGLYYNGLKVLNMAAENDANIAIVGNVRLVGEVP

SEQ ID NO: 6 - protein, 216 aa, synthetic construct
  REGION 1..216: note = Neurexin D141A/ I210A
ASSLGAHHIHHFHGSSKHHSVPIAIYRSPASLRGGHAGTTYIFSKGGGQITYKWPPNDRPSTRADRLAIGFSTVQKEAVLVRVDSSSGLGAYLELHIHQGKIGVKFNVGTDDIAIEESNAIINDGKYHVVRFTRSGGNATLQVDSWPVIERYPAGRQLTAFNSQATIIIGGKEQGQPFQGQLSGLYYNGLKVLNMAAENDANIAIVGNVRLVGEVP

SEQ ID NO: 7 - protein, 844 aa, synthetic construct
  REGION 1..844: note = scFv, synNotch, Gal4/VP64
MALPVTALLLPLALLLHAARPEQKLISEEDLAAADIVLTQTPLSLPVSLGDQASISCRSSQNIVHSNGNTYLEWYLQKPGQSPKLLIYKVSNRFSGVPDRFSGSGSGTDFTLKISRVEAEDLGVYFCFQGSLLPWTFGGGTKLEIKRADAAPTVSGSTSGSGKPGSGEGSEFTSPASSSEVKLMESGGGSVKPGGSLKLSCVASGFTFSDYYMYWVRQTPEKRLEWVATISDGGNYPYYPDSVKGRFTISRDNARNNLYLQMTSLKSEDTAMYYCARGGAYSRSDGRFYYAMDYWGQGTSVTVSSARILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 8 - protein, 384 aa, Homo sapiens
PECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQPCVGCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKR

SEQ ID NO: 9 - protein, 306 aa, Mus musculus
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 10 - protein, 358 aa, Mus musculus
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 11 - protein, 363 aa, synthetic construct
  REGION 1..363: note = Notch
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRR

SEQ ID NO: 12 - protein, 245 aa, Homo sapiens
QLCIQKLMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSGSGVKQTLNFDLLKLAGDVESNPGPVD

SEQ ID NO: 13 - protein, 425 aa, Mus musculus
RPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 14 - protein, 1339 aa, synthetic construct
  REGION 1..1339: note = WT neurexin domain, followed by the Gal4 and FoxP3 after P2A
MALPVTALLLPLALLLHAARPEQKLISEEDLMYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVPGGGGSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSGSGVKQTLNFDLLKLAGDVESNPGPVDMPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 15 - protein, 1339 aa, synthetic construct
  REGION 1..1339: note = S111A neurexin domain, followed by the Gal4 and FoxP3 after P2A
MALPVTALLLPLALLLHAARPEQKLISEEDLMYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPATRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVPGGGGSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSGSGVKQTLNFDLLKLAGDVESNPGPVDMPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 16 - protein, 1339 aa, synthetic construct
  REGION 1..1339: note = D162A neurexin domain, followed by the Gal4 and FoxP3 after P2A
MALPVTALLLPLALLLHAARPEQKLISEEDLMYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDAIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVPGGGGSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSGSGVKQTLNFDLLKLAGDVESNPGPVDMPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 17 - protein, 1339 aa, synthetic construct
  REGION 1..1339: note = I210A neurexin domain, followed by the Gal4 and FoxP3 after P2A
MALPVTALLLPLALLLHAARPEQKLISEEDLMYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTAFNSQATIIIGGKEQGQPFQGQLSGLYYNGLKVLNMAAENDANIAIVGNVRLVGEVPGGGGSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSGSGVKQTLNFDLLKLAGDVESNPGPVDMPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 18 - protein, 1339 aa, synthetic construct
  REGION 1..1339: note = N212A neurexin domain, followed by the Gal4 and FoxP3 after P2A
MALPVTALLLPLALLLHAARPEQKLISEEDLMYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFASQATIIIGGKEQGQPFQGQLSGLYYNGLKVLNMAAENDANIAIVGNVRLVGEVPGGGGSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSGSGVKQTLNFDLLKLAGDVESNPGPVDMPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 19 - protein, 1339 aa, synthetic construct
  REGION 1..1339: note = D141A/ I210A neurexin domain, followed by the Gal4 and FoxP3after P2A
MALPVTALLLPLALLLHAARPEQKLISEEDLMYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGAYLELHIHQGKIGVKFNVGTDDIAIEESNAIINDGKYHVVRFTRSGGNATLQVDSWPVIERYPAGRQLTAFNSQATIIIGGKEQGQPFQGQLSGLYYNGLKVLNMAAENDANIAIVGNVRLVGEVPGGGGSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSGSGVKQTLNFDLLKLAGDVESNPGPVDMPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 20 - protein, 21 aa, synthetic construct
  REGION 1..21: note = signal-peptide on SynNotch construct
MALPVTALLLPLALLLHAARP

SEQ ID NO: 21 - protein, 10 aa, synthetic construct
  REGION 1..10: note = myc-tag
EQKLISEEDL

SEQ ID NO: 22 - protein, 18 aa, synthetic construct
  REGION 1..18: note = signal peptide from tether
MSMLFYTLITAFLIGIQA

SEQ ID NO: 23 - protein, 15 aa, synthetic construct
  REGION 1..15: note = Membrane anchor on tether
GGGGSGGGGSGGGGS

SEQ ID NO: 24 - protein, 316 aa, synthetic construct
  REGION 1..316: note = Tethered neurexin full sequence
MSMLFYTLITAFLIGIQAEQKLISEEDLASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGNNDNERLAIARQRIPYRLGRVVDEWLLDKGRQLTIFNSQATIIIGGKEQGQPFQGQLSGLYYNGLKVLNMAAENDANIAIVGNVRLVGEVPGGGGSGGGGSGGGGSTLVLFGAGFGAVITVVVIVVIIKCFCK

SEQ ID NO: 25 - DNA, 39 bp, synthetic construct
  misc_feature 1..39: note = Primer
  source 1..39: mol_type = other DNA
agcgaggaggatctgatgtaccagaggatgctgaggtgc

SEQ ID NO: 26 - DNA, 34 bp, synthetic construct
  misc_feature 1..34: note = Primer
  source 1..34: mol_type = other DNA
gctgtagtccaggatgggcacctcgcccaccagc

SEQ ID NO: 27 - DNA, 29 bp, synthetic construct
  misc_feature 1..29: note = Primer
  source 1..29: mol_type = other DNA
gccgccgcgatcgccatggcattgcccgt

SEQ ID NO: 28 - DNA, 36 bp, synthetic construct
  misc_feature 1..36: note = Primer
  source 1..36: mol_type = other DNA
gcggccggccgtttatcatgatccgagcatgtccag

SEQ ID NO: 29 - DNA, 34 bp, synthetic construct
  misc_feature 1..34: note = Primer
  source 1..34: mol_type = other DNA
tgatttatgcgtaacgccattttgcaaggcatgg

SEQ ID NO: 30 - DNA, 44 bp, synthetic construct
  misc_feature 1..44: note = Primer
  source 1..44: mol_type = other DNA
gataagcttgatatcgaattttacttgtacagctcgtccatgcc

SEQ ID NO: 31 - DNA, 41 bp, synthetic construct
  misc_feature 1..41: note = Primer
  source 1..41: mol_type = other DNA
atggcgttacgcataaatcaatattggctattggccattgc

SEQ ID NO: 32 - DNA, 39 bp, synthetic construct
  misc_feature 1..39: note = Primer
  source 1..39: mol_type = other DNA
atggcgttactttatagagctcatccatcccaagtgtga

SEQ ID NO: 33 - DNA, 31 bp, synthetic construct
  misc_feature 1..31: note = Primer
  source 1..31: mol_type = other DNA
gctctataaagtaacgccattttgcaaggca

SEQ ID NO: 34 - DNA, 32 bp, synthetic construct
  misc_feature 1..32: note = Primer
  source 1..32: mol_type = other DNA
tcgactctagagtcgcggccgagcatgtccag

SEQ ID NO: 35 - DNA, 34 bp, synthetic construct; identical to SEQ ID NO: 29
  misc_feature 1..34: note = Primer
  source 1..34: mol_type = other DNA

SEQ ID NO: 36 - DNA, 44 bp, synthetic construct; identical to SEQ ID NO: 30
  misc_feature 1..44: note = Primer
  source 1..44: mol_type = other DNA

SEQ ID NO: 37 - DNA, 41 bp, synthetic construct; identical to SEQ ID NO: 31
  misc_feature 1..41: note = Primer
  source 1..41: mol_type = other DNA

SEQ ID NO: 38 - DNA, 39 bp, synthetic construct
  misc_feature 1..39: note = Primer
  source 1..39: mol_type = other DNA
aatcaatgtctttatagagctcatccatcccaagtgtga

SEQ ID NO: 39 - DNA, 53 bp, synthetic construct
  misc_feature 1..53: note = Primer
  source 1..53: mol_type = other DNA
gctctataaagacattgattattgactagttattaatagtaatcaattacggg

SEQ ID NO: 40 - DNA, 32 bp, synthetic construct; identical to SEQ ID NO: 34
  misc_feature 1..32: note = Primer
  source 1..32: mol_type = other DNA

SEQ ID NO: 41 - protein, 107 aa, synthetic construct
  REGION 1..107: note = Light chain variable region of humanized M1
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIK

SEQ ID NO: 42 - protein, 121 aa, synthetic construct
  REGION 1..121: note = Heavy chain variable region of humanized M1
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSS

SEQ ID NO: 43 - DNA, 15968 bp, synthetic construct
  misc_feature 1..15968: note = pREF060 (synNotch producing Gal4 and Gal4-inducible CAR)
  source 1..15968: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattgcataaatcaatattggctattggccattgcatacgttgtatctatatcataacggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttgggccaccatgggaactagtctgctgtgttggatggccctctgcttgttgggcgcagaccacgctgacggccaagttcaattggtccagtctggtgccgaactgaagaagccaggagctagcgtcaaggtgagttgtaagtcttccggataccatttcacgagctactggatgcattgggtaaaacaagcccctggacaaggtctcgagtggataggggtaatacatcctaactctgggtccataaattacaatgaaaagttcgaatccagggttaccatcacagtcgataagtccacttccacggcctatatggaattgagctccctcaggagcgaagatacagctgtatactactgcgctggtgaacgagactccactgaggtgttgcccatggattactggggacaaggcaccactgtaacggtctctagcggtgggagtagtagaagctcatcatctggcggcggcggttcaggcggtggcggcgacgtgcagatcacgcaaagcccgagctcactctccgccagtctgggtgaacgtgcgactataaattgtagagccagcaagagcattaacaagtatctcgcctggtaccagcaaaagccgggcaaggctcctaaactcctcatctattccggatctactctccaaagcggcataccagcgaggttctcaggaagcggatcaggaacggattttacgcttacgatatctagcctcgagccagaggattttgccatgtactattgccaacaacataacgaatatcccttgaccttcggacaagggacgaaactcgagatcaaagattacaaagacgatgacgacaaaaccacaaccccagctcctaggcccccaacacctgcacctaccatcgctagccagcctctgagcctgcgtcccgaggcatgtcgtcctgctgcaggtggggcagtccacacaagaggtctggactttgcatgtgacttttgggtcctcgtagtcgtcggtggcgtcctggcctgttattcccttctggttacggtcgccttcattatattttgggtcaggagtaaacggtctaggggcgggcacagcgactacatgaatatgacgccacgccgccccgggcccacaagaaaacactaccaaccttacgctccacccagggattttgccgcgtaccggagcagggtaaagttcagccgcagtgcggatgcaccagcgtatcaacaaggccagaatcaactgtataatgagctgaacttgggacgacgagaggaatatgatgttctggataaacggcgcgggagagatccagagatgggcggaaagcctagacgtaagaacccccaagaaggcctttataatgagttgcagaaagataagatggccgaagcctattctgaaataggcatgaagggagagcggcggcgtggcaagggtcatgacggtctttaccagggcctctctaccgcaactaaagatacctatgatgcacttcacatgcaagccctgccccctagagagttcgaagggtcagcagcagccgagggacgtggctcattgcttacttgcggtgacgttgaagaaaaccctggccccagcgggatggtaagcaagggagaagaacttttcacgggagtagttcctattctggtggaactggacggggatgtaaacgggcataaattctctgtcagcggggaaggggaaggcgatgcaacatacgggaagcttaccctcaaattcatctgtacgacagggaaacttccagtgccctggcctacacttgtgacaaccctgacttatggggtccaatgtttctccaggtaccctgatcacatgaaacagcatgacttcttcaagagcgcaatgcctgaagggtatgtccaggaacgcacgattttcttcaaggacgatggcaattataaaactagagccgaagtaaaatttgagggagatacattggtaaacaggatcgagctcaaagggatcgatttcaaagaggatgggaacatcctggggcataagttggagtataactataactctcacaatgtctatattatggccgacaagcaaaagaacgggataaaagtcaactttaaaataaggcacaatatagaggacggcagtgtgcaattggcggaccactatcaacaaaataccccgatcggcgacggtccagtgttgctccctgataatcactacctttcaacacaaagtgcacttagtaaagatcccaatgagaagcgggatcacatggtcctgctcgaatttgtcacagcagctggcatcacacttgggatggatgagctctataaataataaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccatggcattgcccgtgaccgccctgctgctgccactggccttgttgctccacgccgcgcggccagaacagaagctgatcagcgaggaggatctgatgtaccagaggatgctgaggtgcggcgccgagctgggcagccccggcggcggcggcggcggcggcggcggcggcggcgccggcggcaggctggccctgctgtggatcgtgcccctgaccctgagcggcctgctgggcgtggcctggggcgccagcagcctgggcgcccaccacatccaccacttccacggcagcagcaagcaccacagcgtgcccatcgccatctacaggagccccgccagcctgaggggcggccacgccggcaccacctacatcttcagcaagggcggcggccagatcacctacaagtggccccccaacgacaggcccagcaccagggccgacaggctggccatcggcttcagcaccgtgcagaaggaggccgtgctggtgagggtggacagcagcagcggcctgggcgactacctggagctgcacatccaccagggcaagatcggcgtgaagttcaacgtgggcaccgacgacatcgccatcgaggagagcaacgccatcatcaacgacggcaagtaccacgtggtgaggttcaccaggagcggcggcaacgccaccctgcaggtggacagctggcccgtgatcgagaggtaccccgccggcaggcagctgaccatcttcaacagccaggccaccatcatcatcggcggcaaggagcagggccagcccttccagggccagctgagcggcctgtactacaacggcctgaaggtgctgaacatggccgccgagaacgacgccaacatcgccatcgtgggcaacgtgaggctggtgggcgaggtgcccatcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatgataaacggccggccgcggtcatagctgtttcctgggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 44 - protein, 834 aa, synthetic construct
  REGION 1..834: note = synNotch (receptor 1) of pREF060
MALPVTALLLPLALLLHAARPEQKLISEEDLMYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVPILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 45 - protein, 767 aa, synthetic construct
  REGION 1..767: note = CAR (receptor 2) of pREF060
MGTSLLCWMALCLLGADHADGQVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSGGSSRSSSSGGGGSGGGGDVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIKDYKDDDDKTTTPAPRPPTPAPTIASQPLSLRPEACRPAAGGAVHTRGLDFACDFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFEGSAAAEGRGSLLTCGDVEENPGPSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 46 - protein, 500 aa, synthetic construct
  REGION 1..500: note = CAR sequence
MGTSLLCWMALCLLGADHADGQVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSGGSSRSSSSGGGGSGGGGDVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIKDYKDDDDKTTTPAPRPPTPAPTIASQPLSLRPEACRPAAGGAVHTRGLDFACDFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 442 aa, Homo sapiens
MYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSGGLANPTRAGGREPYPGSAEVIRESSSTTGMVVGIVAAAALCILILLYAMYKYRNRDEGSYHVDESRNYISNSAQSNGAVVKEKQPSSAKSSNKNKKNKDKEYYV

SEQ ID NO: 48 - protein, 772 aa, synthetic construct
  REGION 1..772: note = Neurexin tether and FoxP3 linked by P2A
MSMLFYTLITAFLIGIQAEQKLISEEDLASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGNNDNERLAIARQRIPYRLGRVVDEWLLDKGRQLTIFNSQATIIIGGKEQGQPFQGQLSGLYYNGLKVLNMAAENDANIAIVGNVRLVGEVPGGGGSGGGGSGGGGSTLVLFGAGFGAVITVVVIVVIIKCFCKGSGVKQTLNFDLLKLAGDVESNPGPVDMPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 49 - DNA, 8342 bp, synthetic construct
  misc_feature 1..8342: note = pREF020- lentiviral vector with synNotch with WT neurexinsequence
  source 1..8342: mol_type = other DNA
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccatggcattgcccgtgaccgccctgctgctgccactggccttgttgctccacgccgcgcggccagaacagaagctgatcagcgaggaggatctgatgtaccagaggatgctgaggtgcggcgccgagctgggcagccccggcggcggcggcggcggcggcggcggcggcggcgccggcggcaggctggccctgctgtggatcgtgcccctgaccctgagcggcctgctgggcgtggcctggggcgccagcagcctgggcgcccaccacatccaccacttccacggcagcagcaagcaccacagcgtgcccatcgccatctacaggagccccgccagcctgaggggcggccacgccggcaccacctacatcttcagcaagggcggcggccagatcacctacaagtggccccccaacgacaggcccagcaccagggccgacaggctggccatcggcttcagcaccgtgcagaaggaggccgtgctggtgagggtggacagcagcagcggcctgggcgactacctggagctgcacatccaccagggcaagatcggcgtgaagttcaacgtgggcaccgacgacatcgccatcgaggagagcaacgccatcatcaacgacggcaagtaccacgtggtgaggttcaccaggagcggcggcaacgccaccctgcaggtggacagctggcccgtgatcgagaggtaccccgccggcaggcagctgaccatcttcaacagccaggccaccatcatcatcggcggcaaggagcagggccagcccttccagggccagctgagcggcctgtactacaacggcctgaaggtgctgaacatggccgccgagaacgacgccaacatcgccatcgtgggcaacgtgaggctggtgggcgaggtgcccatcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatgataaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggccgaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 50 - DNA, 10732 bp, synthetic construct
  misc_feature 1..10732: note = Linearised by PCR backbone sequence
  source 1..10732: mol_type = other DNA
atcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatgagatccttgacttgcggccgcaactcccacctgcaacatgcgtgactgactgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaattctcacgcgtcaagtggagcaaggcaggtggacagtggatcgccgccaccatggcattgcccgtgaccgccctgctgctgccactggccttgttgctccacgccgcgcggccagaacagaagctgatcagcgaggaggatctg

SEQ ID NO: 51 - DNA, 10693 bp, synthetic construct
  misc_feature 1..10693: note = pREF061- handling vector, expresses GFP, lentiviral packingvector
  source 1..10693: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttccccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 52 - DNA, 11517 bp, synthetic construct
  misc_feature 1..11517: note = pREF0123- Tether with Gal4 under auto-cleavable peptide
  source 1..11517: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatccgccaccatgagcatgctgttctataccctgatcaccgcctttctgatcggcatccaggccgagcagaagctgatctccgaggaagatctggcctctagcctgggcgctcaccacatccaccactttcacggcagcagcaagcaccactctgtgcctatcgccatctacagaagccccgcttctctgagaggcggccatgctggaaccacctacatcttctctaaaggcggcggacagatcacctacaagtggcctccaaacgacagacccagcaccagagccgatagactggccatcggcttcagcaccgtgcagaaagaagccgtgctcgtcagagtggacagcagctctggactgggcgactacctggaactgcacatccatcagggcaagatcggcgtgaagttcaacgtgggcaccgacgacattgccatcgaggaaagcaacgccatcatcaacgacggcaagtaccacgtcgtgcggttcacaagaagcggcggcaacgctacactgcaggtcgactcttggcccgtgatcgagagataccctgccggcaacaacgacaacgagagactggctatcgccagacagagaatcccctacagactgggaagagtggtggacgagtggctgctggataagggcagacagctgaccatcttcaatagccaggccaccatcatcatcggcggaaaagagcagggccagcctttccagggacagctgagcggactgtactacaacggcctgaaggtgctgaacatggccgctgagaacgacgccaatatcgctatcgtgggcaacgtgcggctcgtgggagaagttcctggcggcggaggatctggcggaggtggaagtggcggaggcggatctacactggtgctgtttggcgctggcttcggcgccgtgattacagtggtggtcatcgtcgtgatcatcaagtgtttctgcaaagaaggacgcggctccctgcttacgtgtggcgatgttgaagaaaatcctgggccttccggcgccatgaagctgctgagcagcatcgagcaggcctgcgacatctgcaggctgaagaagctgaagtgcagcaaggagaagcccaagtgcgccaagtgcctgaagaacaactgggagtgcaggtacagccccaagaccaagaggagccccctgaccagggcccacctgaccgaggtggagagcaggctggagaggctggagcagctgttcctgctgatcttccccagggaggacctggacatgatcctgaagatggacagcctgcaggacatcaaggccctgctgaccggcctgttcgtgcaggacaacgtgaacaaggacgccgtgaccgacaggctggccagcgtggagaccgacatgcccctgaccctgaggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttccccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 53 - DNA, 7685 bp, synthetic construct
  misc_feature 1..7685: note = pREF044- Gal4-inducible CAR
  source 1..7685: mol_type = other DNA
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataacggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttgggccaccatgggaactagtctgctgtgttggatggccctctgcttgttgggcgcagaccacgctgacggccaagttcaattggtccagtctggtgccgaactgaagaagccaggagctagcgtcaaggtgagttgtaagtcttccggataccatttcacgagctactggatgcattgggtaaaacaagcccctggacaaggtctcgagtggataggggtaatacatcctaactctgggtccataaattacaatgaaaagttcgaatccagggttaccatcacagtcgataagtccacttccacggcctatatggaattgagctccctcaggagcgaagatacagctgtatactactgcgctggtgaacgagactccactgaggtgttgcccatggattactggggacaaggcaccactgtaacggtctctagcggtgggagtagtagaagctcatcatctggcggcggcggttcaggcggtggcggcgacgtgcagatcacgcaaagcccgagctcactctccgccagtctgggtgaacgtgcgactataaattgtagagccagcaagagcattaacaagtatctcgcctggtaccagcaaaagccgggcaaggctcctaaactcctcatctattccggatctactctccaaagcggcataccagcgaggttctcaggaagcggatcaggaacggattttacgcttacgatatctagcctcgagccagaggattttgccatgtactattgccaacaacataacgaatatcccttgaccttcggacaagggacgaaactcgagatcaaagattacaaagacgatgacgacaaaaccacaaccccagctcctaggcccccaacacctgcacctaccatcgctagccagcctctgagcctgcgtcccgaggcatgtcgtcctgctgcaggtggggcagtccacacaagaggtctggactttgcatgtgacttttgggtcctcgtagtcgtcggtggcgtcctggcctgttattcccttctggttacggtcgccttcattatattttgggtcaggagtaaacggtctaggggcgggcacagcgactacatgaatatgacgccacgccgccccgggcccacaagaaaacactaccaaccttacgctccacccagggattttgccgcgtaccggagcagggtaaagttcagccgcagtgcggatgcaccagcgtatcaacaaggccagaatcaactgtataatgagctgaacttgggacgacgagaggaatatgatgttctggataaacggcgcgggagagatccagagatgggcggaaagcctagacgtaagaacccccaagaaggcctttataatgagttgcagaaagataagatggccgaagcctattctgaaataggcatgaagggagagcggcggcgtggcaagggtcatgacggtctttaccagggcctctctaccgcaactaaagatacctatgatgcacttcacatgcaagccctgccccctagagagttcgaagggtcagcagcagccgagggacgtggctcattgcttacttgcggtgacgttgaagaaaaccctggccccagcgggatggtaagcaagggagaagaacttttcacgggagtagttcctattctggtggaactggacggggatgtaaacgggcataaattctctgtcagcggggaaggggaaggcgatgcaacatacgggaagcttaccctcaaattcatctgtacgacagggaaacttccagtgccctggcctacacttgtgacaaccctgacttatggggtccaatgtttctccaggtaccctgatcacatgaaacagcatgacttcttcaagagcgcaatgcctgaagggtatgtccaggaacgcacgattttcttcaaggacgatggcaattataaaactagagccgaagtaaaatttgagggagatacattggtaaacaggatcgagctcaaagggatcgatttcaaagaggatgggaacatcctggggcataagttggagtataactataactctcacaatgtctatattatggccgacaagcaaaagaacgggataaaagtcaactttaaaataaggcacaatatagaggacggcagtgtgcaattggcggaccactatcaacaaaataccccgatcggcgacggtccagtgttgctccctgataatcactacctttcaacacaaagtgcacttagtaaagatcccaatgagaagcgggatcacatggtcctgctcgaatttgtcacagcagctggcatcacacttgggatggatgagctctataaataataaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggccgaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 54 - DNA, 8339 bp, synthetic construct
  misc_feature 1..8339: note = pREF031 - transient expression vector for first receptor(synNotch WT neurexin)
  source 1..8339: mol_type = other DNA
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccatggcattgcccgtgaccgccctgctgctgccactggccttgttgctccacgccgcgcggccagaacagaagctgatcagcgaggaggatctgatgtaccagaggatgctgaggtgcggcgccgagctgggcagccccggcggcggcggcggcggcggcggcggcggcgccggcggcaggctggccctgctgtggatcgtgcccctgaccctgagcggcctgctgggcgtggcctggggcgccagcagcctgggcgcccaccacatccaccacttccacggcagcagcaagcaccacagcgtgcccatcgccatctacaggagccccgccagcctgaggggcggccacgccggcaccacctacatcttcagcaagggcggcggccagatcacctacaagtggccccccaacgacaggcccagcaccagggccgacaggctggccatcggcttcagcaccgtgcagaaggaggccgtgctggtgagggtggacagcagcagcggcctgggcgactacctggagctgcacatccaccagggcaagatcggcgtgaagttcaacgtgggcaccgacgacatcgccatcgaggagagcaacgccatcatcaacgacggcaagtaccacgtggtgaggttcaccaggagcggcggcaacgccaccctgcaggtggacagctggcccgtgatcgagaggtaccccgccggcaggcagctgaccatcttcaacagccaggccaccatcatcatcggcggcaaggagcagggccagcccttccagggccagctgagcggcctgtactacaacggcctgaaggtgctgaacatggccgccgagaacgacgccaacatcgccatcgtgggcaacgtgaggctggtgggcgaggtgcccatcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatgataaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggccgaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 55 - DNA, 14100 bp, synthetic construct
  misc_feature 1..14100: note = pREF043- constitutively expressed Gal4 and CAR under control ofGal4-inducible element
  source 1..14100: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattgcataaatcaatattggctattggccattgcatacgttgtatctatatcataacggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttgggccaccatgggaactagtctgctgtgttggatggccctctgcttgttgggcgcagaccacgctgacggccaagttcaattggtccagtctggtgccgaactgaagaagccaggagctagcgtcaaggtgagttgtaagtcttccggataccatttcacgagctactggatgcattgggtaaaacaagcccctggacaaggtctcgagtggataggggtaatacatcctaactctgggtccataaattacaatgaaaagttcgaatccagggttaccatcacagtcgataagtccacttccacggcctatatggaattgagctccctcaggagcgaagatacagctgtatactactgcgctggtgaacgagactccactgaggtgttgcccatggattactggggacaaggcaccactgtaacggtctctagcggtgggagtagtagaagctcatcatctggcggcggcggttcaggcggtggcggcgacgtgcagatcacgcaaagcccgagctcactctccgccagtctgggtgaacgtgcgactataaattgtagagccagcaagagcattaacaagtatctcgcctggtaccagcaaaagccgggcaaggctcctaaactcctcatctattccggatctactctccaaagcggcataccagcgaggttctcaggaagcggatcaggaacggattttacgcttacgatatctagcctcgagccagaggattttgccatgtactattgccaacaacataacgaatatcccttgaccttcggacaagggacgaaactcgagatcaaagattacaaagacgatgacgacaaaaccacaaccccagctcctaggcccccaacacctgcacctaccatcgctagccagcctctgagcctgcgtcccgaggcatgtcgtcctgctgcaggtggggcagtccacacaagaggtctggactttgcatgtgacttttgggtcctcgtagtcgtcggtggcgtcctggcctgttattcccttctggttacggtcgccttcattatattttgggtcaggagtaaacggtctaggggcgggcacagcgactacatgaatatgacgccacgccgccccgggcccacaagaaaacactaccaaccttacgctccacccagggattttgccgcgtaccggagcagggtaaagttcagccgcagtgcggatgcaccagcgtatcaacaaggccagaatcaactgtataatgagctgaacttgggacgacgagaggaatatgatgttctggataaacggcgcgggagagatccagagatgggcggaaagcctagacgtaagaacccccaagaaggcctttataatgagttgcagaaagataagatggccgaagcctattctgaaataggcatgaagggagagcggcggcgtggcaagggtcatgacggtctttaccagggcctctctaccgcaactaaagatacctatgatgcacttcacatgcaagccctgccccctagagagttcgaagggtcagcagcagccgagggacgtggctcattgcttacttgcggtgacgttgaagaaaaccctggccccagcgggatggtaagcaagggagaagaacttttcacgggagtagttcctattctggtggaactggacggggatgtaaacgggcataaattctctgtcagcggggaaggggaaggcgatgcaacatacgggaagcttaccctcaaattcatctgtacgacagggaaacttccagtgccctggcctacacttgtgacaaccctgacttatggggtccaatgtttctccaggtaccctgatcacatgaaacagcatgacttcttcaagagcgcaatgcctgaagggtatgtccaggaacgcacgattttcttcaaggacgatggcaattataaaactagagccgaagtaaaatttgagggagatacattggtaaacaggatcgagctcaaagggatcgatttcaaagaggatgggaacatcctggggcataagttggagtataactataactctcacaatgtctatattatggccgacaagcaaaagaacgggataaaagtcaactttaaaataaggcacaatatagaggacggcagtgtgcaattggcggaccactatcaacaaaataccccgatcggcgacggtccagtgttgctccctgataatcactacctttcaacacaaagtgcacttagtaaagatcccaatgagaagcgggatcacatggtcctgctcgaatttgtcacagcagctggcatcacacttgggatggatgagctctataaataataaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatgataaacggccggccgcggtcatagctgtttcctgggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttccccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 56 - DNA, 11383 bp, synthetic construct
  misc_feature 1..11383: note = pREF097 - FoxP3-inducible CAR
  source 1..11383: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcgtagctgaagaaaggattcataaatgaagttcaatccttctcatcaaccccagcccacacctccagcaattgaacttgaaaaaaaaaacctggtttgaaaaattaccgcaaactatattgtcatcaaaaaaaaaaaaaaaaaaaaacacttcctatatttgagatgagagaagagagtgctaggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaaggccaccatgggaactagtctgctgtgttggatggccctctgcttgttgggcgcagaccacgctgacggccaagttcaattggtccagtctggtgccgaactgaagaagccaggagctagcgtcaaggtgagttgtaagtcttccggataccatttcacgagctactggatgcattgggtaaaacaagcccctggacaaggtctcgagtggataggggtaatacatcctaactctgggtccataaattacaatgaaaagttcgaatccagggttaccatcacagtcgataagtccacttccacggcctatatggaattgagctccctcaggagcgaagatacagctgtatactactgcgctggtgaacgagactccactgaggtgttgcccatggattactggggacaaggcaccactgtaacggtctctagcggtgggagtagtagaagctcatcatctggcggcggcggttcaggcggtggcggcgacgtgcagatcacgcaaagcccgagctcactctccgccagtctgggtgaacgtgcgactataaattgtagagccagcaagagcattaacaagtatctcgcctggtaccagcaaaagccgggcaaggctcctaaactcctcatctattccggatctactctccaaagcggcataccagcgaggttctcaggaagcggatcaggaacggattttacgcttacgatatctagcctcgagccagaggattttgccatgtactattgccaacaacataacgaatatcccttgaccttcggacaagggacgaaactcgagatcaaagattacaaagacgatgacgacaaaaccacaaccccagctcctaggcccccaacacctgcacctaccatcgctagccagcctctgagcctgcgtcccgaggcatgtcgtcctgctgcaggtggggcagtccacacaagaggtctggactttgcatgtgacttttgggtcctcgtagtcgtcggtggcgtcctggcctgttattcccttctggttacggtcgccttcattatattttgggtcaggagtaaacggtctaggggcgggcacagcgactacatgaatatgacgccacgccgccccgggcccacaagaaaacactaccaaccttacgctccacccagggattttgccgcgtaccggagcagggtaaagttcagccgcagtgcggatgcaccagcgtatcaacaaggccagaatcaactgtataatgagctgaacttgggacgacgagaggaatatgatgttctggataaacggcgcgggagagatccagagatgggcggaaagcctagacgtaagaacccccaagaaggcctttataatgagttgcagaaagataagatggccgaagcctattctgaaataggcatgaagggagagcggcggcgtggcaagggtcatgacggtctttaccagggcctctctaccgcaactaaagatacctatgatgcacttcacatgcaagccctgccccctagagagttcgaagggtcagcagcagcctgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttccccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 57 - DNA, 9788 bp, synthetic construct
  misc_feature 1..9788: note = pREF014 - CAR-encoding plasmid, CAR under SFFV promoter
  source 1..9788: mol_type = other DNA
ctcgagcttattccagatgcgtgcggatggaattcgagctcggtaccatgccaaaagcaaagcgctatcgcgccttacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataataacatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggagggcctcctggcgccgcaccggcccaaggagcccgcgtggttcctggccaccgtcggcgtctcgcccgaccaccagggcaagggtctgggcagcgccgtcgtgctccccggagtggaggccgccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctaggctagcttgactgactgagtcgacaatcaaccttttggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcctggccaacgtgagcaccgtgctgacctccaaatatcgttaagctggagcctgggagccggcctggccctccgccccccccacccccgcagcccacccctggtctttgaataaagtctgagtgagtggccgacagtgcccgtggagttctcgtgacctgaggtgcagggccggcgctagggacacgtccgtgcacgtgccgaggccccctgtgcagctgcaagggacaggcctagccctgcaggcctaactccgcccatcccgcccctaactccgcccagttccgcccattctccgcctcatggctgactaattttttttatttatgcagaggccgaggccgcctcggcctctgagctattccagaagtagtgaggacgcttttttggaggccgaggcttttgcaaagatcgaacaagagacaggacctgcaggttaattaaatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatggccggcctggcgcgccgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaacgcgatcgcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttggctcccgatcgttgcgttacacacacaattactgctgatcgagtgtagccttcgaatgaaagaccccacctgtaggtttggcaagatagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgccctgatcattgtcgatcctaccatccactcgacacacccgccagggcccgcatccaccatcgcagacttatcatggatccgccaccatgggaactagtctgctgtgttggatggccctctgcttgttgggcgcagaccacgctgacggccaagttcaattggtccagtctggtgccgaactgaagaagccaggagctagcgtcaaggtgagttgtaagtcttccggataccatttcacgagctactggatgcattgggtaaaacaagcccctggacaaggtctcgagtggataggggtaatacatcctaactctgggtccataaattacaatgaaaagttcgaatccagggttaccatcacagtcgataagtccacttccacggcctatatggaattgagctccctcaggagcgaagatacagctgtatactactgcgctggtgaacgagactccactgaggtgttgcccatggattactggggacaaggcaccactgtaacggtctctagcggtgggagtagtagaagctcatcatctggcggcggcggttcaggcggtggcggcgacgtgcagatcacgcaaagcccgagctcactctccgccagtctgggtgaacgtgcgactataaattgtagagccagcaagagcattaacaagtatctcgcctggtaccagcaaaagccgggcaaggctcctaaactcctcatctattccggatctactctccaaagcggcataccagcgaggttctcaggaagcggatcaggaacggattttacgcttacgatatctagcctcgagccagaggattttgccatgtactattgccaacaacataacgaatatcccttgaccttcggacaagggacgaaactcgagatcaaaaccacaaccccagctcctaggcccccaacacctgcacctaccatcgctagccagcctctgagcctgcgtcccgaggcatgtcgtcctgctgcaggtggggcagtccacacaagaggtctggactttgcatgtgacttttgggtcctcgtagtcgtcggtggcgtcctggcctgttattcccttctggttacggtcgccttcattatattttgggtcaggagtaaacggtctaggggcgggcacagcgactacatgaatatgacgccacgccgccccgggcccacaagaaaacactaccaaccttacgctccacccagggattttgccgcgtaccggagcagggtaaagttcagccgcagtgcggatgcaccagcgtatcaacaaggccagaatcaactgtataatgagctgaacttgggacgacgagaggaatatgatgttctggataaacggcgcgggagagatccagagatgggcggaaagcctagacgtaagaacccccaagaaggcctttataatgagttgcagaaagataagatggccgaagcctattctgaaataggcatgaagggagagcggcggcgtggcaagggtcatgacggtctttaccagggcctctctaccgcaactaaagatacctatgatgcacttcacatgcaagccctgccccctagagagttcgaagggtcagcagcagccgagggacgtggctcattgcttacttgcggtgacgttgaagaaaaccctggccccagcgggatggtaagcaagggagaagaacttttcacgggagtagttcctattctggtggaactggacggggatgtaaacgggcataaattctctgtcagcggggaaggggaaggcgatgcaacatacgggaagcttaccctcaaattcatctgtacgacagggaaacttccagtgccctggcctacacttgtgacaaccctgacttatggggtccaatgtttctccaggtaccctgatcacatgaaacagcatgacttcttcaagagcgcaatgcctgaagggtatgtccaggaacgcacgattttcttcaaggacgatggcaattataaaactagagccgaagtaaaatttgagggagatacattggtaaacaggatcgagctcaaagggatcgatttcaaagaggatgggaacatcctggggcataagttggagtataactataactctcacaatgtctatattatggccgacaagcaaaagaacgggataaaagtcaactttaaaataaggcacaatatagaggacggcagtgtgcaattggcggaccactatcaacaaaataccccgatcggcgacggtccagtgttgctccctgataatcactacctttcaacacaaagtgcacttagtaaagatcccaatgagaagcgggatcacatggtcctgctcgaatttgtcacagcagctggcatcacacttgggatggatgagctctataaataa

SEQ ID NO: 58 - DNA, 10916 bp, synthetic construct
  misc_feature 1..10916: note = pREF091 - Tether-containing plasmid
  source 1..10916: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatccgccaccatgagcatgctgttctataccctgatcaccgcctttctgatcggcatccaggccgagcagaagctgatctccgaggaagatctggcctctagcctgggcgctcaccacatccaccactttcacggcagcagcaagcaccactctgtgcctatcgccatctacagaagccccgcttctctgagaggcggccatgctggaaccacctacatcttctctaaaggcggcggacagatcacctacaagtggcctccaaacgacagacccagcaccagagccgatagactggccatcggcttcagcaccgtgcagaaagaagccgtgctcgtcagagtggacagcagctctggactgggcgactacctggaactgcacatccatcagggcaagatcggcgtgaagttcaacgtgggcaccgacgacattgccatcgaggaaagcaacgccatcatcaacgacggcaagtaccacgtcgtgcggttcacaagaagcggcggcaacgctacactgcaggtcgactcttggcccgtgatcgagagataccctgccggcaacaacgacaacgagagactggctatcgccagacagagaatcccctacagactgggaagagtggtggacgagtggctgctggataagggcagacagctgaccatcttcaatagccaggccaccatcatcatcggcggaaaagagcagggccagcctttccagggacagctgagcggactgtactacaacggcctgaaggtgctgaacatggccgctgagaacgacgccaatatcgctatcgtgggcaacgtgcggctcgtgggagaagttcctggcggcggaggatctggcggaggtggaagtggcggaggcggatctacactggtgctgtttggcgctggcttcggcgccgtgattacagtggtggtcatcgtcgtgatcatcaagtgtttctgcaaatgagcggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttccccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 59 - DNA, 10570 bp, synthetic construct
  misc_feature 1..10570: note = pREF096 - GFP under FoxP3-inducible promoter
  source 1..10570: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcgtagctgaagaaaggattcataaatgaagttcaatccttctcatcaaccccagcccacacctccagcaattgaacttgaaaaaaaaaacctggtttgaaaaattaccgcaaactatattgtcatcaaaaaaaaaaaaaaaaaaaaacacttcctatatttgagatgagagaagagagtgctaggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggtaagcaagggagaagaacttttcacgggagtagttcctattctggtggaactggacggggatgtaaacgggcataaattctctgtcagcggggaaggggaaggcgatgcaacatacgggaagcttaccctcaaattcatctgtacgacagggaaacttccagtgccctggcctacacttgtgacaaccctgacttatggggtccaatgtttctccaggtaccctgatcacatgaaacagcatgacttcttcaagagcgcaatgcctgaagggtatgtccaggaacgcacgattttcttcaaggacgatggcaattataaaactagagccgaagtaaaatttgagggagatacattggtaaacaggatcgagctcaaagggatcgatttcaaagaggatgggaacatcctggggcataagttggagtataactataactctcacaatgtctatattatggccgacaagcaaaagaacgggataaaagtcaactttaaaataaggcacaatatagaggacggcagtgtgcaattggcggaccactatcaacaaaataccccgatcggcgacggtccagtgttgctccctgataatcactacctttcaacacaaagtgcacttagtaaagatcccaatgagaagcgggatcacatggtcctgctcgaatttgtcacagcagctggcatcacacttgggatggatgagctctataaataaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttccccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 60 - protein, 11 aa, synthetic construct
  REGION 1..11: note = HM1 HVR-L1
RASKSINKYLA

SEQ ID NO: 61 - protein, 7 aa, synthetic construct
  REGION 1..7: note = HM1 HVR-L2
SGSTLQS

SEQ ID NO: 62 - protein, 9 aa, synthetic construct
  REGION 1..9: note = HM1 HVR-L3
QQHNEYPLT

SEQ ID NO: 63 - protein, 10 aa, synthetic construct
  REGION 1..10: note = HM1 HVR-H1
GYHFTSYWMH

SEQ ID NO: 64 - protein, 17 aa, synthetic construct
  REGION 1..17: note = HM1 HVR-H2
VIHPNSGSINYNEKFES

SEQ ID NO: 65 - protein, 12 aa, synthetic construct
  REGION 1..12: note = HM1 HVR-H3
ERDSTEVLPMDY